(19) United States
(12) Patent Application Publication
(10) Pub. No.: US 2025/0360511 A1
(43) Pub. Date: Nov. 27, 2025

(54) SYSTEMS AND METHODS FOR DRY EMBRYO EXPLANT PURIFICATION

(71) Applicant: Monsanto Technology LLC, St. Louis, MO (US)

(72) Inventors: Whitney R. Adams, Mystic, CT (US); Juan Manuel Alvarez, Oxnard, CA (US); Erik Dersch, Fitchburg, WI (US); John Patrick Dibb, Madison, WI (US); Robert Wayne Harnish, Middleton, WI (US); David D. Kelm, Madison, WI (US); Brian J. Martinell, Mt. Horeb, WI (US); Louis J. Meyer, Creve Coeur, MO (US); Anatoly Rivlin, Brooklyn, WI (US); Amanda Marie Rohrer, Oskaloosa, IA (US); Mark Eugene Thompson, Prairie du Sac, WI (US)

(21) Appl. No.: 18/992,906

(22) PCT Filed: Jul. 14, 2023
(86) PCT No.: PCT/US2023/070243

§ 371 Date: Jan. 9, 2025

Related U.S. Application Data
(60) Provisional application No. 63/389,751, filed on Jul. 15, 2022.
(60) Provisional application No. 63/389,759, filed on Jul. 15, 2022.
(60) Provisional application No. 63/389,762, filed on Jul. 15, 2022.
(60) Provisional application No. 63/389,746, filed on Jul. 15, 2022.
(60) Provisional application No. 63/389,781, filed on Jul. 15, 2022.
(60) Provisional application No. 63/389,783, filed on Jul. 15, 2022.
(60) Provisional application No. 63/389,786, filed on Jul. 15, 2022.

(51) Int. Cl. B02C 7/13; B02B 1/06

(52) U.S. Cl. CPC B02C 7/13; B02B 1/06

(57) ABSTRACT

The present disclosure provides novel apparatuses, systems, and methods for purifying dry embryo explants from a preparation of dry plant embryo explants for use in methods of genetic modification. The methods provided by the present disclosure may include one or more steps of sanitizing, drying, milling, coarse width sizing, length sizing aspiration, width and thickness separation, aspiration-classification, or separation using a friction table. The present disclosure further provides a population of purified embryo explants produced using the disclosed apparatuses, systems, and methods.

### CROSS REFERENCE TO RELATED APPLICATION

This application claims the benefit of U.S. Provisional App. Ser. No. 63/389,751, filed Jul. 15, 2022, U.S. Provisional App. Ser. No. 63/389,759, filed Jul. 15, 2022, U.S. Provisional App. Ser. No. 63/389,762, filed Jul. 15, 2022, U.S. Provisional App. Ser. No. 63/389,746, filed Jul. 15, 2022, U.S. Provisional App. Ser. No. 63/389,781, filed Jul. 15, 2022, U.S. Provisional App. Ser. No. 63/389,783, filed Jul. 15, 2022, and U.S. Provisional App. Ser. No. 63/389,786, filed Jul. 15, 2022, the entire contents of each of which are incorporated herein by reference.

### FIELD OF THE INVENTION

The present disclosure relates to apparatuses, systems, and methods for excising and purifying dry embryo explants from plant seeds. Dry embryo explants purified using the apparatuses, systems, and methods described herein are useful in methods of producing genetically modified plants or parts thereof.

### BACKGROUND

Genetic modification of dry embryo explants has been used to produce crop plants which have improved traits or characteristics, such as corn, soybean, cotton, wheat, and canola. There is, however, a continuing need in the art for improved methods of purifying dry embryo explants, which are especially useful in methods of producing genetically modified plants or parts thereof. The use of purified dry embryo explants in methods of genetic modification significantly improves transformation efficiency by decreasing contamination, improving explant health, and providing a sustainable clean culture system from which genetically modified plants or parts thereof can be recovered.

The embodiments described herein provide novel apparatuses, systems, and methods for purifying dry embryo explants for use in methods of genetic modification that overcome many of the challenges and limitations in the art.

### SUMMARY

In some aspects, the present disclosure provides, a method of purifying genetically modifiable dry plant embryo explants, the method comprising: sanitizing a population of plant seeds; milling the population of plant seeds to produce a preparation of dry plant embryo explants comprising meristematic tissue, wherein the preparation comprises a population of dry plant embryo explants and debris material; aspirating the preparation of embryo explants to separate an aspirated fraction of the embryo explants from an aspirated portion of the debris material; and purifying the genetically modifiable dry embryo explants. In some embodiments, the dry plant embryo explants are selected from the group consisting of corn embryo explants, soybean embryo explants, cotton embryo explants, wheat embryo explants, and canola embryo explants.

In some embodiments, the population of plant seeds is a population of corn seeds, and the milling comprises: positioning a first grinding roller and a second grinding roller to define a first gap having a first gap distance between the first roller and the second roller; rotating the first roller about a first axis of rotation and the second roller about a second axis of rotation; passing the population of seeds through the first gap to produce a first preparation of plant embryo explants comprising meristematic tissue; positioning a third grinding roller and a fourth grinding roller to define a second gap having a second gap distance between the third roller and the fourth roller; rotating the third roller about a third axis of rotation and the fourth roller about a fourth axis of rotation; and passing the first preparation of embryo explants through the second gap to produce a second preparation of plant embryo explants comprising meristematic tissue, wherein the first gap distance is about 0.381 mm to about 7.62 mm, about 2.032 mm to about 2.794 mm, or is about 2.54 mm, or wherein the second gap distance is about 0.381 mm to about 7.62 mm, about 0.762 mm to about 1.778 mm, or is about 1.27 cm. In certain embodiments, the population of plant seeds is a population of corn seeds and the aspirating comprises: aspirating within a first vertical chamber, a second vertical chamber, a third vertical chamber, and a fourth vertical chamber with a first upward air flow, a second upward air flow, a third upward air flow, and a fourth upward air flow, wherein the first upward airflow has a first air flow velocity of about 4.5 m/s to about 6.0 m/s, about 5.0 m/s to about 5.5 m/s, or about 5.1 m/s to about 5.3 m/s, wherein the second upward air flow has a second air flow velocity of about 5.5 m/s to about 6.5 m/s or about 5.9 m/s to about 6.3 m/s, wherein the third upward air flow has a third air flow velocity of about 6.5 m/s to about 7.5 m/s, about 7.0 m/s to about 7.5 m/s, or about 7.0 m/s to about 7.3 m/s, and wherein the fourth upward air flow has a fourth air flow velocity of about 9.5 m/s to about 10.5 m/s or about 9.8 m/s to about 10.2 m/s.

In further embodiments, the population of plant seeds is a population of corn seeds and the method further comprises: contacting the preparation of dry plant embryo explants comprising meristematic tissue with a first moving sieve, wherein the first moving sieve comprises a plurality of openings, each having a first physical opening size; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; and separating a second fraction of embryo explants from a second portion of the debris material by length, width, or thickness relative to the second physical opening size, or relative to a second effective opening size, wherein the first moving sieve and the second moving sieve move in a circular, elliptical, or linear motion. The first physical opening size, in some embodiments, is about 500 μm to about 2000 μm, about 800 μm to about 2000 μm, or about 1181 μm. The second physical opening size, in certain embodiments, is about 500 μm to about 1000 μm or about 812 μm. In some embodiments, separating the second fraction is performed prior to the aspirating step.

In particular embodiments, the population of plant seeds is a population of corn seeds and the methods of the present disclosure may further comprise: contacting the second fraction of dry plant embryo explants comprising meristematic tissue with an interior surface of a rotating cylinder, wherein the interior surface comprises a plurality of indentations, the indentations having an indentation size and an indentation shape, and wherein the second fraction comprises a population of dry plant embryo explants and debris material; rotating the rotating cylinder about an axis of rotation, wherein the axis of rotation is substantially parallel to the ground; and separating a cylinder fraction of the plant embryo explants from a cylinder portion of the debris material. Separating the cylinder fraction, is some embodiments, may be performed prior to the aspirating step. In certain embodiments, each indentation size comprises an indentation diameter, an indentation width, an indentation length, or an indentation depth, wherein the indentation diameter, the indentation width, or the indentation length is about 1.50 mm to about 2.75 mm, about 1.75 mm to about 2.50 mm, about 2.00 mm to about 2.25 mm, about 2.00 mm, or about 2.25 mm, or wherein the indentation depth is about 0.25 mm to about 2.00 mm, about 0.50 mm to about 1.75 mm, about 0.75 mm to about 1.25 mm, or about 1.00 mm.

In many embodiments, the population of plant seeds is a population of corn seeds and the methods of the present disclosure may further comprise: contacting the aspirated fraction with a first vibratory screen, wherein the first vibratory screen comprises a plurality of openings, each having a first opening size and a first opening shape, and wherein the aspirated fraction comprises a population of dry plant embryo explants and debris material; vibrating the first vibratory screen to produce a first screen motion, wherein the first screen motion comprises a first horizontal vibratory component; separating a first screen fraction of embryo explants from a first screen portion of the debris material by length, width, or thickness relative to the first opening size or the first opening shape, or by a displacement of the first screen fraction relative to a displacement of the first screen portion of the debris material produced by the first screen motion; contacting the first screen fraction with a second vibratory screen, wherein the second vibratory screen comprises a plurality of openings, each having a second opening size and a second opening shape; vibrating the second vibratory screen to produce a second screen motion, wherein the second screen motion comprises a second horizontal vibratory component; and separating a second screen fraction of embryo explants from a second screen portion of the debris material comprised in the first screen fraction by length, width, or thickness relative to the second opening size or the second opening shape, or by a displacement of the second screen fraction relative to a displacement of the second screen portion of the debris material produced by the second screen motion. The first opening shape or the second opening shape, in some embodiments, is circular, and the first opening size or the second opening size is about 1.3 mm to about 1.6 mm, about 1.4 mm to about 1.5 mm, about 1.3 mm to about 1.5 mm, or about 1.4 mm to about 1.6 mm in diameter, or about 1.3 mm, about 1.4 mm, about 1.5 mm, or about 1.6 mm in diameter. The first opening shape or the second opening shape, in particular embodiments, is oblong, and the first opening size or the second opening size is about 5 mm to about 15 mm, about 6 mm to about 14 mm, about 8 mm to about 12 mm, about 8 mm to about 10 mm, about 9 mm to about 11 mm, about 10 mm to about 12 mm in length, or about 8 mm, about 9 mm, about 10 mm, about 11 mm, or about 12 mm in length, and from about 0.6 mm to about 0.8 mm, about 0.6 mm to about 0.7 mm, or about 0.7 mm to about 0.8 mm in width, or about 0.6 mm, about 0.65 mm, about 0.7 mm, about 0.75 mm, or about 0.8 mm in width.

In some embodiments, the population of plant seeds is a population of corn seeds, and the methods of the present disclosure may further comprise aspirating the second screen fraction of embryo explants to separate a second aspirated fraction of the embryo explants from a second aspirated portion of the debris material, wherein the aspirating comprises: aspirating within a first vertical chamber, a second vertical chamber, a third vertical chamber, and a fourth vertical chamber with a first upward air flow, a second upward air flow, a third upward air flow, and a fourth upward air flow, wherein the first upward airflow has a first air flow velocity of about 4.5 m/s to about 6.0 m/s, about 5.0 m/s to about 5.5 m/s, or about 5.1 m/s to about 5.3 m/s, wherein the second upward air flow has a second air flow velocity of about 5.5 m/s to about 6.5 m/s or about 5.9 m/s to about 6.3 m/s, wherein the third upward air flow has a third air flow velocity of about 6.5 m/s to about 7.5 m/s, about 7.0 m/s to about 7.5 m/s, or about 7.0 m/s to about 7.3 m/s, and wherein the fourth upward air flow has a fourth air flow velocity of about 9.5 m/s to about 10.5 m/s or about 9.8 m/s to about 10.2 m/s.

In certain embodiments, the population of plant seeds is a population of corn seeds and the purifying comprises: contacting the aspirated fraction, the second fraction, the cylinder fraction, the second screen fraction, or the second aspirated fraction with a textured surface of a vibratory platform, wherein the first textured surface of the vibratory platform is substantially planar, and wherein the aspirated fraction, the second fraction, the cylinder fraction, the second screen fraction, or the second aspirated fraction comprises a population of dry plant embryo explants and debris material; vibrating the vibratory platform to produce a first platform motion; and separating a platform fraction of the plant embryo explants from a platform portion of the debris material according to a displacement of the platform fraction relative to a displacement of the platform portion of debris material on the textured surface of the vibratory platform, wherein the vibratory platform comprises a first tilt angle of about 10.0 degrees to about 20.0 degrees, about 10.0 degrees to about 17.0 degrees, about 12.5 degrees to about 15.0 degrees, about 12.7 degrees to about 14.7 degrees, or about 13.7 degrees, and a first pitch angle of about 1.5 degrees to about 3.5 degrees, about 2.0 degrees to about 3.0 degrees, about 2.1 degrees to about 2.6 degrees, about 2.3 degrees, or about 2.4 degrees.

In particular embodiments, the population of plant seeds is a population of soybean seeds, and the milling comprises: positioning a first grinding roller and a second grinding roller to define a first gap having a first gap distance between the first roller and the second roller; rotating the first roller about a first axis of rotation and the second roller about a second axis of rotation; passing the population of seeds through the first gap to produce a first preparation of plant embryo explants comprising meristematic tissue; positioning a third grinding roller and a fourth grinding roller to define a second gap having a second gap distance between the third roller and the fourth roller; rotating the third roller about a third axis of rotation and the fourth roller about a fourth axis of rotation; and passing the first preparation of embryo explants through the second gap to produce a second preparation of plant embryo explants comprising meristematic tissue, wherein the first gap distance is about 0.762 mm to about 6.35 mm, about 3.81 mm to about 5.08 mm, or is about 4.2926 mm, or wherein the second gap distance is about 0.762 mm to about 6.35 mm, about 3.556 mm to about 4.318 mm, or is about 3.937 mm.

In some embodiments, the population of plant seeds is a population of soybean seeds and the aspirating comprises: aspirating within a first vertical chamber, a second vertical chamber, a third vertical chamber, and a fourth vertical chamber with a first upward air flow, a second upward air flow, a third upward air flow, and a fourth upward air flow, wherein the first upward airflow has a first air flow velocity of about 4.0 m/s to about 5.5 m/s or about 4.2 m/s to about 4.9 m/s, wherein the second upward air flow has a second air flow velocity of about 5.0 m/s to about 7.0 m/s or about 5.8 m/s to about 6.7 m/s, wherein the third upward air flow has a third air flow velocity of is about 7.0 m/s to about 8.5 m/s, about 7.5 m/s to about 8.0 m/s, or about 7.7 m/s to about 7.9 m/s, and wherein the fourth upward air flow has a fourth air flow velocity of about 10.5 m/s to about 12.5 m/s, about 10.5 m/s to about 12.0 m/s, or about 10.8 m/s to about 12.0 m/s.

In many embodiments, the population of plant seeds is a population of soybean seeds and the methods of the present disclosure may further comprise: contacting the preparation of dry plant embryo explants comprising meristematic tissue with a first moving sieve, wherein the first moving sieve comprises a plurality of openings, each having a first physical opening size; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; and separating a second fraction of embryo explants from a second portion of the debris material by length, width, or thickness relative to the second physical opening size, or relative to a second effective opening size, wherein the first moving sieve and the second moving sieve move in a circular, elliptical, or linear motion. The first physical opening size, in some embodiments, is about 800 μm to about 2600 μm, about 1600 μm to about 2600 μm, or about 2032 μm. The second physical opening size, in certain embodiments, is about 800 μm to about 1500 μm or about 1181 μm. Separating the second fraction, in some embodiments, may be performed prior to the aspirating step.

In particular embodiments, the population of plant seeds is a population of soybean seeds and the methods of the present disclosure may further comprise: contacting the second fraction of dry plant embryo explants comprising meristematic tissue with an interior surface of a rotating cylinder, wherein the interior surface comprises a plurality of indentations, the indentations having an indentation size and an indentation shape, and wherein the second fraction comprises a population of dry plant embryo explants and debris material; rotating the rotating cylinder about an axis of rotation, wherein the axis of rotation is substantially parallel to the ground; and separating a cylinder fraction of the plant embryo explants from a cylinder portion of the debris material. The separating of the cylinder fraction, in some embodiments, is performed prior to the aspirating step. In certain embodiments, each indentation size comprises an indentation diameter, an indentation width, an indentation length, or an indentation depth, wherein the indentation diameter, the indentation width, or the indentation length is about 2.25 mm to about 3.50 mm, about 2.50 mm to about 3.25 mm, about 2.75 mm to about 3.00 mm, about 2.75 mm, or about 3.00 mm, or wherein the indentation depth is about 0.25 mm to about 2.00 mm, about 0.50 mm to about 1.75 mm, about 0.75 mm to about 1.25 mm, or about 1.00 mm.

In a number of embodiments, the population of plant seeds is a population of soybean seeds and the purifying comprises: contacting the aspirated fraction, the second fraction, or the cylinder fraction with a textured surface of a vibratory platform, wherein the first textured surface of the vibratory platform is substantially planar, and wherein the aspirated fraction, the second fraction, or the cylinder fraction comprises a population of dry plant embryo explants and debris material; vibrating the vibratory platform to produce a first platform motion; and separating a platform fraction of the plant embryo explants from a platform portion of the debris material according to a displacement of the platform fraction relative to a displacement of the platform portion of debris material on the textured surface of the vibratory platform, wherein the vibratory platform comprises a first tilt angle of about 10.0 degrees to about 20.0 degrees, about 10.0 degrees to about 18.0 degrees, about 14.0 degrees to about 20.0 degrees, about 11.0 degrees to about 17.0 degrees, about 11.6 degrees to about 16.6 degrees, about 11.6 degrees to about 12.0 degrees, about 15.8 degrees to about 16.6 degrees, about 11.8 degrees, or about 16.2 degrees, and a first pitch angle of about 1.5 degrees to about 8.0 degrees, about 1.9 degrees to about 7.5 degrees, about 1.9 degrees to about 3.3 degrees, about 4.3 degrees to about 7.5 degrees, about 2.5 degrees, about 2.6 degrees, or about 5.9 degrees.

In certain embodiments, the population of plant seeds is a population of cotton seeds, and the milling comprises: positioning a first grinding plate and a second grinding plate to define a first gap having a first gap distance between the first plate and the second plate; rotating the first plate or the second plate about an axis of rotation; and contacting the population of plant seeds with an interior surface of the first plate and an interior surface of the second plate to produce a first preparation of embryo explants comprising meristematic tissue, wherein the first gap distance is about 2.5 mm to about 4.0 mm or about 3.0 mm to about 3.25 mm.

In some embodiments, the population of plant seeds is a population of cotton seeds and the aspirating comprises: aspirating within a first vertical chamber, a second vertical chamber, a third vertical chamber, and a fourth vertical chamber with a first upward air flow, a second upward air flow, a third upward air flow, and a fourth upward air flow, wherein the first upward airflow has a first air flow velocity of about 5.5 m/s to about 8.0 m/s, about 5.5 m/s to about 7.5 m/s, or about 5.6 m/s to about 7.3 m/s, wherein the second upward air flow has a second air flow velocity of about 6.5 m/s to about 8.5 m/s or about 6.8 m/s to about 8.4 m/s, wherein the third upward air flow has a third air flow velocity of about 8.0 m/s to about 12.5 m/s, about 8.5 m/s to about 12.0 m/s, or about 8.7 m/s to about 11.7 m/s, and wherein the fourth upward air flow has a fourth air flow velocity of about 13.0 m/s to about 20.5 m/s, about 13.5 m/s to about 20.3 m/s, or about 13.7 m/s to about 20.1 m/s.

In particular embodiments, the population of plant seeds is a population of cotton seeds and the methods of the present disclosure may further comprise: contacting the first preparation of embryo explants with a moving plate, wherein the moving plate comprises a proximal end, a distal end, and a plurality of openings located near the distal end, each comprising a first physical opening size, and wherein the first preparation comprises a population of embryo explants and debris material; passing the first preparation through the plurality of openings of the moving plate and contacting a first moving sieve with the first preparation, wherein the first moving sieve comprises a plurality of openings, each having a second physical opening size; and separating a first fraction of embryo explants from a first portion of the debris material by length, width, or thickness relative to the second physical opening size, wherein the moving plate and the first moving sieve move in a linear motion, and wherein the first physical opening size is about 300 μm to about 5000 μm, and the second physical opening size is about 700 μm to about 1300 μm or about 1181 μm.

In many embodiments, the population of plant seeds is a population of cotton seeds and the methods of the present disclosure may further comprise: positioning a third grinding plate and a fourth grinding plate to define a second gap having a second gap distance between the third plate and the fourth plate; rotating the third plate or the fourth plate about an axis of rotation; and contacting the first fraction with an interior surface of the third plate and an interior surface of the fourth plate to produce a second preparation of embryo explants comprising meristematic tissue, wherein the second gap distance is about 0.5 mm to about 2.5 mm or about 1.5 mm. In some embodiments, the population of plant seeds is a population of cotton seeds and the methods of the present disclosure may further comprise: contacting the second preparation with a second moving sieve comprising a plurality of openings, each having a third physical opening size; separating a second fraction of embryo explants from a second portion of the debris material by length, width, or thickness relative to the third physical opening size; contacting the second fraction with a third moving sieve comprising a plurality of openings, each comprising a fourth physical opening size; and separating a third fraction of embryo explants from a third portion of the debris material by length, width, or thickness relative to the fourth physical opening size, wherein the second moving sieve and the third moving sieve move in a linear motion, and wherein the third physical opening size is about 1600 μm to about 2500 μm or about 2032 μm, and the fourth physical opening size is about 700 μm to about 1300 μm, or about 980 μm. The methods of the present disclosure may further comprise, in particular embodiments, applying a cryogenic treatment to the first fraction of embryo explants prior to contacting the first fraction with the third plate and the fourth plate.

In a number of embodiments, the population of plant seeds is a population of cotton seeds and the methods of the present disclosure may further comprise: contacting the aspirated fraction of dry plant embryo explants comprising meristematic tissue with an interior surface of a rotating cylinder, wherein the interior surface comprises a plurality of indentations, the indentations having an indentation size and an indentation shape, and wherein the second fraction comprises a population of dry plant embryo explants and debris material; rotating the rotating cylinder about an axis of rotation, wherein the axis of rotation is substantially parallel to the ground; and separating a cylinder fraction of the plant embryo explants from a cylinder portion of the debris material. In some embodiments, each indentation size comprises an indentation diameter, an indentation width, an indentation length, or an indentation depth, wherein the indentation diameter, the indentation width, or the indentation length is about 2.25 mm to about 3.50 mm, about 2.50 mm to about 3.25 mm, about 2.75 mm to about 3.00 mm, about 2.75 mm, or about 3.00 mm, or wherein the indentation depth is about 0.25 mm to about 2.00 mm, about 0.50 mm to about 1.75 mm, about 0.75 mm to about 1.25 mm, or about 1.00 mm.

In some embodiments, the population of plant seeds is a population of cotton seeds and the purifying comprises: contacting the aspirated fraction, the first fraction, the third fraction, or the cylinder fraction with a textured surface of a vibratory platform, wherein the first textured surface of the vibratory platform is substantially planar, and wherein the aspirated fraction, the first fraction, the third fraction, or the cylinder fraction comprises a population of dry plant embryo explants and debris material; vibrating the vibratory platform to produce a first platform motion; and separating a platform fraction of the plant embryo explants from a platform portion of the debris material according to a displacement of the platform fraction relative to a displacement of the platform portion of debris material on the textured surface of the vibratory platform, wherein the vibratory platform comprises a first tilt angle of about 10.0 degrees to about 22.0 degrees, about 10.0 degrees to about 20.0 degrees, about 10.0 degrees to about 19.0 degrees, about 15.0 degrees to about 22.0 degrees, about 11.0 degrees to about 19.0 degrees, about 11.0 degrees to about 15.0 degrees, about 11.6 degrees to about 14.2 degrees, about 16.0 degrees to about 19.0 degrees, about 16.2 degrees to about 18.3 degrees, about 12.9 degrees, about 17.2 degrees, or about 17.3 degrees, and a first pitch angle of about 1.5 degrees to about 6.0 degrees, about 1.5 degrees to about 5.0 degrees, about 1.8 degrees to about 4.9 degrees, about 1.8 degrees to about 3.3 degrees, about 2.4 degrees to about 4.9 degrees, about 2.5 degrees, about 2.6 degrees, about 3.6 degrees, or about 3.7 degrees.

In particular embodiments, the population of plant seeds is a population of wheat seeds, and the milling comprises: positioning a first grinding roller and a second grinding roller to define a first gap having a first gap distance between the first roller and the second roller; rotating the first roller about a first axis of rotation and the second roller about a second axis of rotation; passing the population of seeds through the first gap to produce a first preparation of plant embryo explants comprising meristematic tissue; positioning a third grinding roller and a fourth grinding roller to define a second gap having a second gap distance between the third roller and the fourth roller; rotating the third roller about a third axis of rotation and the fourth roller about a fourth axis of rotation; and passing the first preparation of embryo explants through the second gap to produce a second preparation of plant embryo explants comprising meristematic tissue, wherein the first gap distance is about 0.2032 mm to about 2.54 mm, about 0.762 mm to about 1.788 mm, or is about 1.2827 mm, or wherein the second gap distance is about 0.2032 mm to about 2.54 mm, about 0.2286 mm to about 0.4572 mm, or is about 0.3683 mm.

In certain embodiments, the population of plant seeds is a population of wheat seeds and the aspirating comprises: aspirating within a first vertical chamber, a second vertical chamber, a third vertical chamber, and a fourth vertical chamber with a first upward air flow, a second upward air flow, a third upward air flow, and a fourth upward air flow, wherein the first upward airflow has a first air flow velocity of about 2.5 m/s to about 4.0 m/s, about 3.0 m/s to about 3.5 m/s, or about 3.0 m/s to about 3.3 m/s, wherein the second upward air flow has a second air flow velocity of about 3.0 m/s to about 5.0 m/s, about 3.5 m/s to about 4.5 m/s, or about 3.8 m/s to about 4.3 m/s, wherein the third upward air flow has a third air flow velocity of about 4.5 m/s to about 6.0 m/s, about 5.0 m/s to about 6.0 m/s, or about 5.1 m/s to about 5.5 m/s, and wherein the fourth upward air flow has a fourth air flow velocity of about 6.5 m/s to about 8.0 m/s, about 7.0 m/s to about 8.0 m/s, or about 7.2 m/s to about 7.7 m/s.

In many embodiments, the population of plant seeds is a population of wheat seeds and the method further comprises: contacting the preparation of dry plant embryo explants comprising meristematic tissue with a first moving sieve, wherein the first moving sieve comprises a plurality of openings, each having a first physical opening size; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; and separating a second fraction of embryo explants from a second portion of the debris material by length, width, or thickness relative to the second physical opening size, or relative to a second effective opening size, wherein the first moving sieve and the second moving sieve move in a circular, elliptical, or linear motion. The separating of the second fraction, in certain embodiments, is performed prior to the aspirating step. In some embodiments, the first physical opening size is about 300 μm to about 1200 μm, about 600 μm to about 1200 μm, or about 864 μm. In particular embodiments, the second physical opening size is about 300 μm to about 900 μm or about 610 μm.

In a number of embodiments, the population of plant seeds is a population of wheat seeds and the methods of the present disclosure may further comprise: contacting the aspirated fraction with a first vibratory screen, wherein the first vibratory screen comprises a plurality of openings, each having a first opening size and a first opening shape, and wherein the aspirated fraction comprises a population of dry plant embryo explants and debris material; vibrating the first vibratory screen to produce a first screen motion, wherein the first screen motion comprises a first horizontal vibratory component; and separating a first screen fraction of embryo explants from a first screen portion of the debris material by length, width, or thickness relative to the first opening size or the first opening shape, or by a displacement of the first screen fraction relative to a displacement of the first screen portion of the debris material produced by the first screen motion. The first opening shape, in particular embodiments, is oblong, and the first opening size is about 5 mm to about 15 mm, about 6 mm to about 14 mm, about 8 mm to about 12 mm, about 8 mm to about 10 mm, about 9 mm to about 11 mm, about 10 mm to about 12 mm in length, or about 8 mm, about 9 mm, about 10 mm, about 11 mm, or about 12 mm in length, and from about 0.6 mm to about 0.8 mm, about 0.6 mm to about 0.7 mm, or about 0.7 mm to about 0.8 mm in width, or about 0.6 mm, about 0.65 mm, about 0.7 mm, about 0.75 mm, or about 0.8 mm in width.

In some embodiments, the population of plant seeds is a population of wheat seeds and the purifying comprises: contacting the aspirated fraction, the second fraction, or the first screen fraction with a textured surface of a first vibratory platform, wherein the first textured surface of the first vibratory platform is substantially planar, and wherein the aspirated fraction, the second fraction, or the first screen fraction comprises a population of dry plant embryo explants and debris material; vibrating the first vibratory platform to produce a first platform motion; and separating a first platform fraction of the plant embryo explants from a first platform portion of the debris material according to a displacement of the platform fraction relative to a displacement of the platform portion of debris material on the textured surface of the first vibratory platform, wherein the first vibratory platform comprises a first tilt angle of about 10.0 degrees to about 20.0 degrees, about 10.0 degrees to about 19.0 degrees, about 12.0 degrees to about 17.0 degrees, about 13.0 degrees to about 16.0 degrees, about 14.0 degrees to about 15.0 degrees, or about 14.5 degrees, and a first pitch angle of about 1.5 degrees to about 8.0 degrees, about 2.0 degrees to about 6.0 degrees, about 3.0 degrees to about 5.0 degrees, about 3.5 degrees to about 4.5 degrees, or about 4.0 degrees. In certain embodiments, the population of plant seeds is a population of wheat seeds and the methods of the present disclosure may further comprise: contacting the first platform fraction with a second textured surface of a second vibratory platform, wherein the second textured surface of the second vibratory platform is substantially planar; vibrating the second vibratory platform to produce a second platform motion; and separating a second platform fraction of the plant embryo explants of the first platform fraction from a second platform portion of the debris material according to a displacement of the second platform fraction relative to a displacement of the second platform portion of debris material on the second textured surface of the second vibratory platform, wherein the second vibratory platform comprises a second tilt angle of about 10.0 degrees to about 16.0 degrees, about 10.0 degrees to about 15.0 degrees, about 11.0 degrees to about 15.0 degrees, about 12.0 degrees to about 14.0 degrees, about 12.5 degrees to about 13.5 degrees, about 12.7 degrees to about 13.1 degrees, or about 12.9 degrees, and a second pitch angle of about 1.5 degrees to about 5.0 degrees, about 1.0 degrees to about 4.0 degrees, about 1.0 degrees to about 3.0 degrees, about 1.5 degrees to about 3.0 degrees, about 1.8 degrees to about 2.6 degrees, or about 2.2 degrees.

In particular embodiments, the population of plant seeds is a population of wheat seeds, and the method further comprises aspirating the population of plant seeds prior to the sanitizing, wherein the aspirating comprises: (a) aspirating within a first functional unit of a vertical chamber the population of plant seeds with a first air flow having a first air flow velocity, wherein the population of plant seeds comprises dry plant embryo explants comprising meristematic tissue and debris material; (b) separating a first aspirated fraction of the plant embryo explants from a first aspirated portion of the debris material within the first functional unit of the vertical chamber according to a displacement of the first aspirated fraction relative to a displacement of the first aspirated portion of the debris material produced by the first air flow within the first functional unit, wherein the first air flow comprises a variable vertical component and a variable horizontal component, wherein the first functional unit of the vertical chamber comprises a first lower partition, a first air input port, and a first air output port, wherein the first lower partition extends inward from a side wall of the vertical chamber to define a first lower advancement port between the first lower partition and an opposite side wall of the vertical chamber, wherein the first air input port comprises an opening in the side wall of the vertical chamber below the first lower partition, and wherein the first air flow at least partially enters the vertical chamber through the first air input port, travels through the first lower advancement port, and exits the vertical chamber through the first air output port; (c) transferring the first aspirated fraction of the plant embryo explants through the first lower advancement port into a second functional unit, wherein the first lower advancement port is between the first functional unit and the second functional unit, and wherein the first functional unit is positioned above the second functional unit, wherein the first aspirated portion of the debris material has been removed from the first aspirated fraction; (d) aspirating within the second functional unit of the vertical chamber the first aspirated fraction of plant embryo explants with a second air flow having a second air flow velocity; (e) separating a second aspirated fraction of the plant embryo explants comprised in the first aspirated fraction from a second aspirated portion of the debris material within the second functional unit of the vertical chamber according to a displacement of the second aspirated fraction relative to a displacement of the second aspirated portion of the debris material produced by the second air flow within the second functional unit, wherein the second air flow comprises a variable vertical component and a variable horizontal component, wherein the second functional unit of the vertical chamber comprises a second lower partition, a second air input port, and a second air output port, wherein the second lower partition extends inward from the side wall of the vertical chamber to define a second lower advancement port between the second lower partition and the opposite side wall of the vertical chamber, wherein the second air input port comprises an opening in the side wall of the vertical chamber below the second lower partition, and wherein the second air flow at least partially enters the vertical chamber through the second air input port, travels through the second lower advancement port, and exits the vertical chamber through the second air output port; (f) transferring the second aspirated fraction of the plant embryo explants through the second lower advancement port into a third functional unit, wherein the second lower advancement port is between the second functional unit and the third functional unit, and wherein the second functional unit is positioned above the third functional unit, wherein the second aspirated portion of the debris material has been removed from the second aspirated fraction; (g) aspirating within the third functional unit of the vertical chamber the second aspirated fraction of plant embryo explants with a third air flow having a third air flow velocity; (h) separating a third aspirated fraction of the plant embryo explants comprised in the second aspirated fraction from a third aspirated portion of the debris material within the third functional unit of the vertical chamber according to a displacement of the third aspirated fraction relative to a displacement of the third aspirated portion of the debris material produced by the third air flow within the third functional unit, wherein the third air flow comprises a variable vertical component and a variable horizontal component, wherein the third functional unit of the vertical chamber comprises a third lower partition, a third air input port, and a third air output port, wherein the third lower partition extends inward from the side wall of the vertical chamber to define a third lower advancement port between the third lower partition and the opposite side wall of the vertical chamber, wherein the third air input port comprises an opening in the side wall of the vertical chamber below the third lower partition, and wherein the third air flow at least partially enters the vertical chamber through the third air input port, travels through the third lower advancement port, and exits the vertical chamber through the third air output port; (i) transferring the third aspirated fraction of the plant embryo explants through the third lower advancement port into a fourth functional unit, wherein the third lower advancement port is between the third functional unit and the fourth functional unit, and wherein the third functional unit is positioned above the fourth functional unit, wherein the third aspirated portion of the debris material has been removed from the third aspirated fraction; (j) aspirating within the fourth functional unit of the vertical chamber the third aspirated fraction of plant embryo explants with a fourth air flow having a fourth air flow velocity; (k) separating a fourth aspirated fraction of the plant embryo explants comprised in the third aspirated fraction from a fourth aspirated portion of the debris material within the fourth functional unit of the vertical chamber according to a displacement of the fourth aspirated fraction relative to a displacement of the fourth aspirated portion of the debris material produced by the fourth air flow within the fourth functional unit, wherein the fourth air flow comprises a variable vertical component and a variable horizontal component, wherein the fourth functional unit of the vertical chamber comprises a fourth lower partition, a fourth air input port, and a fourth air output port, wherein the fourth lower partition extends inward from the side wall of the vertical chamber to define a fourth lower advancement port between the fourth lower partition and the opposite side wall of the vertical chamber, wherein the fourth air input port comprises an opening in the side wall of the vertical chamber below the fourth lower partition, and wherein the fourth air flow at least partially enters the vertical chamber through the fourth air input port, travels through the fourth lower advancement port, and exits the vertical chamber through the fourth air output port; (l) transferring the fourth aspirated fraction of the plant embryo explants through the fourth lower advancement port into a fifth functional unit, wherein the fourth lower advancement port is between the fourth functional unit and the fifth functional unit, and wherein the fourth functional unit is positioned above the fifth functional unit, wherein the fourth aspirated portion of the debris material has been removed from the fourth aspirated fraction; (m) aspirating within the fifth functional unit of the vertical chamber the fourth aspirated fraction of plant embryo explants with a fifth air flow having a fifth air flow velocity; (n) separating a fifth aspirated fraction of the plant embryo explants comprised in the fourth aspirated fraction from a fifth aspirated portion of the debris material within the fifth functional unit of the vertical chamber according to a displacement of the fifth aspirated fraction relative to a displacement of the fifth aspirated portion of the debris material produced by the fifth air flow within the fifth functional unit, wherein the fifth air flow comprises a variable vertical component and a variable horizontal component, wherein the fifth functional unit of the vertical chamber comprises a fifth lower partition, a fifth air input port, and a fifth air output port, wherein the fifth lower partition extends inward from the side wall of the vertical chamber to define a fifth lower advancement port between the fifth lower partition and the opposite side wall of the vertical chamber, wherein the fifth air input port comprises an opening in the side wall of the vertical chamber below the fifth lower partition, and wherein the fifth air flow at least partially enters the vertical chamber through the fifth air input port, travels through the fifth lower advancement port, and exits the vertical chamber through the fifth air output port; (o) transferring the fifth aspirated fraction of the plant embryo explants through the fifth lower advancement port into a sixth functional unit, wherein the fifth lower advancement port is between the fifth functional unit and the sixth functional unit, and wherein the fifth functional unit is positioned above the sixth functional unit, wherein the fifth aspirated portion of the debris material has been removed from the fifth aspirated fraction; (p) aspirating within the sixth functional unit of the vertical chamber the fifth aspirated fraction of plant embryo explants with a sixth air flow having a sixth air flow velocity; (q) separating a sixth aspirated fraction of the plant embryo explants comprised in the fifth aspirated fraction from a sixth aspirated portion of the debris material within the sixth functional unit of the vertical chamber according to a displacement of the sixth aspirated fraction relative to a displacement of the sixth aspirated portion of the debris material produced by the sixth air flow within the sixth functional unit, wherein the sixth air flow comprises a variable vertical component and a variable horizontal component, wherein the sixth functional unit of the vertical chamber comprises a sixth lower partition, a sixth air input port, and a sixth air output port, wherein the sixth lower partition extends inward from the side wall of the vertical chamber to define a lower collection port between the sixth lower partition and the opposite side wall of the vertical chamber, wherein the sixth air input port comprises an opening in the side wall of the vertical chamber below the sixth lower partition, and wherein the sixth air flow at least partially enters the vertical chamber through the sixth air input port, travels through the lower collection port, and exits the vertical chamber through the sixth air output port; and (r) collecting the sixth aspirated fraction of the plant embryo explants from the sixth functional unit, wherein the sixth aspirated portion of the debris material has been removed from the sixth aspirated fraction.

In certain embodiments, the population of plant seeds is a population of canola seeds and the milling comprises: positioning a first grinding roller and a second grinding roller to define a first gap having a first gap distance between the first roller and the second roller; rotating the first roller about a first axis of rotation and the second roller about a second axis of rotation; and passing the population of seeds through the first gap to produce a first preparation of plant embryo explants comprising meristematic tissue; wherein the first gap distance is about 0.508 mm to about 1.016 mm, about 0.508 mm to about 0.762 mm, or is about 0.8509 mm. In many embodiments, the population of plant seeds is a population of canola seeds and the method further comprises: contacting the preparation of dry plant embryo explants comprising meristematic tissue with a first moving sieve, wherein the first moving sieve comprises a plurality of openings, each having a first physical opening size; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; separating a second fraction of embryo explants from a second portion of the debris material by length, width, or thickness relative to the second physical opening size, or relative to a second effective opening size; contacting the second fraction with a third moving sieve, wherein the third moving sieve comprises a plurality of openings, each having a second physical opening size; and separating a third fraction of embryo explants from a third portion of the debris material by length, width, or thickness relative to the second physical opening size, or relative to a second effective opening size, wherein the first moving sieve, the second moving sieve, and the third moving sieve move in a circular, elliptical, or linear motion. The separating the third fraction, in some embodiments, is performed prior to the aspirating step. In certain embodiments, the first physical opening size is about 300 μm to about 1100 μm, about 600 μm to about 1100 μm, about 300 μm to about 1000 μm, about 500 μm to about 1000 μm, or about 864 μm. In some embodiments, the second physical opening size is about 600 μm to about 1000 μm or about 812 μm. In particular embodiments, the third physical opening size is about 300 μm to about 900 μm or about 503 μm. In some embodiments, the population of plant seeds is a population of canola seeds and the methods of the present disclosure may further comprise: separating the first preparation into a first top preparation fraction, a first middle preparation fraction, and a first bottom preparation fraction, wherein the first top preparation fraction is retained on the first moving sieve, the first middle preparation fraction is retained on the second moving sieve, and the first bottom preparation fraction is retained on the third moving sieve. The separating of the preparation, in some embodiments, is performed prior to the aspirating step. In particular embodiments, the population of plant seeds is a population of canola seeds and the methods of the present disclosure may further comprise. positioning the first grinding roller and the second grinding roller to define a first gap having a first gap distance between the first roller and the second roller; rotating the first roller about a first axis of rotation and the second roller about a second axis of rotation; and passing the first top preparation fraction through the first gap to produce a second preparation of plant embryo explants comprising meristematic tissue, wherein the first gap distance is about 0.508 mm to about 1.016 mm, about 0.508 mm to about 0.762 mm, or is about 0.6985 mm. The producing of the second preparation, in certain embodiments, is performed prior to the aspirating step. In some embodiments, the methods of the present disclosure may further comprise: separating the second preparation into a second top preparation fraction, a second middle preparation fraction, and a second bottom preparation fraction, wherein the second top preparation fraction is retained on the first moving sieve, the second middle preparation fraction is retained on the second moving sieve, and the second bottom preparation fraction is retained on the third moving sieve. The separating of the second preparation, in certain embodiments, is performed prior to the aspirating step. In a number of embodiments, the methods of the present disclosure may further comprise: combining the first middle preparation fraction with the second middle preparation fraction to produce a combined middle preparation fraction; or combining the first bottom preparation fraction with the second bottom preparation fraction to produce a combined bottom preparation fraction. In some embodiments, the combining is performed prior to the aspirating step.

In some embodiments, the population of plant seeds is a population of canola seeds and the purifying step comprises aspirating the combined middle preparation fraction or the combined bottom preparation fraction. In certain embodiments, the purifying step may further comprise: contacting the aspirated combined middle preparation fraction or the aspirated combined bottom preparation fraction with a sieve, wherein the sieve comprises a plurality of openings, each having a physical opening size, and wherein the aspirated combined middle preparation fraction or the aspirated combined bottom preparation fraction comprises a population of dry plant embryo explants and debris material; vibrating the sieve; and separating a sieved fraction of embryo explants from a sieved portion of the debris material by length, width, or thickness relative to the physical opening size, wherein the physical opening size is about 300 μm to about 900 μm, about 400 μm to about 800 μm, about 400 μm to about 700 μm, about 400 μm to about 600 μm, about 450 μm to about 550 μm or about 500 μm. In a number of embodiments, the population of plant seeds is a population of canola seeds and the aspirating comprises: aspirating within a first vertical chamber, a second vertical chamber, a third vertical chamber, and a fourth vertical chamber with a first upward air flow, a second upward air flow, a third upward air flow, and a fourth upward air flow, wherein the first upward airflow has a first air flow velocity of about 2.5 to about 4.0 m/s, about 3.0 m/s to about 4.0 m/s, about 3.4 m/s to about 3.8 m/s, or about 3.4 m/s to about 3.6 m/s, wherein the second upward air flow has a second air flow velocity of about 4.0 m/s to about 5.5 m/s, about 4.0 m/s to about 5.0 m/s, about 4.3 m/s to about 4.9 m/s, or about 4.6 m/s to about 4.8 m/s, wherein the third upward air flow has a third air flow velocity of about 5.0 m/s to about 7.0 m/s, about 5.5 m/s to about 6.5 m/s, or about 5.9 m/s to about 6.0 m/s, and wherein the fourth upward air flow has a fourth air flow velocity of about 8.0 m/s to about 9.5 m/s, about 8.5 m/s to about 9.0 m/s, or about 8.8 m/s to about 8.9 m/s.

In certain aspects, the present disclosure provides an apparatus for producing or purifying plant embryo explants from plant seeds, the apparatus comprising at least one component selected from the group consisting of: a seed roller mill, a seed grinder, a siever, a rotating cylinder, an aspirator, a vibratory screen, and a vibratory platform. In some embodiments, an apparatus of the present disclosure may comprise at least two components, at least three components, at least four components, at least five components, or at least six components selected from the group consisting of: a seed roller mill, a seed grinder, a siever, a rotating cylinder, an aspirator, a vibratory screen, and a vibratory platform.

In other aspects, the present disclosure provides a method of producing a preparation of plant embryo explants, the method comprising positioning a first grinding roller and a second grinding roller to define a first gap having a first gap distance between the first roller and the second roller; rotating the first roller about a first axis of rotation and the second roller about a second axis of rotation; and passing a population of plant seeds through the first gap to produce a first preparation of plant embryo explants comprising meristematic tissue, wherein the first gap distance is about 0.10 mm to about 7.62 mm, and wherein the first roller and the second roller each comprise an exterior surface and the exterior surface of the first roller and the exterior surface of the second roller each comprise a plurality of protrusions. In some embodiments, the method may further comprise positioning a third grinding roller and a fourth grinding roller to define a second gap having a second gap distance between the third roller and the fourth roller; rotating the third roller about a third axis of rotation and the fourth roller about a fourth axis of rotation; and passing the first preparation of embryo explants through the second gap to produce a second preparation of plant embryo explants comprising meristematic tissue, wherein the second gap distance is about 0.10 mm to about 7.62 mm, and wherein the third roller and the fourth roller each comprise an exterior surface and the exterior surface of the third roller and the exterior surface of the fourth roller each comprise a plurality of protrusions. In certain embodiments, the first axis of rotation is substantially parallel to the second axis of rotation, and the first axis of rotation and the second axis of rotation are substantially parallel to the ground. In particular embodiments, the third axis of rotation is substantially parallel to the fourth axis of rotation, and the third axis of rotation and the fourth axis of rotation are substantially parallel to the ground. In some embodiments, the first gap distance or the second gap distance is about 0.381 mm to about 7.62 mm, about 0.762 mm to about 6.35 mm, about 0.2032 mm to about 2.54 mm, or about 0.508 mm to about 1.016 mm.

In certain embodiments, the plurality of protrusions of the first roller or the second roller are defined as a plurality of shaped teeth or as a plurality of raised ridges, and the exterior surface of the first roller or the exterior surface of the second roller comprises about 4 to about 20 shaped teeth or about 2 to about 21 raised ridges per 2.54 cm. In particular embodiments, the plurality of protrusions of the third roller or the fourth roller are defined as a plurality of shaped teeth or as a plurality of raised ridges, and the exterior surface of the third roller or the exterior surface of the fourth roller comprises about 4 to about 20 shaped teeth or about 2 to about 21 raised ridges per 2.54 cm. In some embodiments, the exterior surface of the first roller or the exterior surface of the second roller comprises a plurality of shaped teeth. In certain embodiments, the exterior surface of the third roller or the exterior surface of the fourth roller comprises a plurality of shaped teeth. In particular embodiments, the plurality of shaped teeth of the first roller, the second roller, the third roller, or the fourth roller are configured into rows of teeth that run substantially perpendicular to the first axis of rotation, second axis of rotation, third axis of rotation, or fourth axis of rotation, respectively. The shaped teeth of the first roller or the shaped teeth of the second roller, in some embodiments, comprise a sharp surface and a dull surface. The shaped teeth of the third roller or the shaped teeth the fourth roller, in certain embodiments, comprise a sharp surface and a dull surface. The method may comprise, in particular embodiments, contacting the population of plant seeds or the first preparation of embryo explants with the sharp surface of the shaped teeth of the first roller and the sharp surface of the shaped teeth of the second roller. The method may comprise, in some embodiments, contacting the population of plant seeds or the first preparation of embryo explants with the dull surface of the shaped teeth of the first roller and the dull surface of the shaped teeth of the second roller. The method may comprise, in certain embodiments, contacting the population of plant seeds or the first preparation of embryo explants with the sharp surface of the shaped teeth of the first roller and the dull surface of the shaped teeth of the second roller. The method may comprise, in some embodiments, contacting the population of plant seeds or the first preparation of embryo explants with the dull surface of the shaped teeth of the first roller and the sharp surface of the shaped teeth of the second roller. In certain embodiments, the method may comprise contacting the first preparation of embryo explants with the sharp surface of the shaped teeth of the third roller and the sharp surface of the shaped teeth of the fourth roller; contacting the first preparation of embryo explants with the dull surface of the shaped teeth of the third roller and the dull surface of the shaped teeth of the fourth roller; contacting the first preparation of embryo explants with the sharp surface of the shaped teeth of the third roller and the dull surface of the shaped teeth of the fourth roller; or contacting the first preparation of embryo explants with the dull surface of the shaped teeth of the third roller and the sharp surface of the shaped teeth of the fourth roller. In particular embodiments, the plurality of shaped teeth of the first roller, the second roller, the third roller, or the fourth roller each comprise a tooth shape and the tooth shape is selected from the group consisting of a geometric shape, a scalene shape, and a triangular shape. The exterior surface of the first roller or the exterior surface of the second roller, in some embodiments, comprises a plurality of raised ridges. The exterior surface of the third roller or the exterior surface of the fourth roller, in certain embodiments, comprises a plurality of raised ridges. In particular embodiments, the plurality of raised ridges of the first roller, the second roller, the third roller, or the fourth roller are configured to run substantially parallel to the first axis of rotation, the second axis of rotation, the third axis of rotation, or the fourth axis of rotation, respectively.

In some embodiments, the method may comprise rotating the first roller at a first rate of rotation and the second roller at a second rate of rotation, wherein the first rate of rotation and the second rate of rotation are approximately the same, or wherein the first rate of rotation and the second rate of rotation are different. In certain embodiments, the method may comprise rotating the third roller at a third rate of rotation and the fourth roller at a fourth rate of rotation, wherein the third rate of rotation and the fourth rate of rotation are approximately the same, or wherein the third rate of rotation and the fourth rate of rotation are different. In particular embodiments, the first rate of rotation, the second rate of rotation, the third rate of rotation, or the fourth rate of rotation is about 50 rpm to about 1200 rpm, about 50 rpm to about 1000 rpm, about 50 rpm to about 800 rpm, about 50 rpm to about 600 rpm, about 50 rpm to about 400 rpm, about 50 rpm to about 250 rpm, about 50 rpm to about 200 rpm, about 100 rpm to about 250 rpm, about 150 rpm to about 250 rpm, about 50 rpm, about 100 rpm, about 150 rpm, about 200 rpm, about 250 rpm, about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, about 1000 rpm, about 1050 rpm, about 1100 rpm, about 1150 rpm, or about 1200 rpm. In some embodiments, the method may comprise rotating the first roller and the second roller at a rotation rate ratio of about 1:1 to about 10:1, about 1:1 to about 9:1, about 1:1 to about 8:1, about 1:1 to about 7:1, about 1:1 to about 8:1, about 1:1 to about 7:1, about 1:1 to about 6:1, about 1:1 to about 5:1, about 1:1 to about 4:1 about 1:1 to about 3:1, about 1:1 to about 2.5:1, about 1:1 to about 2:1, about 1:1, about 2:1, about 2.5:1, about 3:1, about 4:1, about 5:1, about 6:1, about 7:1, about 8:1, about 9:1, about 10:1 about 1.1:1, about 1.2:1, about 1.3:1, about 1.4:1, about 1.5:1, about 1.6:1, about 1.7:1, about 1.8:1, or about 1.9:1. In certain embodiments, the method may comprise rotating the third roller and the fourth roller at a rotation rate ratio of about 1:1 to about 10:1, about 1:1 to about 9:1, about 1:1 to about 8:1, about 1:1 to about 7:1, about 1:1 to about 8:1, about 1:1 to about 7:1, about 1:1 to about 6:1, about 1:1 to about 5:1, about 1:1 to about 4:1 about 1:1 to about 3:1, about 1:1 to about 2.5:1, about 1:1 to about 2:1, about 1:1, about 2:1, about 2.5:1, about 3:1, about 4:1, about 5:1, about 6:1, about 7:1, about 8:1, about 9:1, about 10:1 about 1.1:1, about 1.2:1, about 1.3:1, about 1.4:1, about 1.5:1, about 1.6:1, about 1.7:1, about 1.8:1, or about 1.9:1.

In particular embodiments, the population of plant seeds comprises a population of corn seeds and the first gap distance is about 0.381 mm to about 7.62 mm, about 2.032 mm to about 2.794 mm, or is about 2.54 mm. In some embodiments population of plant seeds comprises a population of corn seeds and the second gap distance is about 0.381 mm to about 7.62 mm, about 0.762 mm to about 1.778 mm, or is about 1.27 cm. The population of plant seeds, in some embodiments, comprises a population of soybean seeds and the first gap distance is about 0.762 mm to about 6.35 mm, about 3.81 mm to about 5.08 mm, or is about 4.2926 mm. The population of plant seeds, in certain embodiments, comprises a population of soybean seeds and the second gap distance is about 0.762 mm to about 6.35 mm, about 3.556 mm to about 4.318 mm, or is about 3.937 mm. In particular embodiments, the population of plant seeds comprises a population of wheat seeds and the first gap distance is about 0.2032 mm to about 2.54 mm, about 0.762 mm to about 1.788 mm, or is about 1.2827 mm. In particular embodiments, the population of plant seeds comprises a population of wheat seeds and the second gap distance is about 0.2032 mm to about 2.54 mm, about 0.2286 mm to about 0.4572 mm, or is about 0.3683 mm. In some embodiments, the population of plant seeds comprises a population of corn seeds, soybean seeds, or wheat seeds and the method may comprise rotating the first roller, the second roller, the third roller, or the fourth roller at about 150 rpm to about 250 rpm, about 175 rpm to about 225 rpm, or about 190 rpm to about 220 rpm. In certain embodiments, the population of plant seeds comprises a population of corn seeds, soybean seeds, or wheat seeds and the method may comprise rotating the first roller and the second roller at a rotation rate ratio of about 1:1 to about 4:1 or about 1.1:1; or rotating the third roller and the fourth roller at a rotation rate ratio of about 1:1 to about 4:1 or about 1.1:1. In specific embodiments, the method may comprise rotating the first roller at about 213 rpm and rotating the second roller at about 194 rpm; or rotating the third roller at about 213 rpm and rotating the second roller at about 194 rpm. In particular embodiments, the population of plant seeds comprises a population of corn seeds, soybean seeds, or wheat seeds and the exterior surface of the first roller and the exterior surface of the second roller each comprise the plurality of shaped teeth; or the exterior surface of the third roller and the exterior surface of the fourth roller each comprise the plurality of shaped teeth. In some embodiments, the plurality of shaped teeth are scalene shaped; the exterior surface of the first roller and the exterior surface of the second roller each comprise about 4 to about 8 teeth per 2.54 cm; or the exterior surface of the third roller and the exterior surface of the fourth roller each comprise about 4 to about 8 teeth per 2.54 cm. In certain embodiments, the population of plant seeds comprises a population of corn seeds, soybean seeds, or wheat seeds and the method may comprise contacting the population of plant seeds with the sharp surface of the shaped teeth of the first roller and the dull surface of the shaped teeth of the second roller; or contacting the first preparation of embryo explants with the sharp surface of the shaped teeth of the third roller and the dull surface of the shaped teeth of the fourth roller.

In certain embodiments, the population of plant seeds comprises a population of canola seeds and the first gap distance is about 0.508 mm to about 1.016 mm, about 0.508 mm to about 0.762 mm, or is about 0.8509 mm. In particular embodiments, the population of plant seeds comprises a population of canola seeds the method may further comprise positioning the first grinding roller and the second grinding roller to define a first gap having a first gap distance between the first roller and the second roller; rotating the first roller about a first axis of rotation and the second roller about a second axis of rotation; passing the first preparation or a fraction thereof through the first gap to produce a second preparation of plant embryo explants comprising meristematic tissue, wherein the first gap distance is about 0.508 mm to about 1.016 mm, about 0.508 mm to about 0.762 mm, or is about 0.6985 mm. The method may comprise, in certain embodiments, rotating the first roller and the second roller at about 100 rpm to about 400 rpm. The method may comprise, in particular embodiments, rotating the first roller and the second roller at a rotation rate ratio of about 1:1 to about 4:1 or about 2.5:1. The method may comprise, in some embodiments, rotating the first roller at about 340 rpm to about 350 rpm and rotating the second roller at about 130 rpm to about 145 rpm. In certain embodiments, the population of plant seeds comprises a population of canola seeds and the exterior surface of the first roller and the exterior surface of the second roller each comprise the plurality of shaped teeth. In particular embodiments, the population of plant seeds comprises a population of canola seeds and the plurality of shaped teeth are triangular shaped; or the exterior surface of the first roller and the exterior surface of the second roller each comprise about 8 to about 12 teeth per 2.54 cm. The method may comprise, in some embodiments, contacting the population of plant seeds or the first preparation of embryo explants with the sharp surface of the shaped teeth of the first roller and the sharp surface of the shaped teeth of the second roller.

In particular embodiments, the methods provided by the present disclosure may comprise contacting the population of plant seeds or the first preparation of embryo explants with the exterior surface of the first roller and the exterior surface of the second roller approximately simultaneously; or contacting the first preparation of embryo explants with the exterior surface of the third roller and the exterior surface of the fourth roller approximately simultaneously.

In yet other aspects, the present disclosure provides a method of producing a preparation plant embryo explants, the method comprising positioning a first grinding plate and a second grinding plate to define a first gap having a first gap distance between the first plate and the second plate; rotating the first plate or the second plate about an axis of rotation; and contacting a population of plant seeds with an interior surface of the first plate and an interior surface of the second plate to produce a first preparation of embryo explants comprising meristematic tissue, wherein the first gap distance is about 2.5 mm to about 4.0 mm or about 3.0 mm to about 3.25 mm. In some embodiments, the method may further comprise positioning a third grinding plate and a fourth grinding plate to define a second gap having a second gap distance between the third plate and the fourth plate; rotating the third plate or the fourth plate about an axis of rotation; and contacting the first preparation of embryo explants with an interior surface of the third plate and an interior surface of the fourth plate to produce a second preparation of embryo explants comprising meristematic tissue, wherein the second gap distance is about 0.5 mm to about 2.5 mm or is about 1.5 mm. In certain embodiments, the interior surface of the first plate and the interior surface of the second plate each comprise a plurality of grinder teeth; or the interior surface of the third plate and the interior surface of the fourth plate each comprise a plurality of grinder teeth. The grinder teeth of the first plate and the grinder teeth of the second plate, in particular embodiments, may comprise a sharp surface and a dull surface. The grinder teeth of the third plate and the grinder teeth of the fourth plate, in some embodiments, may comprise a sharp surface and a dull surface. The method may comprise, in certain embodiments, contacting the population of plant seeds with the sharp surface of the grinder teeth of the first plate and the sharp surface of the grinder teeth of the second plate; or contacting the first preparation of embryo explants with the sharp surface of the grinder teeth of the third plate and the sharp surface of the grinder teeth of the fourth plate. In some embodiments, the method may comprise contacting the population of plant seeds with the interior surface of the first plate and the interior surface of the second plate approximately simultaneously. Contacting, in certain embodiments, may comprise contacting the first preparation of embryo explants with the interior surface of the third plate and the interior surface of the fourth plate approximately simultaneously. In particular embodiments, the axis of rotation of the first plate, the second plate, the third plate, or the fourth plate is substantially parallel to the ground.

The methods provided by the present disclosure, may comprise, in some embodiments, rotating the first plate at about 200 rpm to about 600 rpm, about 300 rpm to about 500 rpm, or about 400 rpm, wherein the second plate remains approximately stationary; or rotating the second plate at about 200 rpm to about 600 rpm, about 300 rpm to about 500 rpm, or about 400 rpm, wherein the first plate remains approximately stationary. The method, in certain embodiments, may comprise contacting the population of plant seeds with the first plate and the second plate at a rate of about 600 g/min to about 1000 g/min or about 800 g/min. In some embodiments, the grinder teeth of the first plate and the grinder teeth of the second plate each comprise a grinder tooth shape and the grinder tooth shape is selected from the group consisting of a geometric shape, a scalene shape, and a triangular shape; or the grinder teeth of the third plate and the grinder teeth of the fourth plate each comprise a grinder tooth shape and the grinder tooth shape is selected from the group consisting of a geometric shape, a scalene shape, and a triangular shape. In particular embodiments, the first plate and the second plate each comprise about 2 to about 50 or about 2 to about 10 grinder teeth per 2.54 cm; or the third plate and the fourth plate each comprise about 2 to about 50 or about 2 to about 10 grinder teeth per 2.54 cm. The method, in particular embodiments, may comprise rotating the third plate at about 100 rpm to about 200 rpm, about 100 rpm to about 150 rpm, or about 135 rpm, wherein the fourth plate remains approximately stationary; or rotating the fourth plate at about 100 rpm to about 200 rpm, about 100 rpm to about 150 rpm, or about 135 rpm, wherein the third plate remains approximately stationary.

In certain embodiments, the methods provided by the present disclosure may comprise producing a first fraction of the first preparation of embryo explants; and contacting the interior surface of the third plate and the interior surface of the fourth plate with the first fraction of the first preparation. Producing the first fraction of the first preparation may comprise, in particular embodiments, contacting the first preparation of embryo explants with a moving plate, wherein the moving plate comprises a proximal end, a distal end, and a plurality of openings located near the distal end, each comprising a first physical opening size, and wherein the first preparation comprises a population of embryo explants and debris material; passing the first preparation through the plurality of openings of the moving plate and contacting a first moving sieve with the first preparation, wherein the first moving sieve comprises a plurality of openings, each having a second physical opening size; separating the first fraction of the first preparation from a first portion of the debris material by length, width, or thickness relative to the second physical opening size; and collecting the first fraction of the first preparation, wherein the moving plate and the first moving sieve move in a linear motion. In some embodiments, the method may further comprise aspirating the first preparation after contacting the first preparation with the moving plate and prior to contacting the first preparation with the first moving sieve; or aspirating the first preparation after contacting the first preparation with the first moving sieve and prior to separating the first fraction of the first preparation. In certain embodiments, the first physical opening size is about 300 μm to about 5000 μm, about 400 μm to about 4500 μm, about 500 μm to about 4000 μm, about 500 μm to about 3500 μm, about 500 μm to about 3000 μm, or about 500 μm to about 2500 μm; or the second physical opening size is about 700 μm to about 1300 μm, about 1181 μm, or about 980 μm. The method may further comprise, in particular embodiments, applying a cryogenic treatment to the first preparation or the first fraction of the first preparation prior to contacting the first preparation or the first fraction of the first preparation with the third plate and the fourth plate. In some embodiments, the population of plant seeds comprises a population of cotton seeds.

In still yet other aspects, the present disclosure provides a method of purifying genetically modifiable dry plant embryo explants, the method comprising contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; and separating a first fraction of embryo explants from a first portion of the debris material by length, width, and/or thickness relative to the first physical opening size, or relative to a first effective opening size, wherein the first moving sieve moves in a circular, elliptical, and/or linear motion. In some embodiments, the methods provided by the present disclosure may further comprise aspirating the preparation to remove a first aspirated portion of the debris material from the preparation. The first moving sieve, in certain embodiments, is positioned at first slope angle and the first effective opening size is dependent on the first physical opening size and the first slope angle. In particular embodiments, the first slope angle is about 0 degrees to about 40 degrees. The first moving sieve, in some embodiments, comprises a proximal end and a distal end, and the contacting comprises first contacting the preparation with the proximal end, wherein the proximal end is elevated relative to the distal end. In certain embodiments, the preparation travels along the first moving sieve in a general proximal-to-distal direction. In particular embodiments, the motion of the first moving sieve changes gradually from a circular motion to an elliptical motion to a linear motion from the proximal end to the distal end. The first moving sieve, in some embodiments, further comprises a vibratory motion.

The first physical opening size, in particular embodiments, is about 300 μm to about 2600 μm, about 1600 μm to about 2600 μm, about 1600 μm to about 2500 μm, about 800 μm to about 2000 μm, about 800 to about 1500 μm, about 700 μm to about 1300 μm, about 600 μm to about 1200 μm, about 600 μm to about 1100 μm, about 500 μm to about 1000 μm, about 300 to about 1000 μm, or about 300 μm to about 900 μm. In certain embodiments, the population of dry plant embryo explants is a population of dry corn embryo explants, and the first physical opening size is about 500 μm to about 2000 μm, about 800 μm to about 2000 μm, about 500 μm to about 1000 μm, about 1181 μm, or about 812 μm. In some embodiments, the population of dry plant embryo explants is a population of dry soybean embryo explants, and the first physical opening size is about 800 μm to about 2600 μm, about 1600 μm to about 2600 μm, about 800 μm to about 1500 μm, about 2032 μm, or about 1181 μm. In particular embodiments, the population of dry plant embryo explants is a population of dry wheat embryo explants, and the first physical opening size is about 300 μm to about 1200 μm, about 600 μm to about 1200 μm, about 300 μm to about 900 μm, about 864 μm, or about 610 μm. In certain embodiments, the population of dry plant embryo explants is a population of dry canola embryo explants, and the first physical opening size is about 300 μm to about 1100 μm, about 600 μm to about 1100 μm, about 300 μm to about 1000 μm, about 500 μm to about 1000 μm, about 864 μm, about 812 μm, or about 503 μm. In some embodiments, the population of dry plant embryo explants is a population of dry cotton embryo explants, and the first physical opening size is about 700 μm to about 2500 μm, about 1600 μm to about 2500 μm, about 700 μm to about 1300 μm, about 2032 μm, about 1181 μm, or about 980 μm. In particular embodiments of the present disclosure, each opening of the first moving sieve is defined as comprising a geometric shape. The geometric shape, in some embodiments, is selected from the group consisting of a rectangle, a square, a circle, or an oval. The first moving sieve, in certain embodiments, comprises a planar length and the planar length of the first moving sieve is from about 0.5 m to about 4 m, about 1 m to about 3 m, or about 1.5 m to about 2.5 m. In some embodiments, the first moving sieve comprises a planar width and the planar width of the first moving sieve is from about 0.1 m to about 2 m, about 0.25 m to about 2 m, about 0.5 m to about 1.5 m, or about 0.5 m to about 1 m.

In some embodiments of the present disclosure, separating comprises retaining the first portion of the debris material on the first moving sieve and passing the first fraction of embryo explants through the plurality of openings. In certain embodiments of the present disclosure, separating comprises retaining the first fraction of embryo explants on the first moving sieve and passing the first portion of the debris material through the plurality of openings. In particular embodiments, the methods provided by the present disclosure comprise collecting the first fraction of embryo explants at or near the distal end of the first moving sieve.

In some aspects of the present disclosure, the method may further comprise contacting the first fraction with a second moving sieve, wherein the second moving sieve comprises a plurality of openings, each having a second physical opening size; and separating a second fraction of embryo explants from a second portion of the debris material by length, width, and/or thickness relative to the second physical opening size, or relative to a second effective opening size, wherein the second moving sieve moves in a circular, elliptical, and/or linear motion. In certain embodiments, the second moving sieve is positioned at a second slope angle and the second effective opening size is dependent on the second physical opening size and the second slope angle. The contacting, in some embodiments, comprises passing the first fraction through the plurality of openings of the first moving sieve and contacting the second moving sieve with the first fraction. In certain embodiments, the methods provided by the present disclosure may further comprise aspirating the preparation to remove a first aspirated portion of the debris material from the preparation; or aspirating the first fraction to remove a second aspirated portion of the debris material from the first fraction. In particular embodiments, the second slope is about 0 degrees to about 40 degrees. The first fraction of dry embryo explants, in some embodiments, travels along the second moving sieve in a general proximal-to-distal direction. In particular embodiments, the second moving sieve comprises a proximal end and a distal end and the motion of the second moving sieve changes gradually from a circular motion to an elliptical motion to a linear motion from the proximal end to the distal end. In certain embodiments, the second moving sieve further comprises a vibratory motion.

In some embodiments, the second physical opening size is about 300 μm to about 1500 μm, about 800 to about 1500 μm, about 700 μm to about 1300 μm, about 600 μm to about 1200 μm, about 600 μm to about 1100 μm, about 500 μm to about 1000 μm, about 300 to about 1000 μm, or about 300 μm to about 900 μm. In particular embodiments, the first physical opening size is about 600 μm to about 2600 μm and the second physical opening size is about 300 μm to about 1500 μm. The population of dry plant embryo explants, in some embodiments, is a population of dry corn embryo explants, and the first physical opening size is about 800 μm to about 2000 μm or about 1181 μm, and the second physical opening size is about 500 μm to about 1000 μm or about 812 μm. The population of dry plant embryo explants, in certain embodiments, is a population of dry soybean embryo explants, and the first physical opening size is about 1600 μm to about 2600 μm or about 2032 μm, and the second physical opening size is about 800 μm to about 1500 μm or about 1181 μm. The population of dry plant embryo explants, in certain embodiments, is a population of dry wheat embryo explants, and the first physical opening size is about 600 μm to about 1200 μm or about 864 μm, and the second physical opening size is about 300 μm to about 900 μm or about 610 μm. In some embodiments, the population of dry plant embryo explants is a population of dry canola embryo explants, and the first physical opening size is about 600 μm to about 1100 μm or about 864 μm, and the second physical opening size is about 600 μm to about 1000 μm or about 812 μm. In certain embodiments, the population of dry plant embryo explants is a population of dry cotton embryo explants, and the first physical opening size is about 1600 μm to about 2500 μm or about 2032 μm, and the second physical opening size is about 700 μm to about 1300 μm, about 1181 μm, or about 980 μm. In some embodiments, each opening of the second moving sieve is defined as comprising a geometric shape. The geometric shape, in particular embodiments, is selected from the group consisting of a rectangle, a square, a circle, or an oval. In particular embodiments, the second moving sieve comprises a planar length and the planar length is from about 0.5 m to about 4 m, about 1 m to about 3 m, or about 1.5 m to about 2.5 m. In certain embodiments, the second moving sieve comprises a planar width and the planar width is from about 0.1 m to about 2 m, about 0.25 m to about 2 m, about 0.5 m to about 1.5 m, or about 0.5 m to about 1 m.

In certain embodiments of the present disclosure, separating comprises retaining the second portion of the debris material on the second moving sieve and passing the second fraction of embryo explants through the plurality of openings. In some embodiments of the present disclosure, separating comprises retaining the second fraction of embryo explants on the second moving sieve and passing the second portion of the debris material through the plurality of openings. In particular embodiments the methods provided by the present disclosure comprise collecting the second fraction of embryo explants at or near the distal end of the second moving sieve. In some embodiments, the position of the first moving sieve is directly above the position of the second moving sieve. The plane of the first moving sieve, in certain embodiments, is parallel to the plane of the second moving sieve. The first moving sieve and the second moving sieve, in particular embodiments, are structurally connected. In some embodiments, the first moving sieve and the second moving sieve move in unison. In particular embodiments, the motion of the first moving sieve and the second moving sieve is automated and/or motorized. In some embodiments the methods provided by the present disclosure comprise capturing the second fraction on a receiving plate and discharging the second fraction through an output near a distal end of the second moving sieve.

In some embodiments, the purity of the first fraction is increased by about 0.1-fold to about 10.0-fold compared to the purity of the population of dry plant embryo explants in the preparation of dry embryo explants, wherein the purity is defined as the percentage of dry plant embryo explants per particle. In certain embodiments, the purity of the second fraction is increased by about 0.1-fold to about 10.0-fold compared to the purity of the population of dry plant embryo explants in the preparation of dry embryo explants or compared to the purity of the first fraction, wherein the purity is defined as the percentage of dry plant embryo explants per particle.

In certain aspects provided by the present disclosure, the method may further comprise contacting the second fraction with a third moving sieve, wherein the third moving sieve comprises a plurality of openings, each having a third physical opening size; and separating a third fraction of embryo explants from a third portion of the debris material by length, width, and/or thickness relative to the third physical opening size, or relative to a third effective opening size, wherein the third moving sieve moves in a circular, elliptical, and/or linear motion. In some embodiments, the third moving sieve is positioned at a third slope angle and the third effective opening size is dependent on the third physical opening size and the third slope angle. The slope angle, in certain embodiments, is about 0 degrees to about 40 degrees. In particular embodiments, the third moving sieve comprises a proximal end and a distal end and the motion of the third moving sieve changes gradually from a circular motion to an elliptical motion to a linear motion from the proximal end to the distal end. The third moving sieve, in certain embodiments, further comprises a vibratory motion. In some embodiments, the position of the second moving sieve is directly above the position of the third moving sieve. The plane of the second moving sieve, in certain embodiments, is parallel to the plane of the third moving sieve. The second moving sieve and the third moving sieve, in particular embodiments, are structurally connected. The second moving sieve and the third moving sieve, in some embodiments, move in unison. The motion of the second moving sieve and the third moving sieve, in particular embodiments, is automated and/or motorized.

In some embodiments, the third physical opening size is about 300 μm to about 900 μm, about 350 to about 600 μm, or about 503 μm. The population of dry plant embryo explants, in particular embodiments, is a population of dry canola embryo explants, and the first physical opening size is about 600 μm to about 1100 μm or about 864 μm, the second physical opening size is about 600 μm to about 1000 μm or about 812 μm, and the third physical opening size is about 300 μm to about 900 μm or about 503 μm. In certain embodiments, each opening of the third moving sieve is defined as comprising a geometric shape. The geometric shape, in some embodiments, is selected from the group consisting of a rectangle, a square, a circle, or an oval. In particular embodiments, the third moving sieve comprises a planar length and the planar length is from about 0.5 m to about 4 m, about 1 m to about 3 m, or about 1.5 m to about 2.5 m. In certain embodiments, the third moving sieve comprises a planar width and the planar width is from about 0.1 m to about 2 m, about 0.25 m to about 2 m, about 0.5 m to about 1.5 m, or about 0.5 m to about 1 m.

In some embodiments, separating comprises retaining the third portion of the debris material on the third moving sieve and passing the third fraction of embryo explants through the plurality of openings. In certain embodiments, separating comprises retaining the third fraction of embryo explants on the third moving sieve and passing the third portion of the debris material through the plurality of openings. The purity of the third fraction, in particular embodiments, is increased by about 0.1-fold to about 10.0-fold compared to the purity of the population of dry plant embryo explants in the preparation of dry embryo explants, or compared to the purity of the first fraction, or compared to the purity of the second fraction, wherein the purity is defined as the percentage of dry plant embryo explants per particle.

In some aspects the present disclosure provides a method of purifying genetically modifiable dry embryo explants, the method comprising contacting a preparation of dry plant embryo explants comprising meristematic tissue with a moving plate, wherein the moving plate comprises a proximal end, a distal end, and a plurality of openings located near the distal end, each opening comprising a first physical opening size, and wherein the preparation comprises a population of dry embryo explants and debris material; passing the preparation through the plurality of openings of the moving plate and contacting a first moving sieve with the preparation, wherein the first moving sieve comprises a plurality of openings, each having a second physical opening size; and separating a first fraction of embryo explants from a first portion of the debris material by length, width, or thickness relative to the second physical opening size, wherein the moving plate and the first moving sieve move in a linear motion. In certain embodiments, the method provided by the present disclosure further comprises aspirating the preparation to remove a first aspirated portion of the debris material from the preparation. In certain embodiments, the first physical opening size is about 1500 μm to about 2 cm. In some embodiments, the second physical opening size is about 700 μm to about 1300 μm, about 1181 μm, or about 980 μm.

In particular embodiments the methods provided by the present disclosure further comprise contacting the first fraction with a second moving sieve comprising a plurality of openings, each having a third physical opening size; separating a second fraction from a second portion of the debris material by length, width, and/or thickness relative to the third physical opening size; contacting the second fraction with a third moving sieve comprising a plurality of openings, each comprising a fourth physical opening size; and separating a third fraction from a third portion of the debris material by length, width, and/or thickness relative to the fourth physical opening size, wherein the second moving sieve and the third moving sieve move in a linear motion. In some embodiments, the method further comprises aspirating the first fraction to remove a second aspirated portion of the debris material from the first fraction. In certain embodiments, the third physical opening size is about 1600 μm to about 2500 μm or about 2032 μm and the fourth physical opening size is about 700 μm to about 1300 μm, about 1181 μm, or about 980 μm. In some embodiments, the method further comprises applying a cryogenic treatment to the first fraction prior to contacting the first fraction with the second moving sieve. In certain embodiments, the purity of the first fraction is increased by about 0.1-fold to about 5.0-fold compared to the purity of the population of dry plant embryo explants in the preparation of dry embryo explants, wherein the purity is defined as the percentage of dry embryo explants per particle. In particular embodiments, the purity of the third fraction is increased by about 0.1-fold to about 5.0-fold compared to compared to the purity of the population of dry plant embryo explants in the preparation of dry embryo explants, or compared to the purity of the first fraction, wherein the purity is defined as the percentage of dry plant embryo explants per particle.

In other aspects, the present disclosure provides a method of purifying genetically modifiable dry plant embryo explants, the method comprising contacting a preparation of dry plant embryo explants comprising meristematic tissue with an interior surface of a rotating cylinder, wherein the interior surface comprises a plurality of indentations, the indentations having an indentation size and an indentation shape, and wherein the preparation comprises a population of dry plant embryo explants and debris material; rotating the rotating cylinder about an axis of rotation to produce a centrifugal force acting on the preparation, wherein the axis of rotation is substantially parallel to the ground; and separating a fraction of the plant embryo explants of the preparation from a portion of the debris material according to a displacement of the portion of the debris material relative to a displacement of the fraction of plant embryo explants produced by the rotating. In some embodiments, the separating comprises separating the fraction of the plant embryo explants from the portion of the debris material by the relative length, width, shape, or weight of the plant embryo explants and the debris material. In certain embodiments, the indentation shape comprises a shape selected from the group consisting of a geometric shape, a rectangle, a square, a circle, or an oval. The indentation, in particular embodiments, is defined as a depression relative to the interior surface of the rotating cylinder. In some embodiments, the rotating lifts the portion of the debris material from a bottom interior region to a top interior region of the rotating cylinder. The fraction of plant embryo explants, in certain embodiments, remains at the bottom interior region of the rotating cylinder during the rotating. In particular embodiments, the displacement of the fraction of plant embryo explants is less than the displacement of the portion of the debris material. In certain embodiments, the displacement of the fraction of plant embryo explants is defined as a net displacement, and the net displacement of the fraction of plant embryo explants is approximately zero. The rotating, in some embodiments, comprises rotating the rotating cylinder at a rate of about 15 rpm to about 50 rpm, about 20 rpm to about 45 rpm, about 25 rpm to about 40 rpm, about 30 rpm to about 40 rpm, about 35 rpm to about 40 rpm, about 37 rpm, or about 38 rpm.

In some embodiments, the indentation size or the indentation shape of the plurality of indentations is configured, in combination with the centrifugal force acting on the preparation, to maintain the portion of the debris material in greater contact with the interior surface of the rotating cylinder relative to the fraction of plant embryo explants as the rotating lifts the portion of the debris material from the bottom interior region to the top interior region of the rotating cylinder, wherein the greater contact of the debris material with the interior surface of the rotating cylinder results in a greater displacement of the portion of the debris material relative to the displacement of the plant embryo explants. In particular embodiments, the indentation size or the indentation shape of the plurality of indentations in combination with the centrifugal force acting on the preparation acts against the force of gravity to produce the displacement of the portion of the debris material or the displacement of the fraction of plant embryo explants. The indentation size or the indentation shape of the plurality of indentations, in some embodiments, is configured to exclude the plant embryo explants of the fraction of plant embryo explants from the plurality of indentations. The indentation size or the indentation shape of the plurality of indentations, in certain embodiments, is configured to exclude the fraction of plant embryo explants from the plurality of indentations. In particular embodiments, each indentation size comprises an indentation diameter, an indentation width, an indentation length, or an indentation depth. In some embodiments, the indentation diameter, the indentation width, or the indentation length is about 1.00 mm to about 4.00 mm, about 1.25 mm to about 3.75 mm, about 1.50 mm to about 3.50 mm, about 1.75 mm to about 3.50 mm, about 2.00 mm to about 3.25 mm, about 2.25 mm to about 3.00 mm, about 2.50 mm to about 2.75 mm, about 1.25 mm to about 2.75 mm, about 1.50 mm to about 2.50 mm, about 1.75 mm to about 2.50 mm, about 2.00 mm to about 2.50 mm, about 1.75 mm to about 2.25 mm, about 2.00 mm, about 2.25 mm, about 2.75 mm, or about 3.00 mm. In particular embodiments, the indentation depth is about 0.25 mm to about 2.00 mm, about 0.50 mm to about 1.75 mm, about 0.75 mm to about 1.25 mm, or about 1.00 mm.

In particular embodiments, the preparation comprises corn, wheat, soybean, cotton, or canola embryo explants. The preparation comprises corn embryo explants, in certain embodiments, and the indentation diameter, the indentation width, or the indentation length is about 1.50 mm to about 2.75 mm, about 1.75 mm to about 2.50 mm, about 2.00 mm to about 2.25 mm, about 2.00 mm, or about 2.25 mm. The preparation comprises soybean embryo explants, in some embodiments, and the indentation diameter, the indentation width, or the indentation length is about 2.25 mm to about 3.50 mm, about 2.50 mm to about 3.25 mm, about 2.75 mm to about 3.00 mm, about 2.75 mm, or about 3.00 mm. The preparation comprises cotton embryo explants, in particular embodiments, and the indentation diameter, the indentation width, or the indentation length is about 2.25 mm to about 3.50 mm, about 2.50 mm to about 3.25 mm, about 2.75 mm to about 3.00 mm, about 2.75 mm, or about 3.00 mm.

In some embodiments the separating comprises transferring the portion of the debris material from the bottom interior region to the top interior region of the rotating cylinder; and delivering the portion of the debris material to a debris collector, wherein gravity causes the portion of the debris material to fall away from the interior surface of the top interior region of the rotating cylinder and into the debris collector. The fraction of embryo explants, in certain embodiments, remains at or near the bottom interior region of the rotating cylinder during the separating. In particular embodiments, the methods provided by the present disclosure comprise loading the rotating cylinder with a first desired amount of the preparation, wherein the loading comprises contacting the first desired amount with the interior surface of the rotating cylinder at an initial feed rate. The initial feed rate, in certain embodiments, is about 500 g/min to about 2500 g/min, about 1000 g/min to about 2500 g/min, about 1500 g/min to about 2000 g/min, or about 1942 g/min. In some embodiments, the methods provided by the present disclosure further comprise loading the rotating cylinder with a second desired amount of the preparation, wherein the loading comprises contacting the second desired amount with the interior surface of the rotating cylinder at a second feed rate. The second feed rate, in particular embodiments, is about 500 g/min to about 2500 g/min, about 1000 g/min to about 2000 g/min, about 1000 g/min to about 1500 g/min, or about 1271 g/min.

In certain embodiments, the methods provided by the present disclosure further comprise positioning a debris collector configured to receive the portion of the debris material within a hollow center cavity of the rotating cylinder; and collecting the portion of the debris material in the debris collector. The rotating cylinder, in some embodiments, is structurally connected to the debris collector. In particular embodiments, gravity causes the portion of the debris material to fall away from the interior surface at or near the top interior region of the rotating cylinder and into the debris collector. In some embodiments, the methods provided by the present disclosure comprise positioning the debris collector at a preferred location within the hollow center cavity of the rotating cylinder. The debris collector, in certain embodiments, comprises at least one substantially planar surface, a container, or a collection chute.

In particular embodiments, the rotating cylinder has an interior radius (r) measured from the axis of rotation to the interior surface of the rotating cylinder, and the debris collector comprises a top portion and a bottom portion, and the method further comprises positioning the top portion of the debris collector within the hollow center cavity at a distance of about 0.1×(r) to about 0.9×(r), 0.2×(r) to about 0.8×(r), about 0.2×(r) to about 0.7×(r), about 0.3×(r) to about 0.6×(r), or about 0.4×(r) to about 0.6×(r) from the axis of rotation of the rotating cylinder. In some embodiments, the debris collector comprises a top portion and a bottom portion, and the method further comprises positioning a plane of the top portion of the debris collector within the hollow center cavity at an angle of about −5 degrees, about 5 degrees, about −10 degrees, about 10 degrees, about −15 degrees, about 15 degrees, about −20 degrees, about 20 degrees, about −25 degrees, about 25 degrees, about −30 degrees, about 30 degrees, about −35 degrees, about 35 degrees, about −40 degrees, about 40 degrees, about −45 degrees, or about 45 degrees relative to the ground.

In certain embodiments, the methods provided by the present disclosure further comprise collecting the fraction of plant embryo explants. The collecting, in some embodiments, comprises collecting the fraction of plant embryo explants from the bottom interior region of the rotating cylinder. In particular embodiments, the methods provided by the present disclosure further comprise stopping the rotating of the rotating cylinder prior to collecting the fraction of plant embryo explants. In some embodiments, the purity of dry plant embryo explants in the fraction is increased by 0.5-fold to about 10-fold compared to the purity of dry plant embryo explants in the preparation, wherein the purity is defined as the percentage of dry plant embryo explants per particle.

In yet other aspects, the present disclosure provides a method of purifying genetically modifiable dry plant embryo explants, the method comprising aspirating within a first vertical chamber a preparation of dry plant embryo explants comprising meristematic tissue with a first upward air flow having a first air flow velocity of about 1.0 m/s to about 25.0 m/s, about 2.5 m/s to about 25.0 m/s, about 2.5 m/s to about 8.0 m/s, or about 3.1 m/s to about 7.2 m/s, wherein the preparation comprises a population of dry plant embryo explants and debris material; and separating a first fraction of the plant embryo explants of the preparation from a first portion of the debris material according to a displacement of the first fraction relative to a displacement of the first portion of the debris material produced by the first upward air flow within the first vertical chamber. In some embodiments, the method further comprises introducing the preparation into the first vertical chamber above a first aspiration screen positioned within the first vertical chamber, the first aspiration screen comprising a plurality of openings, each comprising a first opening size and a first opening shape, wherein the introducing comprises introducing the preparation into the first vertical chamber prior to aspirating within the first vertical chamber. In certain embodiments, the first aspiration screen comprises a top surface and a bottom surface, and the method comprises contacting the preparation or the population of dry plant embryo explants with the top surface of the first aspiration screen during the aspirating. The first aspiration screen, in particular embodiments, is structurally connected to the first vertical chamber. The first aspiration screen, in some embodiments, comprises a first end and a second end, wherein the first end is elevated relative to the second end to produce a first incline angle relative to the ground. In particular embodiments, the first incline angle is about 10° to about 60°, about 20° to about 60°, about 25° to about 55°, about 30° to about 50°, about 30° to about 45°, about 35° to about 45°, or about 35° to about 40°.

In some embodiments, the introducing comprises introducing the preparation into the first vertical chamber through a first input port positioned above the first aspiration screen. The first end of the first aspiration screen, in certain embodiments, is positioned within the first vertical chamber such that the first end is closer to the first input port compared to the second end, and the first end of the first aspiration screen is elevated relative to the second end to produce the first incline angle. The introducing, in particular embodiments, comprises introducing the preparation into the first vertical chamber from a vibratory feeding unit. The vibratory feeding unit, in some embodiments, is structurally connected to the first vertical chamber, and the vibratory feeding unit, in certain embodiments, produces a vibratory motion that causes movement of the preparation into the first vertical chamber. In some embodiments, the vibratory motion comprises a substantially horizontal vibratory motion. In particular embodiments, the introducing comprises introducing the preparation into the first vertical chamber at a rate of about 1 g/min to about 70 g/min, about 10 g/min to about 60 g/min, about 20 g/min to about 50 g/min, or about 30 g/min to about 40 g/min. In particular embodiments, the first vertical chamber comprises a top portion and a bottom portion, wherein the top portion is above the first aspiration screen, and the bottom portion is below the first aspiration screen, and the first upward air flow passes through the first aspiration screen from the bottom portion of the first vertical chamber to the top portion of the first vertical chamber.

In certain embodiments, the preparation comprises corn, wheat, soybean, cotton, or canola embryo explants. In some embodiments, the population comprises dry corn embryo explants, and the first air flow velocity is about 4.5 m/s to about 6.0 m/s, about 5.0 m/s to about 5.5 m/s, or about 5.1 m/s to about 5.3 m/s. In particular embodiments, the population comprises dry soybean embryo explants, and the first air flow velocity is about 4.0 m/s to about 5.5 m/s or about 4.5 m/s to about 5.0 m/s. In certain embodiments, the population comprises dry cotton embryo explants, and the first air flow velocity is about 5.5 m/s to about 8.0 m/s, about 5.5 m/s to about 7.5 m/s, or about 5.9 m/s to about 7.2 m/s. In some embodiments, the population comprises dry wheat embryo explants, and the first air flow velocity is about 2.5 m/s to about 4.0 m/s, about 3.0 m/s to about 3.5 m/s, or about 3.0 m/s to about 3.3 m/s. In particular embodiments, the population comprises dry canola embryo explants, and the first air flow velocity is about 2.5 to about 4.0 m/s, about 3.0 m/s to about 4.0 m/s, about 3.4 m/s to about 3.8 m/s, or about 3.6 m/s.

In certain embodiments, the method further comprises removing the first portion of the debris material separated from the first fraction through the top portion of the first vertical chamber. The removing, in some embodiments, comprises removing the first portion of the debris material through a first discharge port, wherein the first vertical chamber comprises an interior portion and an exterior portion, and wherein the top portion of the interior portion is in fluid communication with the first discharge port. In particular embodiments, the method further comprises collecting the first portion of the debris material in a first discharge collector, wherein the first vertical chamber comprises an interior portion and an exterior portion, and wherein the top portion of the interior portion is in fluid communication with the first discharge collector. In some embodiments, the top portion of the first vertical chamber is structurally connected to a first turned segment comprising an interior portion and an exterior portion, wherein the interior portion of the first turned segment is in fluid communication with the top portion of the interior portion of the first vertical chamber, and wherein the first upward air flow in the first vertical chamber is redirected to become a first redirected air flow in the first turned segment. The maximum angle between the direction of the first redirected air flow and the first upward air flow, in some embodiments, is at least 90°. In particular embodiments, the first aspiration screen comprises a first end and a second end, the first turned segment comprises a top end and a bottom end, the first end of the first aspiration screen is elevated relative to the second end to produce a first incline angle, and the vertical distance between the first end of the first aspiration screen and the bottom end of the interior portion of the first turned segment is about 12.7 cm to about 76.2 cm, about 12.7 cm to about 63.5 cm, about 12.7 cm to about 50.8 cm, about 25.4 cm to about 50.8 cm, or about 25.4 cm to about 38.1 cm.

In some embodiments, the method further comprises collecting the first fraction of the plant embryo explants from the top surface of the first aspiration screen, wherein the first portion of the debris material has been removed from the first fraction. In certain embodiments, the method further comprises transferring the first fraction of plant embryo explants through a first output port to a first output collector, wherein the first output port is positioned above the first aspiration screen; and collecting the first fraction in the first output collector, wherein the first portion of the debris material has been removed from the first fraction. In particular embodiments, the first aspiration screen comprises a first end and a second end, the second end of the first aspiration screen is positioned within the first vertical chamber such that the second end is closer to the first output port than is the first end, and the first end of the first aspiration screen is elevated relative to the second end to produce the first incline angle.

In some embodiments, the method further comprises transferring the first fraction of the plant embryo explants into a second vertical chamber, wherein the first portion of the debris material has been removed from the first fraction. In certain embodiments, the method further comprises transferring the first fraction of plant embryo explants through a first advancement port to a second vertical chamber, wherein the first advancement comprises an opening between the first vertical chamber and the second vertical chamber; and wherein the first advancement port is positioned above the first aspiration screen, wherein the first portion of the debris material has been removed from the first fraction. In some embodiments, the transferring comprises transferring the first fraction into the second vertical chamber above a second aspiration screen positioned within the second vertical chamber, wherein the second aspiration screen comprising a plurality of openings, each comprising a second opening size and a second opening shape. The first advancement port, in particular embodiments, is positioned above the second aspiration screen, and the transferring comprises transferring the first fraction through the first advancement port into the second vertical chamber above the second aspiration screen. In particular embodiments, the second aspiration screen comprises a first end and a second end, the first end of the second aspiration screen is positioned within the second vertical chamber such that the first end is closer to the first advancement port compared to the second end, and the first end of the second aspiration screen is elevated relative to the second end to produce a second incline angle.

In particular embodiments, the method further comprises aspirating within the second vertical chamber the first fraction with a second upward air flow having a second air flow velocity of about 1.0 m/s to about 25.0 m/s, about 2.5 m/s to about 25.0 m/s, about 3.0 m/s to about 8.5 m/s, or about 4.1 m/s to about 8.2 m/s; and separating a second fraction of the plant embryo explants comprised in the first fraction from a second portion of the debris material according to a displacement of the second fraction relative to a displacement of the second portion of the debris material produced by the second upward air flow within the second vertical chamber. In some embodiments, the method further comprises transferring the first fraction of dry plant embryo explants into the second vertical chamber above a second aspiration screen positioned within the second vertical chamber, the second aspiration screen comprising a plurality of openings, each comprising a second opening size and a second opening shape, wherein the transferring comprises transferring the first fraction into the second vertical chamber prior to aspirating within the second vertical chamber. In particular embodiments, the second aspiration screen comprises a top surface and a bottom surface, and the method comprises contacting the first fraction of dry plant embryo explants with the top surface during the aspirating. The second aspiration screen, in some embodiments, is structurally connected to the second vertical chamber. In certain embodiments, the second aspiration screen comprises a first end and a second end, and the first end is elevated relative to the second end to produce a second incline angle relative to the ground. The second incline angle, in particular embodiments, is about 10° to about 60°, about 20° to about 60°, about 25° to about 55°, about 30° to about 50°, about 30° to about 45°, about 35° to about 45°, or about 35° to about 40°. In some embodiments, the population or first fraction comprises dry corn embryo explants, and the second air flow velocity is about 5.5 to about 6.5 m/s or about 5.9 m/s to about 6.1 m/s. In certain embodiments, the population or first fraction comprises dry soybean embryo explants, and the second air flow velocity is about 5.0 m/s to about 7.0 m/s or about 5.5 m/s to about 6.5 m/s. In particular embodiments, the population or first fraction comprises dry cotton embryo explants, and the second air flow velocity is about 6.5 m/s to about 8.5 m/s or about 6.7 m/s to about 8.2 m/s. In some embodiments, the population or first fraction comprises dry wheat embryo explants, and the second air flow velocity is about 3.0 m/s to about 5.0 m/s, about 3.5 m/s to about 4.5 m/s, or about 3.8 m/s to about 4.3 m/s. In certain embodiments, the population or first fraction comprises dry canola embryo explants, and the second air flow velocity is about 4.0 m/s to about 5.5 m/s, about 4.0 m/s to about 5.0 m/s, about 4.3 m/s to about 4.9 m/s, or about 4.6 m/s. In some embodiments, the second vertical chamber comprises a top portion and a bottom portion, wherein the top portion is above the second aspiration screen, and the bottom portion is below the second aspiration screen, and wherein the second upward air flow passes through the second aspiration screen from the bottom portion of the second vertical chamber to the top portion of the second vertical chamber.

In some embodiments, the method further comprises removing the second portion of the debris material separated from the second fraction through the top portion of the second vertical chamber. In certain embodiments, the removing comprises removing the second portion of the debris material through a second discharge port, wherein the second vertical chamber comprises an interior portion and an exterior portion, and wherein the top portion of the interior portion is in fluid communication with the second discharge port. In particular embodiments, the method further comprises collecting the second portion of the debris material in a second discharge collector, wherein the second vertical chamber comprises an interior portion and an exterior portion, and wherein the top portion of the interior portion is in fluid communication with the second discharge collector. In some embodiments, the top portion of the second vertical chamber is structurally connected to a second turned segment comprising an interior portion and an exterior portion, wherein the interior portion of the second turned segment is in fluid communication with the interior portion of the top portion of the second vertical chamber, wherein the second upward air flow in the second vertical chamber is redirected to become a second redirected air flow in the second turned segment. The maximum angle between the direction of the second redirected air flow and the second upward air flow, in certain embodiments, is at least 90°. In some embodiments, the second aspiration screen comprises a first end and a second end, the second turned segment comprises a top end and a bottom end, the first end of the second aspiration screen is elevated relative to the second end to produce a second incline angle, and the vertical distance between the first end of the second aspiration screen and the bottom end of the interior portion of the second turned segment is about 12.7 cm to about 101.6 cm, about 12.7 cm to about 76.2 cm, about 25.4 cm to about 76.2 cm, about 38.1 cm to about 63.5 cm, or about 40.64 cm to about 55.88 cm.

In particular embodiments, the method further comprises collecting the second fraction of the plant embryo explants from the top surface of the second aspiration screen, wherein the second portion of the debris material has been removed from the second fraction. In certain embodiments, the method further comprises transferring the second fraction of plant embryo explants through a second output port to a second output collector, wherein the second output port is positioned above the second aspiration screen; and collecting the second fraction in the second output collector, wherein the second portion of the debris material has been removed from the second fraction. In particular embodiments, the second aspiration screen comprises a first and a second end, the second end of the second aspiration screen is positioned within the second vertical chamber such that the second end is closer to the second outport port compared to the first end, wherein the first end of the second aspiration screen is elevated relative to the second end to produce a second include angle.

In certain embodiments, the method further comprises transferring the second fraction of the plant embryo explants into a third vertical chamber, wherein the second portion of the debris material has been removed from the second fraction. In some embodiments, the method further comprises transferring the second fraction of the plant embryo explants through a second advancement port into a third vertical chamber, wherein the second advancement port comprises an opening between the second vertical chamber and the third vertical chamber, and wherein the second advancement port is positioned above the second aspiration screen, wherein the second fraction lacks the second portion of the debris material. The transferring, in certain embodiments, comprises transferring the second fraction into the third vertical chamber above a third aspiration screen positioned within the third vertical chamber, wherein the third aspiration screen comprises a plurality of openings, each comprising a third opening size and a third opening shape. In particular embodiments, the second advancement port is positioned above the third aspiration screen, and wherein the transferring comprises transferring the second fraction through the second advancement port and into the third vertical chamber above the third aspiration screen. In some embodiments, the third aspiration screen comprises a first end and a second end, wherein the first end of the third aspiration screen is positioned within the third vertical chamber such that the first end is closer to the second advancement port compared to the second end, and wherein the first end of the third aspiration screen is elevated compared to the second end to produce a third incline angle.

In some embodiments, the method further comprises aspirating within the third vertical chamber the second fraction with a third upward air flow having a third air flow velocity of about 1.0 m/s to about 25.0 m/s, about 2.5 m/s to about 25.0 m/s, about 4.5 m/s to about 12.5 m/s, or about 5.3 m/s to about 11.9 m/s; and separating a third fraction of the plant embryo explants comprised within the second fraction from a third portion of the debris material according to a displacement of the third fraction relative to a displacement of the third portion of the debris material produced by the third upward air flow within the third vertical chamber. In particular embodiments, the method further comprises transferring the second fraction of dry plant embryo explants into the third vertical chamber above a third aspiration screen positioned within the third vertical chamber, the third aspiration screen comprising a plurality of openings, each comprising a third opening size and a third opening shape, wherein the transferring comprises transferring the second fraction into the third vertical chamber prior to aspirating within the third vertical chamber. In some embodiments, the third aspiration screen comprises a top surface and a bottom surface and the method comprises contacting the second fraction with the top surface during the aspirating. The third aspiration screen, in particular embodiments, is structurally connected to the third vertical chamber. The third aspiration screen, in particular embodiments, comprises a first end and a second end, and the first end is elevated relative to the second end to produce a third incline angle. The third incline angle, in some embodiments, is about 10° to about 60°, about 20° to about 60°, about 25° to about 55°, about 30° to about 50°, about 30° to about 45°, about 35° to about 45°, or about 35° to about 40°. In certain embodiments, the population, first fraction or second fraction comprises corn embryo explants, and the third air flow velocity is about 6.5 m/s to about 7.5 m/s, about 7.0 m/s to about 7.5 m/s, or about 7.1 m/s to about 7.3 m/s. In particular embodiments, the population, first fraction or second fraction comprises soybean embryo explants, and the third air flow velocity is about 7.0 m/s to about 8.5 m/s, about 7.5 m/s to about 8.0 m/s, or about 7.6 m/s to about 7.9 m/s. In some embodiments, the population, first fraction or second fraction comprises cotton embryo explants, and the third air flow velocity is about 8.0 m/s to about 12.5 m/s, about 8.5 m/s to about 12.0 m/s, or about 8.8 m/s to about 11.9 m/s. In certain embodiments, the population, first fraction or second fraction comprises wheat embryo explants, and the third air flow velocity is about 4.5 m/s to about 6.0 m/s, about 5.0 m/s to about 6.0 m/s, or about 5.1 m/s to about 5.5 m/s. In particular embodiments, the population, first fraction or second fraction comprises canola embryo explants, and the third air flow velocity is about 5.0 m/s to about 7.0 m/s, about 5.5 m/s to about 6.5 m/s, or about 6.0 m/s. In some embodiments, the third vertical chamber comprises a top portion and a bottom portion, wherein the top portion is above the third aspiration screen, and the bottom portion is below the third aspiration screen, and wherein the third upward air flow passes through the third aspiration screen from the bottom portion of the third vertical chamber to the top portion of the third vertical chamber.

In certain embodiments, the method further comprises removing the third portion of the debris material separated from the third fraction through the top portion of the third vertical chamber. The removing, in some embodiments, comprises removing the third portion of the debris material through a third discharge port, wherein the third vertical chamber comprises an interior portion and an exterior portion, and wherein the top portion of the interior portion is in fluid communication with the third discharge port. In particular embodiments, the method further comprises collecting the third portion of the debris material in a third discharge collector, wherein the third vertical chamber comprises an interior portion and an exterior portion, and wherein the top portion of the interior portion is in fluid communication with the third discharge collector. In some embodiments, the top portion of the third vertical chamber is structurally connected to a third turned segment comprising an interior portion and an exterior portion, wherein the interior portion of the third turned segment is in fluid communication with of the top portion of the interior portion of the third vertical chamber, and wherein the third upward air flow in the third vertical chamber is redirected to become a third redirected air flow in the third turned segment. The maximum angle between the direction of the third redirected air flow and the third upward air flow, in particular embodiments, is at least 90°. In some embodiments, the third aspiration screen comprises a first end and a second end, the third turned segment comprises a top end and a bottom end, the first end of the third aspiration screen is elevated relative to the second end to produce a third incline angle, and the vertical distance between the first end of the third aspiration screen and the bottom end of the interior portion of the third turned segment is about 25.4 cm to about 152.4 cm, about 25.4 cm to about 127 cm, about 25.4 cm to about 101.6 cm, about 38.1 cm to about 88.9 cm, about 50.8 cm to about 76.2 cm, or about 55.88 cm to about 71.12 cm.

In particular embodiments, the method further comprises collecting the third fraction of the plant embryo explants from the top surface of the third aspiration screen, wherein the third portion of the debris material has been removed from the third fraction. In certain embodiments, the method further comprises transferring the third fraction of plant embryo explants through a third output port to a third output collector, wherein the third output collector is positioned above the third aspiration screen; and collecting the third fraction in the third output collector, wherein the third portion of the debris material has been removed from the third fraction. In some embodiments, the third aspiration screen comprises a first end and a second end, the second end of the third aspiration screen is positioned within the third vertical chamber such that the second end is closer to the third output port compared to the first end, and the first end of the third aspiration screen is elevated relative to the second end to produce the third incline angle. In certain embodiments, the method further comprises transferring the third fraction of plant embryo explants into a fourth vertical chamber, wherein the third portion of the debris material has been removed from the third fraction. In particular embodiments, transferring the third fraction of the plant embryo explants through a third advancement port into a fourth vertical chamber, wherein the third advancement port comprises an opening between the third vertical chamber and the fourth vertical chamber, and wherein the third advancement port is positioned above the third aspiration screen, wherein the third portion of the debris material has been removed from the third fraction. The transferring, in some embodiments, comprises transferring the third fraction into the fourth vertical chamber above a fourth aspiration screen positioned within the fourth vertical chamber, wherein the fourth aspiration screen comprises a plurality of openings, each comprising a fourth opening size and a fourth opening shape. In certain embodiments, the third advancement port is positioned above the fourth aspiration screen, and the transferring comprises transferring the third fraction through the third advancement port into the fourth vertical chamber above the fourth aspiration screen. In particular embodiments, the fourth aspiration screen comprises a first end and a second end, the first end of the fourth aspiration screen is positioned within the fourth vertical chamber such that the first end is closer to the third advancement port compared to the second end, and the first end of the fourth aspiration screen is elevated relative to the second end to produce a fourth incline angle.

In some embodiments, the method further comprises aspirating within a fourth vertical chamber the third fraction with a fourth upward air flow having a fourth air flow velocity of about 1.0 m/s to about 25.0 m/s, about 2.5 m/s to about 25.0 m/s, about 6.5 m/s to about 20.5 m/s, or about 7.5 m/s to about 19.9 m/s; and separating a fourth fraction of the plant embryo explants comprised within the third fraction from a fourth portion of the debris material according to a displacement of the fourth fraction relative to a displacement of the fourth portion of the debris material produced by the fourth upward air flow within the fourth vertical chamber. In particular embodiments, the method further comprises transferring the third fraction of dry plant embryo explants into the fourth vertical chamber above a fourth aspiration screen positioned within the fourth vertical chamber, the fourth aspiration screen comprising a plurality of openings, each comprising a fourth opening size and a fourth opening shape, wherein the transferring comprises transferring the third fraction into the fourth vertical chamber prior to aspirating within the fourth vertical chamber. In certain embodiments, the fourth aspiration screen comprises a top surface and a bottom surface, and the method comprises contacting the fourth fraction of the dry plant embryo explants with the top surface during the aspirating. The fourth aspiration screen, in particular embodiments, is structurally connected to the fourth vertical chamber. The fourth aspiration screen, in some embodiments, comprises a first end and a second end and the first end is elevated relative to the second end to produce a fourth incline angle relative to the ground. The fourth incline, in some embodiments, angle is about 10° to about 60°, about 20° to about 60°, about 25° to about 55°, about 30° to about 50°, about 30° to about 45°, about 35° to about 45°, or about 35° to about 40°. In certain embodiments, the population, first fraction, second fraction or third fraction comprises corn embryo explants, and the fourth air flow velocity is about 9.5 m/s to about 10.5 m/s or about 9.7 m/s to about 10.1 m/s. In particular embodiments, the population, first fraction, second fraction or third fraction comprises soybean embryo explants, and the fourth air flow velocity is about 10.5 m/s to about 12.5 m/s, about 10.5 m/s to about 12.0 m/s, or about 10.8 m/s to about 11.9 m/s. In some embodiments, the population, first fraction, second fraction or third fraction comprises cotton embryo explants, and the fourth air flow velocity is about 13.0 m/s to about 20.5 m/s, about 13.5 m/s to about 20.0 m/s, or about 13.7 m/s to about 19.9 m/s. In certain embodiments, the population, first fraction, second fraction or third fraction comprises wheat embryo explants, and the fourth air flow velocity is about 6.5 m/s to about 8.0 m/s, about 7.0 m/s to about 8.0 m/s, or about 7.2 m/s to about 7.7 m/s. In particular embodiments, the population, first fraction, second fraction or third fraction comprises canola embryo explants, and the fourth air flow velocity is about 8.0 m/s to about 9.5 m/s, about 8.5 m/s to about 9.0 m/s, or about 8.8 m/s. In some embodiments, the fourth vertical chamber comprises a top portion and a bottom portion, wherein the top portion is above the fourth aspiration screen, and the bottom portion is below the fourth aspiration screen, and wherein the fourth upward air flow passes through the fourth aspiration screen from the bottom portion of the fourth vertical chamber to the top portion of the fourth vertical chamber.

In particular embodiments, the method further comprises removing the fourth portion of the debris material separated from the fourth fraction through the top portion of the fourth vertical chamber. The removing, in certain embodiments, comprises removing the fourth portion of the debris material through a fourth discharge port, wherein the fourth vertical chamber comprises an interior portion and an exterior portion, and wherein the top portion of the interior portion is in fluid communication with the fourth discharge port. In some embodiments, the method further comprises collecting the fourth portion of the debris material in a fourth discharge collector, wherein the fourth vertical chamber comprises an interior portion and an exterior portion, and wherein the top portion of the interior portion is in fluid communication with the fourth discharge collector. In particular embodiments, the top portion of the fourth vertical chamber is structurally connected to a fourth turned segment comprising an interior portion and an exterior portion, wherein the interior portion of the fourth turned segment is in fluid communication with the interior portion of the top portion of the fourth vertical chamber, and wherein the fourth upward air flow in the fourth vertical chamber is redirected to become a fourth redirected air flow in the fourth turned segment. The maximum angle between the direction of the fourth redirected air flow and the fourth upward air flow, in certain embodiments, is at least 90°. In some embodiments, the fourth aspiration screen comprises a first end and a second end, wherein the fourth turned segment comprises a top end and a bottom end, wherein the first end of the fourth aspiration screen is elevated relative to the second end to produce a fourth incline angle, and wherein the vertical distance between the first end of the fourth aspiration screen and the bottom end of the interior portion of the fourth turned segment is about 12.7 cm to about 152.4 cm, about 25.4 cm to about 127 cm, about 38.1 cm to about 114.3 cm, about 50.8 cm to about 101.6 cm, about 63.5 cm to about 88.9 cm, or about 68.58 cm to about 83.82 cm.

In certain embodiments, the method further comprises collecting the fourth fraction of the plant embryo explants from the top surface of the fourth aspiration screen, wherein the fourth portion of the debris material has been removed from the fourth fraction. In some embodiments, the method further comprises transferring the fourth fraction of plant embryo explants through a fourth output port to a fourth output collector, wherein the fourth output port is positioned above the fourth aspiration screen; and collecting the fourth fraction in the fourth output collector, wherein the fourth portion of the debris material has been removed from the fourth fraction. The fourth aspiration screen, in particular embodiments, comprises a first end and a second end, wherein the second end of the fourth aspiration screen is positioned within the fourth vertical chamber such that the second end is closer to the fourth output port compared to the first end, and wherein the first end of the fourth aspiration screen is elevated relative to the second end to produce the fourth incline angle.

In particular embodiments, the first vertical chamber, the second vertical chamber, the third vertical chamber, or the fourth vertical chamber has an average horizontal cross-sectional area of about 32.258 cm² to about 645.16 cm², about 32.258 cm² to about 322.58 cm², about 64.516 cm² to about 322.58 cm², or about 96.774 cm² to about 258.064 cm². In some embodiments, the first opening shape, the second opening shape, the third opening shape, or the fourth opening shape is selected from the group consisting of a geometric shape, a rectangle, a square, a circle, and an oval. In certain embodiments, the first opening size, the second opening size, the third opening size, or the fourth opening size comprises a first opening diameter, a second opening diameter, a third opening diameter, or a fourth opening diameter; a first opening width, a second opening width, a third opening width, or a fourth opening width; or a first opening length, a second opening length, a third opening length, or a fourth opening length. In some embodiments, the first opening diameter, the first opening length, the first opening width, the second opening diameter, the second opening length, the second opening width, the third opening diameter, the third opening length, the third opening width, or the fourth opening diameter, the fourth opening length, or the fourth opening width is about 10 μm to about 400 μm, 20 μm to about 300 μm, 20 μm to about 200 μm, about 20 μm to about 150 μm, about 20 μm to about 120 μm, about 30 μm to about 120 μm, about 40 μm to about 120 μm, about 50 μm to about 110 μm, about 60 μm to about 100 μm, about 70 μm to about 90 μm, or about 75 μm to about 85 μm. The first discharge port, the second discharge port, the third discharge port, or the fourth discharge port, in some embodiments, is defined as a common or continuous discharge port. The first discharge collector, the second discharge collector, the third discharge collector, or the fourth discharge collector, in particular embodiments, is defined as a common or continuous discharge collector. In certain embodiments, the purity of the first fraction, the second fraction, the third fraction, or the fourth fraction is increased by about 0.5-fold to about 40-fold compared to the purity of embryo explants in the preparation, wherein the purity is defined as the percentage of dry embryo explants per particle In some embodiments, the purity of the fourth fraction is increased by about 0.5-fold to about 55-fold compared to the purity of the embryo explants in the preparation, or compared to the purity of the first fraction, the second fraction, or the third fraction, wherein the purity is defined as the percentage of dry embryo explants per particle.

In some embodiments, the first fraction of plant embryo explants demonstrates a first fraction buoyancy and the first portion of the debris material demonstrates a first debris buoyancy in the first upward air flow against the force of gravity, wherein the first fraction buoyancy and the first debris buoyancy are different, and wherein the first fraction buoyancy and the first debris buoyancy result in a different displacement of the first fraction compared to the displacement of the first portion of the debris material. In certain embodiments, the second fraction of plant embryo explants demonstrates a second fraction buoyancy and the second portion of the debris material demonstrates a second debris buoyancy in the second upward air flow against the force of gravity, wherein the second fraction buoyancy and the second debris buoyance are different, and wherein the second fraction buoyancy and the second debris buoyancy result in a different displacement of the second fraction compared to the displacement of the second portion of the debris material. In particular embodiments, the third fraction of plant embryo explants demonstrates a third fraction buoyancy and the third portion of the debris material demonstrates a third debris buoyancy in the third upward air flow against the force of gravity, wherein the third fraction buoyancy and the third debris buoyancy are different, and wherein the third fraction buoyancy and the third debris buoyancy result in a different displacement of the third fraction compared the displacement of the third portion of the debris material. In some embodiments, the fourth fraction of plant embryo explants demonstrates a fourth fraction buoyancy and the fourth portion of the debris material demonstrates a fourth debris buoyancy in the fourth upward air flow against the force of gravity, wherein the fourth fraction buoyancy and the fourth debris buoyancy are different, and wherein the fourth fraction buoyancy and the fourth debris buoyancy result in a different displacement of the fourth fraction compared to the displacement of the fourth portion of the debris material.

In yet other aspects, the present disclosure provides, a method of purifying genetically modifiable dry plant embryo explants, the method comprising aspirating within a vertical chamber a preparation of dry plant embryo explants comprising meristematic tissue with an upward flow having an air velocity of about 2.0 m/s to about 10.0 m/s, wherein the preparation comprises a population of dry plant embryo explants and debris material; and separating a fraction of the plant embryo explants of the preparation from a portion of the debris material according to a displacement of the fraction relative to a displacement of the portion of the debris material produced by the air flow within the vertical chamber, wherein the vertical chamber is in fluid communication with a turned segment a waste collector. In some embodiments, the method further comprises introducing the preparation into the vertical chamber above an aspiration screen positioned within an input compartment, the aspiration screen comprising a plurality of openings, each comprising an opening size and an opening shape, wherein the introducing comprises introducing the preparation into the vertical chamber prior to aspirating within the vertical chamber. In certain embodiments, the aspiration screen comprises a top surface and a bottom surface, and the method comprises contacting the preparation or the population of dry plant embryo explants with the top surface of the aspiration screen during the aspirating. The turned segment, in particular embodiments, is structurally connected to the vertical chamber. The waste collector, in certain embodiments, is structurally connected to the vertical chamber. The input compartment, in some embodiments, is structurally connected to the vertical chamber. In particular embodiments, the introducing comprises introducing the preparation into the vertical chamber through the input compartment. The preparation, in certain embodiments, comprises corn, wheat, soybean, cotton, or canola embryo explants. In some embodiments, the preparation comprises wheat embryo explants and the air velocity is about 2.5 m/s to about 8.5 m/s, about 3.0 m/s to about 8.0 m/s, or about 3.0 m/s to about 7.8 m/s. In particular embodiments, the preparation comprises canola embryo explants and the air velocity is about 3.0 m/s to about 10.0 m/s, about 3.0 m/s to about 9.5 m/s, or about 3.4 m/s to about 9.1 m/s. In certain embodiments, the vertical chamber is above the aspiration screen, and the upward air flow passes through the aspiration screen to the vertical chamber.

In certain embodiments, the method further comprises removing the portion of the debris material separated from the fraction of plant embryo explants through the turned segment. The vertical chamber, in some embodiments, comprises an interior portion and an exterior portion, wherein the interior portion is in fluid communication with the turned segment. In particular embodiments, the method further comprises collecting the portion of the debris material in the waste collector, wherein the vertical chamber comprises an interior portion and an exterior portion, and wherein the interior portion is in fluid communication with the waste collector. In certain embodiments, the vertical chamber is structurally connected to the turned segment, the turned segment comprises an interior portion and an exterior portion, the interior portion of the turned segment is in fluid communication with the interior portion of the vertical chamber, and the upward air flow in the vertical chamber is redirected to become a redirected air flow in the turned segment. The maximum angle between the direction of the redirected air flow and the upward air flow, in some embodiments, is at least 90°. In particular embodiments, the turned segment comprises a top end and a bottom end, and the vertical distance between the aspiration screen and the bottom end of the interior portion of the turned segment is about 20 cm to about 120 cm, about 20 cm to about 100 cm, about 30 cm to about 90 cm, about 40 cm to about 80 cm, about 50 cm to about 70 cm, or about 55 cm to about 65 cm.

In some embodiments, the method further comprises collecting the fraction of the plant embryo explants from the top surface of the aspiration screen, wherein the portion of the debris material has been removed from the fraction. In certain embodiments, the method further comprises transferring the fraction of plant embryo explants through an output port to an output collector, wherein the output port is positioned above the aspiration screen; and collecting the fraction in the output collector, wherein the portion of the debris material has been removed from the fraction. The vertical chamber, in some embodiments, has an average horizontal cross-sectional area of about 10.0 cm² to about 100.0 cm², about 10.0 cm² to about 90.0 cm², about 10.0 cm² to about 80.0 cm², about 10.0 cm² to about 70.0 cm², about 10.0 cm² to about 60.0 cm², about 10.0 cm² to about 50.0 cm², about 10.0 cm² to about 60.0 cm², about 10.0 cm² to about 50.0 cm², about 10.0 cm² to about 40.0 cm², about 10.0 cm² to about 30.0 cm², about 15.0 cm² to about 30.0 cm², about 20.0 cm² to about 30.0 cm², or about 22.0 cm² to about 26.0 cm². The opening shape, in certain embodiments, is selected from the group consisting of a geometric shape, a rectangle, a square, a circle, and an oval. The opening size, in particular embodiments, comprises an opening diameter, an opening width, or an opening length. In some embodiments, the opening diameter, the opening length, or the opening width is about 10 μm to about 500 μm, about 10 μm to about 400 μm, 20 μm to about 300 μm, 20 μm to about 200 μm, about 20 μm to about 150 μm, about 20 μm to about 120 μm, about 30 μm to about 120 μm, about 40 μm to about 120 μm, about 50 μm to about 110 μm, about 60 μm to about 100 μm, about 70 μm to about 90 μm, or about 75 μm to about 85 μm. In particular embodiments, the fraction of plant embryo explants demonstrates a fraction buoyancy, and the portion of the debris material demonstrates a debris buoyancy in the upward air flow against the force of gravity, wherein the fraction buoyancy and the debris buoyancy are different, and wherein the fraction buoyancy and the debris buoyancy result in a different displacement of the fraction compared to the displacement of the portion of the debris material. In certain embodiments, the purity of the fraction is increased by about 0.5-fold to about 55-fold compared to the purity of embryo explants in the preparation, wherein the purity is defined as the percentage of dry embryo explants per particle.

In still yet other aspects, the present disclosure provides a method of purifying genetically modifiable dry plant embryo explants, the method comprising aspirating within a first functional unit of a vertical chamber a preparation of dry plant embryo explants comprising meristematic tissue with a first air flow having a first air flow velocity, wherein the preparation comprises a population of dry plant embryo explants and debris material; and separating a first fraction of the plant embryo explants of the preparation from a first portion of the debris material within the first functional unit of the vertical chamber according to a displacement of the first fraction relative to a displacement of the first portion of the debris material produced by the first air flow within the first functional unit, wherein the first air flow comprises a variable vertical component and a variable horizontal component, wherein the first functional unit of the vertical chamber comprises a first lower partition, a first air input port, and a first air output port, wherein the first lower partition extends inward from a side wall of the vertical chamber to define a first lower advancement port between the first lower partition and an opposite side wall of the vertical chamber, wherein the first air input port comprises an opening in the side wall of the vertical chamber below the first lower partition, and wherein the first air flow at least partially enters the vertical chamber through the first air input port, travels through the first lower advancement port, and exits the vertical chamber through the first air output port. In some embodiments, the first air output port comprises an opening in the opposite side wall of the vertical chamber positioned above the first lower partition or the first air input port. In certain embodiments, the first functional unit of the vertical chamber further comprises a first upper partition, wherein the first upper partition extends inward from the opposite side wall of the vertical chamber to define a first upper advancement port between the first upper partition and the side wall of the vertical chamber, wherein the first upper partition is positioned above the first lower partition or the first air input port. The first air output port, in particular embodiments, is positioned below the first upper partition. In some embodiments, the method further comprises introducing the preparation of dry plant embryo explants into the first functional unit of the vertical chamber. In particular embodiments, the method further comprises introducing the preparation of dry plant embryo explants into the first functional unit of the vertical chamber above the first upper partition. In certain embodiments, the method comprising contacting the preparation of dry plant embryo explants or a portion thereof with a top surface of the first upper partition before gravity causes the preparation or the portion thereof to fall through the first upper advancement port. The method, in some embodiments, comprises transferring the first fraction of plant embryo explants through the first lower advancement port by gravity. The method, in particular embodiments, comprises contacting the preparation of dry plant embryo explants or the portion thereof with a top surface of the first lower partition before transferring the first fraction through the first lower advancement port by gravity.

In some embodiments, the method further comprises removing the first portion of the debris material separated from the first fraction through the first air output port. The first portion of the debris material, in certain embodiments, travels with the first air flow through the first air output port. In particular embodiments, the first functional unit of the vertical chamber further comprises a first air intake partition, wherein the first air intake partition extends inward from the side wall of the vertical chamber to further define the first lower advancement port between the first air intake partition and the opposite side wall of the vertical chamber, wherein the first air input port is positioned above the first air intake partition such that the first air flow at least partially entering the vertical chamber through the first air input port is channeled between the first lower partition and the first air intake partition. The first upper partition, in some embodiments, extends inward from the opposite side wall at a first upper slope angle. The first lower partition, in certain embodiments, extends inward from the side wall at a first lower slope angle. The first air intake partition, in particular embodiments, extends inward from the side wall at a first intake slope angle. In some embodiments, the first upper slope angle, the first lower slope angle, or the first intake slope angle is a negative angle relative to horizontal. The vertical chamber, in certain embodiments, comprises a center cavity. In some embodiments, the first upper partition comprises a first end and a second end, the first end is attached to the opposite side wall and the second end extends into the center cavity of the vertical chamber, and the first end is elevated relative to the second end to create a downward slope angle from the opposite side wall. In particular embodiments, the first lower partition comprises a first end and a second end, the first end is attached to the side wall and the second end extends into the center cavity of the vertical chamber, and the first end is elevated relative to the second end to create a downward slope angle from the side wall. In certain embodiments, the first air intake partition comprises a first end and a second end, the first end is attached to the side wall and the second end extends into the center cavity of the vertical chamber, and the first end is elevated relative to the second end to create a downward slope angle from the side wall.

In certain embodiments, the method further comprises collecting the first fraction of the plant embryo explants from the first functional unit, wherein the first portion of the debris material has been removed from the first fraction. In particular embodiments, the method further comprises transferring the first fraction of the plant embryo explants through the first lower advancement port into a second functional unit, wherein the first lower advancement port is between the first functional unit and the second functional unit, and wherein the first functional unit is positioned above the second functional unit, and wherein the first portion of the debris material has been removed from the first fraction.

In some embodiments, the method further comprises aspirating within the second functional unit of the vertical chamber the first fraction of plant embryo explants with a second air flow having a second air flow velocity; and separating a second fraction of the plant embryo explants comprised in the first fraction from a second portion of the debris material within the second functional unit of the vertical chamber according to a displacement of the second fraction relative to a displacement of the second portion of the debris material produced by the second air flow within the second functional unit, wherein the second air flow comprises a variable vertical component and a variable horizontal component, wherein the second functional unit of the vertical chamber comprises a second lower partition, a second air input port, and a second air output port, wherein the second lower partition extends inward from the side wall of the vertical chamber to define a second lower advancement port between the second lower partition and the opposite side wall of the vertical chamber, wherein the second air input port comprises an opening in the side wall of the vertical chamber below the second lower partition, and wherein the second air flow at least partially enters the vertical chamber through the second air input port, travels through the second lower advancement port, and exits the vertical chamber through the second air output port. The second air output port, in some embodiments, comprises an opening in the opposite side wall of the vertical chamber positioned above the second lower partition or the second air input port. In particular embodiments, the second functional unit of the vertical chamber further comprises a second upper partition, wherein the second upper partition extends inward from the opposite side wall of the vertical chamber, and wherein the second upper partition is positioned above the second lower partition or the second air input port. The second air output port, in certain embodiments, is positioned below the second upper partition. In some embodiments, the method further comprises introducing the first fraction of dry plant embryo explants into the second functional unit of the vertical chamber. In particular embodiments, the method further comprises introducing the first fraction of dry plant embryo explants into the second functional unit of the vertical chamber above the second upper partition. In some embodiments, the method comprises contacting the first fraction of dry plant embryo explants or a portion thereof with a top surface of the second upper partition before gravity causes the first fraction or the portion thereof to fall through the first lower advancement port. In certain embodiments, the method comprises transferring the second fraction of plant embryo explants through the second lower advancement port by gravity. In particular embodiments, the method comprises contacting the first fraction of dry plant embryo explants or the portion thereof with a top surface of the second lower partition before transferring the second fraction through the second lower advancement port by gravity.

In certain embodiments, the method further comprises removing the second portion of the debris material separated from the second fraction through the second air output port. The second portion of the debris material, in some embodiments, travels with the second air flow through the second air output port. In particular embodiments, the second functional unit of the vertical chamber further comprises a second air intake partition, wherein the second air intake partition extends inward from the side wall of the vertical chamber to further define the second lower advancement port between the second air intake partition and the opposite side wall of the vertical chamber, wherein the second air input port is positioned above the second air intake partition such that the second air flow at least partially entering the vertical chamber through the second air input port is channeled between the second lower partition and the second air intake partition. In some embodiments, the second upper partition extends inward from the opposite side wall at a second upper slope angle. In certain embodiments, the second lower partition extends inward from the side wall at a second lower slope angle. In particular embodiments, and the second air intake partition extends inward from the side wall at a second intake slope angle. In some embodiments, the second upper slope angle, the second lower slope angle, or the second intake slope angle is a negative angle relative to horizontal. In particular embodiments, the second upper partition comprises a first end and a second end, wherein the first end is attached to the opposite side wall and the second end extends into the center cavity of the vertical chamber, wherein the first end is elevated relative to the second end to create a downward slope angle from the opposite side wall. In certain embodiments, the second lower partition comprises a first end and a second end, wherein the first end is attached to the side wall and the second end extends into the center cavity of the vertical chamber, wherein the first end is elevated relative to the second end to create a downward slope angle from the side wall. In some embodiments, the second air intake partition comprises a first end and a second end, wherein the first end is attached to the side wall and the second end extends into the center cavity of the vertical chamber, wherein the first end is elevated relative to the second end to create a downward slope angle from the side wall. In particular embodiments, the method further comprises collecting the second fraction of the plant embryo explants from the second functional unit, wherein the second portion of the debris material has been removed from the second fraction. In certain embodiments, the method further comprises transferring the second fraction of the plant embryo explants through the second lower advancement port into a third functional unit, wherein the second lower advancement port is between the second functional unit and the third functional unit, and wherein the second functional unit is positioned above the third functional unit, wherein the second portion of the debris material has been removed from the second fraction.

In particular embodiments, the method further comprises aspirating within the third functional unit of the vertical chamber the second fraction of plant embryo explants with a third air flow having a third air flow velocity; and separating a third fraction of the plant embryo explants comprised in the second fraction from a third portion of the debris material within the third functional unit of the vertical chamber according to a displacement of the third fraction relative to a displacement of the third portion of the debris material produced by the third air flow within the third functional unit, wherein the third air flow comprises a variable vertical component and a variable horizontal component, wherein the third functional unit of the vertical chamber comprises a third lower partition, a third air input port, and a third air output port, wherein the third lower partition extends inward from the side wall of the vertical chamber to define a third lower advancement port between the third lower partition and the opposite side wall of the vertical chamber, wherein the third air input port comprises an opening in the side wall of the vertical chamber below the third lower partition, and wherein the third air flow at least partially enters the vertical chamber through the third air input port, travels through the third lower advancement port, and exits the vertical chamber through the third air output port. In some embodiments, the third air output port comprises an opening in the opposite side wall of the vertical chamber positioned above the third lower partition or the third air input port. The third functional unit of the vertical chamber, in certain embodiments, further comprises a third upper partition, wherein the third upper partition extends inward from the opposite side wall of the vertical chamber, and wherein the third upper partition is positioned above the third lower partition or the third air input port. The third air output port, in particular embodiments, is positioned below the third upper partition. In some embodiments, the method further comprises introducing the second fraction of dry plant embryo explants into the third functional unit of the vertical chamber. In certain embodiments, the method further comprises introducing the second fraction of dry plant embryo explants into the third functional unit of the vertical chamber above the third upper partition. The method, in certain embodiments, comprises contacting the second fraction of dry plant embryo explants or a portion thereof with a top surface of the third upper partition before gravity causes the second fraction or the portion thereof to fall through the second lower advancement port.

The method, in particular embodiments, comprises transferring the third fraction of plant embryo explants through the third lower advancement port by gravity. In some embodiments, the method comprises contacting the second fraction of dry plant embryo explants or the portion thereof with a top surface of the third lower partition before transferring the third fraction through the third lower advancement port by gravity. In particular embodiments, the method comprises removing the third portion of the debris material separated from the third fraction through the third air output port. The third portion of the debris material, in certain embodiments, travels with the third air flow through the third air output port. In some embodiments, the third functional unit of the vertical chamber further comprises a third air intake partition, wherein the third air intake partition extends inward from the side wall of the vertical chamber to further define the third lower advancement port between the third air intake partition and the opposite side wall of the vertical chamber, wherein the third air input port is positioned above the third air intake partition such that the third air flow at least partially entering the vertical chamber through the third air input port is channeled between the third lower partition and the third air intake partition. The third upper partition, in certain embodiments, extends inward from the opposite side wall at a third upper slope angle. The third lower partition, in some embodiments, extends inward from the side wall at a third lower slope angle. The third air intake partition, in particular embodiments, extends inward from the side wall at a third intake slope angle. In some embodiments, the third upper slope angle, the third lower slope angle, or the third intake slope angle is a negative angle relative to horizontal. In particular embodiments, the third upper partition comprises a first end and a second end, wherein the first end is attached to the opposite side wall and the second end extends into the center cavity of the vertical chamber, wherein the first end is elevated relative to the second end to create a downward slope angle from the opposite side wall. In certain embodiments, the third lower partition comprises a first end and a second end, wherein the first end is attached to the side wall and the second end extends into the center cavity of the vertical chamber, wherein the first end is elevated relative to the second end to create a downward slope angle from the side wall. In some embodiments, the third air intake partition comprises a first end and a second end, wherein the first end is attached to the side wall and the second end extends into the center cavity of the vertical chamber, wherein the first end is elevated relative to the second end to create a downward slope angle from the side wall.

In some embodiments, the method further comprises collecting the third fraction of the plant embryo explants from the third functional unit, wherein the third portion of the debris material has been removed from the third fraction. In certain embodiments, the method further comprises transferring the third fraction of the plant embryo explants through the third lower advancement port into a fourth functional unit, wherein the third lower advancement port is between the third functional unit and the fourth functional unit, and wherein the third functional unit is positioned above the fourth functional unit, wherein the third portion of the debris material has been removed from the third fraction.

In particular embodiments, the method further comprises aspirating within the fourth functional unit of the vertical chamber the third fraction of plant embryo explants with a fourth air flow having a fourth air flow velocity; and separating a fourth fraction of the plant embryo explants comprised in the third fraction from a fourth portion of the debris material within the fourth functional unit of the vertical chamber according to a displacement of the fourth fraction relative to a displacement of the fourth portion of the debris material produced by the fourth air flow within the fourth functional unit, wherein the fourth air flow comprises a variable vertical component and a variable horizontal component, wherein the fourth functional unit of the vertical chamber comprises a fourth lower partition, a fourth air input port, and a fourth air output port, wherein the fourth lower partition extends inward from the side wall of the vertical chamber to define a fourth lower advancement port between the fourth lower partition and the opposite side wall of the vertical chamber, wherein the fourth air input port comprises an opening in the side wall of the vertical chamber below the fourth lower partition, and wherein the fourth air flow at least partially enters the vertical chamber through the fourth air input port, travels through the fourth lower advancement port, and exits the vertical chamber through the fourth air output port. The fourth air output port, in some embodiments, comprises an opening in the opposite side wall of the vertical chamber positioned above the fourth lower partition or the fourth air input port. In certain embodiments, the fourth functional unit of the vertical chamber further comprises a fourth upper partition, wherein the fourth upper partition extends inward from the opposite side wall of the vertical chamber, and wherein the fourth upper partition is positioned above the fourth lower partition or the fourth air input port. The fourth air output port, in particular embodiments, is positioned below the fourth upper partition.

In some embodiments, the method further comprises introducing the third fraction of dry plant embryo explants into the fourth functional unit of the vertical chamber. In certain embodiments, the method further comprises introducing the third fraction of dry plant embryo explants into the fourth functional unit of the vertical chamber above the fourth upper partition. In particular embodiments, the method further comprises contacting the third fraction of dry plant embryo explants or a portion thereof with a top surface of the fourth upper partition before gravity causes the third fraction or the portion thereof to fall through the third lower advancement port. The method comprises, in some embodiments, transferring the fourth fraction of plant embryo explants through the fourth lower advancement port by gravity. In certain embodiments, the method comprises contacting the third fraction of dry plant embryo explants or the portion thereof with a top surface of the fourth lower partition before transferring the fourth fraction through the fourth lower advancement port by gravity. The method further comprises, in particular embodiments, removing the fourth portion of the debris material separated from the fourth fraction through the fourth air output port. The fourth portion of the debris material, in some embodiments, travels with the fourth air flow through the fourth air output port.

In certain embodiments, the fourth functional unit of the vertical chamber further comprises a fourth air intake partition, wherein the fourth air intake partition extends inward from the side wall of the vertical chamber to further define the fourth lower advancement port between the fourth air intake partition and the opposite side wall of the vertical chamber, wherein the fourth air input port is positioned above the fourth air intake partition such that the fourth air flow at least partially entering the vertical chamber through the fourth air input port is channeled between the fourth lower partition and the fourth air intake partition. The fourth upper partition, in some embodiments, extends inward from the opposite side wall at a fourth upper slope angle. The fourth lower partition, in particular embodiments, extends inward from the side wall at a fourth lower slope angle. The fourth air intake partition, in certain embodiments, extends inward from the side wall at a fourth intake slope angle. In some embodiments, the fourth upper slope angle, the fourth lower slope angle, or the fourth intake slope angle is a negative angle relative to horizontal. In particular embodiments, the fourth upper partition comprises a first end and a second end, wherein the first end is attached to the opposite side wall and the second end extends into the center cavity of the vertical chamber, wherein the first end is elevated relative to the second end to create a downward slope angle from the opposite side wall. In certain embodiments, the fourth lower partition comprises a first end and a second end, wherein the first end is attached to the side wall and the second end extends into the center cavity of the vertical chamber, wherein the first end is elevated relative to the second end to create a downward slope angle from the side wall. In some embodiments, the fourth air intake partition comprises a first end and a second end, wherein the first end is attached to the side wall and the second end extends into the center cavity of the vertical chamber, wherein the first end is elevated relative to the second end to create a downward slope angle from the side wall. In some embodiments, the method further comprises collecting the fourth fraction of the plant embryo explants from the fourth functional unit, wherein the fourth portion of the debris material has been removed from the fourth fraction. In particular embodiments, the method further comprises transferring the fourth fraction of the plant embryo explants through the fourth lower advancement port into a fifth functional unit, wherein the fourth lower advancement port is between the fourth functional unit and the fifth functional unit, and wherein the fourth functional unit is positioned above the fifth functional unit, wherein the fourth portion of the debris material has been removed from the fourth fraction.

In particular embodiments, the method further comprises aspirating within the fifth functional unit of the vertical chamber the fourth fraction of plant embryo explants with a fifth air flow having a fifth air flow velocity; and separating a fifth fraction of the plant embryo explants comprised in the fourth fraction from a fifth portion of the debris material within the fifth functional unit of the vertical chamber according to a displacement of the fifth fraction relative to a displacement of the fifth portion of the debris material produced by the fifth air flow within the fifth functional unit, wherein the fifth air flow comprises a variable vertical component and a variable horizontal component, wherein the fifth functional unit of the vertical chamber comprises a fifth lower partition, a fifth air input port, and a fifth air output port, wherein the fifth lower partition extends inward from the side wall of the vertical chamber to define a fifth lower advancement port between the fifth lower partition and the opposite side wall of the vertical chamber, wherein the fifth air input port comprises an opening in the side wall of the vertical chamber below the fifth lower partition, and wherein the fifth air flow at least partially enters the vertical chamber through the fifth air input port, travels through the fifth lower advancement port, and exits the vertical chamber through the fifth air output port. The fifth air output port, in some embodiments, comprises an opening in the opposite side wall of the vertical chamber positioned above the fifth lower partition or the fifth air input port. In certain embodiments, the fifth functional unit of the vertical chamber further comprises a fifth upper partition, wherein the fifth upper partition extends inward from the opposite side wall of the vertical chamber, and wherein the fifth upper partition is positioned above the fifth lower partition or the fifth air input port. The fifth air output port, in particular embodiments, is positioned below the fifth upper partition.

In certain embodiments, the method further comprises introducing the fourth fraction of dry plant embryo explants into the fifth functional unit of the vertical chamber. In some embodiments, the method further comprises introducing the fourth fraction of dry plant embryo explants into the fifth functional unit of the vertical chamber above the fifth upper partition. In particular embodiments, the method comprises contacting the fourth fraction of dry plant embryo explants or a portion thereof with a top surface of the fifth upper partition before gravity causes the fourth fraction or the portion thereof to fall through the fourth lower advancement port. In some embodiments, the method comprises transferring the fifth fraction of plant embryo explants through the fifth lower advancement port by gravity. In certain embodiments, the method comprises contacting the fourth fraction of dry plant embryo explants or the portion thereof with a top surface of the fifth lower partition before transferring the fifth fraction through the fifth lower advancement port by gravity. The method further comprises, in some embodiments, removing the fifth portion of the debris material separated from the fifth fraction through the fifth air output port. The fifth portion of the debris material, in particular embodiments, travels with the fifth air flow through the fifth air output port.

In some embodiments, the fifth functional unit of the vertical chamber further comprises a fifth air intake partition, wherein the fifth air intake partition extends inward from the side wall of the vertical chamber to further define the fifth lower advancement port between the fifth air intake partition and the opposite side wall of the vertical chamber, wherein the fifth air input port is positioned above the fifth air intake partition such that the fifth air flow at least partially entering the vertical chamber through the fifth air input port is channeled between the fifth lower partition and the fifth air intake partition. The fifth upper partition, in certain embodiments, extends inward from the opposite side wall at a fifth upper slope angle. The fifth lower partition, in particular embodiments, extends inward from the side wall at a fifth lower slope angle. The fifth air intake partition, in some embodiments, extends inward from the side wall at a fifth intake slope angle. In certain embodiments, the fifth upper slope angle, the fifth lower slope angle, or the fifth intake slope angle is a negative angle relative to horizontal. The fifth upper partition, in some embodiments, comprises a first end and a second end, wherein the first end is attached to the opposite side wall and the second end extends into the center cavity of the vertical chamber, wherein the first end is elevated relative to the second end to create a downward slope angle from the opposite side wall. The fifth lower partition, in certain embodiments, comprises a first end and a second end, wherein the first end is attached to the side wall and the second end extends into the center cavity of the vertical chamber, wherein the first end is elevated relative to the second end to create a downward slope angle from the side wall. The fifth air intake partition, in particular embodiments, comprises a first end and a second end, wherein the first end is attached to the side wall and the second end extends into the center cavity of the vertical chamber, wherein the first end is elevated relative to the second end to create a downward slope angle from the side wall.

In particular embodiments, the method further comprises collecting the fifth fraction of the plant embryo explants from the fifth functional unit, wherein the fifth portion of the debris material has been removed from the fifth fraction. In certain embodiments, the method further comprises transferring the fifth fraction of the plant embryo explants through the fifth lower advancement port into a sixth functional unit, wherein the fifth lower advancement port is between the fifth functional unit and the sixth functional unit, and wherein the fifth functional unit is positioned above the sixth functional unit, wherein the fifth portion of the debris material has been removed from the fifth fraction.

The method, in some embodiments, further comprises aspirating within the sixth functional unit of the vertical chamber the fifth fraction of plant embryo explants with a sixth air flow having a sixth air flow velocity; and separating a sixth fraction of the plant embryo explants comprised in the fifth fraction from a sixth portion of the debris material within the sixth functional unit of the vertical chamber according to a displacement of the sixth fraction relative to a displacement of the sixth portion of the debris material produced by the sixth air flow within the sixth functional unit, wherein the sixth air flow comprises a variable vertical component and a variable horizontal component, wherein the sixth functional unit of the vertical chamber comprises a sixth lower partition, a sixth air input port, and a sixth air output port, wherein the sixth lower partition extends inward from the side wall of the vertical chamber to define a lower collection port between the sixth lower partition and the opposite side wall of the vertical chamber, wherein the sixth air input port comprises an opening in the side wall of the vertical chamber below the sixth lower partition, and wherein the sixth air flow at least partially enters the vertical chamber through the sixth air input port, travels through the lower collection port, and exits the vertical chamber through the sixth air output port. In certain embodiments, the sixth air output port comprises an opening in the opposite side wall of the vertical chamber positioned above the sixth lower partition or the sixth air input port. In particular embodiments, the sixth functional unit of the vertical chamber further comprises a sixth upper partition, wherein the sixth upper partition extends inward from the opposite side wall of the vertical chamber, and wherein the sixth upper partition is positioned above the sixth lower partition or the sixth air input port. The sixth air output port, in some embodiments, is positioned below the sixth upper partition.

The method, in particular embodiments, further comprises introducing the fifth fraction of dry plant embryo explants into the sixth functional unit of the vertical chamber. The method, in certain embodiments, further comprises introducing the fifth fraction of dry plant embryo explants into the sixth functional unit of the vertical chamber above the sixth upper partition. The method comprises, in certain embodiments, contacting the fifth fraction of dry plant embryo explants or a portion thereof with a top surface of the sixth upper partition before gravity causes the fifth fraction or the portion thereof to fall through the fifth lower advancement port. In some embodiments, the method comprises transferring the sixth fraction of plant embryo explants through the lower collection port by gravity. The method further comprises, in some embodiments, removing the sixth portion of the debris material separated from the sixth fraction through the sixth air output port. The sixth portion of the debris material, in particular embodiments, travels with the sixth air flow through the sixth air output port.

In some embodiments, the sixth functional unit of the vertical chamber further comprises a sixth air intake partition, wherein the sixth air intake partition extends inward from the side wall of the vertical chamber to further define the lower collection port between the sixth air intake partition and the opposite side wall of the vertical chamber, wherein the sixth air input port is positioned above the sixth air intake partition such that the sixth air flow at least partially entering the vertical chamber through the sixth air input port is channeled between the sixth lower partition and the sixth air intake partition. The sixth upper partition, in certain embodiments, extends inward from the opposite side wall at a sixth upper slope angle. The sixth lower partition, in particular embodiments, extends inward from the side wall at a sixth lower slope angle. The sixth air intake partition, in some embodiments, extends inward from the side wall at a sixth intake slope angle. In certain embodiments, the sixth upper slope angle, the sixth lower slope angle, or the sixth intake slope angle is a negative angle relative to horizontal. In some embodiments, the sixth upper partition comprises a first end and a second end, wherein the first end is attached to the opposite side wall and the second end extends into the center cavity of the vertical chamber, wherein the first end is elevated relative to the second end to create a downward slope angle from the opposite side wall. In certain embodiments, the sixth lower partition comprises a first end and a second end, wherein the first end is attached to the side wall and the second end extends into the center cavity of the vertical chamber, wherein the first end is elevated relative to the second end to create a downward slope angle from the side wall. In particular embodiments, the sixth air intake partition comprises a first end and a second end, wherein the first end is attached to the side wall and the second end extends into the center cavity of the vertical chamber, wherein the first end is elevated relative to the second end to create a downward slope angle from the side wall. The method further comprises, in particular embodiments, collecting the sixth fraction of the plant embryo explants from the sixth functional unit, wherein the sixth portion of the debris material has been removed from the sixth fraction. The method further comprises, in certain embodiments, transferring the sixth fraction of the plant embryo explants through the lower collection port; and collecting the sixth fraction, wherein the sixth portion of the debris material has been removed from the sixth fraction.

In particular embodiments, the first upper slope angle, the first lower slope angle, the first intake slope angle, the second upper slope angle, the second lower slope angle, the second intake slope angle, the third upper slope angle, the third lower slope angle, the third intake slope angle, the fourth upper slope angle, the fourth lower slope angle, the fourth intake slope angle, the fifth upper slope angle, the fifth lower slope angle, the fifth intake slope angle, the sixth upper slope angle, the sixth lower slope angle, or the sixth intake slope angle is about −20 degrees to about −50 degrees, about −25 degrees to about −45 degrees, or about −30 degrees to about −40 degrees relative to horizontal. In certain embodiments, the first air velocity, the second air velocity, the third air velocity, the fourth air velocity, the fifth air velocity, or the sixth air velocity is 1.0 m/s to about 25.0 m/s, about 2.5 m/s to about 25.0 m/s, about 2.5 m/s to about 20.0 m/s, about 2.5 m/s to about 15.0 m/s, about 2.5 m/s to about 10.0 m/s, about 5.0 m/s to about 25.0 m/s, about 10.0 m/s to about 25.0 m/s, or about 15.0 m/s to about 25.0 m/s. The method comprises, in some embodiments, aspirating the preparation, the first fraction, the second fraction, the third fraction, the fourth fraction, or the fifth fraction with the first air flow, the second air flow, the third air flow, the fourth air flow, the fifth air flow, or the sixth air flow that enters the vertical chamber at an angle of about −20 degrees to about −50 degrees, about −25 degrees to about −45 degrees, or about −30 degrees to about −40 degrees relative to horizontal.

In some embodiments, the purity of the first fraction, second fraction, third fraction, fourth fraction, fifth fraction, or sixth fraction is increased by about 0.5-fold to about 40-fold compared to the purity of the plant embryo explants in the preparation, wherein the purity is defined as the percentage of dry plant embryo explants per particle. In certain embodiments, the purity of the sixth fraction is increased by about 0.5-fold to about 40-fold compared to the purity of the embryo explants in the preparation, or compared to the purity of the first fraction, the second fraction, the third fraction, the fourth fraction, or the fifth fraction, wherein the purity is defined as the percentage of dry embryo explants per particle.

In particular embodiments, the first air output port, the second air output port, the third air output port, the fourth air output port, the fifth air output port, or the sixth air output port is in fluid communication with a discharge channel, and wherein the first air flow, the second air flow, the third air flow the fourth, the fifth air flow, or the sixth air flow travels through the first air output port, the second air output port, the third air output port, the fourth air output port, the fifth air output port, or the sixth air output port and into the discharge channel. In some embodiments, the first function unit, the second functional unit, the third function unit, the fourth functional unit, the fifth functional unit, or the sixth functional unit has an average horizontal cross-sectional area of about 32.258 cm² to about 645.16 cm², about 64.516 cm² to about 516.128 cm², about 129.302 cm² to about 387.096 cm², about 193.548 cm² to about 322.58 cm², or about 225.806 cm² to about 290.3222. The method, in certain embodiments, comprises introducing the preparation into the first functional unit from a vibratory feeding unit. The introducing, in some embodiments, comprises introducing the preparation into the first functional unit of the vertical chamber at a rate of about 1 g/min to about 70 g/min, about 10 g/min to about 60 g/min, about 20 g/min to about 50 g/min, or about 30 g/min to about 40 g/min.

In some aspects, the present disclosure provides, a method of purifying genetically modifiable dry embryo explants, the method comprising contacting a preparation of dry plant embryo explants comprising meristematic tissue with a first vibratory screen, wherein the first vibratory screen comprises a plurality of openings, each having a first opening size and a first opening shape, and wherein the preparation comprises a population of dry plant embryo explants, and debris material; vibrating the first vibratory screen to produce a first screen motion, wherein the first screen motion comprises a horizontal vibratory component; and separating a first fraction of embryo explants from a first portion of the debris material by length, width, or thickness relative to the first opening size or the first opening shape, or by a displacement of the first fraction relative to a displacement of the first portion of the debris material produced by the first screen motion. In certain embodiments, the first opening shape is circular. The first opening size, in some embodiments, is from about 1.3 mm to about 1.6 mm, about 1.4 mm to about 1.5 mm, about 1.3 mm to about 1.5 mm, or about 1.4 mm to about 1.6 mm in diameter, or about 1.3 mm, about 1.4 mm, about 1.5 mm, or about 1.6 mm in diameter. The first vibratory screen, in particular embodiments, comprises from about 50 to about 200 openings per 6.4516 cm², about 100 to about 200 openings per 6.4516 cm², or about 125 to about 150 openings per 6.4516 cm². In some embodiments, the first opening shape is oblong. The first opening size, in certain embodiments, is from about 5 mm to about 15 mm, about 6 mm to about 14 mm, about 8 mm to about 12 mm, about 8 mm to about 10 mm, about 9 mm to about 11 mm, about 10 mm to about 12 mm in length, or about 8 mm, about 9 mm, about 10 mm, about 11 mm, or about 12 mm in length, and from about 0.6 mm to about 0.8 mm, about 0.6 mm to about 0.7 mm, or about 0.7 mm to about 0.8 mm in width, or about 0.6 mm, about 0.65 mm, about 0.7 mm, about 0.75 mm, or about 0.8 mm in width. The first vibratory screen, in particular embodiments, comprises from about 5 to about 100 openings per 6.4516 cm², about 10 to about 50 openings per 6.4516 cm², or about 15 to about 35 openings per 6.4516 cm².

In some embodiments, the horizontal vibratory component of the first screen motion comprises a displacement amplitude from about 4.0 mm to about 6.0 mm, about 4.5 mm to about 5.75 mm, about 4.7 mm to about 5.6 mm, about 4.7 mm, about 4.75 mm, about 4.8 mm, about 4.85 mm, about 4.9 mm, about 4.95 mm, about 5.0 mm, about 5.05 mm, about 5.1 mm, about 5.15 mm, about 5.2 mm, about 5.25 mm, about 5.3 mm, about 5.35 mm, about 5.4 mm, about 5.45 mm, about 5.5 mm, about 5.55 mm, or about 5.6 mm. The plane of the first vibratory screen, in certain embodiments, is horizontal relative to the ground and the horizontal vibratory component of the first screen motion is in the plane of the first vibratory screen and changes direction within the plane over time. In particular embodiments, the first screen motion comprises a vertical vibratory component. The vertical vibratory component, in certain embodiments, comprises a displacement amplitude from about 4.0 mm to about 8.0 mm, about 4.0 mm to about 7.5 mm, about 4.5 mm to about 8.0 mm, about 4.5 mm to about 7.5 mm, about 4.7 mm to about 7.2 mm, or about 4.7 mm, about 4.75 mm, about 4.8 mm, about 4.85 mm, about 4.9 mm, about 4.95 mm, about 5.0 mm, about 5.05 mm, about 5.1 mm, about 5.15 mm, about 5.2 mm, about 5.25 mm, about 5.3 mm, about 5.35 mm, about 5.4 mm, about 5.45 mm, about 5.5 mm, about 5.55 mm, about 5.6 mm, about 5.65 mm, about 5.7 mm, about 5.75 mm, about 5.8 mm, about 5.85 mm, about 5.9 mm, about 5.95 mm, about 6.0 mm, about 6.05 mm, about 6.1 mm, about 6.15 mm, about 6.2 mm, about 6.25 mm, about 6.3 mm, about 6.35 mm, about 6.4 mm, about 6.45 mm, about 6.5 mm, about 6.55 mm, about 6.6 mm, about 6.65 mm, about 6.7 mm, about 6.75 mm, about 6.8 mm, about 6.85 mm, about 6.9 mm, about 6.95 mm, about 7.0 mm, about 7.05 mm, about 7.1 mm, about 7.15 mm, or about 7.2 mm. In some embodiments, the plane of the first vibratory screen is horizontal relative to the ground and the vertical vibratory component of the first screen motion is perpendicular to the plane. The horizontal vibratory component and the vertical vibratory component of the first screen motion, in certain embodiments, have the same vibration frequency.

In some embodiments of present disclosure, the method comprises vibrating the first vibratory screen at a frequency of about 1 Hz to about 200 Hz, about 2 Hz to about 200 Hz, about 2 Hz to about 175 Hz, about 4 Hz to about 170 Hz, about 5 Hz to about 150 Hz, about 5 Hz to about 125 Hz, about 5 Hz to about 100 Hz, about 5 Hz to about 90 Hz, about 5 Hz to about 80 Hz, about 5 Hz to about 70 Hz, about 5 Hz to about 60 Hz, about 5 Hz to about 50 Hz, about 5 Hz to about 40 Hz, about 5 Hz to about 30 Hz, about 5 Hz to about 25 Hz, about 10 Hz to about 25 Hz, about 10 Hz to about 25 Hz, about 5 Hz, about 10 Hz, about 15 Hz, about 20 Hz, about 25 Hz, about 30 Hz, about 35 Hz, about 40 Hz, about 45 Hz, about 50 Hz, about 55 Hz, or about 60 Hz.

In certain embodiments, the methods provided by the present disclosure further comprise contacting the first fraction with a second vibratory screen, wherein the second vibratory screen comprises a plurality of openings, each having a second opening size and a second opening shape; and vibrating the second vibratory screen to produce a second screen motion, wherein the second screen motion comprises a horizontal vibratory component; and separating a second fraction of embryo explants from a second portion of the debris material comprised in the first fraction by length, width, or thickness relative to the second opening size or the second opening shape, or by a displacement of the second fraction relative to a displacement of the second portion of the debris material produced by the second screen motion. In some embodiments, the second opening shape is circular. The second opening size, in particular embodiments, is from about 1.3 mm to about 1.6 mm, about 1.4 mm to about 1.5 mm, about 1.3 mm to about 1.5 mm, or about 1.4 mm to about 1.6 mm in diameter, or about 1.3 mm, about 1.4 mm, about 1.5 mm, or about 1.6 mm in diameter. In certain embodiments, the second vibratory screen comprises from about 50 to about 200 openings per 6.4516 cm², about 100 to about 200 openings per 6.4516 cm², or about 125 to about 150 openings per 6.4516 cm². The second opening shape, in some embodiments, is oblong. In particular embodiments, the second opening size is from about 5 mm to about 15 mm, about 6 mm to about 14 mm, about 8 mm to about 12 mm, about 8 mm to about 10 mm, about 9 mm to about 11 mm, about 10 mm to about 12 mm in length, or about 8 mm, about 9 mm, about 10 mm, about 11 mm, or about 12 mm in length, and from about 0.6 mm to about 0.8 mm, about 0.6 mm to about 0.7 mm, or about 0.7 mm to about 0.8 mm in width, or about 0.6 mm, about 0.65 mm, about 0.7 mm, about 0.75 mm, or about 0.8 mm in width. The second vibratory screen, in certain embodiments, comprises from about 5 to about 100 openings per 6.4516 cm², about 10 to about 50 openings per 6.4516 cm², or about 15 to about 35 openings per 6.4516 cm².

The horizontal vibratory component of the second screen motion, in some embodiments, has a displacement amplitude from about 4.0 mm to about 6.0 mm, about 4.5 mm to about 5.75 mm, about 4.7 mm to about 5.6 mm, about 4.7 mm, about 4.75 mm, about 4.8 mm, about 4.85 mm, about 4.9 mm, about 4.95 mm, about 5.0 mm, about 5.05 mm, about 5.1 mm, about 5.15 mm, about 5.2 mm, about 5.25 mm, about 5.3 mm, about 5.35 mm, about 5.4 mm, about 5.45 mm, about 5.5 mm, about 5.55 mm, or about 5.6 mm. In particular embodiments, the plane of the second vibratory screen is horizontal relative to the ground and the horizontal vibratory component of the second screen motion is in the plane of the second vibratory screen and changes direction within the plane over time. The second screen motion, in some embodiments, comprises a vertical vibratory component. The vertical vibratory component of the second screen motion, in certain embodiments, comprises a displacement amplitude from about 4.0 mm to about 8.0 mm, about 4.0 mm to about 7.5 mm, about 4.5 mm to about 8.0 mm, about 4.5 mm to about 7.5 mm, about 4.7 mm to about 7.2 mm, about 4.7 mm, about 4.75 mm, about 4.8 mm, about 4.85 mm, about 4.9 mm, about 4.95 mm, about 5.0 mm, about 5.05 mm, about 5.1 mm, about 5.15 mm, about 5.2 mm, about 5.25 mm, about 5.3 mm, about 5.35 mm, about 5.4 mm, about 5.45 mm, about 5.5 mm, about 5.55 mm, about 5.6 mm, about 5.65 mm, about 5.7 mm, about 5.75 mm, about 5.8 mm, about 5.85 mm, about 5.9 mm, about 5.95 mm, about 6.0 mm, about 6.05 mm, about 6.1 mm, about 6.15 mm, about 6.2 mm, about 6.25 mm, about 6.3 mm, about 6.35 mm, about 6.4 mm, about 6.45 mm, about 6.5 mm, about 6.55 mm, about 6.6 mm, about 6.65 mm, about 6.7 mm, about 6.75 mm, about 6.8 mm, about 6.85 mm, about 6.9 mm, about 6.95 mm, about 7.0 mm, about 7.05 mm, about 7.1 mm, about 7.15 mm, or about 7.2 mm. In some embodiments, the plane of the second vibratory screen is horizontal relative to the ground and the vertical vibratory component of the second screen motion is perpendicular to the plane. The horizontal vibratory component and the vertical vibratory component of the second screen motion, in particular embodiments, have the same frequency.

In certain embodiments, the methods provided by the present disclosure may further comprise vibrating the second vibratory screen at a frequency of about 1 Hz to about 200 Hz, about 2 Hz to about 200 Hz, about 2 Hz to about 175 Hz, about 4 Hz to about 170 Hz, about 5 Hz to about 150 Hz, about 5 Hz to about 125 Hz, about 5 Hz to about 100 Hz, about 5 Hz to about 90 Hz, about 5 Hz to about 80 Hz, about 5 Hz to about 70 Hz, about 5 Hz to about 60 Hz, about 5 Hz to about 50 Hz, about 5 Hz to about 40 Hz, about 5 Hz to about 30 Hz, about 5 Hz to about 25 Hz, about 10 Hz to about 25 Hz, about 10 Hz to about 25 Hz, about 5 Hz, about 10 Hz, about 15 Hz, about 20 Hz, about 25 Hz, about 30 Hz, about 35 Hz, about 40 Hz, about 45 Hz, about 50 Hz, about 55 Hz, or about 60 Hz.

The first vibratory screen and the second vibratory screen, in some embodiments, are structurally connected and move in unison. In certain embodiments, the plane of the second vibratory screen is parallel to the plane of the first vibratory screen. The position of the first vibratory screen, in particular embodiments, is directly above the position of the second vibratory screen. In some embodiments, the first screen motion is the same as the second screen motion.

Vibrating the first vibratory screen, in certain embodiments, comprises rotating at least one weight about the center of a motion generator, wherein the motion generator is structurally connected with the at least one weight and the first vibratory screen. Vibrating the second vibratory screen, in some embodiments, comprises rotating at least one weight about the center of a motion generator, wherein the motion generator is structurally connected with the at least one weight and the second vibratory screen. In particular embodiments, the motion generator comprises a motor comprising an axis of rotation, wherein the axis of rotation is perpendicular to the plane of the first vibratory screen, wherein the motion generator is structurally connected with a first weight and a second weight, and wherein the first weight is positioned above the motion generator and the second weight is positioned below the motion generator. The lead angle, in some embodiments, between the first weight and the second weight is from about 0° to about 90°, from about 15° to about 75°, from about 30° to about 60°, from about 40° to about 50°, about 10°, about 15°, about 20°, about 25°, about 30°, about 35°, about 40°, about 45°, about 50°, about 55°, about 60°, about 65°, about 70°, about 75°, about 80°, about 85°, or about 90°. In some embodiments, the methods provided by the present disclosure further comprise rotating the at least one weight about the center of the motion generator at about 400 to about 10,000 rotations per minute (rpm) or about 400 to about 3,600 rpm.

Separating the first fraction, in certain embodiments, comprises retaining the first portion of the debris material on the first vibratory screen and passing the first fraction of embryo explants through the plurality of openings. Separating the second fraction, in some embodiments, comprises retaining the second portion of the debris material on the second vibratory screen and passing the second fraction of embryo explants through the plurality of openings. In particular embodiments, the first vibratory screen comprises a proximal end, a distal end, and a center, and the contacting comprises contacting the preparation with the first vibratory screen at or near the center.

In some embodiments, the purity of dry embryo explants in the first fraction is increased by from about 0.1-fold to about 10-fold, about 1-fold to about 8-fold, or about 2-fold to about 5-fold, compared to the purity of dry embryo explants in the preparation, wherein the purity is defined as the percentage of dry embryo explants per particle. The purity of dry embryo explants in the second fraction, in certain embodiments, is increased by about 0.1-fold to about 10-fold, about 1-fold to about 8-fold, or about 2-fold to about 5-fold compared to the purity of dry embryo explants in the preparation or compared to the purity of dry embryo explants in the first fraction, wherein the purity is defined as the percentage of dry embryo explants per particle.

In other aspects, the present disclosure provides a method of purifying genetically modifiable dry plant embryo explants, the method comprising contacting a preparation of dry plant embryo explants comprising meristematic tissue with a first textured surface of a first vibratory platform, wherein the first textured surface of the first vibratory platform is substantially planar, and wherein the preparation comprises a population of dry plant embryo explants and debris material; vibrating the first vibratory platform to produce a first platform motion; and separating a first fraction of the plant embryo explants from a first portion of the debris material according to a displacement of the first fraction relative to a displacement of the first portion of debris material on the first textured surface of the first vibratory platform. In some embodiments, the first vibratory platform has a first proximal edge and a first distal edge, wherein the first proximal edge of the first vibratory platform is elevated relative to the first distal edge of the first vibratory platform. In certain embodiments, the first vibratory platform comprises a first pitch axis and a first tilt axis, wherein the first pitch axis intersects the first proximal edge and the first distal edge of the first vibratory platform, wherein the first tilt axis is perpendicular to the first pitch axis, wherein the first vibratory platform is positioned at a first compound angle relative to the ground, wherein the first compound angle comprises a first pitch angle and a first tilt angle, wherein the first pitch angle is along the first pitch axis, and wherein the first tilt angle is along the first tilt axis. In particular embodiments, the first vibratory platform has a first upper edge and a first lower edge, wherein the first tilt angle intersects the first upper edge and the first lower edge, and wherein the first upper edge of the first vibratory platform is elevated relative to the first lower edge of the first vibratory platform. The first tilt angle, in some embodiments, is about 8.0 degrees to about 25.0 degrees, about 9.0 degrees to about 25.0 degrees, about 10.0 degrees to about 25.0 degrees, about 8.0 degrees to about 22.0 degrees, about 10.0 degrees to about 20.0 degrees, about 11.0 degrees to about 19.0 degrees, about 12.7 degrees to about 14.7 degrees, about 15.8 degrees to about 16.6 degrees, about 11.6 degrees to about 12.0 degrees, about 16.2 degrees to about 18.3 degrees, about 11.6 degrees to about 14.2 degrees, about 12.5 degrees to about 17.3 degrees, about 10.9 degrees to about 14.9 degrees, or about 17.5 degrees to about 21.5 degrees, or about 11.8 degrees, about 17.2 degrees, about 12.9 degrees, about 13.7 degrees, or about 14.5 degrees. The first pitch angle, in certain embodiments, is about 1.4 degrees to about 9.0 degrees, about 1.5 degrees to about 8.0 degrees, about 2.1 degrees to about 2.6 degrees, about 4.3 degrees to about 7.5 degrees, about 1.9 degrees to about 3.25 degrees, about 2.4 degrees to about 4.9 degrees, about 1.8 degrees to about 3.25 degrees, about 2.0 degrees to about 6.0 degrees, about 1.0 degrees to about 4.2 degrees, or about 1.5 degrees to about 4.5 degrees, or about 2.3 degrees, about 5.9 degrees, about 2.6 degrees, about 3.6 degrees, about 2.5 degrees, about 2 degrees, or about 4 degrees.

In some embodiments, the population comprises corn embryo explants, and the first tilt angle is about 10.0 degrees to about 20.0 degrees, about 10.0 degrees to about 17.0 degrees, about 12.5 degrees to about 15.0 degrees, about 12.7 degrees to about 14.7 degrees or about 13.7 degrees, and the first pitch angle is about 1.5 degrees to about 3.5 degrees, about 2.0 degrees to about 3.0 degrees, about 2.1 degrees to about 2.6 degrees, about 2.3 degrees, or about 2.4 degrees. In certain embodiments, the population comprises soybean embryo explants, and the first tilt angle is about 10.0 degrees to about 20.0 degrees, about 10.0 degrees to about 18.0 degrees, about 14.0 degrees to about 20.0 degrees, about 11.0 degrees to about 17.0 degrees, about 11.6 degrees to about 16.6 degrees, about 11.6 degrees to about 12.0 degrees, about 15.8 degrees to about 16.6 degrees, about 11.8 degrees, or about 16.2 degrees, and the first pitch angle is about 1.5 degrees to about 8.0 degrees, about 1.9 degrees to about 7.5 degrees, about 1.9 degrees to about 3.3 degrees, about 4.3 degrees to about 7.5 degrees, about 2.5 degrees, about 2.6 degrees, or about 5.9 degrees. In particular embodiments, the population comprises cotton embryo explants, and the first tilt angle is about 10.0 degrees to about 22.0 degrees, about 10.0 degrees to about 20.0 degrees, about 10.0 degrees to about 19.0 degrees, about 15.0 degrees to about 22.0 degrees, about 11.0 degrees to about 19.0 degrees, about 11.0 degrees to about 15.0 degrees, about 11.6 degrees to about 14.2 degrees, about 16.0 degrees to about 19.0 degrees, about 16.2 degrees to about 18.3 degrees, about 12.9 degrees, about 17.2 degrees, or about 17.3 degrees, and the first pitch angle is about 1.5 degrees to about 6.0 degrees, about 1.5 degrees to about 5.0 degrees, about 1.8 degrees to about 4.9 degrees, about 1.8 degrees to about 3.3 degrees, about 2.4 degrees to about 4.9 degrees, about 2.5 degrees, about 2.6 degrees, about 3.6 degrees, or about 3.7 degrees. In some embodiments, the population comprises wheat embryo explants, and the first tilt angle is about 10.0 degrees to about 20.0 degrees, about 10.0 degrees to about 19.0 degrees, about 12.0 degrees to about 17.0 degrees, about 13.0 degrees to about 16.0 degrees, about 14.0 degrees to about 15.0 degrees, or about 14.5 degrees, and the first pitch angle is about 1.5 degrees to about 8.0 degrees, about 2.0 degrees to about 6.0 degrees, about 3.0 degrees to about 5.0 degrees, about 3.5 degrees to about 4.5 degrees, or about 4.0 degrees.

The method, in some embodiments, further comprises contacting the first fraction with a second textured surface of a second vibratory platform, wherein the second textured surface of the second vibratory platform is substantially planar; vibrating the second vibratory platform to produce a second platform motion; and separating a second fraction of the plant embryo explants of the first fraction from a second portion of the debris material according to a displacement of the second fraction relative to a displacement of the second portion of debris material on the second textured surface of the second vibratory platform. In particular embodiments, the second vibratory platform has a second proximal edge and a second distal edge, wherein the second proximal edge of the second vibratory platform is elevated relative to the second distal edge of the second vibratory platform. In certain embodiments, the second vibratory platform comprises a second pitch axis and a second tilt axis, wherein the second pitch axis intersects the second proximal edge and the second distal edge of the second vibratory platform, wherein the second tilt axis is perpendicular to the second pitch axis, wherein the second vibratory platform is positioned at a second compound angle relative to the ground, wherein the second compound angle comprises a second pitch angle and a second tilt angle, wherein the second pitch angle is along the second pitch axis, and wherein the second tilt angle is along the second tilt axis. In some embodiments, the second vibratory platform has a second upper edge and a second lower edge, wherein the second tilt angle intersects the second upper edge and the second lower edge, and wherein the second upper edge of the second vibratory platform is elevated relative to the second lower edge of the second vibratory platform. The second tilt angle, in certain embodiments, is about 8.0 degrees to about 25.0 degrees, about 9.0 degrees to about 25.0 degrees, about 10.0 degrees to about 25.0 degrees, about 8.0 degrees to about 22.0 degrees, about 10.0 degrees to about 20.0 degrees, about 11.0 degrees to about 19.0 degrees, about 12.7 degrees to about 14.7 degrees, about 15.8 degrees to about 16.6 degrees, about 11.6 degrees to about 12.0 degrees, about 16.2 degrees to about 18.3 degrees, about 11.6 degrees to about 14.2 degrees, about 12.5 degrees to about 17.3 degrees, about 10.9 degrees to about 14.9 degrees, or about 17.5 degrees to about 21.5 degrees, or about 11.8 degrees, about 17.2 degrees, about 12.9 degrees, about 13.7 degrees, or about 14.5 degrees. The second pitch angle, in particular embodiments, is about 1.4 degrees to about 9.0 degrees, about 1.5 degrees to about 8.0 degrees, about 2.1 degrees to about 2.6 degrees, about 4.3 degrees to about 7.5 degrees, about 1.9 degrees to about 3.25 degrees, about 2.4 degrees to about 4.9 degrees, about 1.8 degrees to about 3.25 degrees, about 2.0 degrees to about 6.0 degrees, about 1.0 degrees to about 4.2 degrees, or about 1.5 degrees to about 4.5 degrees, or about 2.3 degrees, about 5.9 degrees, about 2.6 degrees, about 3.6 degrees, about 2.5 degrees, about 2 degrees, or about 4 degrees. In certain embodiments, the population comprises wheat embryo explants and the second tilt angle is about 10.0 degrees to about 16.0 degrees, about 10.0 degrees to about 15.0 degrees, about 11.0 degrees to about 15.0 degrees, about 12.0 degrees to about 14.0 degrees, about 12.5 degrees to about 13.5 degrees, about 12.7 degrees to about 13.1 degrees, or about 12.9 degrees, and the second pitch angle is about 1.5 degrees to about 5.0 degrees, about 1.0 degrees to about 4.0 degrees, about 1.0 degrees to about 3.0 degrees, about 1.5 degrees to about 3.0 degrees, about 1.8 degrees to about 2.6 degrees, or about 2.2 degrees.

In particular embodiments, the first platform motion or the second platform motion comprises a substantially horizontal vibratory component. In some embodiments, the first platform motion or the second platform motion is linear. The first platform motion, in certain embodiments, is along the first tilt axis. The second platform motion, in particular embodiments, is along the second tilt axis. In certain embodiments, the first platform motion or the second platform motion has a vibrational frequency of about 1 Hz to about 500 Hz, about 10 Hz to about 400 Hz, about 20 Hz to about 300 Hz, about 30 Hz to about 250 Hz, about 40 Hz to about 200 Hz, about 50 Hz to about 150 Hz, about 55 Hz to about 125 Hz, about 60 Hz to about 120 Hz, about 50 Hz, about 60 Hz, about 70 Hz, about 80 Hz, about 90 Hz, about 100 Hz, about 110 Hz, about 120 Hz, about 130 Hz, about 140 Hz, or about 150 Hz. In some embodiments, the first platform motion or the second platform motion has a vibrational amplitude of greater than zero (0) mm and less than 2.0 mm or about 0.05 mm to about 1.0 mm, about 0.05 mm to about 0.5 mm, about 0.1 mm to about 0.5 mm, or about 0.05 mm to about 0.2 mm.

The preparation or the population, in certain embodiments, comprises corn, wheat, soybean, cotton, or canola embryo explants. The method comprises, in particular embodiments, initially contacting the preparation with the first textured surface of the first vibratory platform at a first platform contact location. The first platform contact location, in specific embodiments, is at or near the first proximal edge of the first vibratory platform. In some embodiments, the displacement of the first fraction of plant embryo explants comprises a first fraction displacement range, and the first fraction displacement range comprises a first fraction pitch distance component and a first fraction tilt distance component. In certain embodiments, the displacement of the first portion of the debris material comprises a first portion displacement range, and the first portion displacement range comprises a first portion pitch distance component and a first portion tilt distance component. The first portion pitch distance component, in some embodiments, is less than the first fraction pitch distance component. The first portion pitch distance component, in certain embodiments, is greater than the first fraction pitch distance component. The first portion tilt distance component, in particular embodiments, is less than the first fraction tilt distance component. The first portion tilt distance component, in some embodiments, is greater than the first fraction tilt distance component.

The method comprises, in particular embodiments, initially contacting the first fraction with the second textured surface of the second vibratory platform at a second platform contact location. The second platform contact location, in some embodiments, is at or near the second proximal edge of the second vibratory platform. In certain embodiments, the displacement of the second fraction of plant embryo explants comprises a second fraction displacement range, wherein the second fraction displacement range comprises a second fraction pitch distance component and a second fraction tilt distance component. In particular embodiments, the displacement of the second portion of debris material comprises a second portion displacement range, wherein the second portion displacement range comprises a second portion pitch distance component and a second portion tilt distance. The second portion pitch distance component, in certain embodiments, is less than the second fraction pitch distance component. The second portion pitch distance component, in some embodiments, is greater than the second fraction pitch distance component. The second portion tilt distance component, in particular embodiments, is less than the second fraction tilt distance component. The second portion tilt distance component, in certain embodiments, is greater than the second fraction tilt distance component.

The method, in some embodiments, further comprises collecting the first fraction of plant embryo explants. The method, in particular embodiments, further comprises collecting the first fraction of plant embryo explants in a first fraction collector. In certain embodiments, the first fraction of plant embryo explants falls into the first fraction collector from a first fraction distal location on the first distal edge of the first vibratory platform. The method further comprises, in some embodiments, collecting the first portion of debris material in a first portion collector. In particular embodiments, the first portion of debris material falls into the first portion collector from a first portion distal location on the first distal edge of the first vibratory platform. The first fraction distal location, in certain embodiments, is positioned closer to the first lower edge of the first vibratory platform than is the first portion distal location. The first fraction distal location, in some embodiments, is positioned closer to the first platform contact location of the first vibratory platform than is the first portion distal location. The first fraction distal location, in particular embodiments, is positioned closer to the first upper edge of the first vibratory platform than is the first portion distal location. The first portion distal location, in certain embodiments, is positioned closer to the first lower edge of the first vibratory platform than is the first fraction distal location. The first portion distal location, in particular embodiments, is positioned closer to the first platform contact location of the first vibratory platform than is the first fraction distal location. The first portion distal location, in some embodiments, is positioned closer to the first upper edge of the first vibratory platform than is the first fraction distal location.

The method, in particular embodiments, comprises contacting the preparation with the first textured surface of the first vibratory platform at a rate of about 1.0 g/min to about 5.0 g/min, about 2.0 g/min to about 4.0 g/min, or about 3.0 g/min of preparation. In some embodiments, the first vibratory platform comprises a first pitch dimension from the first proximal edge to the first distal edge through a first center point of the first vibratory platform and along or parallel to the first pitch axis, and a first tilt dimension from the first upper edge to the first lower edge through the first center point of the first vibratory platform and along or parallel to the first tilt axis. The first pitch dimension, in certain embodiments, is about 12.7 cm to about 127 cm, about 12.7 cm to about 76.2 cm, about 12.7 cm to about 50.8 cm, about 12.7 cm to about 38.1 cm, about 12.7 cm to about 25.4 cm, or about 25.4 cm to about 38.1 cm. The first tilt dimension, in particular embodiments, is about 12.7 cm to about 127 cm, about 12.7 cm to about 76.2 cm, about 12.7 cm to about 63.5 cm, about 12.7 cm to about 50.8 cm, about 25.4 cm to about 50.8 cm, or about 25.4 cm to about 38.1 cm. In some embodiments, a distance measured from the first upper edge to the first lower edge of the first vibratory platform at or near the first proximal edge of the first vibratory platform is less than a distance measured from the first upper edge to the first lower edge of the first vibratory platform at or near the first distal edge of the first vibratory platform.

The method, in certain embodiments, further comprises collecting the second fraction of plant embryo explants. The method, in particular embodiments, further comprises collecting the second fraction of plant embryo explants in a second fraction collector. The second fraction of plant embryo explants, in some embodiments, falls into the second fraction collector from a second fraction distal location on the second distal edge of the second vibratory platform. The method, in certain embodiments, further comprises collecting the second portion of the debris material in a second portion collector. The second portion of debris material, in particular embodiments, falls into the second portion collector from a second portion distal location on the second distal edge of the second vibratory platform. In some embodiments, the second fraction distal location is closer to the second lower edge of the second vibratory platform than is the second portion distal location. In certain embodiments, the second fraction distal location is closer to the second platform contact location of the second vibratory platform than is the second portion distal location. In particular embodiments, the second fraction distal location is closer to the second upper edge of the second vibratory platform than is the second portion distal location. In some embodiments, the second portion distal location is closer to the second lower edge of the second vibratory platform than is the second fraction distal location. In certain embodiments, the second portion distal location is closer to the second platform contact location of the second vibratory platform than is the second fraction distal location. In particular embodiments, the second portion distal location is closer to the second upper edge of the second vibratory platform than is the second fraction distal location.

The method comprises, in some embodiments, contacting the first fraction with the second textured surface of the second vibratory platform at a rate of about 1.0 g/min to about 5.0 g/min, about 2.0 g/min to about 4.0 g/min, or about 3.0 g/min of first fraction. In certain embodiments, the second vibratory platform comprises a second pitch dimension from the second proximal edge to the second distal edge through a second center point of the second vibratory platform and along or parallel to the second pitch axis, and a second tilt dimension from the second upper edge to the second lower edge through the second center point of the second vibratory platform and along or parallel to the second tilt axis. The second pitch dimension, in particular embodiments, is about 12.7 cm to about 127 cm, about 12.7 cm to about 76.2 cm, about 12.7 cm to about 50.8 cm, about 12.7 cm to about 38.1 cm, about 12.7 cm to about 25.4 cm, or about 25.4 cm to about 38.1 cm. The second tilt dimension, in some embodiments, is about 12.7 cm to about 127 cm, about 12.7 cm to about 76.2 cm, about 12.7 cm to about 63.5 cm, about 12.7 cm to about 50.8 cm, about 25.4 cm to about 50.8 cm, or about 25.4 cm to about 38.1 cm. In certain embodiments, a distance measured from the second upper edge to the second lower edge of the second vibratory platform at or near the second proximal edge of the second vibratory platform is less than a distance measured from the second upper edge to the second lower edge of the second vibratory platform at or near the second distal edge of the second vibratory platform.

In particular embodiments, the substantially planar shape of the first vibratory platform or of the second vibratory platform is selected from the group consisting of a square, a rectangle, a rhombus, a triangle, a trapezoid, a circle, an oval, a polygonal shape, and a non-polygonal shape. In certain embodiments, the first upper edge of the first vibratory platform or the first lower edge of the first vibratory platform is upwardly curled. In some embodiments, the second upper edge of the second vibratory platform or the second lower edge of the second vibratory platform is upwardly curled. In particular embodiments, the first textured surface or the second textured surface is a sandpaper surface, a vinyl surface, a plasma coated surface, a cork surface, a fabric surface, a rubber surface, or a plastic surface. In certain embodiments, the first textured surface or the second textured surface comprises an 80-150 grit sandpaper, or an 80 grit sandpaper, a 90 grit sandpaper, a 100 grit sandpaper, a 110 grit sandpaper, a 120 grit sandpaper, a 130 grit sandpaper, a 140 grit sandpaper, or a 150 grit sandpaper. In some embodiments, the first textured surface or the second textured surface comprises a plurality of adhered granules, each adhered granule having a granule size and a granule shape. The granule shape, in particular embodiments, is selected from the group consisting of a three-dimensional geometric or irregular shape, a rectangular prism, a cube, a sphere, or an ovoid. The granule size, in certain embodiments, comprises a granule diameter, a granule width, a granule length, or a granule depth, and the granule diameter, granule width, granule length, or granule depth is about 50 μm to about 300 μm, about 50 μm to about 250 μm, about 50 μm to about 200 μm, about 90 μm to about 190 μm, about 50 μm, about 60 μm, about 70 μm about 80 μm, about 90 μm, about 100 μm, about 110 μm, about 115 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm. In some embodiments, the first textured surface or the second textured surface is structurally adhered to a top surface of the first vibratory platform or to a top surface of the second vibratory platform.

The preparation, in certain embodiments, comprises corn embryo explants, and the first textured surface or the second textured surface comprises granules having an average diameter, width, length, or depth of about 90 μm to about 190 μm. The preparation, in particular embodiments, comprises soybean, cotton, or wheat embryo explants and the first textured surface or the second textured surface comprises granules having an average diameter, width, length, or depth of about 50 μm to about 250 μm. In some embodiments, the first platform or second platform comprises about 50 to about 400, about 50 to about 350, about 50 to about 300, about 50 to about 250, about 60 to about 200, about 80 to about 150, about 200, about 190, about 180, about 170, about 160, about 150, about 140, about 130, about 120, about 110, about 100, about 90, about 80, about 70, about 60, or about 50 granules per 6.4516 cm².

In some embodiments, the purity of the first fraction is increased by from about 1.5-fold to about 5-fold, about 2-fold to about 5-fold, about 2-fold to about 4-fold, or about 2-fold, about 3-fold, or about 4-fold compared to the purity of the embryo explants in the preparation, wherein the purity is defined as the percentage of dry embryo explants per weight of sample or as the percentage of dry embryo explants per particle. In certain embodiments, the purity of the second fraction is increased by about 1.5-fold to about 10-fold, about 1.5-fold to about 7.5-fold, 5-fold to about 10-fold, 2-fold to about 10-fold, 3-fold to about 10-fold, 4-fold to about 8-fold, about 1.5-fold to about 5-fold, about 2-fold to about 5-fold, or about 2-fold to about 4-fold, or about 2-fold, about 3-fold, about 4-fold, about 5-fold, or about 6-fold compared to the purity of dry embryo explants in the preparation, or compared to the purity of the first fraction, wherein the purity is defined as the percentage of dry embryo explants per weight of sample or as a percentage of dry embryo explants per particle.

In yet other aspects, provided herein is a method of purifying genetically modifiable dry plant embryo explants, the method comprising: the combination of at least two steps selected from the group consisting of: seed sanitizing, seed milling, coarse width sizing, length sizing, aspirating, width and thickness separation, separation using a friction table, and floating in an aqueous solution. Two or more steps of the present disclosure may combined in any order, wherein any step may be performed before, or after, any other step.

In still yet other aspects, the present disclosure provides a method of purifying genetically modifiable dry plant embryo explants, the method comprising: positioning a first grinding roller and a second grinding roller to define a first gap having a first gap distance between the first roller and the second roller; rotating the first roller about a first axis of rotation and the second roller about a second axis of rotation; passing a population of plant seeds through the first gap to produce a first preparation of plant embryo explants comprising meristematic tissue; contacting the first preparation of dry plant embryo explants comprising meristematic tissue with a first moving sieve, wherein the first moving sieve comprises a plurality of openings, each having a first physical opening size, and wherein the first preparation comprises a population of dry plant embryo explants and debris material; and separating a first fraction of embryo explants from a first portion of the debris material by length, width, or thickness relative to the first physical opening size, or relative to a first effective opening size, wherein the first gap distance is about 0.10 mm to about 7.62 mm, wherein the first roller and the second roller each comprise an exterior surface and the exterior surface of the first roller and the exterior surface of the second roller each comprise a plurality of protrusions, and wherein the first moving sieve moves in a circular, elliptical, or linear motion. Provided herein, in some embodiments, is a method of purifying genetically modifiable dry plant embryo explants, the method comprising: positioning a first grinding roller and a second grinding roller to define a first gap having a first gap distance between the first roller and the second roller; rotating the first roller about a first axis of rotation and the second roller about a second axis of rotation; passing a population of plant seeds through the first gap to produce a first preparation of plant embryo explants comprising meristematic tissue; positioning a third grinding roller and a fourth grinding roller to define a second gap having a second gap distance between the third roller and the fourth roller; rotating the third roller about a third axis of rotation and the fourth roller about a fourth axis of rotation; passing the first preparation of embryo explants through the second gap to produce a second preparation of plant embryo explants comprising meristematic tissue; contacting the second preparation of dry plant embryo explants comprising meristematic tissue with a first moving sieve, wherein the first moving sieve comprises a plurality of openings, each having a first physical opening size, and wherein the second preparation comprises a population of dry plant embryo explants and debris material; and separating a first fraction of embryo explants from a first portion of the debris material by length, width, or thickness relative to the first physical opening size, or relative to a first effective opening size, wherein the second gap distance is about 0.10 mm to about 7.62 mm, wherein the third roller and the fourth roller each comprise an exterior surface and the exterior surface of the third roller and the exterior surface of the fourth roller each comprise a plurality of protrusions, and wherein the first moving sieve moves in a circular, elliptical, or linear motion. The methods of the present disclosure may, in particular embodiments, further comprise contacting the first fraction with a second moving sieve, wherein the second moving sieve comprises a plurality of openings, each having a second physical opening size; and separating a second fraction of embryo explants from a second portion of the debris material remaining in the first fraction by length, width, or thickness relative to the second physical opening size, or relative to a second effective opening size, wherein the second moving sieve moves in a circular, elliptical, or linear motion. In some embodiments, the methods of the present disclosure may further comprise: contacting the second fraction with a third moving sieve, wherein the third moving sieve comprises a plurality of openings, each having a third physical opening size; and separating a third fraction of embryo explants from a third portion of the debris material remaining in the second fraction by length, width, or thickness relative to the third physical opening size, or relative to a third effective opening size, wherein the third moving sieve moves in a circular, elliptical, or linear motion. The methods of the present disclosure, in particular embodiments may further comprise: separating the first preparation into a first top preparation fraction, a first middle preparation fraction, and a first bottom preparation fraction, wherein the first top preparation fraction is retained on the first moving sieve, the first middle preparation fraction is retained on the second moving sieve, and the first bottom preparation fraction is retained on the third moving sieve. In some embodiments, the methods of the present disclosure may further comprise: positioning the first grinding roller and the second grinding roller to define a first gap having a first gap distance between the first roller and the second roller; rotating the first roller about a first axis of rotation and the second roller about a second axis of rotation; and passing the first top preparation fraction through the first gap to produce a second preparation of plant embryo explants comprising meristematic tissue, wherein the first gap distance is about 0.508 mm to about 1.016 mm, about 0.508 mm to about 0.762 mm, or is about 0.6985 mm.

In some aspects, the present disclosure provides a method of purifying genetically modifiable dry plant embryo explants, the method comprising: positioning a first grinding plate and a second grinding plate to define a first gap having a first distance between the first plate and the second plate; rotating the first plate or the second plate about an axis of rotation; contacting a population of plant seeds with an interior surface of the first plate and an interior surface of the second plate to produce a first preparation of embryo explants comprising meristematic tissue; contacting the first preparation of embryo explants with a moving plate, wherein the moving plate comprises a proximal end, a distal end, and a plurality of openings located near the distal end, each comprising a first physical opening size, and wherein the first preparation comprises a population of embryo explants and debris material; passing the first preparation through the plurality of openings of the moving plate and contacting a first moving sieve with the first preparation, wherein the first moving sieve comprises a plurality of openings, each having a second physical opening size; and separating a first fraction of embryo explants from a first portion of the debris material by length, width, or thickness relative to the second physical opening size, wherein the first gap distance is about 2.5 mm to about 4.0 mm or about 3.0 mm to about 3.25 mm, and wherein the moving plate and the first moving sieve move in a linear motion. Provided herein, in certain embodiments, is a method further comprising: positioning a third grinding plate and a fourth grinding plate to define a second gap having a second gap distance between the third plate and the fourth plate; rotating the third plate or the fourth plate about an axis of rotation; contacting the first fraction with an interior surface of the third plate and an interior surface of the fourth plate to produce a second preparation of embryo explants comprising meristematic tissue; contacting the second preparation with a second moving sieve comprising a plurality of openings, each having a third physical opening size; separating a second fraction of embryo explants from a second portion of the debris material by length, width, or thickness relative to the third physical opening size; contacting the second fraction with a third moving sieve comprising a plurality of openings, each comprising a fourth physical opening size; and separating a third fraction of embryo explants from a third portion of the debris material by length, width, or thickness relative to the fourth physical opening size, wherein the second gap distance is about 0.5 mm to about 2.5 mm or is about 1.5 mm, and wherein the second moving sieve and the third moving sieve move in a linear motion.

In other aspects, the present disclosure provides a method comprising: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction of dry plant embryo explants comprising meristematic tissue with an interior surface of a rotating cylinder, wherein the interior surface comprises a plurality of indentations, the indentations having an indentation size and an indentation shape, and wherein the first fraction comprises a population of dry plant embryo explants and debris material; rotating the rotating cylinder about an axis of rotation to produce a centrifugal force acting on the first fraction, wherein the axis of rotation is substantially parallel to the ground; and separating a first cylinder fraction of the plant embryo explants from a first cylinder portion of the debris material according to a displacement of the first cylinder portion of the debris material relative to a displacement of the first cylinder fraction of plant embryo explants produced by the rotating, wherein the first moving sieve moves in a circular, elliptical, or linear motion. In particular embodiments, provided herein is a method comprising: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; separating a second fraction of embryo explants from a second portion of the debris material remaining in the first fraction by length, width, or thickness relative to the second physical opening size, or relative to a second effective opening size; contacting the second fraction of dry plant embryo explants comprising meristematic tissue with an interior surface of a rotating cylinder, wherein the interior surface comprises a plurality of indentations, the indentations having an indentation size and an indentation shape, and wherein the second fraction comprises a population of dry plant embryo explants and debris material; rotating the rotating cylinder about an axis of rotation to produce a centrifugal force acting on the second fraction, wherein the axis of rotation is substantially parallel to the ground; and separating a first cylinder fraction of the plant embryo explants from a first cylinder portion of the debris material according to a displacement of the first cylinder portion of the debris material relative to a displacement of the first cylinder fraction of plant embryo explants produced by the rotating, wherein the first moving sieve and the second moving sieve move in a circular, elliptical, or linear motion. In particular embodiments, the present disclosure provides a method of purifying genetically modifiable dry plant embryo explants, the method comprising: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; separating a second fraction of embryo explants from a second portion of the debris material remaining in the first fraction by length, width, or thickness relative to the second physical opening size, or relative to a second effective opening size; contacting the second fraction with a third moving sieve, wherein the third moving sieve comprises a plurality of openings, each having a third physical opening size; separating a third fraction of embryo explants from a third portion of the debris material remaining in the second fraction by length, width, or thickness relative to the third physical opening size, or relative to a third effective opening size; contacting the third fraction of dry plant embryo explants comprising meristematic tissue with an interior surface of a rotating cylinder, wherein the interior surface comprises a plurality of indentations, the indentations having an indentation size and an indentation shape, and wherein the third fraction comprises a population of dry plant embryo explants and debris material; rotating the rotating cylinder about an axis of rotation to produce a centrifugal force acting on the third fraction, wherein the axis of rotation is substantially parallel to the ground; and separating a first cylinder fraction of the plant embryo explants from a first cylinder portion of the debris material according to a displacement of the first cylinder portion of the debris material relative to a displacement of the first cylinder fraction of plant embryo explants produced by the rotating, wherein the first moving sieve, the second moving sieve, and the third moving sieve move in a circular, elliptical, or linear motion.

In yet other aspects, the present disclosure provides a method of purifying genetically modifiable dry plant embryo explants, the method comprising: contacting a preparation of dry plant embryo explants comprising meristematic tissue with a moving plate, wherein the moving plate comprises a proximal end, a distal end, and a plurality of openings located near the distal end, each comprising a first physical opening size, and wherein the first preparation comprises a population of embryo explants and debris material; passing the preparation through the plurality of openings of the moving plate and contacting a first moving sieve with the first preparation, wherein the first moving sieve comprises a plurality of openings, each having a second physical opening size; separating a first fraction of embryo explants from a first portion of the debris material by length, width, or thickness relative to the second physical opening size; contacting the first fraction of dry plant embryo explants comprising meristematic tissue with an interior surface of a rotating cylinder, wherein the interior surface comprises a plurality of indentations, the indentations having an indentation size and an indentation shape, and wherein the first fraction comprises a population of dry plant embryo explants and debris material; rotating the rotating cylinder about an axis of rotation to produce a centrifugal force acting on the first fraction, wherein the axis of rotation is substantially parallel to the ground; and separating a first cylinder fraction of the plant embryo explants from a first cylinder portion of the debris material according to a displacement of the first cylinder portion of the debris material relative to a displacement of the first cylinder fraction of plant embryo explants produced by the rotating, wherein the moving plate and the first moving sieve move in a linear motion. In particular embodiments, provided herein is a method comprising: contacting a preparation of dry plant embryo explants comprising meristematic tissue with a moving plate, wherein the moving plate comprises a proximal end, a distal end, and a plurality of openings located near the distal end, each comprising a first physical opening size, and wherein the first preparation comprises a population of embryo explants and debris material; passing the preparation through the plurality of openings of the moving plate and contacting a first moving sieve with the first preparation, wherein the first moving sieve comprises a plurality of openings, each having a second physical opening size; separating a first fraction of embryo explants from a first portion of the debris material by length, width, or thickness relative to the second physical opening size; positioning a third grinding plate and a fourth grinding plate to define a second gap having a second gap distance between the third plate and the fourth plate; rotating the third plate or the fourth plate about an axis of rotation; contacting the first fraction with an interior surface of the third plate and an interior surface of the fourth plate to produce a second preparation of embryo explants comprising meristematic tissue; contacting the second preparation with a second moving sieve comprising a plurality of openings, each having a third physical opening size; separating a second fraction of embryo explants from a second portion of the debris material by length, width, or thickness relative to the third physical opening size; contacting the second fraction with a third moving sieve comprising a plurality of openings, each comprising a fourth physical opening size; separating a third fraction of embryo explants from a third portion of the debris material by length, width, or thickness relative to the fourth physical opening size; contacting the third fraction of dry plant embryo explants comprising meristematic tissue with an interior surface of a rotating cylinder, wherein the interior surface comprises a plurality of indentations, the indentations having an indentation size and an indentation shape, and wherein the third fraction comprises a population of dry plant embryo explants and debris material; rotating the rotating cylinder about an axis of rotation to produce a centrifugal force acting on the first fraction, wherein the axis of rotation is substantially parallel to the ground; and separating a first cylinder fraction of the plant embryo explants from a first cylinder portion of the debris material according to a displacement of the first cylinder portion of the debris material relative to a displacement of the first cylinder fraction of plant embryo explants produced by the rotating, wherein the second gap distance is about 0.5 mm to about 2.5 mm or is about 1.5 mm, and wherein the moving plate, the first moving sieve, the second moving sieve, and the third moving sieve move in a linear motion.

In still yet other aspects, the present disclosure provides a method comprising: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; and aspirating the first fraction of embryo explants to obtain a first aspirated fraction of plant embryo explants, wherein the first moving sieve moves in a circular, elliptical, or linear motion. The methods provided by the present disclosure, in particular embodiments, may comprise: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; separating a second fraction of embryo explants from a second portion of the debris material remaining in the first fraction by length, width, or thickness relative to the second physical opening size, or relative to a second effective opening size; and aspirating the second fraction of embryo explants to obtain a first aspirated fraction of plant embryo explants, wherein the first moving sieve and the second moving sieve move in a circular, elliptical, or linear motion. In certain embodiments, the methods of the present disclosure may comprise: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; separating a second fraction of embryo explants from a second portion of the debris material remaining in the first fraction by length, width, or thickness relative to the second physical opening size, or relative to a second effective opening size; contacting the second fraction with a third moving sieve, wherein the third moving sieve comprises a plurality of openings, each having a third physical opening size; separating a third fraction of embryo explants from a third portion of the debris material remaining in the second fraction by length, width, or thickness relative to the third physical opening size, or relative to a third effective opening size; and aspirating the third fraction of embryo explants to obtain a first aspirated fraction of plant embryo explants, wherein the first moving sieve, the second moving sieve, and the third moving sieve move in a circular, elliptical, or linear motion.

In some aspects, the present disclosure provides a method of purifying genetically modifiable dry plant embryo explants, the method comprising: contacting a preparation of dry plant embryo explants comprising meristematic tissue with a moving plate, wherein the moving plate comprises a proximal end, a distal end, and a plurality of openings located near the distal end, each comprising a first physical opening size, and wherein the first preparation comprises a population of embryo explants and debris material; passing the preparation through the plurality of openings of the moving plate and contacting a first moving sieve with the first preparation, wherein the first moving sieve comprises a plurality of openings, each having a second physical opening size; separating a first fraction of embryo explants from a first portion of the debris material by length, width, or thickness relative to the second physical opening size; and aspirating the first fraction of embryo explants to obtain a first aspirated fraction of plant embryo explants, wherein the moving plate and the first moving sieve move in a linear motion. The methods of the present disclosure, in some embodiments, may comprise: contacting a preparation of dry plant embryo explants comprising meristematic tissue with a moving plate, wherein the moving plate comprises a proximal end, a distal end, and a plurality of openings located near the distal end, each comprising a first physical opening size, and wherein the first preparation comprises a population of embryo explants and debris material; passing the preparation through the plurality of openings of the moving plate and contacting a first moving sieve with the first preparation, wherein the first moving sieve comprises a plurality of openings, each having a second physical opening size; separating a first fraction of embryo explants from a first portion of the debris material by length, width, or thickness relative to the second physical opening size; positioning a third grinding plate and a fourth grinding plate to define a second gap having a second gap distance between the third plate and the fourth plate; rotating the third plate or the fourth plate about an axis of rotation; contacting the first fraction with an interior surface of the third plate and an interior surface of the fourth plate to produce a second preparation of embryo explants comprising meristematic tissue; contacting the second preparation with a second moving sieve comprising a plurality of openings, each having a third physical opening size; separating a second fraction of embryo explants from a second portion of the debris material by length, width, or thickness relative to the third physical opening size; contacting the second fraction with a third moving sieve comprising a plurality of openings, each comprising a fourth physical opening size; separating a third fraction of embryo explants from a third portion of the debris material by length, width, or thickness relative to the fourth physical opening size; and aspirating the third fraction of embryo explants to obtain a first aspirated fraction of plant embryo explants, wherein the second gap distance is about 0.5 mm to about 2.5 mm or is about 1.5 mm, and wherein the moving plate, the first moving sieve, the second moving sieve, and the third moving sieve move in a linear motion.

In other aspects, the present disclosure provides a method of purifying genetically modifiable dry plant embryo explants, the method comprising: contacting a preparation of dry plant embryo explants comprising meristematic tissue with a first moving sieve, wherein the first moving sieve comprises a plurality of openings, each having a first sieve physical opening size, and wherein the preparation comprises a population of dry plant embryo explants and debris material; separating a first fraction of embryo explants from a first portion of the debris material by length, width, or thickness relative to the first sieve physical opening size, or relative to a first sieve effective opening size; contacting the first fraction with a first vibratory screen, wherein the first vibratory screen comprises a plurality of openings, each having a first screen opening size and a first screen opening shape, and wherein the first fraction comprises a population of dry plant embryo explants, and the debris material; vibrating the first vibratory screen to produce a first screen motion, wherein the first screen motion comprises a first horizontal vibratory component; and separating a first screen fraction of embryo explants from a first screen portion of the debris material by length, width, or thickness relative to the first screen opening size or the first screen opening shape, or by a displacement of the first screen fraction relative to a displacement of the first screen portion of the debris material produced by the first screen motion, wherein the first moving sieve moves in a circular, elliptical, or linear motion. In some embodiments, the methods of the present disclosure may comprise: contacting a preparation of dry plant embryo explants comprising meristematic tissue with a first moving sieve, wherein the first moving sieve comprises a plurality of openings, each having a first sieve physical opening size, and wherein the preparation comprises a population of dry plant embryo explants and debris material; separating a first fraction of embryo explants from a first portion of the debris material by length, width, or thickness relative to the first sieve physical opening size, or relative to a first sieve effective opening size; contacting the first fraction with a second moving sieve, wherein the second moving sieve comprises a plurality of openings, each having a second sieve physical opening size; separating a second fraction of embryo explants from a second portion of the debris material remaining in the first fraction by length, width, or thickness relative to the second sieve physical opening size, or relative to a second sieve effective opening size; contacting the second fraction with a first vibratory screen, wherein the first vibratory screen comprises a plurality of openings, each having a first screen opening size and a first screen opening shape, and wherein the second fraction comprises a population of dry plant embryo explants, and the debris material; vibrating the first vibratory screen to produce a first screen motion, wherein the first screen motion comprises a first horizontal vibratory component; and separating a first screen fraction of embryo explants from a first screen portion of the debris material by length, width, or thickness relative to the first screen opening size or the first screen opening shape, or by a displacement of the first screen fraction relative to a displacement of the first screen portion of the debris material produced by the first screen motion, wherein the first moving sieve and the second moving sieve move in a circular, elliptical, or linear motion. In certain embodiments, the methods of the present disclosure may comprise: contacting a preparation of dry plant embryo explants comprising meristematic tissue with a first moving sieve, wherein the first moving sieve comprises a plurality of openings, each having a first sieve physical opening size, and wherein the preparation comprises a population of dry plant embryo explants and debris material; separating a first fraction of embryo explants from a first portion of the debris material by length, width, or thickness relative to the first sieve physical opening size, or relative to a first sieve effective opening size; contacting the first fraction with a second moving sieve, wherein the second moving sieve comprises a plurality of openings, each having a second sieve physical opening size; separating a second fraction of embryo explants from a second portion of the debris material remaining in the first fraction by length, width, or thickness relative to the second sieve physical opening size, or relative to a second sieve effective opening size; contacting the second fraction with a third moving sieve, wherein the third moving sieve comprises a plurality of openings, each having a third sieve physical opening size; separating a third fraction of embryo explants from a third portion of the debris material remaining in the second fraction by length, width, or thickness relative to the third sieve physical opening size, or relative to a third sieve effective opening size; contacting the third fraction with a first vibratory screen, wherein the first vibratory screen comprises a plurality of openings, each having a first screen opening size and a first screen opening shape, and wherein the third fraction comprises a population of dry plant embryo explants, and the debris material; vibrating the first vibratory screen to produce a first screen motion, wherein the first screen motion comprises a first horizontal vibratory component; and separating a first screen fraction of embryo explants from a first screen portion of the debris material by length, width, or thickness relative to the first screen opening size or the first screen opening shape, or by a displacement of the first screen fraction relative to a displacement of the first screen portion of the debris material produced by the first screen motion, wherein the first moving sieve, the second moving sieve, and the third moving sieve move in a circular, elliptical, or linear motion.

In yet other aspects, the present disclosure provides contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction with a first textured surface of a first vibratory platform, wherein the first textured surface of the first vibratory platform is substantially planar, and wherein the first fraction comprises a population of dry plant embryo explants and debris material; vibrating the first vibratory platform to produce a first platform motion; and separating a first platform fraction of the plant embryo explants from a first platform portion of the debris material according to a displacement of the first platform fraction relative to a displacement of the first platform portion of debris material on the first textured surface of the first vibratory platform, wherein the first moving sieve moves in a circular, elliptical, or linear motion. The methods of the present disclosure, in particular embodiments, may comprise: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; separating a second fraction of embryo explants from a second portion of the debris material remaining in the first fraction by length, width, or thickness relative to the second physical opening size, or relative to a second effective opening size; contacting the second fraction with a first textured surface of a first vibratory platform, wherein the first textured surface of the first vibratory platform is substantially planar, and wherein the second fraction comprises a population of dry plant embryo explants and debris material; vibrating the first vibratory platform to produce a first platform motion; and separating a first platform fraction of the plant embryo explants from a first platform portion of the debris material according to a displacement of the first platform fraction relative to a displacement of the first platform portion of debris material on the first textured surface of the first vibratory platform, wherein the first moving sieve and the second moving sieve move in a circular, elliptical, or linear motion. In certain embodiments, the methods of the present disclosure may comprise: contacting a preparation of dry plant embryo explants comprising meristematic tissue with a first moving sieve, wherein the first moving sieve comprises a plurality of openings, each having a first physical opening size, and wherein the preparation comprises a population of dry plant embryo explants and debris material; separating a first fraction of embryo explants from a first portion of the debris material by length, width, or thickness relative to the first physical opening size, or relative to a first effective opening size; contacting the first fraction with a second moving sieve, wherein the second moving sieve comprises a plurality of openings, each having a second physical opening size; separating a second fraction of embryo explants from a second portion of the debris material remaining in the first fraction by length, width, or thickness relative to the second physical opening size, or relative to a second effective opening size; contacting the second fraction with a third moving sieve, wherein the third moving sieve comprises a plurality of openings, each having a third physical opening size; separating a third fraction of embryo explants from a third portion of the debris material remaining in the second fraction by length, width, or thickness relative to the third physical opening size, or relative to a third effective opening size; contacting the third fraction with a first textured surface of a first vibratory platform, wherein the first textured surface of the first vibratory platform is substantially planar, and wherein the third fraction comprises a population of dry plant embryo explants and debris material; vibrating the first vibratory platform to produce a first platform motion; and separating a first platform fraction of the plant embryo explants from a first platform portion of the debris material according to a displacement of the first platform fraction relative to a displacement of the first platform portion of debris material on the first textured surface of the first vibratory platform, wherein the first moving sieve, the second moving sieve, and the third moving sieve move in a circular, elliptical, or linear motion.

In still yet other aspects, the present disclosure provides a method comprising: contacting a preparation of dry plant embryo explants comprising meristematic tissue with a moving plate, wherein the moving plate comprises a proximal end, a distal end, and a plurality of openings located near the distal end, each comprising a first physical opening size, and wherein the first preparation comprises a population of embryo explants and debris material; passing the preparation through the plurality of openings of the moving plate and contacting a first moving sieve with the first preparation, wherein the first moving sieve comprises a plurality of openings, each having a second physical opening size; separating a first fraction of embryo explants from a first portion of the debris material by length, width, or thickness relative to the second physical opening size; contacting the first fraction with a first textured surface of a first vibratory platform, wherein the first textured surface of the first vibratory platform is substantially planar, and wherein the first fraction comprises a population of dry plant embryo explants and debris material; vibrating the first vibratory platform to produce a first platform motion; and separating a first platform fraction of the plant embryo explants from a first platform portion of the debris material according to a displacement of the first platform fraction relative to a displacement of the first platform portion of debris material on the first textured surface of the first vibratory platform, wherein the moving plate and the first moving sieve move in a linear motion. In particular embodiments, the methods of the present disclosure may comprise: contacting a preparation of dry plant embryo explants comprising meristematic tissue with a moving plate, wherein the moving plate comprises a proximal end, a distal end, and a plurality of openings located near the distal end, each comprising a first physical opening size, and wherein the first preparation comprises a population of embryo explants and debris material; passing the preparation through the plurality of openings of the moving plate and contacting a first moving sieve with the first preparation, wherein the first moving sieve comprises a plurality of openings, each having a second physical opening size; separating a first fraction of embryo explants from a first portion of the debris material by length, width, or thickness relative to the second physical opening size; positioning a third grinding plate and a fourth grinding plate to define a second gap having a second gap distance between the third plate and the fourth plate; rotating the third plate or the fourth plate about an axis of rotation; contacting the first fraction with an interior surface of the third plate and an interior surface of the fourth plate to produce a second preparation of embryo explants comprising meristematic tissue; contacting the second preparation with a second moving sieve comprising a plurality of openings, each having a third physical opening size; separating a second fraction of embryo explants from a second portion of the debris material by length, width, or thickness relative to the third physical opening size; contacting the second fraction with a third moving sieve comprising a plurality of openings, each comprising a fourth physical opening size; separating a third fraction of embryo explants from a third portion of the debris material by length, width, or thickness relative to the fourth physical opening size; contacting the third fraction with a first textured surface of a first vibratory platform, wherein the first textured surface of the first vibratory platform is substantially planar, and wherein the third fraction comprises a population of dry plant embryo explants and debris material; vibrating the first vibratory platform to produce a first platform motion; and separating a first platform fraction of the plant embryo explants from a first platform portion of the debris material according to a displacement of the first platform fraction relative to a displacement of the first platform portion of debris material on the first textured surface of the first vibratory platform, wherein the second gap distance is about 0.5 mm to about 2.5 mm or is about 1.5 mm, and wherein the moving plate, the first moving sieve, the second moving sieve, and the third moving sieve move in a linear motion.

In some aspects, the present disclosure provides a method comprising: aspirating a preparation or a selected fraction of dry plant embryo explants comprising meristematic tissue to obtain a selected aspirated fraction of plant embryo explants, wherein the preparation or the selected fraction comprises a population of dry plant embryo explants and debris material, and wherein the selected aspirated fraction is a first aspirated fraction, a second aspirated fraction, a third aspirated fraction, a fourth aspirated fraction, a fifth aspirated fraction, or a sixth aspirated fraction; contacting the selected aspirated fraction with a first vibratory screen, wherein the first vibratory screen comprises a plurality of openings, each having a first opening size and a first opening shape, and wherein the selected fraction comprises a population of dry plant embryo explants, and the debris material; vibrating the first vibratory screen to produce a first screen motion, wherein the first screen motion comprises a first horizontal vibratory component; and separating a first screen fraction of embryo explants from a first screen portion of the debris material by length, width, or thickness relative to the first opening size or the first opening shape, or by a displacement of the first screen fraction relative to a displacement of the first screen portion of the debris material produced by the first screen motion.

In other aspects, the present disclosure provides, a method comprising: aspirating a preparation or a selected fraction of dry plant embryo explants comprising meristematic tissue to obtain a selected aspirated fraction of plant embryo explants, wherein the preparation or the selected fraction comprises a population of dry plant embryo explants and debris material, and wherein the selected aspirated fraction is a first aspirated fraction, a second aspirated fraction, a third aspirated fraction, a fourth aspirated fraction, a fifth aspirated fraction, or a sixth aspirated fraction; contacting the selected aspirated fraction with a first textured surface of a first vibratory platform, wherein the first textured surface of the first vibratory platform is substantially planar, and wherein the selected aspirated fraction comprises a population of dry plant embryo explants and debris material; vibrating the first vibratory platform to produce a first platform motion; and separating a first platform fraction of the plant embryo explants from a first platform portion of the debris material according to a displacement of the first platform fraction relative to a displacement of the first platform portion of debris material on the first textured surface of the first vibratory platform.

In certain embodiments of the present disclosure, the methods provided herein may comprise or may further comprise: sanitizing the population of plant seeds prior to passing the population through the first gap; or sanitizing the population of plant seeds prior to contacting the population with the interior surface of the first plate and the interior surface of the second plate. In some embodiments, the methods of the present disclosure may comprise or may further comprise: drying the population of plant seeds to a desired moisture content prior to passing the population through the first gap; or drying the population of plant seeds to a desired moisture content prior to contacting the population with the interior surface of the first plate and the interior surface of the second plate.

The methods of the present disclosure, in particular embodiments, may comprise or may further comprise: separating the first preparation into a first top preparation fraction, a first middle preparation fraction, and a first bottom preparation fraction, wherein the first top preparation fraction is retained on the first moving sieve, the first middle preparation fraction is retained on the second moving sieve, and the first bottom preparation fraction is retained on the third moving sieve. In particular embodiments, the methods of the present disclosure may comprise or further comprise: separating the second preparation into a second top preparation fraction, a second middle preparation fraction, and a second bottom preparation fraction, wherein the second top preparation fraction is retained on the first moving sieve, the second middle preparation fraction is retained on the second moving sieve, and the second bottom preparation fraction is retained on the third moving sieve. The methods of the present disclosure, in particular embodiments, may comprise or further comprise: combining the first middle preparation fraction with the second middle preparation fraction to produce a combined middle preparation fraction; or combining the first bottom preparation fraction with the second bottom preparation fraction to produce a combined bottom preparation fraction.

In a number of embodiments, the methods of the present disclosure may comprise or further comprise: contacting a preparation of plant seeds, a preparation of plant embryo explants, or any purified fraction thereof with an interior surface of a rotating cylinder, wherein the interior surface comprises a plurality of indentations, the indentations having an indentation size and an indentation shape, and wherein the preparation of plant seeds, the preparation of plant embryo explants, or the any purified fraction thereof comprises a population of dry plant embryo explants and debris material; rotating the rotating cylinder about an axis of rotation to produce a centrifugal force acting on the preparation of plant seeds, the preparation of plant embryo explants, or the any purified fraction thereof, wherein the axis of rotation is substantially parallel to the ground; and separating a first cylinder fraction of the plant embryo explants from a first cylinder portion of the debris material according to a displacement of the first cylinder portion of the debris material relative to a displacement of the first cylinder fraction of plant embryo explants produced by the rotating.

In many embodiments, the methods of the present disclosure may comprise or further comprise: aspirating a preparation of plant seeds, a preparation of plant embryo explants, or any purified fraction thereof to obtain an aspirated fraction of plant embryo explants. The aspirated fraction, in some embodiments, may be a first aspirated fraction, a second aspirated fraction, a third aspirated fraction a fourth aspirated fraction, a fifth aspirated fraction, or a sixth aspirated fraction.

The methods of the present disclosure, in some embodiments, may comprise or further comprise: contacting a preparation of plant seeds, a preparation of plant embryo explants, or any purified fraction thereof with a first vibratory screen, wherein the first vibratory screen comprises a plurality of openings, each having a first opening size and a first opening shape, and wherein the preparation of plant seeds, the preparation of plant embryo explants, or the any purified fraction thereof comprises a population of dry plant embryo explants, and the debris material; vibrating the first vibratory screen to produce a first screen motion, wherein the first screen motion comprises a first horizontal vibratory component; and separating a first screen fraction of embryo explants from a first screen portion of the debris material by length, width, or thickness relative to the first opening size or the first opening shape, or by a displacement of the first screen fraction relative to a displacement of the first screen portion of the debris material produced by the first screen motion. In certain embodiments, the methods of the present disclosure may further comprise: contacting the first screen fraction with a second vibratory screen, wherein the second vibratory screen comprises a plurality of openings, each having a second opening size and a second opening shape; vibrating the second vibratory screen to produce a second screen motion, wherein the second screen motion comprises a second horizontal vibratory component; and separating a second screen fraction of embryo explants from a second screen portion of the debris material comprised in the first screen fraction by length, width, or thickness relative to the second opening size or the second opening shape, or by a displacement of the second screen fraction relative to a displacement of the second screen portion of the debris material produced by the second screen motion.

The present disclosure, in additional embodiments, provides a method comprising or further comprising: contacting a preparation of plant seeds, a preparation of plant embryo explants, or any purified fraction thereof with a first textured surface of a first vibratory platform, wherein the first textured surface of the first vibratory platform is substantially planar, and wherein the preparation of plant seeds, the preparation of plant embryo explants, or the any purified fraction thereof comprises a population of dry plant embryo explants and debris material; vibrating the first vibratory platform to produce a first platform motion; and separating a first platform fraction of the plant embryo explants from a first platform portion of the debris material according to a displacement of the first platform fraction relative to a displacement of the first platform portion of debris material on the first textured surface of the first vibratory platform. The methods provided herein, in particular embodiments, may further comprise: contacting the first platform fraction with a second textured surface of a second vibratory platform, wherein the second textured surface of the second vibratory platform is substantially planar; vibrating the second vibratory platform to produce a second platform motion; and separating a second platform fraction of the plant embryo explants of the first platform fraction from a second platform portion of the debris material according to a displacement of the second platform fraction relative to a displacement of the second platform portion of debris material on the second textured surface of the second vibratory platform.

In particular embodiments, the methods of the present disclosure may comprise or further comprise: floating a preparation of plant seeds, a preparation of plant embryo explants, or any purified fraction thereof in an aqueous solution. In certain embodiments, the methods of the of the present disclosure may further comprise: collecting any purified fraction of plant embryo explants. The methods of the present disclosure, in some embodiments, may comprise applying a cryogenic treatment to a preparation of plant seeds, a preparation of plant embryo explants, or any purified fraction thereof.

In particular embodiments, the population of embryo explants comprises a population of corn, wheat, soybean, cotton, or canola embryo explants. The population of embryo explants, in some embodiments, is prepared at least in part by milling a population of dry or mature plant seeds. The population of dry or mature plant seeds, in certain embodiments, comprises a population of corn, wheat, soybean, cotton, or canola seeds. In some embodiments, the population of dry plant or mature plant seeds has an average internal moisture content of about 3% to about 25%. The population of embryo explants, in particular embodiments, is prepared by excising a plurality of embryo explants from a population of plant seeds and drying the plurality of embryo explants to a desired moisture content. In particular embodiments, the embryo explants do not germinate and remain viable and competent for genetic modification. The embryo explants, in some embodiments, have an internal moisture content of about 3% to about 25%. In certain embodiments, the embryo explants comprise the apical portion of the embryo axis lacking the radical.

### BRIEF DESCRIPTION OF DRAWINGS

FIG. 1 (A-D) shows a diagram of certain embodiments of a seed roller mill and a seed grinder for excising dry embryo explants from plant seeds.

FIG. 2 (A-D) illustrates different types of roll cut orientations.

FIG. 3 shows a diagram of one embodiment of a siever for purifying dry embryo explants comprised within a preparation of plant embryo explants.

FIG. 4 shows a diagram of one embodiment of a siever for purifying dry embryo explants comprised within a preparation of plant embryo explants.

FIG. 5 shows a diagram of one embodiment of a rotating cylinder for purifying dry embryo explants comprised within a preparation of plant embryo explants.

FIG. 6 shows a diagram of one embodiment of an apparatus for aspirating and purifying dry embryo explants comprised within a preparation of plant embryo explants.

FIG. 7 shows a diagram of one embodiment of an apparatus for aspirating and purifying dry embryo explants comprised within a preparation of plant embryo explants.

FIG. 8 (A-B) shows a diagram of one embodiment of an apparatus for aspirating and purifying dry embryo explants comprised within a preparation of plant embryo explants.

FIG. 9 shows a diagram of one embodiment of an apparatus comprising a vibratory screen for purifying dry embryo explants comprised within a preparation of plant embryo explants.

FIG. 10 (A-C) shows a diagram of one embodiment of an apparatus comprising a vibratory platform comprising a textured surface for purifying dry embryo explants comprised within a preparation of plant embryo explants.

FIG. 11 illustrates an example of a workflow for preparing and purifying corn dry embryo explants from a preparation of plant embryo explants.

FIG. 12 illustrates an example of a workflow for preparing and purifying soybean dry embryo explants from a preparation of plant embryo explants.

FIG. 13 illustrates an example of a workflow for preparing and purifying cotton dry embryo explants from a preparation of plant embryo explants.

FIG. 14 illustrates an example of a workflow for preparing and purifying wheat dry embryo explants from a preparation of plant embryo explants.

FIG. 15 illustrates an example of a workflow for preparing and purifying canola dry embryo explants from a preparation of plant embryo explants.

### DETAILED DESCRIPTION

The following is a detailed description provided to aid those skilled in the art in practicing the embodiments disclosed herein. Modifications and variations to the embodiments described herein can be made without departing from the spirit or scope of the present disclosure. Apparatuses, systems, and methods are provided for purifying dry embryo explants for genetic modification, which may include one or more steps of sanitizing, drying, milling, coarse width sizing, length sizing aspiration, width and thickness separation, aspiration-classification, or separation using a friction table as described herein.

The present disclosure therefore provides apparatuses, systems, and methods for purifying dry embryo explants from plant seeds. Such embryo explants may be produced by removing seed parts from plant seeds and isolating the embryo explants from debris material to obtain a purified population of genetically modifiable dry embryo explants. As used herein, “debris material” includes any undesired material that may be present in a sample or preparation, which may include any material other than meristem-containing or meristematic embryo explants, non-seed plant material, dust, and other non-meristematic parts of the seed, such as all or part of the cotyledon, endosperm, and/or seed coat. The present disclosure represents a substantial advance in the art, as it provides methods for producing populations of dry embryo explants that are significantly more efficient in generating genetically modified plants or parts compared to populations of dry embryo explants that have existed to date. The present disclosure further provides apparatuses, systems, and methods which improve the workflow associated with producing genetically modified plants or plant parts from dry embryo explants. Purified explants, as described herein, significantly improve the efficiency at which genetically modified plants or plant parts are generated at least by decreasing contamination, improving explant health, and providing a sustainable, clean culture systems from which genetically modified plants and plant parts may be recovered.

Any embodiment discussed herein with respect to one aspect of the disclosure applies to other aspects as well, unless specifically noted. Any embodiment or aspect of the present disclosure may be combined with any other embodiment or aspect, unless specifically noted.

### A. Dry Embryo Explant Preparations

In one aspect, the present disclosure provides apparatuses, systems, and methods for excising and purifying dry embryo explants from plant seeds. Such purified dry embryo explants are useful in methods of producing genetically modified plants or plant parts. Preparations of plant embryo explants comprising a population of dry embryo explants and debris material may be produced from seeds by applying mechanical force, for example by cutting, grinding, scraping, crushing, or wounding, the seeds. Seeds for use according to the present disclosure may be harvested from plants grown in a field, greenhouse, controlled environment, or growth chamber, and may be mature or immature seeds, but may preferably be mature seeds. Examples of seeds for use in the compositions, systems, and methods provided include, but are not limited to, monocot seeds, dicot seeds, corn seeds, soybean seeds, wheat seeds, cotton seeds, and canola seeds. Examples of dry embryo explants for use in the compositions, systems, and methods provided include, but are not limited to, monocot embryo explants, dicot embryo explants, corn embryo explants, soybean embryo explants, wheat embryo explants, cotton embryo explants, and canola embryo explants. Use of mature seeds may provide the benefits or advantages of improved seed storage, explant preparation, and/or culturing. Examples of monocot plants, seeds, or explants that may be used according to present embodiments include those derived from any plant species within the Poaceae or Gramineae family of monocot or cereal plants and grasses, which may include any Zea genus corn or maize species, such as Zea mays, any Oryza genus or rice species, such as Oryza sativa, any Triticum genus or wheat species, such as Triticum aestivum or Triticum turgidum var durum, any Hordeum genus or barley species, such as Hordeum vulgare, any Avena genus or oat species, such as Avena sativa, any Sorghum genus or sorghum species, such as Sorghum bicolor or Sorghum vulgare, any Secale genus or rye species, such as Secale cereale, any Saccharum genus or sugarcane species, or any Setaria, Pennisetum, Eleusine, Echinochloa, or Panicum genus or millet species, such as Setaria virdis, Setaria italica, Pennisetum glaucum, Eleusine coracana, Echinochloa frumentacea, Panicum sumatrense, or Panicum miliaceum. Examples of dicot plants, seeds, and explants that may be used according to the present embodiments include those derived from any plant species within, for example, the family Fabaceae, Malvaceae, or Brassicaceae, which may include any Glycine genus or soybean species, such as Glycine max, any Gossypium genus or cotton species, such as Gossypium arboretum, Gossypium herbaceum, Gossypium raimondii, Gossypium thurberi, Gossypium barbadense, Gossypium hirsutum, Gossypium darwinii, Gossypium mustelinum, Gossypium tomentosum, Gossypioides brevilanatum, or Gossypioides kirkii, Medicago genus or alfalfa species, such as Medicago sativa, or any Brassica genus species, such as Brassica napus, Brassica rapa, or Brassica juncea. Other examples of dicot plants, seeds, and explants that may be used according to the present embodiments include other leguminous plants, such as beans, peas, peanuts, lentils, chickpeas, clover, sunflower (Helianthus annuus), safflower (Carthamus tinctorius), oil palm (Elaeis spp.), sesame (Sesamum spp.), coconut (Cocos spp.), tobacco (Nicotiana tabacum), potato (Solanum tuberosum), sweet potato (Ipomoea batatus), cassava (Manihot esculenta), coffee (Coffea spp.), tea (Camellia spp.), fruit trees, such as apple (Malus spp.), Prunus spp., such as plum, apricot, peach, and cherry, pear (Pyrus spp.), fig (Ficus casica), banana (Musa spp.), citrus trees (Citrus spp.), cocoa (Theobroma cacao), avocado (Persea americana), olive (Olea europaea), almond (Prunus amygdalus), walnut (Juglans spp.), strawberry (Fragaria spp.), watermelon (Citrullus lanatus), pepper (Capsicum spp.), sugar beet (Beta vulgaris), grape (Vitis, Muscadinia), tomato (Lycopersicon esculentum, Solanum lycopersicum), and cucumber (Cucumis sativis).

According to some embodiments, apparatuses, systems, and methods are provided for purifying explants from a preparation of plant embryo explants comprising a population of explants and debris material. Populations of explants produced by the apparatuses, systems, and methods of the present disclosure are also provided herein. As used herein, the term “explant” or “seed embryo explant” refers to a plant part or plant tissue that is capable of being genetically modified and subsequently regenerated into a genetically modified plant or plant part. An “explant” or “seed embryo explant” may refer to any part of a plant seed, which may comprise at least a portion of a plant seed embryo. An “explant” or “seed embryo explant” may comprise an embryo explant excised from a plant seed that may comprise at least a part of an embryo meristematic tissue. Alternatively, an “explant” or “seed embryo explant” may refer to a partially opened plant seed that may be produced by any suitable mechanical process. As used in reference to an explant or seed embryo explant, “partially opened” refers to an altered state of a plant seed that has one or more openings or fissures in the plant seed. Such openings or fissures may be introduced by a mechanical force, such as squeezing, crushing, rolling, pressing, or extruding. An explant or seed embryo explant that is a whole or intact plant seed or a crushed, deformed or partially opened plant seed may in many cases have its seed coat removed. An explant may be defined, in one aspect or embodiment, as comprising meristematic tissue or embryonic meristem tissue, which contains plant cells that can differentiate or develop to produce multiple plant structures including, but not limited to, stem, roots, leaves, germ line tissue, shoots or multiple shoots, and seeds. Indeed, an embryo explant may be defined as comprising all or part of a seed embryo removed from other non-embryonic seed tissues and further comprising all or part of a meristematic tissue or embryonic meristem tissue. In some embodiments, the present disclosure provides embryo explants comprising the apical portion of the embryo axis lacking the radical. In certain embodiments, the present disclosure provides embryo explants which do not germinate and remain viable and competent for genetic modification. As used herein a “population of embryo explants” refers to a group of explants from the same plant species. The population of explants, in some embodiments, may include explants having the same or a different genotype. In certain embodiments, the genotype of the explants within the population may be known or may be unknown. In specific embodiments, the population of embryo explants may refer to a group of embryo explants which includes embryo explants of at least two different plant genotypes. As used herein, a “genetically modified” plant, plant part, plant tissue, explant, or plant cell comprises a genetic modification or transgene introduced into the genome of the plant, plant part, plant tissue, explant, or plant cell through genetic engineering, which may be via a genetic transformation or a genome editing technique. As used herein, a “transgenic” plant, plant part, plant tissue, explant or plant cell has an exogenous or heterologous nucleic acid sequence, polynucleotide, expression cassette, or transgene integrated into the genome of the plant, plant part, plant tissue, explant, or plant cell. In certain embodiments, explants according to this disclosure may be produced manually or using an automated process. For example, seed tissues may be removed from a seed by cutting, grinding, scraping, crushing, wounding, or any other similar process. Manual or automated methods for removal of unnecessary seed parts may also be carried out. A fluid, non-limiting examples of which include compressed air, other gases, and liquids, can be used to separate explants from debris during explant purification. The present disclosure provides different unit operations for producing and/or purifying genetically modifiable plant embryo explants. A person of ordinary skill in the art viewing the specification would understand that the unit operations described herein may be combined into a single apparatus for producing and/or purifying plant embryo explants. A person of ordinary skill in the art would further understand that the unit operations described in the present disclosure may be performed manually or by an automated process.

Embryo explants may be excised from dry, dried, or wet seeds. Mature plant seeds may become drier as part of their normal maturation process, although seeds may be further dried prior to explant excision and/or explants may be dried following excision from seeds. Dry or dried excised plant embryo explants may be immediately used for genetic modification or may be stored for a period of time for later use. Explant preparation may further comprise drying the seed and/or explant to a desired moisture content. Drying the seed and/or explant to such a desired moisture content may improve excision, storage, and/or use of the seed and/or explant, depending upon the initial moisture content of the seed or explant. Following excision, the explant may be purified or separated from other seed material and debris by rinsing, floatation, or other methods known in the art. In certain embodiments, the present disclosure provides a seed or explant having an internal moisture of about 3% to about 25%, about 3% to about 20%, about 3% to about 15%, about 3% to about 10%, about 5% to about 10%, including about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or about 25% internal moisture, including all ranges and values derivable therebetween. An explant may be produced from a mature seed having a moisture content as described herein. In particular embodiments, the moisture content of the seed or explant may be measured prior to or after explant excision, prior to or after explant storage, during explant storage, prior to explant rehydration, and/or prior to genetic modification or transformation.

In one aspect, any embryo explant may be prepared or used according to the embodiments of the present disclosure. In particular embodiments, the embryo explant may be a mature embryo explant or an immature embryo explant. In some embodiments, the mature embryo explant is a dry excised embryo explant. Dry excised explants may be taken from seeds and used almost directly as targets for transformation or genetic modification. In some embodiments, dry excised explants may be taken from mature dry seeds and used as targets for transformation or genetic modification with perhaps only minimal wetting, hydration, or pre-culturing steps. In further embodiments, wet, dried wet, or wet excised embryo explants may be used as a target for transformation or genetic modification. As used herein “wet” embryo explants refer to dry excised explants subjected to wetting, hydration, imbibition, or other minimal culturing steps prior to transformation or genetic modification. As used herein “dried wet” embryo explants refer to embryo explants which are primed for germination by wetting and then dried to arrest germination. As used herein “wet excised” explants refer to explants excised from imbibed or hydrated seeds. A wet embryo explant is hydrated or imbibed after excision from a seed, whereas a wet excised embryo explant is excised from an already hydrated or imbibed seed. As used herein a “callus” refers to a proliferating mass of unorganized, undifferentiated and/or dedifferentiated plant cells or tissue.

Explants for use according to the present disclosure may be genetically modified at various times after isolation, excision, or removal from seed. In some embodiments, explants may have been removed from seeds for less than a day, for example, from about 1 to about 24 hours, such as about 1, 2, 3, 4, 5, 6, 7, 8, 9, 10, 11, 12, 13, 14, 15, 16, 17, 18, 19, 20, 21, 22, 23 or 24 hours prior to use. In particular embodiments, explants may be stored for longer periods, including days, weeks, months, or years prior to use. Methods and parameters for drying, storing, and germinating seed are known in the art (e.g., U.S. Pat. No. 8,362,317, specifically incorporated herein by reference in its entirety, Senaratna et al., 1983, Pl. Physiol. 72:620-624, 1983; Vertucci and Roos, 1990, Pl. Physiol. 90:1019-1023, 1990; Chai et al., 1998, Seed Science Research 8 (Supplement 1): 23-28, 1998). Any conditions may be used as desired, including incubation or storage at temperatures, for example, of about −80° C. to about 60° C.

The present disclosure may in certain aspects involve sterilization of seeds or explants. Sterilization can include contacting seed or explant material with various liquid or gases that serve to reduce or eliminate the presence of viable bacterial or fungal contaminants that could otherwise interfere with seed or embryo viability. Sterilization by application of liquid may also hydrate or partially hydrate the plant seeds, explants, embryos, or tissues and serve the purpose of priming or hydrating the seeds, explants, embryos, or tissues prior to transformation, editing, or further culturing. Methods for sterilization include, but are not limited to, the use of chlorine gas, ozone, solutions of bleach or alcohol, ultraviolet light, temperatures of −20° C. or lower, and exposure to a temperature higher than 40° C.

Purification of dry embryo explants may be measured and determined, in some embodiments of the present disclosure, by calculating the percentage of dry embryo explants per particle present in the sample. The methods and systems described herein may, for example, increase the purity of dry embryo explants in the sample by at least 0.1-fold, 0.2-fold, 0.3-fold, 0.4-fold, 0.5-fold, 0.6-fold, 0.7-fold, 0.8 fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, or 95-fold, including all ranges and values derivable therebetween. The methods and systems provided by the present disclosure may, in certain embodiments, increase the purity of dry embryo explants in a sample by about 0.1-fold to about 95-fold, about 1-fold to about 90-fold, about 1-fold to about 80-fold, about 1-fold to about 75-fold, about 1-fold to about 70-fold, about 1-fold to about 60-fold, about 1-fold to about 50-fold, about 1-fold to about 40-fold, about 1-fold to about 30-fold, about 1-fold to about 20-fold, about 1-fold to about 10-fold, about 1-fold to about 5-fold, about 2-fold to about 20-fold, about 2-fold to about 10-fold, about 2-fold to about 5-fold, about 0.5-fold, about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, or about 95-fold, including all ranges and values derivable therebetween.

### B. Seed Milling

In one aspect, the present disclosure provides apparatuses and systems for excising and purifying embryo explants from plant seeds. Embodiments disclosed herein may include, for example, an apparatus for producing a preparation comprising embryo explants comprising meristematic tissue and debris material from a population of plant seeds, the apparatus comprising at least one pair of grinding rollers having shaped teeth or raised ridges as described herein. In some embodiments, the apparatus may comprise at least two pair of grinding rollers. Grinding rollers may be made from any material comprising physical characteristics which permit embryo explant excision without damage during seed milling. Non limiting examples of such material include stainless steel, steel, and ceramic.

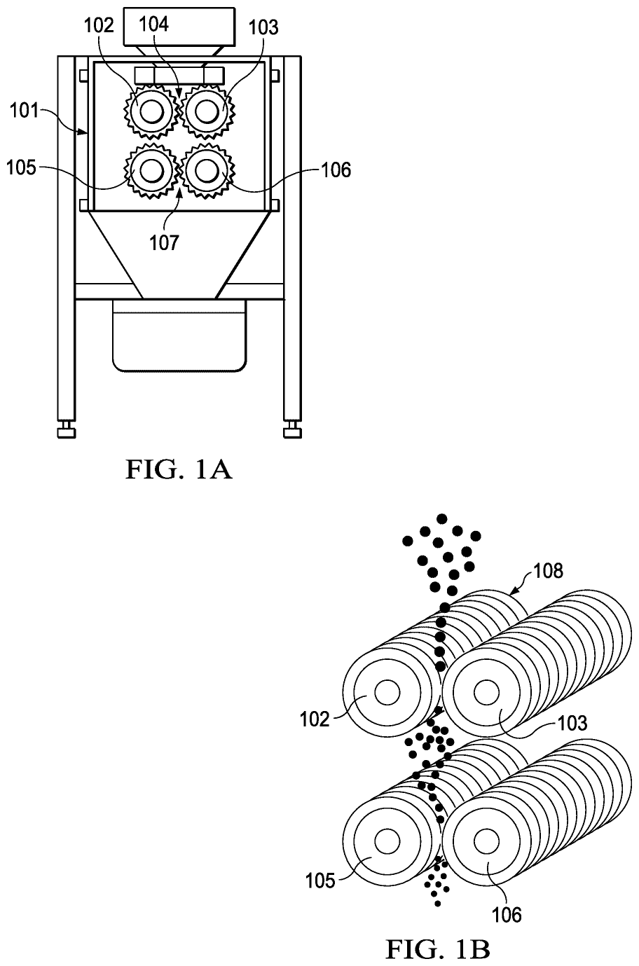

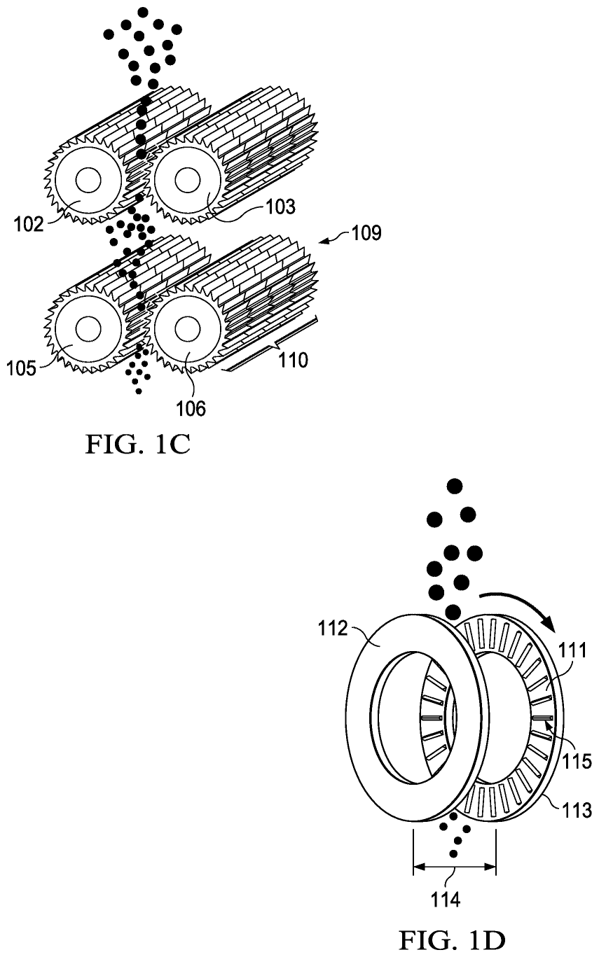

FIG. 1A is a diagram showing an apparatus having housing unit 101 to receive plant seeds; a first grinding roller 102 and a second grinding roller 103 attached to the housing unit 101 to grind the plant seeds; a first gap distance 104 between the first grinding roller 102 and the second grinding roller 103; a third grinding roller 105 and a fourth grinding roller 106 attached to the housing unit 101 to grind the plant seeds; and a second gap distance 107 between the third grinding roller and the fourth grinding roller. FIG. 1B is a diagram showing the process of contacting plant seeds with the exterior surface of a first grinding roller 102, a second grinding roller 103, a third grinding roller 105, and a fourth grinding roller 106, the exterior surface of each grinding roller comprising a plurality of raised ridges 108. FIG. 1C is a diagram showing the process of contacting plant seeds with the exterior surface of a first grinding roller 102, a second grinding roller 103, a third grinding roller 105, and a fourth grinding roller 106, the exterior surface of each grinding roller comprising a plurality of shaped teeth 109 which are configured into teeth rows, an example of which is shown in 110. FIG. 1D is a diagram showing the process of contacting plant seeds with the interior surface 111 of two grinding plates 112 and 113 positioned to produce a gap distance 114, the interior surface 111 of each grinding plate comprising a plurality of grinder teeth 115.

The International System of Units is used throughout the present disclosure, however, one of ordinary skill in the art could convert such units to the Imperial System. For example, 2.54 cm is equal to 1 inch, 25.4 mm is equal to 1 inch, and 1 in² is equal to 6.4516 cm².

In another aspect, the present disclosure provides a method of producing a preparation of plant embryo explants, the method comprising positioning a first grinding roller and a second grinding roller to define a first gap having a first gap distance between the first roller and the second roller; rotating the first roller about a first axis of rotation and the second roller about a second axis of rotation; and passing a population of plant seeds through the first gap to produce a first preparation of plant embryo explants comprising meristematic tissue, wherein the first gap distance is about 0.10 mm to about 7.62 mm, and wherein the first roller and the second roller each comprise an exterior surface and the exterior surface of the first roller and the exterior surface of the second roller each comprise a plurality of protrusions. As used herein in reference to a pair of grinding rollers, the term “gap distance” refers to the point of nearest contact between two grinding rollers. In some embodiments, the gap distance may be the distance measured between a peak of a protrusion of one grinding roller and a peak of a protrusion of another grinding roller.

In some embodiments, the method may further comprise positioning a third grinding roller and a fourth grinding roller to define a second gap having a second gap distance between the third roller and the fourth roller; rotating the third roller about a third axis of rotation and the fourth roller about a fourth axis of rotation; and passing the first preparation of embryo explants through the second gap to produce a second preparation of plant embryo explants comprising meristematic tissue, wherein the second gap distance is about 0.10 mm to about 7.62 mm, and wherein the third roller and the fourth roller each comprise an exterior surface and the exterior surface of the third roller and the exterior surface of the fourth roller each comprise a plurality of protrusions. As used herein the term “grinding roller” or “roller” refers to a substantially cylindrical member configured to rotate about the axis of rotation. A grinding roller may be used, for example, to cut, grind, scrape, crush, crack, and/or wound seeds. In some embodiments, a grinding roller may be structurally connected to the plurality of protrusions. As used herein structural components which are “structurally connected” are in direct or indirect structural contact with each other. For example, any structural component may be considered structurally connected to any other structural component if the components are each in contact with one or more shared structural components of an apparatus. In some embodiments, the plurality of protrusions may be defined as a plurality of shaped teeth or as a plurality of raised ridges. In particular embodiments, the first, second, third, and/or fourth axis of rotation is substantially parallel to the ground. In certain embodiments, the first axis of rotation is substantially parallel to the second axis of rotation. The third axis of rotation, in some embodiments, is substantially parallel to the third axis of rotation. As used herein with regard to the axis of rotation of a grinding roller the term “substantially parallel” refers to an axis of rotation that is essentially parallel to the ground but may, in some embodiments, vary by about +/−15 degrees. In specific embodiments, the axis rotation of a grinding roller may be essentially parallel to the ground but may vary by about +/−1 degree, about +/−2 degrees, about +/−3 degrees, about +/−4 degrees, about +/−5 degrees, about +/−6 degrees, about +/−7 degrees, about +/−8 degrees, about +/−9 degrees, about +/−10 degrees, about +/−11 degrees, about +/−12 degrees, about +/−13 degrees, about +/−14 degrees, or about +/−15 degrees, including all ranges and values derivable therebetween.

The gap distance between the rollers may be modified depending on the size and characteristics of the seed to be milled. The first gap distance or the second gap distance, in certain embodiments, is about 0.381 mm to about 7.62 mm, about 0.762 mm to about 6.35 mm, about 0.2032 mm to about 2.54 mm, or about 0.508 mm to about 1.016 mm, including all ranges and values derivable therebetween. In certain embodiments, the first gap distance or the second gap distance may be about 0.381 mm to about 7.62 mm, about 0.508 mm to about 6.35 mm, about 0.635 mm to about 5.08 mm, about 0.762 mm to about 3.81 mm, about 1.016 mm to about 3.175 mm, about 1.27 mm to about 3.175 mm, about 1.524 mm to about 3.048 mm, about 1.778 mm to about 2.921 mm, about 2.032 mm to about 2.794 mm, about 2.286 mm to about 2.794 mm, about 0.762 mm to about 3.175 mm, about 0.762 mm to 2.794 mm, about 0.762 mm to about 2.54 mm, about 0.762 mm to about 2.286 mm, about 0.762 mm to about 2.032 mm, about 0.762 mm to about 1.778 mm, about 1.106 mm to about 1.524 mm, about 0.762 mm to about 6.35 mm, about 1.27 mm to about 6.35 mm, about 1.778 mm to about 6.35 mm, about 2.286 mm to about 6.35 mm, about 2.54 mm to about 6.35 mm, about 3.048 mm to about 5.842 mm, about 3.556 mm to about 5.334 mm, about 3.556 mm to about 5.08 mm, about 3.81 mm to about 5.08 mm, about 4.064 mm to about 4.572 mm, about 2.54 mm to about 5.08 mm, about 3.048 mm to about 5.334 mm, about 3.556 mm to about 4.826 mm, about 3.556 mm to about 4.318 mm, about 3.556 mm to about 4.064 mm, about 0.2032 mm to about 2.54 mm, about 0.254 mm to about 2.286 mm, about 0.508 mm to about 2.032 mm, about 0.762 mm to about 1.788 mm, about 1.016 mm to about 1.524 mm, about 0.2032 mm to about 2.032 mm, about 0.2032 mm to about 1.524 mm, about 0.2032 mm to about 1.524 mm, about 0.2032 mm to about 1.016 mm, about 0.2032 mm to about 0.508 mm, about 0.2286 mm to about 0.4572 mm, about 0.254 mm to about 0.4064 mm, about 0.254 mm to about 1.524 mm, about 0.508 mm to about 1.27 mm, about 0.508 mm to about 1.016 mm, about 0.762 mm to about 1.016 mm, about 0.762 mm to about 0.889 mm, about 0.508 mm to about 0.762 mm, or about 0.635 mm to about 0.762, including all ranges and values derivable therebetween. Contacting, in particular embodiments, may comprise contacting the population of plant seeds or a preparation thereof with the exterior surface of the first roller and the exterior surface of the second roller approximately simultaneously; and/or contacting the preparation with the exterior surface of the third roller and the exterior surface of the fourth roller approximately simultaneously.

In certain embodiments, the present disclosure provides a method comprising contacting a population of plant seeds or a preparation thereof with the exterior surface of one or more grinding rollers as described herein, wherein the exterior surface of the grinding roller comprises about 4 to about 20, about 4 to about 6, about 4 to about 8, about 4 to about 10, about 4 to about 12, about 6 to about 10, about 6 to about 12, about 6 to about 14, or about 8 to about 12 shaped teeth, or about 2 to about 21, about 2 to about 15, about 2 to about 10, about 2 to about 5, about 5 to about 21, about 5 to about 15, about 5 to about 10, about 10 to about 21, or about 10 to about 15 raised ridges per 2.54 cm. The exterior surface for example, may comprise about 4, 5, 6, 7, 8, 9, 10, 11, 12, 13, 14, 15, 16, 17, 18, 19, or 20 shaped teeth per 2.54 cm, including all ranges and values derivable therebetween, or about 2, 3, 4, 5, 6, 7, 8, 9, 10, 11, 12, 13, 14, 15, 16, 17, 18, 19, 20, or 21 raised ridges per 2.54 cm, including all ranges and values derivable therebetween. In some embodiments, the plurality of shaped teeth may be configured into teeth rows as shown in FIG. 1C. The orientation of the teeth rows or the raised ridges may, in particular embodiments, be configured to run substantially parallel to the axis of rotation as shown in FIG. 1B or substantially perpendicular to the axis of rotation as shown in FIG. 1C. The shaped teeth of the grinding rollers, in certain embodiments of the present disclosure, may comprise a sharp surface and a dull surface, and the shaped teeth may be oriented on the exterior surface of the grinding rollers in a manner configured to produce a roll cut orientation that is sharp to sharp, dull to dull, sharp to dull, or dull to sharp.

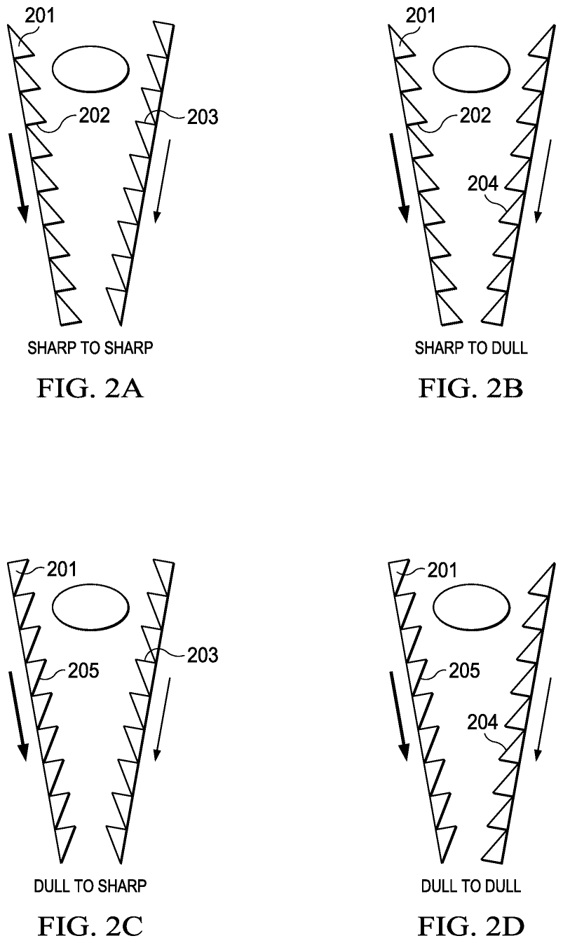

Roll cut orientations are illustrated in FIG. 2 (A-D). FIG. 2A is a diagram showing a sharp to sharp roll cut orientation. As shown in FIG. 2A, the shaped teeth 201 are oriented such that the sharp surface of the shaped teeth of the first roller 202 and the sharp surface of the shaped teeth of the second roller 203 first contact the plant seeds. FIG. 2B is a diagram showing a sharp to dull roll cut orientation. As shown in FIG. 2B the shaped teeth 201 are oriented such that the sharp surface of the shaped teeth of the first roller 202 and the dull surface of the shaped teeth of the second roller 204 first contact the plant seeds. FIG. 2C is a diagram showing a dull to sharp roll cut orientation. As shown in FIG. 2C, the shaped teeth 201 are oriented such that the dull surface of the shaped teeth of the first roller 205 and the sharp surface of the shaped teeth of the second roller 203 contact the plant seeds. FIG. 2D is a diagram showing a dull to dull roll cut orientation. As shown in FIG. 2D, the shaped teeth 201 are oriented such that the dull surface of the shaped teeth of the first roller 205 and the dull surface of the shaped teeth of the second roller 204 contact the plant seeds. In some embodiments, the first roller rotates faster than the second roller, as represented by the arrow to the outside of the first roller being larger than the arrow to the outside of the second roller in FIG. 2A, FIG. 2B, and FIG. 2C.

The number and orientation of the shaped teeth of the grinding rollers may be further described in terms of a roll cut identification, such as 8AS, 20ST, or LaPage Cut©. In a roll cut identification, the number indicates the number of teeth per 2.54 cm of two rollers at the point of near contact and the letters indicate the shape of the teeth. For example, 8AS would equal 8 teeth per 2.54 cm of two rollers at the point of near contact, or 4 teeth per 2.54 cm on a first roller and 4 teeth per 2.54 cm on a second roller. With regard to the shape of the teeth, AS indicates Alice Sharp teeth, which have a scalene shape, and ST indicates Saw Tooth teeth, which have an isosceles or equilateral triangle shape. Any tooth shape known in the art may be used according to the embodiments of the present disclosure. In LaPage Cut©, the rollers comprise one or more raised ridges. See, for example, FIG. 1B. The roll cut identification may be adjusted depending on the characteristics of the seed to be milled. In certain embodiments, the roll cut identification may be, for example, AS, multiple AS, ST, flat, or another shape with about 4 to about 20 teeth per 2.54 cm.

The methods of the present disclosure, in specific embodiments, may comprise contacting the population or a preparation thereof with the sharp surface of the shaped teeth of the first roller and the sharp surface of the shaped teeth of the second roller, with the dull surface of the shaped teeth of the first roller and the dull surface of the shaped teeth of the second roller, with the sharp surface of the shaped teeth of the first roller and the dull surface of the shaped teeth of the second roller, or with the dull surface of the shaped teeth of the first roller and the sharp surface of the shaped teeth of the second roller. In some embodiments, the methods of the present disclosure may comprise contacting the first preparation with the sharp surface of the shaped teeth of the third roller and the sharp surface of the shaped teeth of the fourth roller, with the dull surface of the shaped teeth of the third roller and the dull surface of the shaped teeth of the fourth roller, with the sharp surface of the shaped teeth of the third roller and the dull surface of the shaped teeth of the fourth roller, or with the dull surface of the shaped teeth of the third roller and the sharp surface of the shaped teeth of the fourth roller. In specific embodiments, the population of plant seeds or a preparation thereof may be first contacted with the sharp surface or the dull surface of the shaped teeth of the first, second, third, and/or fourth roller as described herein before contacting additional surfaces of the respective roller.

In particular embodiments of the present disclosure, the method may comprise rotating a grinding roller as described herein at a rate of rotation. In specific embodiments, the rate of rotation is measured at the exterior surface of the roller. The rate of rotation, for example, may be about 50 rpm to about 1200 rpm, about 50 rpm to about 1000 rpm, about 50 rpm to about 800 rpm, about 50 rpm to about 600 rpm, about 50 rpm to about 400 rpm, about 50 rpm to about 250 rpm, about 50 rpm to about 200 rpm, about 100 rpm to about 250 rpm, about 100 rpm to about 400 rpm, about 130 rpm to about 145 rpm, about 150 rpm to about 250 rpm, about 175 rpm to about 225 rpm, about 190 rpm to about 220 rpm, about 320 rpm to about 360 rpm, about 340 rpm to about 350 rpm, about 50 rpm, about 100 rpm, about 138 rpm, about 150 rpm, about 194 rpm, about 200 rpm, about 213 rpm, about 250 rpm, about 300 rpm, about 345 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, about 1000 rpm, about 1050 rpm, about 1100 rpm, about 1150 rpm, or about 1200 rpm, including all ranges and values derivable therebetween. The first, second, third, and/or fourth grinding rollers, in specific embodiments, may rotate at the same rate of rotation or may rotate at different rates of rotation. In specific embodiments, the first, second, third, and/or fourth grinding rollers may rotate at a rotation rate ratio of about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1 about 1:1 to about 3:1, about 1:1 to about 2.5:1, about 1:1 to about 2:1, about 1:1, about 2:1, about 2.5:1, about 3:1, about 4:1, about 5:1, about 6:1, about 7:1, about 8:1, about 9:1, about 10:1 about 1.1:1, about 1.2:1, about 1.3:1, about 1.4:1, about 1.5:1, about 1.6:1, about 1.7:1, about 1.8:1, or about 1.9:1, including all ranges and values derivable therebetween. As used herein the term “rotation rate ratio” refers to the rotation rate of one grinding roller relative to the rotation rate of another grinding roller.

In some embodiments, the present disclosure further provides a method comprising contacting a population corn seeds or a preparation thereof with a first, second, third, and/or fourth grinding roller as described herein, wherein the first gap distance is about 0.381 mm to about 7.62 mm, about 0.508 mm to about 6.35 mm, about 0.635 mm to about 5.08 mm, about 0.762 mm to about 3.81 mm, about 1.016 mm to about 3.175 mm, about 1.27 mm to about 3.175 mm, about 1.524 mm to about 3.048 mm, about 1.778 mm to about 2.921 mm, about 2.032 mm to about 2.794 mm, about 2.286 mm to about 2.794 mm, or is about 2.54 mm, including all ranges and values derivable therebetween; or wherein the second gap distance is about 0.381 mm to about 7.62 mm, about 0.508 mm to about 6.35 mm, about 0.635 mm to about 5.08 mm, about 0.762 mm to about 3.81 mm, about 0.762 mm to about 3.175 mm, about 0.762 mm to 2.794 mm, about 0.762 mm to about 2.54 mm, about 0.762 mm to about 2.286 mm, about 0.762 mm to about 2.032 mm, about 0.762 mm to about 1.778 mm, about 1.106 mm to about 1.524 mm, or is about 1.27 cm, including all ranges and values derivable therebetween. In certain embodiments, the present disclosure further provides a method comprising contacting a population of soybean seeds or a preparation thereof with a first, second, third, and/or fourth grinding roller as described herein, wherein the first gap distance is about 0.762 mm to about 6.35 mm, about 1.27 mm to about 6.35 mm, about 1.778 mm to about 6.35 mm, about 2.286 mm to about 6.35 mm, about 2.54 mm to about 6.35 mm, about 3.048 mm to about 5.842 mm, about 3.556 mm to about 5.334 mm, about 3.556 mm to about 5.08 mm, about 3.81 mm to about 5.08 mm, about 4.064 mm to about 4.572 mm, or is about 4.2926 mm; or wherein the second gap distance about 0.762 mm to about 6.35 mm, about 1.27 mm to about 6.35 mm, about 1.778 mm to about 6.35 mm, about 2.286 mm to about 6.35 mm, about 2.54 mm to about 6.35 mm, about 2.54 mm to about 5.08 mm, about 3.048 mm to about 5.334 mm, about 3.556 mm to about 4.826 mm, about 3.556 mm to about 4.318 mm, about 3.556 mm to about 4.064 mm, or is about 3.937 mm. In specific embodiments, the present disclosure further provides a method comprising contacting a population of wheat seeds or a preparation thereof with a first, second, third, and/or fourth grinding roller as described herein, wherein the first gap distance is about 0.2032 mm to about 2.54 mm, about 0.254 mm to about 2.286 mm, about 0.508 mm to about 2.032 mm, about 0.762 mm to about 1.788 mm, about 1.016 mm to about 1.524 mm, or is about 1.2827 mm; or wherein the second gap distance is about 0.2032 mm to about 2.54 mm, about 0.2032 mm to about 2.032 mm, about 0.2032 mm to about 1.524 mm, about 0.2032 mm to about 1.524 mm, about 0.2032 mm to about 1.016 mm, about 0.2032 mm to about 0.508 mm, about 0.2286 mm to about 0.4572 mm, about 0.254 mm to about 0.4064 mm, or is about 0.3683 mm. In particular embodiments, the present disclosure further provides a method comprising contacting a population of canola seeds or a preparation thereof with a first, second, third, and/or fourth grinding roller as described herein, wherein the first and/or second gap distance is about 0.508 mm to about 1.016 mm, about 0.762 mm to about 1.016 mm, about 0.762 mm to about 0.889 mm, about 0.508 mm to about 0.762 mm, about 0.635 mm to about 0.762, about 0.8509 mm, or about 0.6985 mm. In particular embodiments, a population plant seeds may be contacted with a first and a second grinding roller to obtain a preparation of embryo explants. A preparation of embryo explants, in particular embodiments, may then be contacted with the first and second grinding rollers to obtain a further preparation of embryo explants.

Embodiments of the present disclosure may include an apparatus for producing a preparation comprising embryo explants and debris material, wherein the apparatus comprises at least two grinding plates, at least one of which rotates relative to the other grinding plate (FIG. 1D). Grinding plates may be made from any material comprising physical characteristics which permit embryo explant excision without damage during seed milling. Non limiting examples of such materials include stainless steel, steel, ceramic, and titanium alloy.

In another aspect, the present disclosure provides a method of producing a preparation of plant embryo explants, the method comprising positioning a first grinding plate and a second grinding plate to define a first gap distance at a point of near contact between the first plate and the second plate; rotating the first plate or the second plate about an axis of rotation; and contacting a population of plant seeds with an interior surface of the first plate and an interior surface of the second plate to produce a first preparation of embryo explants comprising meristematic tissue, wherein the first gap distance is about 0.5 mm to about 5.0 mm or about 3.0 mm to about 3.25 mm, including all ranges and values derivable therebetween. In certain embodiments, the first gap distance may be about 0.5 mm to about 5.0 mm, about 0.5 mm to about 2.5 mm, about 2.5 mm to about 4.0 mm, about 2.75 mm to about 3.75 mm, about 2.75 mm to about 3.50 mm, about 3.0 mm to about 3.25 mm, or about 1.5 mm, including all ranges and values derivable therebetween. As used herein in reference to a pair of grinding plates, the term “gap distance” refers to the point of nearest contact between two grinding plates. In specific embodiments, the gap distance between two grinding plates, as described herein, may be measured at the point of nearest contact between the outer circumference of one grinding plate and the outer circumference of the other grinding plate. In some embodiments, the gap distance may be the distance measured between a peak of a grinder tooth of one grinding plate and a peak of a grinder tooth of another grinding plate. As used herein the term “grinding plate” or “plate” refers to a substantially planar member comprising an interior surface and an exterior surface. A grinding plate or plate may, in some embodiments, be configured to rotate about an axis of rotation. The axis of rotation may, in certain embodiments, be substantially perpendicular or substantially parallel to the ground. As used herein with regard to the axis of rotation of a grinding plate the term “substantially parallel” refers to an axis of rotation that is essentially parallel to the ground but may, in some embodiments, vary by about +/−15 degrees. In particular embodiments, the axis of rotation of the grinding plate may be essentially parallel to the ground but may vary by about +/−1 degree, about +/−2 degrees, about +/−3 degrees, about +/−4 degrees, about +/−5 degrees, about +/−6 degrees, about +/−7 degrees, about +/−8 degrees, about +/−9 degrees, about +/−10 degrees, about +/−11 degrees, about +/−12 degrees, about +/−13 degrees, about +/−14 degrees, or about +/−15 degrees, including all ranges and values derivable therebetween. As used herein with regard to the axis of rotation of a grinding plate the term “substantially perpendicular” refers to an axis of rotation that is essentially perpendicular to the ground but may, in some embodiments, vary by about +/−15 degrees. In certain embodiments, the axis of rotation of the grinding plate may be essentially perpendicular to the ground by may vary by about +/−1 degree, about +/−2 degrees, about +/−3 degrees, about +/−4 degrees, about +/−5 degrees, about +/−6 degrees, about +/−7 degrees, about +/−8 degrees, about +/−9 degrees, about +/−10 degrees, about +/−11 degrees, about +/−12 degrees, about +/−13 degrees, about +/−14 degrees, or about +/−15 degrees, including all ranges and values derivable therebetween. The interior surface of a grinding plate may, in specific embodiments, comprise a textured surface. The textured surface of a grinding plate may be, for example, any surface which is not a smooth surface. The interior surface of a grinding plate may, in certain embodiments, be structurally connected to a plurality of grinder teeth. The interior surface of the grinding plate may comprise, in certain embodiments, any surface which is able cut, grind, scrape, crush, crack, and/or wound seeds without causing damage to the embryo explants. In particular embodiments, the interior surface of the grinding plate may be made using any material having a Rockwell C scale of about 20 to about 60, including all ranges and values derivable therebetween. A grinding plate as described herein may be made using any material which is able cut, grind, scrape, crush, crack, and/or wound seeds without causing damage to the embryo explants. Non-limiting examples of such materials include stainless steel, steel, hardened steel, carbon steel, aluminum, ceramic, and titanium alloy. In particular embodiments, the surface of the grinding plate may be heat-treated or have a coating such as titanium oxide or silica. In some embodiments, the interior surface of the first plate comprises the textured surface and the interior surface of the second plate comprises the textured surface. Contacting, in particular embodiments, may comprise contacting the population of plant seeds or a preparation thereof with the interior surface of the first plate and the interior surface of the second plate approximately simultaneously. In specific embodiments, contacting may comprise contacting the population of plant seeds or a preparation thereof with the interior surface of two grinding plates, as described herein, at a rate of 100 g/min to about 1500 g/min, about 200 g/min to about 1400 g/min, about 300 g/min to about 1300 g/min, about 400 g/min to about 1200 g/min, about 600 g/min to about 1000 g/min, or about 800 g/min, including all ranges and values derivable therebetween. In particular embodiments, the rotating may comprise rotating the first plate at about 100 rpm to about 1000 rpm, about 200 rpm to about 800 rpm, about 200 rpm to about 600 rpm, about 300 rpm to about 500 rpm, or about 400 rpm, including all ranges and values derivable therebetween, wherein the second plate remains approximately stationary; or rotating the second plate at about 100 rpm to about 1000 rpm, about 200 rpm to about 800 rpm, about 200 rpm to about 600 rpm, about 300 rpm to about 500 rpm, or about 400 rpm, including all ranges and values derivable therebetween, wherein the first plate remains approximately stationary.

In specific embodiments, the present disclosure provides a method further comprising positioning a third grinding plate and a fourth grinding plate to define a second gap distance at a point of near contact between the third plate and the fourth plate; rotating the third plate or the fourth plate about an axis of rotation; and contacting the first preparation of embryo explants with an interior surface of the third plate and an interior surface of the fourth plate to produce a second preparation of embryo explants comprising meristematic tissue, wherein the second gap distance is about 0.5 mm to about 5.0 mm, about 0.5 mm to about 2.5 mm, or is about 1.5 mm, including all ranges and values derivable therebetween. In certain embodiments, the second gap distance may be about 0.5 mm to about 3.0 mm, about 0.5 mm to about 2.5 mm, about 0.75 mm to about 2.25 mm, about 1.0 mm to about 2.0 mm, about 1.25 mm to about 1.75 mm, or about 1.5 mm, including all ranges and values derivable therebetween. The population of plant seeds, in specific embodiments, may comprise cotton seeds. In particular embodiments, the axis of rotation is substantially perpendicular to the ground. The axis of rotation, in some embodiments, may be substantially parallel to the ground.

According to certain embodiments of the present disclosure, grinder teeth, as described herein, may comprise a sharp surface and a dull surface. Contacting, in specific embodiments, may comprise contacting the population of plant seeds or a preparation thereof with the sharp surface of the grinder teeth of one plate and the sharp surface of the grinder teeth of another plate. Contacting, in certain embodiments, may comprise contacting the population of plant seeds or a preparation thereof with the sharp surface of the grinder teeth of one plate and the dull surface of the grinder teeth of another plate. In some embodiments, contacting may comprise contacting the population of plant seeds or a preparation thereof with the dull surface of the grinder teeth of one plate and the dull surface of the grinder teeth of another plate. In specific embodiments, the population of plant seeds or a preparation thereof may be first contacted with the sharp surface or the dull surface of the grinder teeth of the first, second, third, and/or fourth grinding plate as described herein before contacting additional surfaces of the respective plate. The plurality of grinder teeth, in certain embodiments, may each comprise a grinder tooth shape. Any grinder tooth shape known in the art may be used according to the embodiments of the present disclosure, non-limiting examples of which include a scalene shape, a triangular shape, and a geometric shape. A grinding plate according to the present disclosure may be structurally connected to, in specific embodiments, about 2 to about 50, about 2 to about 45, about 2 to about 40, about 2 to about 35, about 2 to about 30, about 2 to about 25, about 2 to about 20, about 2 to about 15, about 2 to about 10, about 2 to about 5, about 5 to about 10, about 10 to about 15, about 15 to about 20, about 20 to about 30, about 30 to about 40, or about 40 to about 50 grinder teeth per 2.54 cm, including all ranges and values derivable therebetween. In some embodiments, the grinding plate may be structurally connected to about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 grinder teeth per 2.54 cm, including all ranges and values derivable therebetween. The number of grinder teeth per 2.54 cm may be measured at any position on the surface of the grinding plate. In particular embodiments, the number of grinder teeth per 2.54 cm may be measured at or near the center, at or near the middle region, or at or near the outer circumference of the grinding plate. In particular embodiments, the number of grinder teeth per 2.54 cm may be the same at or near the center and at or near the outer circumference of the grinding plate. The number of grinder teeth per 2.54 cm, in some embodiments, may be different at or near the center and at or near the outer circumference of the grinding plate. In certain embodiments, the grinding plate may be structurally connected to about 6 to about 8 grinder teeth per 2.54 cm at or near the center and about 4 to about 6 grinder teeth per 2.54 cm at or near the outer circumference. The grinding plate, in particular embodiments, may be structurally connected to about 2 to about 4 grinder teeth per 2.54 cm at or near the center and about 6 to about 8 grinder teeth per 2.54 cm at or near the outer circumference.

Rotating, in some embodiments, may comprise rotating the third plate at about 50 rpm to about 300 rpm, about 100 rpm to about 200 rpm, about 100 rpm to about 150 rpm, about 125 rpm to about 150 rpm, or about 135 rpm, including all ranges and values derivable therebetween, wherein the fourth plate remains approximately stationary; or rotating the fourth plate at about 50 rpm to about 300 rpm, about 100 rpm to about 200 rpm, about 100 rpm to about 150 rpm, about 125 rpm to about 150 rpm, or about 135 rpm, including all ranges and values derivable therebetween, wherein the third plate remains stationary.

In particular embodiments, the present disclosure further provides a method comprising producing a fraction of a preparation of plant embryo explants; and contacting the interior surface of the third plate and the interior surface of the fourth plate with the fraction. The fraction of the preparation may be prepared, in some embodiments, by contacting the preparation with a moving plate, wherein the moving plate comprises a proximal end, a distal end, and a plurality of openings located near the distal end, each comprising a first physical opening size; passing the preparation through the plurality of openings of the moving plate and contacting a first moving sieve with the preparation, wherein the first moving sieve comprises a plurality of openings, each having a second physical opening size; separating the fraction of the preparation from a portion of the debris material by length, width, or thickness relative to the second physical opening size; and collecting the fraction of the preparation, wherein the moving plate and the first moving sieve move in a linear motion. In some embodiments, a method of preparing the fraction may comprise aspirating the preparation to remove an aspirated portion of the debris material. In particular embodiments, a method of preparing the fraction of the preparation may further comprise aspirating the preparation after contacting the preparation with the moving plate and prior to contacting the preparation with the first moving sieve; or aspirating the preparation after contacting the preparation with the first moving sieve and prior to separating the fraction of the preparation. The first physical opening size, in some embodiments, may be about 300 μm to about 5000 μm, about 400 μm to about 4500 μm, about 500 μm to about 4000 μm, about 500 μm to about 3500 μm, about 500 μm to about 3000 μm, or about 500 μm to about 2500 μm; or the second physical opening size, in certain embodiments, may be about 700 μm to about 1300 μm, about 1181 μm, or about 980 μm. In specific embodiments, the methods provided by the present disclosure may further comprise applying a cryogenic treatment to the preparation or the fraction of the preparation prior to contacting the preparation or the fraction of the preparation with the third plate and the fourth plate.

### C. Coarse Width Sizing

Embodiments of the present disclosure may include an apparatus for purifying dry embryo explants, wherein the apparatus comprises at least one moving sieve as described herein. As used herein the term “sieve” refers to a generally planar member comprising a top surface, a bottom surface, and a plurality of openings. The plurality of openings, in some embodiments, may be approximately evenly distributed along the plane of the sieve. The plurality of openings of a sieve, as described herein, may be configured, for example, to allow some particles of a preparation to pass through, while retaining other particles of the preparation on the top surface of the sieve. In some embodiments, the sieve may be generally oriented along a single horizontal, vertical, or diagonal plane. A sieve for use according to the methods of the present disclosure may comprise, in some embodiments, a mesh screen. In certain embodiments, the present disclosure provides a moving sieve, wherein the motion of the sieve is motorized and/or automated. A sieve for use according to the present disclosure may be made from any material which allows for the separation of embryo explants from debris material without causing damage to the embryo explants, non-limiting examples of which include stainless steel, steel, tin, aluminum, and brass.

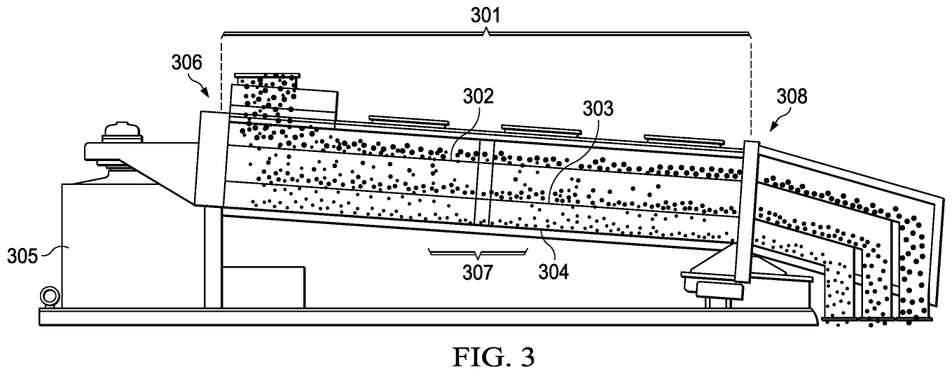

FIG. 3 is a diagram showing an apparatus having a housing unit 301 to receive a preparation comprising embryo explants; a first 302, a second 303, and a third 304 moving sieve structurally connected to the interior surface of the housing unit to separate a fraction of embryo explants from a portion of the debris material; and a motor 305 attached to the exterior surface of the housing unit 301 configured to move the housing unit 301 and the first 302, second 303, and third 304 moving sieves in a circular, elliptical, and/or linear motion within the plane of the first 302, second 303, and third 304 sieves. In some embodiments, the first 302, second 303, and/or third 304 moving sieves comprise a proximal end 306, a center region 307, and a distal end 308 and the proximal end 306 of the first 302, second 303, and/or third 304 moving sieve may be elevated relative to the distal end 308 to produce a slope angle. In certain embodiments, the preparation comprising embryo explants may enter the housing unit 301 and contact the first moving sieve 302 near the proximal end 306. In particular embodiments, the first 302, second 303, and/or third 304 moving sieves may move in a substantially circular motion near the proximal end 306, a substantially elliptical motion near the center region 307, and a substantially linear motion near the distal end 308.

As used herein the term “fraction” refers to purified preparation of embryo explants. For example, a fraction of embryo explants has been separated from a portion of the debris material present in a preparation comprising embryo explants. As used herein “debris material” refers to any material which is discarded during purification. Debris material does not predominantly include embryo explants, but some embryo explants may be present in the debris material.

In yet another aspect, the present disclosure provides a method of purifying genetically modifiable dry embryo explants comprising contacting a preparation of dry plant embryo explants comprising meristematic tissue with a moving sieve comprising a plurality of openings, wherein the preparation comprises a population of dry plant embryo explants and debris material; and separating a first fraction of embryo explants from a first portion of the debris material. In some embodiments, the sieve may comprise a plurality of openings, each of which may comprise a physical opening size and an effective opening size. The shape of each of the plurality openings may, in certain embodiments, be any geometric shape, non-limiting examples of which include a rectangle, a square, a circle, or an oval. As used herein the term “physical opening size” refers the physical dimensions of the opening. As used herein the term “effective opening size” refers to the effective dimensions of the opening, which are dependent on the physical opening size and the slope angle at which the sieve is positioned. “Slope angle” as used herein refers to the angle of position relative to the ground. “Ground” as used herein refers to a direction which is perpendicular to the direction of gravity. The slope angle may be, in certain embodiments, about 0 degrees to about 40 degrees, about 0 degrees, about 5 degrees, about 10 degrees, about 15 degrees, about 20 degrees, about 25 degrees, about 30 degrees, about 35 degrees, or about 40 degrees, including all ranges and values derivable therebetween. In some embodiment, the slope angle may be about 0 degrees to about −40 degrees, about 0 degrees, about −5 degrees, about −10 degrees, about −15 degrees, about −20 degrees, about −25 degrees, about −30 degrees, about −35 degrees, or about −40 degrees, including all ranges and values derivable therebetween. The effective opening size, for example, may be smaller than the physical opening size as the slope angle is less than or greater than 0 degrees, but may be approximately equal to the physical opening size, when the slope angle is approximately 0 degrees. In some embodiments, a sieve as described herein may comprise a proximal end and a distal end and the proximal end of the sieve may be elevated relative to the distal end to produce the slope angle. The preparation of dry embryo explants, in certain embodiments, may be first contacted with a sieve, as described herein, at or near the proximal end.

In particular embodiments, the preparation of dry embryo explants may be contacted with a sieve, as described herein, at a rate of about 1500 g/min to about 4000 g/min, about 2000 g/min to about 3500 g/min, about 2000 g/min to about 3000 g/min, about 2500 g/min to about 3000 g/min, about 2500 g/min, about 2600 g/min, about 2700 g/min, about 2800 g/min, about 2900 g/min, about 3000 g/min, about 3100 g/min, about 3200 g/min, about 3300 g/min, about 3400 g/min, or about 3500 g/min, including all ranges and values derivable therebetween.

In certain embodiments of the present disclosure, methods for separating embryo explants comprising the use of a first, second, and/or third moving sieve are provided. In some embodiments, the plane of the first moving sieve is substantially parallel to the plane of the second and/or third moving sieves. As used herein with regard to the planes of two or more moving sieves the term “substantially parallel” refers to a situation where the plane of one sieve and the plane of another sieve are essentially parallel but may, in some embodiments, vary by about +/−15 degrees. In some embodiments, the plane of one sieve and the plane of another sieve are essentially parallel but may vary by about +/−1 degree, about +/−2 degrees, about +/−3 degrees, about +/−4 degrees, about +/−5 degrees, about +/−6 degrees, about +/−7 degrees, about +/−8 degrees, about +/−9 degrees, about +/−10 degrees, about +/−11 degrees, about +/−12 degrees, about +/−13 degrees, about +/−14 degrees, or about +/−15 degrees, including all ranges and values derivable therebetween. In specific embodiments, the first moving sieve is positioned directly above the second moving sieve, and/or the second moving sieve is positioned directly above the third moving sieve. In certain embodiments, the first, second, and/or third moving sieves are structurally connected and move together in unison. As used herein sieves which are “structurally connected” are in direct or indirect contact with each other. Two or more sieves may be considered structurally connected, for example, if they are each in contact with one or more shared structural components of an apparatus. As used herein sieves which move in “unison,” move simultaneously. In some embodiments, sieves which move in unison may also have approximately the same circular, elliptical, and/or linear motion. In some embodiments, the first, second, and/or third moving sieve comprises a proximal end and a distal end, the proximal end of which is elevated relative to the distal end. The preparation comprising embryo explants and debris material, in certain embodiments, is first contacted with the proximal end of the first, second, and/or third moving sieve. In certain embodiments, the preparation travels along the first, second, and/or third moving sieve in a general proximal-to-distal direction.

In one aspect of the present disclosure, a method for purifying genetically modifiable dry embryo explants is provided, wherein the method comprises contacting a preparation of dry embryo explants with a first moving sieve, as described herein, and separating a first fraction of embryo explants from a first portion of the debris material. In some embodiments, the method may further comprise contacting the first fraction of embryo explants with a second moving sieve, as described herein, and separating a second fraction of embryo explants from a second portion of the debris material. In particular embodiments, the method may additionally comprise contacting the second fraction of embryo explants with a third moving sieve, as described herein, and separating a third fraction of embryo explants from a third portion of the debris material. In specific embodiments, the methods described herein may further comprise aspirating the preparation, first fraction, second fraction, and/or third fraction to remove a first, second, and/or third aspirated portion of the debris material. The first, second, and/or third moving sieves may, in some embodiments, have the same physical opening size and/or effective opening size. In particular embodiments, the first, second, or third moving sieves may each have a different physical opening size and/or effective opening size. The physical opening size of the first moving sieve may be, for example, about 300 μm to about 2600 μm, about 1600 μm to about 2600 μm, about 1600 μm to about 2500 μm, about 800 μm to about 2000 μm, about 800 to about 1500 μm, about 700 μm to about 1300 μm, about 600 μm to about 1200 μm, about 600 μm to about 1100 μm, about 500 μm to about 1000 μm, about 300 to about 1000 μm, or about 300 μm to about 900 μm, including all ranges and values derivable therebetween. The physical opening size for the second moving sieve may be, for example, about 300 μm to about 1500 μm, about 800 to about 1500 μm, about 700 μm to about 1300 μm, about 600 μm to about 1200 μm, about 600 μm to about 1100 μm, about 500 μm to about 1000 μm, about 300 to about 1000 μm, or about 300 μm to about 900 μm, including all ranges and values derivable therebetween. The physical opening size for the third moving sieve may be, for example, about 300 μm to about 900 μm, about 350 to about 600 μm, or about 500 μm, including all ranges and values derivable therebetween. For purification of corn embryo explants, in certain embodiments, the physical opening size of the first moving sieve may be about 500 μm to about 2000 μm, about 800 μm to about 2000 μm, about 500 μm to about 1000 μm, about 1181 μm, or about 812 μm and/or the physical opening size of the second moving sieve may be about 500 μm to about 1000 μm or about 812 μm. For purification of soybean embryo explants, in some embodiments, the physical opening size of the first moving sieve may be about 800 μm to about 2600 μm, about 1600 μm to about 2600 μm, about 800 μm to about 1500 μm, about 2032 μm, or about 1181 μm, and/or the physical opening size of the second moving sieve may be about 800 μm to about 1500 μm or about 1181 μm. For purification of wheat embryo explants, in particular embodiments, the physical opening size of the first moving sieve may be about 300 μm to about 1200 μm, about 600 μm to about 1200 μm, about 300 μm to about 900 μm, about 864 μm, or about 610 μm, and/or the physical opening of the second moving sieve may be about 300 μm to about 900 μm or about 610 μm. For purification of canola embryo explants, in some embodiments, the first physical opening size may be about 300 μm to about 1100 μm, about 600 μm to about 1100 μm, about 300 μm to about 1000 μm, about 500 μm to about 1000 μm, about 864 μm, about 812 μm, or about 503 μm, the second physical opening size may be about 600 μm to about 1000 μm or about 812 μm, and/or the third physical opening size may be about 300 μm to about 900 μm or about 503 μm. For purification of cotton embryo explants, in specific embodiments, the first physical opening size may be about 700 μm to about 2500 μm, about 1600 μm to about 2500 μm, about 700 μm to about 1300 μm, about 2032 μm, about 1181 μm, or about 980 μm, and/or the second physical opening size may be about 700 μm to about 1300 μm, about 1181 μm, or about 980 μm.

In particular embodiments of the present disclosure, methods are provided herein for separating a first, second, and/or third fraction of embryo explants from debris material. In some embodiments, the first, second, and/or third fraction of embryo explants may be passed through the plurality of openings of the first, second, and/or third moving sieve, while the first, second, and/or third portion of the debris material is retained on the surface of the first, second, and/or third moving sieve. In certain embodiments, the first, second, and/or third portion of the debris material is passed through the plurality of openings of the first, second, and/or third moving sieve, while the first, second, and/or third fraction of embryo explants is retained on the surface of the first, second, or third moving sieve. In specific embodiments, the methods of the present disclosure comprise collecting the second fraction of embryo explants at or near the distal end of the second moving sieve. In particular embodiments, the second fraction of embryo explants is captured on a receiving plate and discharged through an output near the distal end of the second moving sieve.

In some embodiments, the present disclosure provides a sieve which moves in a circular, elliptical, and/or linear motion within the plane of the sieve. The sieve may move, in certain embodiments, in a gyratory-reciprocating motion, which gradually changes from a circular motion to an elliptical motion to an approximate straight-line motion. The circular motion may be used, for example, to spread material across the full width of the sieve surface, promoting stratification of the material. The elliptical motion may be used, for example, to further stratify the material, and the linear sifting motion may be used, for example, to remove near-size particles and improve sieving efficiency. In particular embodiments, the sieve may further comprise a vibratory motion.

Embodiments of the present disclosure may include an apparatus for purifying dry embryo explants, wherein the apparatus comprises at least one moving plate and at least one moving sieve as described herein. As used herein a “moving plate” refers to a substantially planar member that comprises a plurality of openings, wherein the plurality of openings are unevenly distributed along the plane of the moving plate. In specific embodiments, the moving plate may comprise a proximal end and a distal end and the plurality of openings may be generally located at or near the proximal end or at or near the distal end.

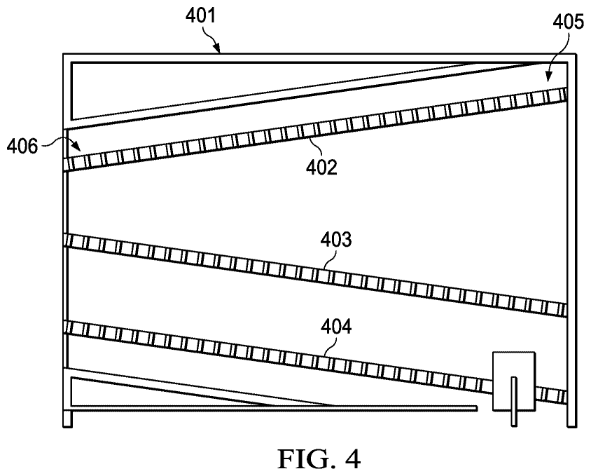

FIG. 4 is a diagram showing an apparatus having a housing unit 401 to receive a preparation of dry embryo explants; and a first 402, a second 403, and a third 404 moving sieve attached to the interior surface of the housing unit to separate a fraction of embryo explants from a portion of the debris material. In certain embodiments, the first 402, second 403, and/or third 404 moving sieves may be positioned at a slope angle as described herein. In particular embodiments, the first 402, second 403, and/or third 404 moving sieve may be replaced by a moving plate comprising a proximal end 405, a distal end 406, and a plurality of openings located near the distal end.

In another aspect, the present disclosure provides a method of purifying genetically modifiable dry embryo explants comprising passing a preparation of dry embryo explants comprising meristematic tissue and debris material through a moving plate comprising a plurality of openings located at the distal end and contacting the preparation with a first moving sieve to separate a first fraction of embryo explants from a first portion of the debris material. In some embodiments, the plurality of openings of the moving plate have a first physical opening size and the first physical opening size may be about 300 μm to about 5000 μm, about 400 μm to about 4500 μm, about 500 μm to about 4000 μm, about 500 μm to about 3500 μm, about 500 μm to about 3000 μm, or about 500 μm to about 2500 μm, including all ranges and values derivable therebetween. In certain embodiments, the first moving sieve comprises a plurality of openings having a second opening size and the second opening size is about 700 μm to about 1300 μm, about 1181 μm, or about 980 μm, including all ranges and values derivable therebetween. The method may further comprise, in some embodiments, contacting the first fraction of embryo explants with a second moving sieve to separate a least second fraction of embryo explants from a second portion of the debris material. The physical opening size of the second moving sieve may be, for example, about 1600 μm to about 2500 μm or about 2032 μm, including all ranges and values derivable therebetween. The method may additionally comprise, in particular embodiments, contacting the second fraction with a third moving sieve to separate a third fraction of embryo explants from the debris material. The physical opening size of the third moving sieve may be, for example, about 700 μm to about 1300 μm, about 1181 μm, or about 980 μm, including all ranges and values derivable therebetween. In certain embodiments, the methods disclosed herein may further comprise a step of applying a cryogenic treatment to the first fraction of embryo explants prior to contacting the first fraction with the second moving sieve. The method may comprise, for example, any method of applying a cryogenic treatment known in the art, examples of which include, but are not limited to, submersion in liquid nitrogen and exposure to a temperature equal to or less than about 0° C.

In some embodiments, the plane of one moving plate or sieve and the plane of another moving plate or sieve may be positioned relative to each other at an angle of about −45 degrees, about −40 degrees, about −35 degrees, about −30 degrees, about −25 degrees, about −20 degrees, about −15 degrees, about −10 degrees, about −5 degrees, about 0 degrees, about 45 degrees, about 40 degrees, about 35 degrees, about 30 degrees, about 25 degrees, about 20 degrees, about 15 degrees, about 10 degrees, or about 5 degrees, including all ranges and values derivable therebetween. In certain embodiments, the moving plate and/or the moving sieves are structurally connected and move together in unison. As used herein moving plates or sieves which are “structurally connected” are in direct or indirect contact with each other. Two or more moving plates or sieves may be considered structurally connected, for example, if they are each in contact with one or more shared structural components of an apparatus. As used herein moving plates or sieves which move in “unison,” move simultaneously. In some embodiments, moving plates or sieves which move in unison may also have approximately the same circular, elliptical, and/or linear motion. In some embodiments, the moving plate, the first moving sieve, and/or the second moving sieve proximal end and a distal end. In some embodiments, the proximal end of the moving plate is elevated relative to the distal end. In particular embodiments, the distal end of the first and/or the second moving sieve is elevated relative to the proximal end. In some embodiments, the proximal end of the first and/or second moving sieves is elevated relative to the distal end. The preparation comprising embryo explants and debris material, in certain embodiments, is first contacted with the proximal end of the moving plate. In certain embodiments, the preparation travels along the moving plate in a general proximal-to-distal direction. In some embodiments, the preparation travels along the first and/or second moving sieves in a general distal-to-proximal direction or in a general proximal-to-distal direction. The preparation, in particular embodiments, moves from the elevated end of the moving plate, the first moving sieve, and/or the second moving sieve toward the end which is not elevated.

### D. Length Sizing

In still yet another aspect, the present disclosure provides a method of purifying dry embryo explants comprising contacting a preparation of dry embryo explants comprising meristematic tissue with an interior surface of a rotating cylinder, wherein the interior surface comprises a plurality of indentations, the indentations having an indentation size and an indentation shape, wherein the preparation comprises a population of dry plant embryo explants and debris material; rotating the rotating cylinder about an axis of rotation to produce a centrifugal force acting on the preparation, wherein the axis of rotation is substantially parallel to the ground; and separating a fraction of the plant embryo explants of the preparation from a portion of the debris material according to a displacement of the portion of the debris material relative to a displacement of the fraction of plant embryo explants produced by the rotating. As used herein the term “indentation” refers to depression relative to the interior surface of the rotating cylinder. As used herein with regard to the axis of rotation of a rotating cylinder the term “substantially parallel” refers to an axis of rotation that is essentially parallel to the ground but may, in some embodiments, vary by about +/−15 degrees. In particular embodiments, the axis of rotation of the rotating cylinder is essentially parallel to the ground but may vary by about +/−1 degree, about +/−2 degrees, about +/−3 degrees, about +/−4 degrees, about +/−5 degrees, about +/−6 degrees, about +/−7 degrees, about +/−8 degrees, about +/−9 degrees, about +/−10 degrees, about +/−11 degrees, about +/−12 degrees, about +/−13 degrees, about +/−14 degrees, or about +/−15 degrees, including all ranges and values derivable therebetween. In some embodiments, the axis of rotation of the rotating cylinder may be about +/−1 degree to facilitate the distribution of material along the bottom interior region of the rotating cylinder. As used herein the term “centrifugal force” refers to an apparent force that acts outward on the preparation moving around a center, which arises from the preparation's inertia. The centrifugal force acting on the fraction of plant embryo explants present in the preparation may, for example, be different than the centrifugal force acting on the portion of the debris material due to the different inertias of the fraction of plant embryo explants relative to the portion of the debris material. A “displacement” as used in reference to the displacement of materials produced by the rotating cylinder refers to the movement of the fraction of plant embryo explants or the portion of the debris material present in the preparation from its starting position within a hollow center cavity of the rotating cylinder. In some embodiments, the displacement of the fraction of plant embryo explants is less than the displacement of the portion of the debris material. In certain embodiments, the displacement of the fraction of plant embryo explants is defined as a net displacement, and the net displacement of the fraction of plant embryo explants is approximately zero. A “net displacement” as used herein refers to the resultant distance between the initial and final positions of the plant embryo explants or the portion of the debris material within a hollow center cavity of the rotating cylinder. For example, the fraction of embryo explants may tumble back and forth within the hollow center cavity, resulting in a small displacement of the fraction, however the net displacement of the fraction of embryo explants may be approximately zero.

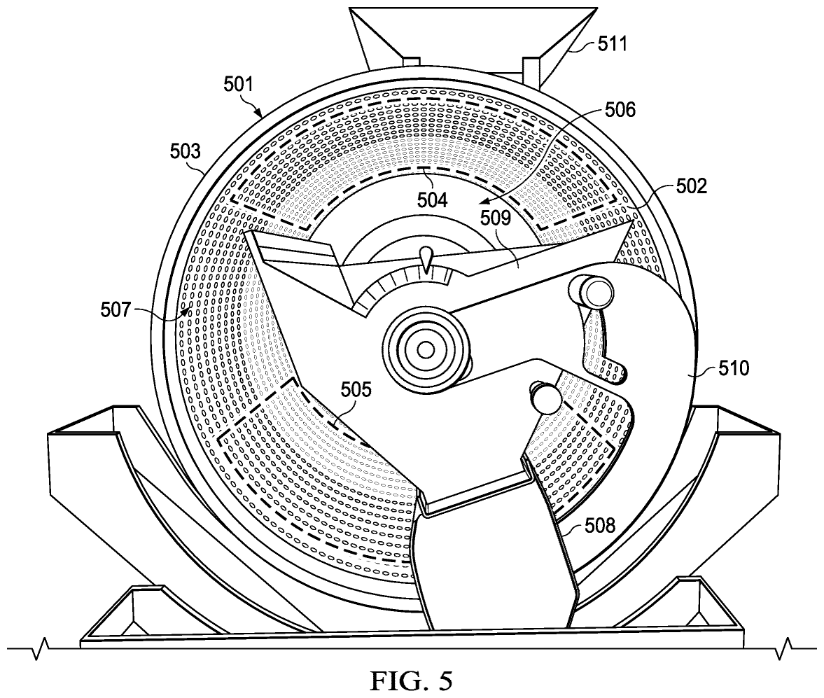

FIG. 5 is a diagram showing an apparatus having a rotating cylinder 501 to receive a preparation of plant embryo explants, the rotating cylinder 501 comprising an interior surface 502, an exterior surface 503, a top interior region 504, a bottom interior region 505, and a hollow center cavity 506; the interior surface 502 of the rotating cylinder 501 comprising a plurality of indentations 507 configured to maintain the portion of the debris material in greater contact with the interior surface 502 of the rotating cylinder 501 relative to the fraction of plant embryo explants; a debris chute 508 to remove the portion of the debris material from the debris collector 509 configured to collect debris material, wherein the debris chute 508 comprises a first end and a second end and the debris collector 509 comprises a top portion and a bottom portion, the first end of the debris chute 508 being in fluid communication with the bottom portion of the debris collector 509; an adjustable arm 510 to position the debris collector 509 at a preferred angle within the hollow center cavity 506 of the rotating cylinder 501, wherein the adjustable arm 510 comprises a first end and a second end, the first end being structurally connected to the debris collector 509; and a feeding unit 511 attached to the rotating cylinder 501 to facilitate the transfer of a preparation of plant embryo explants to the interior surface 502 of the rotating cylinder 501.

In some embodiments, the separating may comprise separating the fraction of the plant embryo explants from the portion of the debris material by the relative length, width, shape, or weight of the plant embryo explants and the debris material. The indentations may be of any geometric shape, non-limiting examples of which include a rectangle, a square, a circle, or an oval. The indentation size and/or the indentation shape may be modified according to the characteristics of the explants to be purified. In some embodiments, each indentation of the plurality of indentations may have approximately the same size and/or approximately the same shape. In particular embodiments, the indentations of the plurality of indentations may have a plurality of indentations sizes and/or a plurality of indentation shapes. In some embodiments, the rotating lifts the portion of the debris material from a bottom interior region to a top interior region of the rotating cylinder. The fraction of plant embryo explants, in certain embodiments, remains at the bottom interior region of the rotating cylinder during the rotating. The indentation size or the indentation shape, in certain embodiments, may be configured, in combination with the centrifugal force acting on the preparation, to maintain the portion of the debris material in greater contact with the interior surface of the rotating cylinder relative to the fraction of plant embryo explants as the rotation of the rotating cylinder lifts the portion of the debris material from a bottom interior region to a top interior region of the rotating cylinder. This greater contact of the debris material with the interior surface of the rotating cylinder may, in some embodiments, result in an increased displacement or an increased net displacement of the portion of the debris material relative to the displacement of the plant embryo explants. For example, an indentation size or an indentation shape may be chosen such that the indentation size or the indentation shape holds the portion of the debris material and lifts the debris material upward from a bottom interior region to a top interior region of the rotating cylinder during rotation. In particular embodiments, the indentation size or the indentation shape of the plurality of indentations is configured to exclude the fraction of plant embryo explants from the plurality of indentations. For example, the chosen indentation size may be too small to accommodate the plant embryo explants or the chosen indentation shape may be unable to accommodate the plant embryo explants, such that the plant embryo explants maintain less contact with the interior surface of the rotating cylinder during rotation. As such, in certain embodiments, the fraction of plant embryo explants may not be lifted from the bottom interior region to the top interior region of rotating cylinder during rotation. In particular embodiments, the fraction embryo explants may be slightly lifted from the bottom interior region of the rotating cylinder, however, the decreased contact with the interior surface results in the fraction falling away from the interior surface prior to reaching the top interior region. In some embodiments, the indentation size or the indentation shape of the plurality of indentations in combination with the centrifugal force acting on the preparation acts against the force of gravity to produce the displacement of the portion of the debris material or the displacement of the fraction of plant embryo explants.

In certain embodiments, each indentation size comprises an indentation diameter, an indentation width, an indentation length, or an indentation depth. The indentation diameter, indentation width, or indentation length may be, in some embodiments, about 0.50 mm to about 5.00 mm, about 0.75 mm to about 4.75 mm, about 1.00 mm to about 4.50 mm, about 1.00 mm to about 4.00 mm, about 1.25 mm to about 3.75 mm, about 1.50 mm to about 3.50 mm, about 1.75 mm to about 3.50 mm, about 2.00 mm to about 3.25 mm, about 2.25 mm to about 3.00 mm, about 2.50 mm to about 2.75 mm, about 1.25 mm to about 2.75 mm, about 1.50 mm to about 2.50 mm, about 1.75 mm to about 2.50 mm, about 2.00 mm to about 2.50 mm, about 1.75 mm to about 2.25 mm, about 0.50 mm, about 0.75 mm, about 1.00 mm, about 1.25 mm, about 1.50 mm, about 1.75 mm, about 2.00 mm, about 2.25 mm, about 2.50 mm, about 2.75 mm, about 3.00 mm, about 3.25 mm, about 3.50 mm, about 3.75 mm, about 4.00 mm, about 4.25 mm, about 4.50 mm, about 4.75 mm, or about 5.00 mm, including all ranges and values derivable therebetween. The indentation depth may be, in particular embodiments, about 0.25 mm to about 2.00 mm, about 0.50 mm to about 1.75 mm, about 0.75 mm to about 1.25 mm, about 0.25 mm, about 0.50 mm, about 0.75 mm, about 1.00 mm, about 1.25 mm, about 1.50 mm, about 1.75 mm, or about 2.00 mm, including all ranges and values derivable therebetween.

In certain embodiments, an indentation diameter, an indentation width, or an indentation length of about 1.25 mm to about 3.00 mm, about 1.50 mm to about 2.75 mm, about 1.75 mm to about 2.50 mm, about 2.00 mm to about 2.25 mm, about 1.25 mm, about 1.50 mm, about 1.75 mm, about 2.00 mm, about 2.25 mm, about 2.50 mm, about 2.75 mm, or about 3.00 mm may be used to purify corn embryo explants, including all ranges and values derivable therebetween. In some embodiments, an indentation diameter, an indentation width, or an indentation length of about 2.00 mm to about 4.00 mm, about 2.00 mm to about 3.75 mm, about 2.25 mm to about 3.50 mm, about 2.50 mm to about 3.25 mm, about 2.75 mm to about 3.00 mm, about 2.00 mm, about 2.25 mm, about 2.50 mm, about 2.75 mm, about 3.00 mm, about 3.25 mm, about 3.50 mm, about 3.75 mm, or about 4.00 mm may be used to purify soybean or cotton embryo explants, including all ranges and values derivable therebetween. The indentation size may be modified according to the characteristics of the embryo explants to be purified. For example, plant seeds or plant embryo explants may be sorted according to size and/or shape and an appropriate indentation size and/or shape may be selected according to the size and/or shape characteristics of the seeds or embryo explants. In particular embodiments, an indentation size and/or shape may be selected which excludes the embryo explants from the plurality of indentations.

In some embodiments, the rotating cylinder may rotate at about 10 rpm to about 100 rpm, about 10 rpm to about 90 rpm, about 10 rpm to about 80 rpm, about 10 rpm to about 70 rpm, about 10 rpm to about 60 rpm, about 10 rpm to about 50 rpm, about 15 rpm to about 50 rpm, about 20 rpm to about 45 rpm, about 25 rpm to about 40 rpm, about 30 rpm to about 40 rpm, about 35 rpm to about 40 rpm, about 10 rpm, about 15 rpm, about 20 rpm, about 25 rpm, about 30 rpm, about 31 rpm, about 32 rpm, about 33 rpm, about 34 rpm, about 35 rpm, about 36 rpm, about 37 rpm, about 38 rpm, about 39 rpm, about 40 rpm, about 41 rpm, about 42 rpm, about 43 rpm, about 44 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, or about 100 rpm, including all ranges and values derivable therebetween.

In some embodiments, the embryo explant preparation may be initially fed into the rotating cylinder at a higher rate to at least partially load the cylinder. The initial feeding rate may, in certain embodiments, be about 500 g/min to about 2500 g/min, about 750 g/min to about 2500 g/min, about 1000 g/min to about 2250 g/min, about 1250 g/min to about 2250 g/min, about 1400 g/min to about 2250 g/min, about 1500 g/min to about 2250 g/min, about 1600 g/min to about 2250 g/min, about 1700 g/min to about 2250 g/min, about 1800 g/min to about 2100 g/min, about 1900 g/min to about 2100 g/min, about 1900 g/min to about 2000 g/min, about 1925 g/min to about 2000 g/min, about 1925 g/min to about 1975 g/min, or about 1942 g/min, including all ranges and values derivable therebetween. After the initial loading, the embryo explant preparation may be fed into the rotating cylinder, in some embodiments, at a slower rate. This slower feeding rate may be useful to balance the inflow and outflow of material into and out of the rotating cylinder. This second slower feeding rate may be, in particular embodiments, 500 g/min to about 2500 g/min, about 750 g/min to about 2250 g/min, about 750 g/min to about 2000 g/min, about 750 g/min to about 1500 g/min, about 800 g/min to about 1400 g/min, about 900 g/min to about 1300 g/min, about 1000 g/min to about 2000 g/min, about 1000 g/min to about 1500 g/min, about 1000 g/min to about 1400 g/min, about 1000 g/min to about 1300 g/min, about 1100 g/min to about 1300 g/min, about 1200 g/min to about 1300 g/min, about 1250 g/min to about 1300 g/min, or about 1271 g/min, including all ranges and values derivable therebetween. The feed rate may be modified depending on the ratio of the debris material present in the output material. If the ratio of the debris material present in the output material is high, then the feed rate can be slowed to improve the purity. A faster feed rate is generally preferred for faster processing and increased output, if the ratio of the debris material is within an acceptable range.

In certain embodiments of the present disclosure, the rotating cylinder may comprise a top interior region, a bottom interior region, and a hollow center cavity. In some embodiments, the rotating cylinder may be structurally connected to a debris collector. As used herein the term “debris collector” refers to a component capable of collecting debris material. In particular embodiments, the debris collect may comprise a substantially planar surface, a container, or a collection chute. The debris collector may be, for example, a flat plate or surface on which debris material may be collected. A “container” as used herein in reference to a debris collector refers to a wide, open container. A container may be of any appropriate geometrical shape, non-limiting examples of which include a cylinder, a sphere, a triangular prism, a cube, and a cone. As used herein a “collection chute” refers to a hollow component used for holding or transporting the debris material. In some embodiments, the methods provided by the present disclosure may comprise positioning a debris collector within the hollow center cavity of the rotating cylinder; and collecting the portion of the debris material in the debris collector. In some embodiments, gravity causes the portion of the debris material to fall away from the interior surface at or near the top interior region of the rotating cylinder and into the debris collector. In some embodiments, the methods provided by the present disclosure may comprise transferring the portion of the debris material from the bottom interior region to the top interior region of the rotating cylinder; and delivering the portion of the debris material to a debris collector, wherein gravity causes the portion of the debris material to fall away from the interior surface of the top interior region of the rotating cylinder and into the debris collector. The fraction of embryo explants, in certain embodiments, remains at or near the bottom interior region of the rotating cylinder during the separating.

In particular embodiments, the present disclosure provides a method further comprising positioning the debris collector at a preferred location within the hollow center cavity of the rotating cylinder. In certain embodiments, the rotating cylinder has an interior radius (r) measured from the axis of rotation to the interior surface of the rotating cylinder, the debris collector comprises a top portion and a bottom portion, and the methods provided by the present disclosure further comprise positioning the top portion of the debris collector within the hollow center cavity at a distance of about 0.1×(r) to about 0.9×(r), 0.2×(r) to about 0.8×(r), about 0.2×(r) to about 0.7×(r), about 0.3×(r) to about 0.6×(r), about 0.4×(r) to about 0.6×(r), about 0.1×(r), about 0.2×(r), about 0.3×(r), about 0.4×(r), about 0.5×(r), about 0.6×(r), about 0.7×(r) about 0.8×(r), or about 0.9×(r) from the axis of rotation of the rotating cylinder, including all ranges and values derivable therebetween. In certain embodiments, the debris collector may be positioned at a distance described herein below the axis of rotation or above the axis of rotation. The position of the debris collector within the hollow center cavity of the rotating cylinder will determine how thick the material bed will become at the bottom interior region of the rotating cylinder. In some embodiments, the debris collector may be positioned at an angle relative to the ground within the hollow cavity of the rotating cylinder. The debris collector, in specific embodiments, may comprise a top portion and a bottom portion and the top portion of the debris collector may be positioned at an angle of about −5 degrees to about −45 degrees, about 5 degrees to about 45 degrees, about −5 degrees to about −40 degrees, about 5 degrees to about 40 degrees, about −5 degrees to about −35 degrees, about 5 degrees to about 35 degrees, about −5 degrees to about −30 degrees, about 5 degrees to about 30 degrees, about −5 degrees to about −25 degrees, about 5 degrees to about 25 degrees, about −5 degrees to about −20 degrees, about 5 degrees to about 20 degrees, about −5 degrees to about −15 degrees, about 5 degrees to about 15 degrees, about −10 degrees to about −20 degrees, about 10 degrees to about 20 degrees, about −5 degrees, about 5 degrees, about −10 degrees, about 10 degrees, about −15 degrees, about 15 degrees, about −20 degrees, about 20 degrees, about −25 degrees, about 25 degrees, about −30 degrees, about 30 degrees, about −35 degrees, about 35 degrees, about −40 degrees, about 40 degrees, about −45 degrees, or about 45 degrees relative to the ground. In certain embodiments, the debris collector may comprise a substantially planar surface and the top portion of the debris collector refers to the top surface of the substantially planar surface. In some embodiments, the debris collector may comprise a container, as described herein, the container may have a plane which extends across the open portion of the container from one side of the container to another side of the container. The top portion of the container, in some embodiments, may refer to this plane of the container. In particular embodiments, the debris collector may comprise a collection chute as described herein, and the collection chute may have a plane which extends across the open portion of the collection chute from one side of the collection chute to another side of the collection chute. The top portion of the collection chute, in some embodiments, may refer to this plane of the collection chute.

The methods provided by the present disclosure may, in some embodiments, further comprise collecting the fraction of embryo explants. In certain embodiments, the fraction of plant embryo explants is collected from the bottom interior region of the rotating cylinder. The methods provided by the present disclosure, in particular embodiments, may further comprise stopping the rotating of the rotating cylinder prior to collecting the fraction of plant embryo explants.

### E. Aspiration

Embodiments of the present disclosure include a method of purifying genetically modifiable dry plant embryo explants, the method comprising aspirating within a vertical chamber a preparation of dry plant embryo explants comprising meristematic tissue or a fraction thereof with an upward air flow having an air flow velocity of about 1.0 m/s to about 25.0 m/s, wherein the preparation or fraction thereof comprises a population of dry plant embryo explants and debris material; and separating a fraction of the plant embryo explants from a portion of the debris material according to a displacement of the fraction relative to a displacement of the portion of the debris material produced by the upward air flow within the vertical chamber. In particular embodiments, a preparation of dry embryo explants or a fraction thereof may be aspirated within a first, a second, a third, and/or a fourth vertical chamber in order to separate a fraction of embryo explants from a portion of the debris material. A first, second, third, and/or fourth fraction of plant embryo explants may, in some embodiments, demonstrate a fraction buoyancy and the portion of the debris material may demonstrate a debris buoyancy in the upward air flow against the force of gravity, wherein the fraction buoyancy and the debris buoyancy are different, and wherein the fraction buoyancy and the debris buoyancy result in a different displacement of the fraction compared to the displacement of the portion of the debris material. As used herein the term “air” refers to any gas or mixture of gases that may be used for aspiration. Non-limiting examples of gases that may be used alone or in a mixture of gases for aspiration include nitrogen, hydrogen, oxygen, argon, carbon dioxide, and helium.

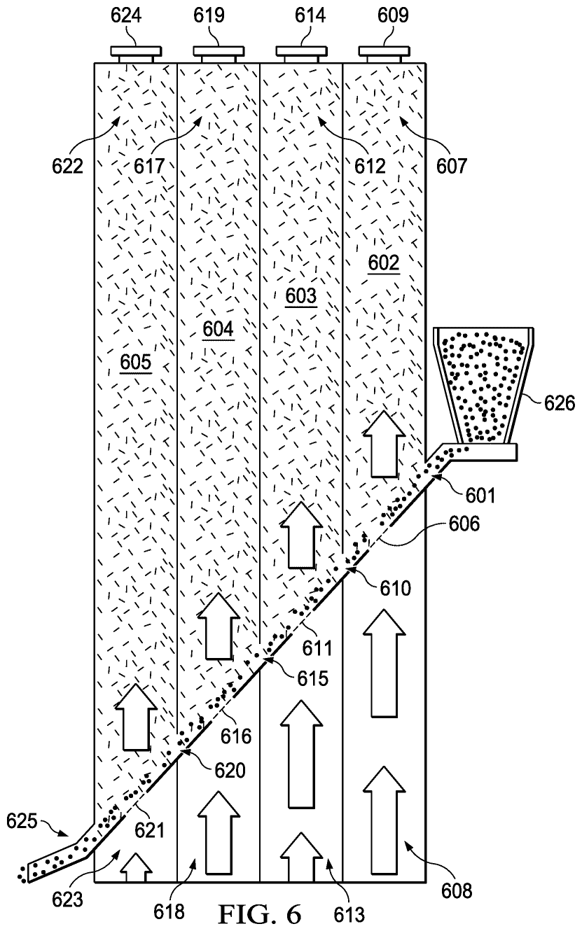

FIG. 6 is a diagram showing an apparatus having an input port 601 configured to introduce a preparation comprising dry embryo explants into a first aspiration chamber 602 configured to aspirate the preparation, the apparatus additionally having a second aspiration chamber 603, a third aspiration chamber 604, and a fourth aspiration chamber 605 configured to aspirate the preparation; the first aspiration chamber 602 having a first aspiration screen 606, a top portion 607 positioned above the first aspiration screen 606, and a bottom portion 608 positioned below the first aspiration screen 606, wherein the top portion 607 of the first aspiration chamber 602 is in fluid communication with a first discharge port 609 to facilitate the discharge of a first portion of the debris material from the first aspiration chamber 602, wherein the first aspiration chamber 602 has a first advancement port 610 to facilitate the transfer of a fraction of embryo explants to the second aspiration chamber 603; the second aspiration chamber 603 having a second aspiration screen 611, a top portion 612 positioned above the second aspiration screen 611, and a bottom portion 613 positioned below the second aspiration screen 611, wherein the top portion 612 of the second aspiration chamber 603 is in fluid communication with a second discharge port 614 to facilitate the discharge of a second portion of the debris material from the second aspiration chamber 603, wherein the second aspiration chamber 603 has a second advancement port 615 to facilitate the transfer of a fraction of embryo explants to the third aspiration chamber 604; the third aspiration chamber 604 having a third aspiration screen 616, a top portion 617 positioned above the third aspiration screen 616, and a bottom portion 618 positioned below the third aspiration screen 616, wherein the top portion 617 of the third aspiration chamber 604 is in fluid communication with a third discharge port 619 to facilitate the discharge of a third portion of the debris material from the third aspiration chamber 604, wherein the third aspiration chamber 604 has a third advancement port 620 to facilitate the transfer of a fraction of embryo explants to the fourth aspiration chamber 605; the fourth aspiration chamber 605 having a fourth aspiration screen 621, a top portion 622 positioned above the fourth aspiration screen 621, and a bottom portion 623 positioned below the fourth aspiration screen 621, wherein the top portion 622 of the fourth aspiration chamber 605 is in fluid communication with a fourth discharge port 624 to facilitate the discharge of a fourth portion of the debris material from the fourth aspiration chamber 605, wherein the fourth aspiration chamber 605 has a output collector 625 to collect a fraction of plant embryo explants; the apparatus further having a vibrating feeding unit 626 in fluid communication with the input port 601 to facilitate the transfer of the preparation through the input port 601 and into the first aspiration chamber 602.

In one aspect, the present disclosure provides a method of purifying genetically modifiable explants comprising aspirating a preparation comprising dry embryo explants or a fraction thereof in order to separate the embryo explants from a portion of the debris material. In particular embodiments, the present disclosure provides a method of purifying genetically modifiable dry embryo explants comprising aspirating a preparation within a first, second, third, and/or fourth vertical chamber with an upward air flow having an air velocity of about 1.0 m/s to about 25.0 m/s, about 2.5 m/s to about 25.0 m/s, about 2.5 m/s to about 8.0 m/s, about 3.1 m/s to about 7.2 m/s, about 3.0 m/s to about 8.5 m/s, about 4.1 m/s to about 8.2 m/s, about 4.5 m/s to about 12.5 m/s, about 5.3 m/s to about 11.9 m/s, about 6.5 m/s to about 20.5 m/s, or about 7.5 m/s to about 19.9 m/s, including all ranges and values derivable therebetween; and separating a first, second, third, and/or fourth fraction of embryo explants from at least a first, second, third, and/or fourth portion of the debris material.

In particular embodiments, the methods provided by the present disclosure may further comprise introducing the preparation or a fraction thereof into the first, second, third, and/or fourth vertical chamber above a first, second, third, and/or fourth aspiration screen positioned within the first, second, third, and/or fourth vertical chamber, the first, second, third, and/or fourth aspiration screen comprising a plurality of openings, each comprising an opening size and an opening shape. The opening shape may be any geometric shape, non-limiting examples of which include a rectangle, a sequence, a circle, and an oval. In some embodiments, the opening size may comprise an opening diameter, an opening width, and/or an opening length. The opening diameter, opening width, and/or opening length of the first, second, third, and/or fourth aspiration screen may be, in certain embodiments, about 10 μm to about 500 μm, about 10 μm to about 400 μm, 20 μm to about 300 μm, 20 μm to about 200 μm, about 20 μm to about 150 μm, about 20 μm to about 120 μm, about 30 μm to about 120 μm, about 40 μm to about 120 μm, about 50 μm to about 110 μm, about 60 μm to about 100 μm, about 70 μm to about 90 μm, or about 75 μm to about 85 μm, including all ranges and values derivable therebetween. The first, second, third, and/or fourth aspiration screen may, in some embodiments, comprise a top surface and a bottom surface, and the methods provided by the present disclosure may include contacting the preparation of embryo explants or a fraction thereof with the top surface of the first, second, third, and/or fourth aspiration screen. In particular embodiments, the first, second, third, and/or fourth aspiration screen may be structurally connected to the first, second, third, and/or fourth vertical chamber, respectively. The first, second, third, and/or fourth aspiration screen, in certain embodiments, comprises a first end and a second end, and the first end is elevated relative to the second end to produce a first, second, third, and/or fourth incline angle relative to the ground. The first, second, third, and/or fourth incline angle may be, in some embodiments, about 10° to about 60°, about 20° to about 60°, about 25° to about 55°, about 30° to about 50°, about 30° to about 45°, about 35° to about 45°, about 35° to about 40°, about 10°, about 15°, about 20°, about 25°, about 30°, about 31°, about 32°, about 33°, about 34°, about 35°, about 36°, about 37°, about 38°, about 39°, or about 40°, including all ranges and values derivable therebetween.

In some embodiments, the preparation may be introduced into the first vertical chamber through a first input port positioned above the first aspiration screen. The first end of the first aspiration screen may be, in certain embodiments, positioned within the first vertical chamber such that the first end is closer to the first input port compared to the second end, and the first end of the first aspiration screen may be elevated relative to the second end to produce the first incline angle. In certain embodiments, the first, second, third, and/or fourth vertical chamber may comprise a top portion and a bottom portion, wherein the top portion is above the first, second, third, and/or fourth aspiration screen, and the bottom portion is below the first, second, third, and/or fourth aspiration screen, respectively. The upward air flow, in certain embodiments, passes through the first, second, third, and/or fourth aspiration screen from the bottom portion of the first, second, third, and/or fourth vertical chamber to the top portion of the first, second, third, and/or vertical chamber. In some embodiments, introducing the preparation into the first vertical chamber may comprise introducing from a vibratory feeding unit. The vibratory feeding unit may, in some embodiments, be structurally connected to the first vertical chamber, and the vibratory feeding unit may produce a vibratory motion that causes movement of the preparation into the first vertical chamber. The vibratory motion of the vibratory feeding unit may, in certain embodiments, comprise a substantially horizontal vibratory motion. In certain embodiments, the method comprises introducing the preparation to the first chamber at a rate of about 1 g/min to about 70 g/min, about 10 g/min to about 60 g/min, about 20 g/min to about 50 g/min, about 30 g/min to about 40 g/min, about 1 g/min, about 5 g/min, about 10 g/min, about 15 g/min, about 20 g/min, about 25 g/min, about 30 g/min, about 35 g/min, or about 40 g/min, including all ranges and values derivable therebetween.

The upward air flow of the first vertical chamber may have an air velocity, in some embodiments, of about 1.0 m/s to about 25.0 m/s, about 2.5 m/s to about 25.0 m/s, about 2.5 m/s to about 20.0 m/s, about 2.5 m/s to about 17.5 m/s, about 2.5 m/s to about 15.0 m/s, about 2.5 m/s to about 12.5 m/s, about 2.5 m/s to about 10.0 m/s, about 2.5 m/s to about 9.0 m/s, about 2.5 m/s to about 8.0 m/s, or about 3.1 m/s to about 7.2 m/s, including all ranges and values derivable therebetween. The upward air flow velocity of the first chamber may be, for example, about 1.0 m/s, about 1.5 m/s, about 2.0 m/s, about 2.5 m/s, about 2.6 m/s, about 2.7 m/s, about 2.8 m/s, about 2.9 m/s, about 3.0 m/s, about 3.1 m/s, about 3.2 m/s, about 3.3 m/s, about 3.4 m/s, about 3.5 m/s, about 3.6 m/s, about 3.7 m/s, about 3.8 m/s, about 3.9 m/s, about 4.0 m/s, about 4.1 m/s, about 4.2 m/s, about 4.3 m/s, about 4.4 m/s, about 4.5 m/s, about 4.6 m/s, about 4.7 m/s, about 4.8 m/s, about 4.9 m/s, about 5.0 m/s, about 5.1 m/s, about 5.2 m/s, about 5.3 m/s, about 5.4 m/s, about 5.5 m/s, about 5.6 m/s, about 5.7 m/s, about 5.8 m/s, about 5.9 m/s, about 6.0 m/s, about 6.1 m/s, about 6.2 m/s, about 6.3 m/s, about 6.4 m/s, about 6.5 m/s, about 6.6 m/s, about 6.7 m/s, about 6.8, m/s, about 6.9 m/s, about 7.0 m/s, about 7.1 m/s, about 7.2 m/s, about 7.3 m/s, about 7.4 m/s, about 7.5 m/s, about 7.6 m/s, about 7.7 m/s, about 7.8 m/s, about 7.9 m/s, about 8.0 m/s, about 8.1 m/s, about 8.2, m/s, about 8.3 m/s, about 8.4 m/s, about 8.5 m/s, about 8.6 m/s, about 8.7 m/s, about 8.8 m/s, about 8.9 m/s, about 9.0 m/s, about 9.1 m/s, about 9.2 m/s, about 9.3 m/s, about 9.4 m/s, about 9.5 m/s, about 9.6 m/s, about 9.7 m/s, about 9.8 m/s, about 9.9 m/s, or about 10.0 m/s, including all ranges and values derivable therebetween.

The upward air flow of the second vertical chamber may have an air velocity, in certain embodiments, of about 1.0 m/s to about 25.0 m/s, about 2.5 m/s to about 25.0 m/s, about 3.0 m/s to about 20.0 m/s, about 3.0 m/s to about 17.5 m/s, about 3.0 m/s to about 15.0 m/s, about 3.0 m/s to about 12.5 m/s, about 3.0 m/s to about 10 m/s, about 3.0 m/s to about 9.0 m/s, about 3.0 m/s to about 8.5 m/s, or about 4.1 m/s to about 8.2 m/s, including all ranges and values derivable therebetween. The upward air flow velocity of the second vertical chamber may be, for example, about 1.0 m/s, about 1.5 m/s, about 2.0 m/s, about 2.5 m/s, about 2.6 m/s, about 2.7 m/s, about 2.8 m/s, about 2.9 m/s, about 3.0 m/s, about 3.1 m/s, about 3.2 m/s, about 3.3 m/s, about 3.4 m/s, about 3.5 m/s, about 3.6 m/s, about 3.7 m/s, about 3.8 m/s, about 3.9 m/s, about 4.0 m/s, about 4.1 m/s, about 4.2 m/s, about 4.3 m/s, about 4.4 m/s, about 4.5 m/s, about 4.6 m/s, about 4.7 m/s, about 4.8 m/s, about 4.9 m/s, about 5.0 m/s, about 5.1 m/s, about 5.2 m/s, about 5.3 m/s, about 5.4 m/s, about 5.5 m/s, about 5.6 m/s, about 5.7 m/s, about 5.8 m/s, about 5.9 m/s, about 6.0 m/s, about 6.1 m/s, about 6.2 m/s, about 6.3 m/s, about 6.4 m/s, about 6.5 m/s, about 6.6 m/s, about 6.7 m/s, about 6.8, m/s, about 6.9 m/s, about 7.0 m/s, about 7.1 m/s, about 7.2 m/s, about 7.3 m/s, about 7.4 m/s, about 7.5 m/s, about 7.6 m/s, about 7.7 m/s, about 7.8 m/s, about 7.9 m/s, about 8.0 m/s, about 8.1 m/s, about 8.2 m/s, about 8.3, m/s, about 8.4 m/s, about 8.5 m/s, about 8.6 m/s, about 8.7 m/s, about 8.8 m/s, about 8.9 m/s, about 9.0 m/s, about 9.1 m/s, about 9.2 m/s, about 9.3 m/s, about 9.4 m/s, about 9.5 m/s, about 9.6 m/s, about 9.7 m/s, about 9.8 m/s, about 9.9 m/s, or about 10.0 m/s, including all ranges and values derivable therebetween.

In some embodiments, the upward air flow of the third vertical chamber may have an air velocity of about 1.0 m/s to about 25.0 m/s, about 2.5 m/s to about 25.0 m/s, about 3.0 m/s to about 20.0 m/s, about 3.5 m/s to about 17.5 m/s, about 4.0 m/s to about 15.0 m/s, about 4.5 m/s to about 12.5 m/s, about 5.0 m/s to about 12.5 m/s, or about 5.3 m/s to about 11.9 m/s, including all ranges and values derivable therebetween. The upward air flow velocity of the third vertical chamber may be, for example, about 3.5 m/s, about 3.6 m/s, about 3.7 m/s, about 3.8 m/s, about 3.9 m/s, about 4.0 m/s, about 4.1 m/s, about 4.2 m/s, about 4.3 m/s, about 4.4 m/s, about 4.5 m/s, about 4.6 m/s, about 4.7 m/s, about 4.8 m/s, about 4.9 m/s, about 5.0 m/s, about 5.1 m/s, about 5.2 m/s, about 5.3 m/s, about 5.4 m/s, about 5.5 m/s, about 5.6 m/s, about 5.7 m/s, about 5.8 m/s, about 5.9 m/s, about 6.0 m/s, about 6.1 m/s, about 6.2 m/s, about 6.3 m/s, about 6.4 m/s, about 6.5 m/s, about 6.6 m/s, about 6.7 m/s, about 6.8, m/s, about 6.9 m/s, about 7.0 m/s, about 7.1 m/s, about 7.2 m/s, about 7.3 m/s, about 7.4 m/s, about 7.5 m/s, about 7.6 m/s, about 7.7 m/s, about 7.8 m/s, about 7.9 m/s, about 8.0 m/s, about 8.1 m/s, about 8.2 m/s, about 8.3, m/s, about 8.4 m/s, about 8.5 m/s, about 8.6 m/s, about 8.7 m/s, about 8.8 m/s, about 8.9 m/s, about 9.0 m/s, about 9.1 m/s, about 9.2 m/s, about 9.3 m/s, about 9.4 m/s, about 9.5 m/s, about 9.6 m/s, about 9.7 m/s, about 9.8 m/s, about 9.9 m/s, about 10.0 m/s, about 10.1 m/s, about 10.2 m/s, about 10.3 m/s, about 10.4 m/s, about 10.5 m/s, about 10.6 m/s about 10.7 m/s, about 10.8 m/s, about 10.9 m/s, about 11.0 m/s, about 11.1 m/s, about 11.2 m/s, about 11.3 m/s, about 11.4 m/s, about 11.5 m/s, about 11.6 m/s, about 11.7 m/s, about 11.8 m/s, about 11.9 m/s, about 12.0 m/s, about 12.1 m/s, about 12.2 m/s, about 12.3 m/s, about 12.4 m/s, about 12.5 m/s, about 12.6 m/s, about 12.7 m/s, about 12.8 m/s, about 12.9 m/s, about 13.0 m/s, about 13.1 m/s, about 13.2 m/s, about 13.3 m/s, about 13.4 m/s, about 13.5 m/s, about 13.6 m/s, about 13.7 m/s, about 13.8 m/s, about 13.9 m/s, or about 14.0 m/s, including all ranges and values derivable therebetween.

In certain embodiments, the upward air flow of the fourth vertical chamber may have an air velocity of about 1.0 m/s to about 25.0 m/s, about 2.5 m/s to about 25.0 m/s, about 3.0 m/s to about 25.0 m/s, about 4.0 m/s to about 25.0 m/s, about 5.0 m/s to about 22.5 m/s, about 6.0 m/s to about 22.5 m/s, about 6.5 m/s to about 20.5 m/s, or about 7.5 m/s to about 19.9 m/s, including all ranges and values derivable therebetween. The upward air flow velocity of the fourth vertical chamber may have, for example, an air velocity of about 5.0 m/s, about 5.1 m/s, about 5.2 m/s, about 5.3 m/s, about 5.4 m/s, about 5.5 m/s, about 5.6 m/s, about 5.7 m/s, about 5.8 m/s, about 5.9 m/s, about 6.0 m/s, about 6.1 m/s, about 6.2 m/s, about 6.3 m/s, about 6.4 m/s, about 6.5 m/s, about 6.6 m/s, about 6.7 m/s, about 6.8, m/s, about 6.9 m/s, about 7.0 m/s, about 7.1 m/s, about 7.2 m/s, about 7.3 m/s, about 7.4 m/s, about 7.5 m/s, about 7.6 m/s, about 7.7 m/s, about 7.8 m/s, about 7.9 m/s, about 8.0 m/s, about 8.1 m/s, about 8.2 m/s, about 8.3, m/s, about 8.4 m/s, about 8.5 m/s, about 8.6 m/s, about 8.7 m/s, about 8.8 m/s, about 8.9 m/s, about 9.0 m/s, about 9.1 m/s, about 9.2 m/s, about 9.3 m/s, about 9.4 m/s, about 9.5 m/s, about 9.6 m/s, about 9.7 m/s, about 9.8 m/s, about 9.9 m/s, about 10.0 m/s, about 10.1 m/s, about 10.2 m/s, about 10.3 m/s, about 10.4 m/s, about 10.5 m/s, about 10.6 m/s about 10.7 m/s, about 10.8 m/s, about 10.9 m/s, about 11.0 m/s, about 11.1 m/s, about 11.2 m/s, about 11.3 m/s, about 11.4 m/s, about 11.5 m/s, about 11.6 m/s, about 11.7 m/s, about 11.8 m/s, about 11.9 m/s, about 12.0 m/s, about 12.1 m/s, about 12.2 m/s, about 12.3 m/s, about 12.4 m/s, about 12.5 m/s, about 12.6 m/s, about 12.7 m/s, about 12.8 m/s, about 12.9 m/s, about 13.0 m/s, about 13.1 m/s, about 13.2 m/s, about 13.3 m/s, about 13.4 m/s, about 13.5 m/s, about 13.6 m/s, about 13.7 m/s, about 13.8 m/s, about 13.9 m/s, about 14.0 m/s, about 14.1 m/s, about 14.2 m/s, about 14.3 m/s, about 14.4 m/s about 14.5 m/s, about 14.6 m/s, about 14.7 m/s, about 14.8 m/s, about 14.9 m/s, about 15.0 m/s about 15.1 m/s, about 15.2 m/s, about 15.3 m/s, about 15.4 m/s, about 15.5 m/s about 15.6 m/s, about 15.7 m/s, about 15.8 m/s, about 15.9 m/s, about 16.0 m/s, about 16.1 m/s, about 16.2 m/s, about 16.3 m/s, about 16.4 m/s, about 16.5 m/s, about 16.6 m/s, about 16.7 m/s, about 16.8 m/s, about 16.9 m/s, about 17.0 m/s, about 17.1 m/s, about 17.2 m/s, about 17.3 m/s, about 17.4 m/s, about 17.5 m/s, about 17.6 m/s, about 17.7 m/s, about 17.8 m/s, about 17.9 m/s, about 18.0 m/s, about 18.1 m/s, about 18.2 m/s, about 18.3 m/s, about 18.4 m/s, about 18.5 m/s, about 18.6 m/s, about 18.7 m/s, about 18.8 m/s, about 18.9 m/s, about 19.0 m/s, about 19.1 m/s, about 19.2 m/s, about 19.3 m/s, about 19.4 m/s, about 19.5 m/s, about 19.6 m/s, about 19.7 m/s, about 19.8 m/s, about 19.9 m/s, about 20.0 m/s, about 20.1 m/s, about 20.2 m/s, about 20.3 m/s, about 20.4 m/s, about 20.5 m/s, about 20.6 m/s, about 20.7 m/s, about 20.8 m/s, about 20.9 m/s, about 21.0 m/s, about 21.1 m/s, about 21.2 m/s, about 21.3 m/s, about 21.4 m/s, about 21.5 m/s, about 21.6 m/s, about 21.7 m/s, about 21.8 m/s, about 21.9 m/s, or about 22.0 m/s, including all ranges and values derivable therebetween.

The air velocity of the first, second, third, and/or fourth vertical chamber may be adjusted according to the characteristics of the explants to be purified. In particular embodiments, the explants to be purified are corn embryo explants and the air velocity of the first vertical chamber may be about 4.5 m/s to about 6.0 m/s, about 5.0 m/s to about 5.5 m/s, or about 5.1 m/s to about 5.3 m/s, the air velocity of the second chamber may be about 5.5 to about 6.5 m/s or about 5.9 m/s to about 6.1 m/s, the air velocity of the third chamber may be about 6.5 m/s to about 7.5 m/s, about 7.0 m/s to about 7.5 m/s, or about 7.1 m/s to about 7.3 m/s, and/or the air velocity of the fourth chamber may be about 9.5 m/s to about 10.5 m/s or about 9.7 m/s to about 10.1 m/s. In other embodiments, the explants to be purified are soybean embryo explants and the air velocity of the first vertical chamber may be about 4.0 m/s to about 5.5 m/s or about 4.5 m/s to about 5.0 m/s, the air velocity of the second chamber may be about 5.0 m/s to about 7.0 m/s or about 5.5 m/s to about 6.5 m/s, the air velocity of the third chamber may be 7.0 m/s to about 8.5 m/s, about 7.5 m/s to about 8.0 m/s, or about 7.6 m/s to about 7.9 m/s, and/or the air velocity of the fourth chamber may be about 10.5 m/s to about 12.5 m/s, about 10.5 m/s to about 12.0 m/s, or about 10.8 m/s to about 11.9 m/s. In yet other embodiments, the explants to be purified are cotton embryo explants and the air velocity of the first vertical chamber may be about 5.5 m/s to about 8.0 m/s, about 5.5 m/s to about 7.5 m/s, or about 5.9 m/s to about 7.2 m/s, the air velocity of the second chamber may be about 6.5 m/s to about 8.5 m/s or about 6.7 m/s to about 8.2 m/s, the air velocity of the third chamber may be about 8.0 m/s to about 12.5 m/s, about 8.5 m/s to about 12.0 m/s, or about 8.8 m/s to about 11.9 m/s, and/or the air velocity of the fourth chamber may be about 13.0 m/s to about 20.5 m/s, about 13.5 m/s to about 20.0 m/s, or about 13.7 m/s to about 19.9 m/s. In still yet other embodiments, the explants to be purified are wheat embryo explants and the air velocity of the first vertical chamber may be about 2.5 m/s to about 4.0 m/s, about 3.0 m/s to about 3.5 m/s, or about 3.0 m/s to about 3.3 m/s, the air velocity of the second chamber may be about 3.0 m/s to about 5.0 m/s, about 3.5 m/s to about 4.5 m/s, or about 3.8 m/s to about 4.3 m/s, the air velocity of the third chamber may be about 4.5 m/s to about 6.0 m/s, about 5.0 m/s to about 6.0 m/s, or about 5.1 m/s to about 5.5 m/s, and/or the air velocity of the fourth chamber may be about 6.5 m/s to about 8.0 m/s, about 7.0 m/s to about 8.0 m/s, or about 7.2 m/s to about 7.8 m/s. In certain embodiments, the explants to be purified are canola embryo explants and the air velocity of the first vertical chamber may be about 2.5 to about 4.0 m/s, about 3.0 m/s to about 4.0 m/s, about 3.4 m/s to about 3.8 m/s, or about 3.6 m/s, the air velocity of the second chamber may be about 4.0 m/s to about 5.5 m/s, about 4.0 m/s to about 5.0 m/s, about 4.3 m/s to about 4.9 m/s, or about 4.6 m/s, the air velocity of the third chamber may be about 5.0 m/s to about 7.0 m/s, about 5.5 m/s to about 6.5 m/s, or about 6.0 m/s, and/or the air velocity of the fourth chamber may be about 8.0 m/s to about 9.5 m/s, about 8.5 m/s to about 9.0 m/s, or about 8.8 m/s.

The methods provided by the present disclosure may, in certain embodiments, further include removing a portion of the debris material separated from a fraction of plant embryo explants through the top portion of the first, second, third, and/or fourth vertical chamber. In some embodiments, the portion of the debris material may be removed through a first, a second, a third, and/or a fourth discharge port, wherein the first, second, third, and/or fourth vertical chamber comprises an interior portion and an exterior portion, and wherein the top portion of the interior portion of the first vertical chamber is in fluid communication with the first discharge port, the top portion of the interior portion of the second vertical chamber is in fluid communication with the second discharge port, the top portion of the interior portion of the third vertical chamber is in fluid communication with the third discharge port, and/or the top portion of the interior portion of the fourth vertical chamber is in fluid communication with the fourth discharge port. As used herein the term “discharge port” refers to an opening configured to discharge a portion of the debris material. In particular embodiments, the discharge port may be a common or continuous discharge port. A common or continuous discharge port may refer, for example, to a discharge port which allows for continuous discharge of debris material without intervention. As used herein the phrase “in fluid communication” refers to the instance wherein two or more areas or components are in fluid communication or are capable of being in fluid communication. For example, two or more areas or components may be in fluid communication with each other through an unobstructed passageway connecting the areas or components. Two or more areas or components may also be in fluid communication, for example, through an obstructed passageway that comprises, for example, a valve, wherein fluid communication can be established between the areas upon actuating the valve. In certain embodiments, the methods provided by the present disclosure may further comprise collecting a portion of the debris material in a first, second, third, and/or fourth discharge collector, wherein the first, second, third, and/or fourth vertical chamber comprises an interior portion and an exterior portion, and wherein the top portion of the interior portion of the first vertical chamber is in fluid communication with the first discharge collector, the top portion of the interior portion of the second vertical chamber is in fluid communication with the second discharge collector, the top portion of the interior portion of the third vertical chamber is in fluid communication with third discharge collection, and/or the top portion of the interior portion of the fourth vertical chamber is in fluid communication with the fourth discharge collector. As used herein the term “discharge collector” refers to a component capable of collecting debris material. In some embodiments, the discharge collector may be a common or continuous discharge collector. A common or continuous discharge collector may refer, for example, to a discharge collector in which is configured to continuously collect discharged debris material from a particular location. Non-limiting examples of a discharge collector that may be used according to the embodiments of the present disclosure include container or a collection chute. Non-limiting types of containers that may be used as a discharge collector include a bottle, a receptacle, a tube, a cannister, or a bag. A container may be of any appropriate geometrical shape, non-limiting examples of which include a cylinder, a sphere, a triangular prism, a cube, and a cone. A collection chute may, in some embodiments, be a hollow component used for holding or transporting the debris material.

In certain embodiments, the top portion of the first, second, third, and/or fourth vertical chamber may be structurally connected to a turned segment comprising an interior portion and an exterior portion, wherein the interior portion of the first turned segment is in fluid communication with the top portion of the interior portion of the first vertical chamber, the interior portion of the second turned segment is in fluid communication with the top portion of the interior portion of the second vertical chamber, wherein the interior portion of the third turned segment is in fluid communication with the top portion of the interior portion of the third vertical chamber, and/or the interior portion of the fourth turned segment is in fluid communication with the top portion of the interior portion of the fourth vertical chamber. In some embodiments, the upward airflow in the first, second, third, and/or fourth vertical chamber is redirected to become a redirected airflow in the first, second, third, and/or fourth turned segment. The maximum angle between the direction of the redirected airflow and the upward airflow, in particular embodiments, is at least 90°. In some embodiments, the maximum angle between the redirected airflow and the upward airflow is about 90° to about 180°, about 100° to about 180°, about 110° to about 180°, about 120° to about 180°, about 130° to about 180°, about 140° to about 180°, about 150° to about 180°, about 90°, about 95°, about 100°, about 105°, about 110°, about 115°, about 120°, about 125°, about 130°, about 135°, about 140°, about 145°, about 150°, about 155°, about 160°, about 165°, about 170°, about 175°, or about 180°, including all ranges and values derivable therebetween.

In certain embodiments, the first aspiration screen comprises a first end and a second end, the first turned segment comprises a top end and a bottom end, the first end of the first aspiration screen is elevated relative to the second end to produce a first incline angle, and the vertical distance between the first end of the first aspiration screen and the bottom end of the interior portion of the first turned segment is about 12.7 cm to about 76.2 cm, about 12.7 cm to about 63.5 cm, about 12.7 cm to about 50.8 cm, about 12.7 cm to about 38.1 cm, about 25.4 cm to about 76.2 cm, about 25.4 cm to about 50.8 cm, about 25.4 cm to about 38.1 cm, about 12.7 cm, 15.24 cm, about 17.78 cm, about 20.32 cm, about 22.86 cm, about 25.4 cm, about 27.94 cm, about 30.48 cm, about 33.02 cm, about 35.56 cm, about 38.1 cm, about 40.64 cm, about 43.18 cm, about 45.72 cm, about 48.26 cm, about 50.8 cm, about 57.15 cm, about 63.5 cm, about 69.85 cm, or about 76.2 cm, including all ranges and values derivable therebetween. In some embodiments, the second aspiration screen comprises a first end and a second end, the second turned segment comprises a top end and a bottom end, the first end of the second aspiration screen is elevated relative to the second end to produce a second incline angle, and the vertical distance between the first end of the second aspiration screen and the bottom end of the interior portion of the second turned segment is about 12.7 cm to about 101.6 cm, about 12.7 cm to about 76.2 cm, about 12.7 cm to about 63.5 cm, about 12.7 cm to about 50.8 cm, about 12.7 cm to about 38.1 cm, about 25.4 cm to about 76.2 cm, about 25.4 cm to about 63.5 cm, about 38.1 cm to about 63.5 cm, about 40.64 cm to about 55.88 cm, about 12.7 cm, about 25.4 cm, about 17.78 cm, about 20.32 cm, about 22.86 cm, about 25.4 cm, about 27.94 cm, about 30.48 cm, about 33.02 cm, about 35.56 cm, about 38.1 cm, 40.64 cm, about 43.18 cm, about 45.72 cm, about 48.26 cm, about 50.8 cm, 53.34 cm, about 55.88 cm, about 58.42 cm, about 60.96 cm, about 63.5 cm, about 66.04 cm, about 68.58 cm, about 71.12 cm, about 73.66 cm, about 76.2 cm, about 82.55 cm, about 88.9 cm, about 95.25 cm, or about 101.6 cm, including all ranges and values derivable therebetween. In particular embodiments, the third aspiration screen comprises a first end and a second end, the third turned segment comprises a top end and a bottom end, the first end of the third aspiration screen is elevated relative to the second end to produce a third incline angle, and the vertical distance between the first end of the third aspiration screen and the bottom end of the interior portion of the third turned segment is about 25.4 cm to about 152.4 cm, about 25.4 cm to about 127 cm, about 25.4 cm to about 101.6 cm, about 25.4 cm to about 88.9 cm, about 38.1 cm to about 88.9 cm, about 50.8 cm to about 76.2 cm, about 55.88 cm to about 71.12 cm, about 25.4 cm, about 38.1 cm, about 50.8 cm, about 53.34 cm, about 55.88 cm, about 58.42 cm, about 60.96 cm, about 63.5 cm, about 66.04 cm, about 68.58 cm, about 71.12 cm, about 73.66 cm, about 76.2 cm, about 82.55 cm, about 88.9 cm, about 95.25 cm, about 101.6 cm, about 107.95 cm, about 114.3 cm, about 120.65 cm, about 127 cm, about 133.35 cm, about 139.7 cm, about 146.05 cm, or about 152.4 cm, including all ranges and values derivable therebetween. In some embodiments, the fourth aspiration screen comprises a first end and a second end, the fourth turned segment comprises a top end and a bottom end, the first end of the fourth aspiration screen is elevated relative to the second end to produce a fourth incline angle, and the vertical distance between the first end of the fourth aspiration screen and the bottom end of the interior portion of the fourth turned segment is about 12.7 cm to about 152.4 cm, about 25.4 cm to about 152.4 cm, about 25.4 cm to about 127 cm, about 38.1 cm to about 127 cm, about 38.1 cm to about 114.3 cm, about 50.8 cm to about 101.6 cm, about 63.5 cm to about 88.9 cm, about 68.58 cm to about 83.82 cm, about 12.7 cm, about 38.1 cm, about 50.8 cm, about 57.15 cm, about 63.5 cm, about 66.04 cm, about 68.58 cm, about 71.12 cm, about 73.66 cm, about 76.2 cm, about 78.74 cm, about 81.28 cm, about 83.82 cm, about 86.36 cm, about 88.9 cm, about 91.44 cm, about 93.98 cm, about 96.52 cm, about 99.06 cm, about 101.6 cm, about 107.95 cm, about 114.3 cm, about 120.65 cm, about 127 cm, about 133.35 cm, about 139.7 cm, about 146.05 cm, or about 152.4 cm, including all ranges and values derivable therebetween.

In particular embodiments, the methods provided by the present disclosure may further comprise collecting the first fraction, the second fraction, the third fraction, and/or the fourth fraction of the plant embryo explants from the top surface of the first aspiration screen, the second aspiration screen, the third aspiration screen, and/or the fourth aspiration screen, respectively. In certain embodiments, the methods provided by the present disclosure may further comprise transferring the first fraction, the second fraction, the third fraction, and/or the fourth fraction of plant embryo explants through an output port to an output collector and collecting the fraction. In some embodiments, the first output port is positioned above the first aspiration screen, the second output port is positioned above the second aspiration screen, the third output port is positioned above the third aspiration screen, and/or the fourth output port is positioned above the fourth aspiration screen. As used herein the term “output port” refers to an opening configured to transfer a fraction of embryo explants to an output collector. As used herein the term “output collector” refers to a component capable of collecting a fraction of embryo explants. Non-limiting examples of an output collector that may be used according to the embodiments of the present disclosure include container or a collection chute. Non-limiting types of containers that may be used as an output collector include a bottle, a receptacle, a tube, a cannister, or a bag. A container may be of any appropriate geometrical shape, non-limiting examples of which include a cylinder, a sphere, a triangular prism, a cube, and a cone. A collection chute may, in some embodiments, be a hollow component used for holding or transporting the fraction of embryo explants. In some embodiments, the first portion of the debris material has been removed from the first fraction, the second portion of the debris material has been removed from the second fraction, the third portion of the debris material has been removed from the third fraction, and/or the fourth portion of the debris material has been removed from the fourth fraction prior to the collecting. The first, second, third, and/or fourth aspiration screen, in certain embodiments, comprises a first end and a second end, wherein the second end of the aspiration screen is positioned within the vertical chamber such that the second end is closer to the output port than is the first end, and wherein the first end of the first aspiration screen is elevated relative to the second end to produce an incline angle.

The methods provided by the present disclosure, in particular embodiments, may further comprise transferring the first fraction of the plant embryo explants into a second vertical chamber, transferring the second fraction of the plant embryo explants into a third vertical chamber, and/or transferring the third fraction of the plant embryo explants into a fourth vertical chamber, wherein the first portion of the debris material has been removed from the first fraction, the second portion of the debris material has been removed from the second fraction, and/or the third portion of the debris material has been removed from the third fraction. In particular embodiments, the first fraction, the second fraction, and/or the third fraction may be transferred to the second vertical chamber, the third vertical chamber, and/or the fourth vertical chamber, respectively, through an advancement port. As used herein the term “advancement port” refers to a passageway configured to transfer material from one chamber or functional unit to another chamber or functional unit. In some embodiments, the first advancement port comprises an opening between the first vertical chamber and the second vertical chamber, the second advancement port comprises an opening between the second vertical chamber and the third vertical chamber, and/or the third advancement port comprises an opening between the third vertical chamber and the fourth vertical chamber. In particular embodiments, the first advancement port is positioned above the first aspiration screen, the second advancement port is positioned above the second aspiration screen, and/or the third advancement port is positioned above the third aspiration screen. In certain embodiments, the first fraction is transferred into the second vertical chamber above the second aspiration screen, the second fraction is transferred into the third vertical chamber above the third aspiration screen, and/or the third fraction is transferred into the fourth vertical chamber above the fourth aspiration screen. In some embodiments, the first advancement port is positioned above the second aspiration screen, the second advancement port is positioned above the third aspiration screen, and/or the third advancement port is positioned above the fourth aspiration screen. The second, third, and/or fourth aspiration screen, in some embodiments, comprises a first end and a second end, wherein the first end of the second, third, and/or fourth aspiration screen is positioned within the second, third, and/or fourth vertical chamber, respectively, such that the first end is closer to the first, second, and/or third advancement port compared to the second end, and wherein the first end of the second, third, and/or fourth aspiration screen is elevated relative to the second end to produce an incline angle as described herein.

In particular embodiments, the first vertical chamber, the second vertical chamber, the third vertical chamber, and/or the fourth vertical chamber has an average horizontal cross-sectional area of about 32.258 cm² to about 645.16 cm², about 32.258 cm² to about 516.128 cm², about 32.258 cm² to about 387.096 cm², about 32.258 cm² to about 322.58 cm², about 64.516 cm² to about 645.16 cm², about 64.516 cm² to about 516.128 cm², about 64.516 cm² to about 387.096 cm², about 64.516 cm² to about 322.58 cm², about 96.774 cm² to about 258.064 cm², about 96.774 cm² to about 225.806 cm², about 129.032 cm² to about 193.548 cm², or about 129.032 cm² to about 161.29 cm², including all ranges and values derivable therebetween.

Embodiments of the present disclosure include a method of purifying genetically modifiable dry plant embryo explants, comprising aspirating within a vertical chamber a preparation of dry plant embryo explants comprising meristematic tissue with an upward flow having an air velocity of about 1.0 m/s to about 25.0 m/s, about 1.0 m/s to about 20.0 m/s, about 1.0 m/s to about 15.0 m/s, about 1.0 m/s to about 10.0 m/s, about 2.0 m/s to about 25.0 m/s, about 2.0 m/s to about 20.0 m/s, about 2.0 m/s to about 15.0 m/s, or about 2.0 m/s to about 10.0 m/s, wherein the preparation comprises a population of dry plant embryo explants and debris material; and separating a fraction of the plant embryo explants of the preparation from a portion of the debris material according to a displacement of the fraction relative to a displacement of the portion of the debris material produced by the air flow within the vertical chamber, wherein the vertical chamber in fluid communication with a turned segment a waste collector. The turned segment, in some embodiments, is structurally connected to the vertical chamber. The waste collector, in particular embodiments, is structurally connected to the vertical chamber.

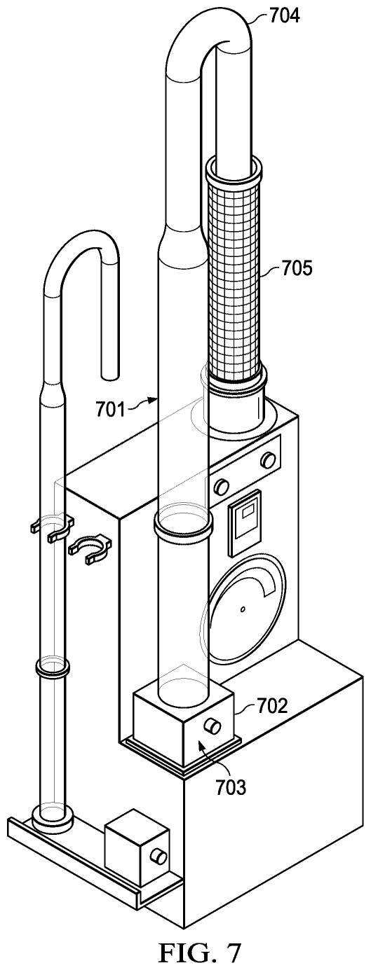

FIG. 7 is a diagram showing an apparatus having an aspiration chamber 701 to aspirate a preparation of dry embryo explants; an input compartment 702 having an input drawer 703 configured to open and to introduce the preparation into the aspiration chamber 701; the aspiration chamber 701 being in fluid communication with a turned segment 704 and a waste collector 705, wherein the turned segment 704 is configured to facilitate the transfer of a portion of the debris material from the aspiration chamber 701 to the waste receptacle 705 and to inhibit debris material from returning to the chamber 701 once the debris material has passed through the turned segment 704, and wherein the waste receptacle 705 is configured to collect the portion of the debris material. In specific embodiments, the preparation is introduced into the input compartment above an aspiration screen (not shown).

In some aspects, the upward air flow may have an air velocity of about 1.0 m/s to about 25.0 m/s, about 1.0 m/s to about 20.0 m/s, about 1.0 m/s to about 15.0 m/s, about 1.0 m/s to about 10.0 m/s, about 2.0 m/s to about 25.0 m/s, about 2.0 m/s to about 20.0 m/s, about 2.0 m/s to about 15.0 m/s, about 2.0 m/s to about 10.0 m/s, about 3.0 m/s to about 10.0 m/s, about 3.0 m/s to about 9.5 m/s, about 3.0 m/s to about 9.0 m/s, about 3.0 m/s to about 8.0 m/s, about 3.0 m/s to about 7.8 m/s, about 3.4 m/s to about 9.1 m/s, about 2.0 m/s, about 2.1 m/s, about 2.2 m/s, about 2.3 m/s, about 2.4 m/s, about 2.5 m/s, about 2.6 m/s, about 2.7 m/s, about 2.8 m/s, about 2.9 m/s, about 3.0 m/s, about 3.1 m/s, about 3.2 m/s, about 3.3 m/s, about 3.4 m/s, about 3.5 m/s, about 3.6 m/s, about 3.7 m/s, about 3.8 m/s, about 3.9 m/s, about 4.0 m/s, about 4.1 m/s, about 4.2 m/s, about 4.3 m/s, about 4.4 m/s, about 4.5 m/s, about 4.6 m/s, about 4.7 m/s, about 4.8 m/s, about 4.9 m/s, about 5.0 m/s, about 5.1 m/s, about 5.2 m/s, about 5.3 m/s, about 5.4 m/s, about 5.5 m/s, about 5.6 m/s, about 5.7 m/s, about 5.8 m/s, about 5.9 m/s, about 6.0 m/s, about 6.1 m/s, about 6.2 m/s, about 6.3 m/s, about 6.4 m/s, about 6.5 m/s, about 6.6 m/s, about 6.7 m/s, about 6.8, m/s, about 6.9 m/s, about 7.0 m/s, about 7.1 m/s, about 7.2 m/s, about 7.3 m/s, about 7.4 m/s, about 7.5 m/s, about 7.6 m/s, about 7.7 m/s, about 7.8 m/s, about 7.9 m/s, about 8.0 m/s, about 8.1 m/s, about 8.2 m/s, about 8.3, m/s, about 8.4 m/s, about 8.5 m/s, about 8.6 m/s, about 8.7 m/s, about 8.8 m/s, about 8.9 m/s, about 9.0 m/s, about 9.1 m/s, about 9.2 m/s, about 9.3 m/s, about 9.4 m/s, about 9.5 m/s, about 9.6 m/s, about 9.7 m/s, about 9.8 m/s, about 9.9 m/s, or about 10.0 m/s, including all ranges and values derivable therebetween. The air velocity of the chamber may be adjusted according to the characteristics of the explants to be purified. In some embodiments, the preparation comprises wheat embryo explants and the air velocity is about 2.5 m/s to about 8.5 m/s, about 3.0 m/s to about 8.0 m/s, or about 3.0 m/s to about 7.8 m/s. In certain embodiments, the preparation comprises canola embryo explants and the air velocity is about 3.0 m/s to about 10.0 m/s, about 3.0 m/s to about 9.5 m/s, or about 3.4 m/s to about 9.1 m/s.

In some embodiments, the methods provided by the present disclosure may further comprise introducing the preparation into the vertical chamber above an aspiration screen positioned within an input compartment, the aspiration screen comprising a plurality of openings, each comprising an opening size and an opening shape, as described herein. In specific embodiments, the preparation or the population of dry embryo explants is contacted with the top surface of the aspiration screen during the aspirating, as described herein. In some embodiments, the upward air flow passes through the aspiration screen to the vertical chamber. The input compartment, in certain embodiments, is structurally connected to the vertical chamber. In some embodiments, the aspiration screen is structurally connected to the input compartment.

In particular embodiments, the methods provided by the present disclosure further comprise removing the portion of the debris material separated from the fraction of plant embryo explants through the turned segment. In some embodiments, the vertical chamber comprises an interior portion and an exterior portion, the interior portion of the vertical chamber is in fluid communication with the turned segment. In certain embodiments, the methods provided by the present disclosure may further comprise collecting the portion of the debris in the waste collector, wherein the vertical chamber comprises an interior portion and an exterior portion, the waste collector comprises an interior portion and an exterior portion, and wherein the interior portion of the vertical chamber is in fluid communication with the interior portion of the waste collector. The turned segment, in particular embodiments, may comprise an interior portion and an exterior portion, wherein the interior portion of the turned segment is in fluid communication with the interior portion of the vertical chamber, and wherein the upward air flow in the vertical chamber is redirected to become a redirected air flow in the turned segment. In particular embodiments, the maximum angle between the direction of the redirected air flow and the upward air flow is at least 90°. In some embodiments, the maximum angle between the redirected airflow and the upward airflow is about 90° to about 180°, about 100° to about 180°, about 110° to about 180°, about 120° to about 180°, about 130° to about 180°, about 140° to about 180°, about 150° to about 180°, about 90°, about 95°, about 100°, about 105°, about 110°, about 115°, about 120°, about 125°, about 130°, about 135°, about 140°, about 145°, about 150°, about 155°, about 160°, about 165°, about 170°, about 175°, or about 180°, including all ranges and values derivable therebetween. In particular embodiments, the turned segment comprises a top end and a bottom end, and the vertical distance between the aspiration screen and the bottom end of the interior portion of the turned segment is about 20 cm to about 120 cm, about 20 cm to about 100 cm, about 30 cm to about 90 cm, about 40 cm to about 80 cm, about 50 cm to about 70 cm, or about 55 cm to about 65 cm, including all ranges and values derivable therebetween.

In some embodiments, the methods provided by the present disclosure may further comprise collecting the fraction of the plant embryo explants from the top surface of the aspiration screen. In particular embodiments, the methods provided by the present disclosure may further comprise transferring the fraction of plant embryo explants through an output port, as described herein, to an output collector, as described herein, wherein the output port is positioned above the aspiration screen; and collecting the fraction in the output collector.

In particular embodiments, the vertical chamber has an average horizontal cross-sectional area of about 10.0 cm² to about 100.0 cm², about 10.0 cm² to about 90.0 cm², about 10.0 cm² to about 80.0 cm², about 10.0 cm² to about 70.0 cm², about 10.0 cm² to about 60.0 cm², about 10.0 cm² to about 50.0 cm², about 10.0 cm² to about 60.0 cm², about 10.0 cm² to about 50.0 cm², about 10.0 cm² to about 40.0 cm², about 10.0 cm² to about 30.0 cm², about 15.0 cm² to about 30.0 cm², about 20.0 cm² to about 30.0 cm², or about 22.0 cm² to about 26.0 cm², including all ranges and values derivable therebetween.

In particular embodiments, the waste collector comprises a plurality of collector openings, each opening comprising a collector opening shape and a collector opening size. The collector opening shape and the collector opening size may be modified according to the characteristics of the embryo explants to be purified. The collector opening shape may be any geometric shape, non-limiting examples of which include a rectangle, a square, a circle, and an oval. In some embodiments, the collector opening size may comprise a collector opening diameter, a collector opening width, and/or a receptacle opening length. The diameter, width, and/or length of the collector opening, in some embodiments, may be about 10 μm to about 500 μm, about 10 μm to about 400 μm, 20 μm to about 300 μm, 20 μm to about 200 μm, about 20 μm to about 150 μm, about 20 μm to about 120 μm, about 30 μm to about 120 μm, about 40 μm to about 120 μm, about 50 μm to about 110 μm, about 60 μm to about 100 μm, about 70 μm to about 90 μm, or about 75 μm to about 85 μm, including all ranges and values derivable therebetween.

Embodiments of the present disclosure include a method of purifying genetically modifiable dry plant embryo explants, the method comprising aspirating within a functional unit of a vertical chamber a preparation of dry plant embryo explants comprising meristematic tissue or a fraction thereof with an air flow having an air flow velocity, wherein the preparation or fraction thereof comprises a population of dry plant embryo explants and debris material; and separating a fraction of the plant embryo explants from a portion of the debris material within the functional unit of the vertical chamber according to a displacement of the fraction relative to a displacement of the portion of the debris material produced by the air flow within the functional unit, wherein the air flow comprises a variable vertical component and a variable horizontal component, and wherein the functional unit of the vertical chamber comprises a lower partition, an air input port, and an air output port. In some embodiments, the lower partition extends inward from a side wall of the vertical chamber to define a lower advancement port between the lower partition and an opposite side wall of the vertical chamber. In certain embodiments, the air input port comprises an opening in the side wall of the vertical chamber below the lower partition. The air flow at least partially enters the vertical chamber, in some embodiments, through the air input port, travels through the lower advancement port, and exits the vertical chamber through the air output port. In certain embodiments, a functional unit as described herein may further comprise an air intake partition, wherein the intake partition extends inward from the side wall of the vertical chamber to further define the lower advancement port between the air intake partition and the opposite side wall of the vertical chamber. In some embodiments, the air input port is positioned above the air intake partition such that the air flow at least partially entering the vertical chamber through the air input port is channeled between the lower partition and the air intake partition. In particular embodiments, a preparation of dry embryo explants or a fraction thereof may be aspirated within a first, a second, a third, a fourth, a fifth, and/or a sixth functional unit in order to separate a fraction of embryo explants from a portion of the debris material. The first, second, third, fourth, fifth, and/or sixth functional unit may, in certain embodiments, comprise a first, second, third, fourth, fifth, and/or sixth lower partition, air input port, and air output port, respectively. In some embodiments, the first, second, third, fourth, and/or fifth lower partition may extend inward from a side wall of the vertical chamber to define a first, second, third, fourth, and/or fifth lower advancement port, respectively. The first, second, third, fourth, fifth, and/or sixth functional units, in some embodiments, may further comprise a first, second, third, fourth, fifth, and/or sixth air intake partition, respectively.

As used herein the term “partition” refers to a substantially planar member that extends inward from a side wall or an opposite side wall of a vertical chamber as described herein. A “functional unit” as used herein refers to a region of a vertical chamber which is defined by one or more partitions. A functional unit of a vertical chamber as described herein may be, in some embodiments, in fluid communication with one or more additional functional units. In particular embodiments, air, embryo explants, and/or debris material may move from one functional unit to another functional unit, and a portion of the debris material may be removed from a fraction of plant embryo explants in each functional unit. As used herein the term “air input port” refers to an opening configured to allow air to flow into the vertical chamber. As used herein the term “air output port” refers to an opening configured to discharge air from the vertical chamber. In particular embodiments, a portion of the debris material may also be discharged through the air output port.

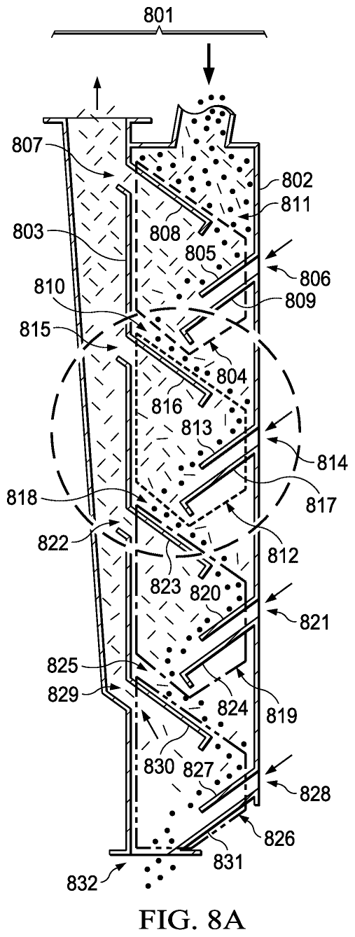

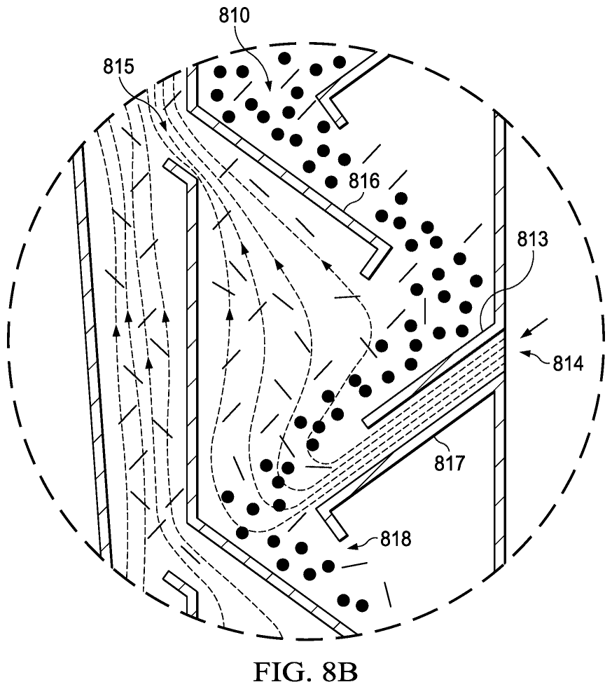

FIG. 8A is diagram showing a vertical chamber 801 having a side wall 802, an opposite side wall 803, and a first functional unit 804, the first functional unit 804 having a first lower partition 805, a first air input port 806, a first air output port 807, a first upper partition 808, and a first air intake partition 809, wherein the first lower partition 805 extends inward from the side wall 802 of the vertical chamber 801 to define a first lower advancement port 810 between the first lower partition 805 and the opposite side wall 803 of the vertical chamber 801, wherein the first upper partition 802 extends inward from the opposite side wall 803 of the vertical chamber 801 to define a first upper advancement port 811 between the first upper partition 808 and the side wall 802 of the vertical chamber 801; the vertical chamber 801 further has a second functional unit 812, the second functional 812 unit having a second lower partition 813, a second air input port 814, a second air output port 815, a second upper partition 816, and a second air intake partition 817, wherein the second lower partition 813 extends inward from the side wall 802 of the vertical chamber 801 to define a second lower advancement port 818 between the second lower partition 813 and the opposite side wall 803 of the vertical chamber 801; the vertical chamber 801 further has a third functional unit 819, the third functional unit 819 having a third lower partition 820, a third air input port 821, a third air output port 822, a third upper partition 823, and a third air intake partition 824, wherein the third lower partition 820 extends inward from the side wall 802 of the vertical chamber 801 to define a third lower advancement port 825 between the third lower partition 820 and the opposite side wall 803 of the vertical chamber 801; the vertical chamber 801 further has a fourth functional unit 826, the fourth functional unit 826 having a fourth lower partition 827, a fourth air input port 828, a fourth air output port 829, a fourth upper partition 830, and a fourth air intake partition 831, wherein the fourth lower partition 827 extends inward from the side wall 802 of the vertical chamber 801 to define a lower collection port 832 between the fourth lower partition 827 and the opposite side wall 803 of the vertical chamber 801. FIG. 8B is a diagram showing a zoomed in view of the area shown by the dashed circle from FIG. 8A, FIG. 8B shows the first lower advancement port 810, the second lower partition 813, the second air input port 814, the second air output port 815, the second upper partition 816, the second air intake partition 817, and the second lower advancement port 818. As provided by the present disclosure, the vertical chamber, in certain embodiments, may comprise at least 1, at least 2, at least 3, at least 4, at least 5, or at least 6 functional units.

In some embodiments, a functional unit of the vertical chamber may further comprise an upper partition, wherein the upper partition extends inward from the opposite side wall of the vertical chamber. The upper partition, in certain embodiments, is positioned above the lower partition or the input port. The air output port, in some embodiments, is positioned below the upper partition. The first, second, third, fourth, fifth, and/or sixth functional units, in some embodiments, may comprise a first, second, third, fourth, fifth, and/or sixth upper partition, respectively. In particular embodiments, the first upper partition extends inward from the opposite side wall of the vertical chamber to define an upper advancement port between the first upper partition and the side wall of the vertical chamber. The methods provided by the present disclosure, in some embodiments, may comprise introducing the preparation of dry plant embryo explants into the first functional unit of the vertical chamber. In certain embodiments, the preparation of plant embryo explants is introduced into the first functional unit above the first upper partition. The methods provided by the present disclosure, in some embodiments, may further comprise contacting the preparation of dry plant embryo explants or a portion thereof with a top surface of the first upper partition before gravity causes the preparation or the portion thereof to fall through the first upper advancement port. The methods provided by the present disclosure may, in some embodiments, further comprise introducing the first fraction into the second functional unit, the second fraction into the third functional unit, the third fraction into the fourth functional unit, the fourth fraction into the fifth functional unit, and/or the fifth fraction into the sixth functional unit. In some embodiments, the first fraction is introduced above the second upper partition, the second fraction is introduced above the third upper partition, the third fraction is introduced above the fourth upper partition, the fourth fraction is introduced above the fifth upper partition, and/or the fifth fraction is introduced above the sixth upper partition. The first fraction of dry plant embryo explants or a portion thereof, in some embodiments, is contacted with a top surface of the second upper partition before gravity causes the first fraction or the portion thereof to fall through the first lower advancement port. The second fraction of dry plant embryo explants or a portion thereof, in particular embodiments, is contacted with a top surface of the third upper partition before gravity causes the second fraction or the portion thereof to fall through the second lower advancement port. The third fraction of dry plant embryo explants or a portion thereof, in certain embodiments, is contacted with a top surface of the fourth upper partition before gravity causes the third fraction or the portion thereof to fall through the third lower advancement port. The fourth fraction of dry plant embryo explants or a portion thereof, in some embodiments, is contacted with a top surface of the fifth upper partition before gravity causes the fourth fraction or the portion thereof to fall through the fourth lower advancement port. The fifth fraction of dry plant embryo explants or a portion thereof, in particular embodiments, is contacted with a top surface of the sixth upper partition before gravity causes the fifth fraction or the portion thereof to fall through the fifth lower advancement port.

In some embodiments, the methods provided by the present disclosure may further comprise transferring the first fraction of the plant embryo explants through the first lower advancement port into a second functional unit, wherein the first lower advancement port is between the first functional unit and the second functional unit, and wherein the first functional unit is positioned above the second functional unit. Embodiments of the present disclosure may further comprise transferring the second fraction of the plant embryo explants through the second lower advancement port into a third functional unit, wherein the second lower advancement port is between the second functional unit and the third functional unit, and wherein the second functional unit is positioned above the third functional unit. The present disclosure further provides embodiments comprising transferring the third fraction of the plant embryo explants through the third lower advancement port into a fourth functional unit, wherein the third lower advancement port is between the third functional unit and the fourth functional unit, and wherein the third functional unit is positioned above the fourth functional unit. In particular embodiments, the methods provided by the present disclosure may further comprise transferring the fourth fraction of the plant embryo explants through the fourth lower advancement port into a fifth functional unit, wherein the fourth lower advancement port is between the fourth functional unit and the fifth functional unit, and wherein the fourth functional unit is positioned above the fifth functional unit. Embodiments of the present disclosure may further comprise transferring the fifth fraction of the plant embryo explants through the fifth lower advancement port into a sixth functional unit, wherein the fifth lower advancement port is between the fifth functional unit and the sixth functional unit, and wherein the fifth functional unit is positioned above the sixth functional unit.

In some embodiments, an upper partition extends inward from the opposite side wall at an upper slope angle, a lower partition extends inward from the side wall at a lower slope angle, and/or an air intake partition extends inward from the side wall at an intake slope angle. The upper slope angle, the lower slope angle, and/or the intake slope angle, in certain embodiments, is a negative angle relative to horizontal. The vertical chamber, in some embodiments, comprises a center cavity. In particular embodiments, an upper partition comprises a first end and a second end, the first end is attached to the opposite side wall, the second end extends into the center cavity of the vertical chamber, and the first end is elevated relative to the second end to create a downward slope angle from the opposite side wall. A lower partition, in some embodiments, comprises a first end and a second end, the first end is attached to the side wall, the second end extends into the center cavity of the vertical chamber, and the first end is elevated relative to the second end to create a downward slope angle from the side wall. An air intake partition, in particular embodiments, comprises a first end and a second end, the first end is attached to the side wall, the second end extends into the center cavity of the vertical chamber, and the first end is elevated relative to the second end to create a downward slope angle from the side wall. The upper slope angle, the lower slope angle, and/or the intake slope angle may be, in particular embodiments, about −20 degrees to about −50 degrees, about −25 degrees to about −45 degrees, about −30 degrees to about −40 degrees, about −20 degrees, about −25 degrees, about −30 degrees, about −31 degrees, about −32 degrees, about −33 degrees, about −34 degrees, about −35 degrees, about −36 degrees, about −37 degrees, about −38 degrees, about −39 degrees, about −40 degrees, about −45 degrees, or about −50 degrees relative to horizontal, including all ranges and values derivable therebetween.

In particular embodiments, the methods provided by the present disclosure may further comprise transferring the first fraction of embryo explants through the first lower advancement port, the second fraction of embryo explants through the second lower advancement port, the third fraction of embryo explants through the third lower advancement port, the fourth fraction of embryo explants through the fourth lower advancement port, or the fifth fraction of embryo explants through the fifth lower advancement port by gravity. The preparation of dry plant embryo explants or the portion thereof, in some embodiments, may be contacted with a top surface of the first lower partition before transferring the first fraction through the first lower advancement port. The first fraction of embryo explants or a portion thereof, in certain embodiments, may be contacted with the top surface of the second lower partition before transferring the second fraction through the second lower advancement port. The second fraction of dry plant embryo explants or the portion thereof may be contacted, in specific embodiments, with a top surface of the third lower partition before transferring the third fraction through the third lower advancement port. The third fraction of dry plant embryo explants or the portion thereof, in certain embodiments, may be contacted with a top surface of the fourth lower partition before transferring the fourth fraction through the fourth lower advancement port. The fourth fraction of dry plant embryo explants or the portion thereof, in some embodiments, may be contacted with a top surface of the fifth lower partition before transferring the fifth fraction through the fifth lower advancement port.

Embodiments of the present disclosure may comprise collecting the first fraction of the plant embryo explants from the first functional unit, wherein the first portion of the debris material has been removed from the first fraction; collecting the second fraction of the plant embryo explants from the second functional unit, wherein the second portion of the debris material has been removed from the second fraction; collecting the third fraction of the plant embryo explants from the third functional unit, wherein the third portion of the debris material has been removed from the third fraction; collecting the fourth fraction of the plant embryo explants from the fourth functional unit, wherein the fourth portion of the debris material has been removed from the fourth fraction; collecting the fifth fraction of the plant embryo explants from the fifth functional unit, wherein the fifth portion of the debris material has been removed from the fifth fraction; or collecting the sixth fraction of the plant embryo explants from the sixth functional unit, wherein the sixth portion of the debris material has been removed from the sixth fraction. In some embodiments, the first, second, third, fourth, fifth, and/or sixth functional units may comprise a lower collection port. A “lower collection port” as used herein refers to an opening configured to transfer a fraction of embryo explants to a lower collector. In certain embodiments, a lower partition extends inward from the side wall of the vertical chamber to define the lower collection port between the lower partition and the opposite side wall of the vertical chamber. A “lower collector” as used herein refers to refers to a component capable of collecting a fraction of embryo explants. Non-limiting examples of a lower collector that may be used according to the embodiments of the present disclosure include a container or a collection chute, as described herein. Embodiments of the present disclosure may further comprise transferring the first fraction of plant embryo explants through the first lower collection port by gravity; transferring the second fraction of plant embryo explant through the second lower collection port by gravity; transferring the third fraction of plant embryo explants through the third lower collection port by gravity; transferring the fourth fraction of plant embryo explants through the fourth lower collection port by gravity; transferring the fifth fraction of plant embryo explants through the fifth lower collection port by gravity; or transferring the sixth fraction of embryo explants through the sixth collection port by gravity.

In some embodiments, the methods provided by the present disclosure may further comprise removing the first portion of the debris material separated from the first fraction through the first air output port, the second portion of the debris material separated from the second fraction through the second air output port, the third portion of the debris material separated from the third fraction through the third air output port, the fourth portion of the debris material separated from the fourth fraction through the fourth air output port, the fifth portion of the debris material separated from the fifth fraction through the fifth air output port, and/or the sixth portion of the debris material separated from the sixth fraction through the sixth air output port. The portion of the debris material, in certain embodiments, may travel with the air flow through the air output port. In particular embodiments, the first air output port, the second air output port, the third air output port, the fourth air output port, the fifth air output port, and/or the sixth air output port is in fluid communication with a discharge channel, and the first air flow, the second air flow, the third air flow the fourth, the fifth air flow, or the sixth air flow travels through the first air output port, the second air output port, the third air output port, the fourth air output port, the fifth air output port, or the sixth air output port and into the discharge channel.

The air flow characteristics, non-limiting examples of which include air velocity and air flow angle, may, in some embodiments, be different within each functional unit of the vertical chamber. In other embodiments, the air flow characteristics may be the same within each functional unit of the vertical chamber. The vertical chamber, in certain embodiments, may comprise at least two, at least three, at least four, at least five, or at least six functional units. In some embodiments, the air flow characteristics of at least two, at least three, at least four, at least five, or at least six of the functional units of the vertical chamber may be the same. In yet other embodiments, the air flow characteristics of at least one, at least two, at least three, at least four, at least five, or at least six of the functional units of the vertical chamber may be different. The air flow characteristics of each of the functional units within the chamber may depend, for example, on the velocity and/or on the angle of the air flow when it enters the functional unit. In some embodiments, a lower slope angle and/or an intake slope angle, as described herein may alter the air flow characteristics of the air flow within one or more functional units of the vertical chamber.

In specific embodiments, the air flow may enter a functional unit of the vertical chamber at an angle of about −20 degrees to about −50 degrees, about −25 degrees to about −45 degrees, about −30 degrees to about −40 degrees, about −20 degrees, about −25 degrees, about −30 degrees, about −31 degrees, about −32 degrees, about −33 degrees, about −34 degrees, about −35 degrees, about −36 degrees, about −37 degrees, about −38 degrees, about −39 degrees, about −40 degrees, about −45 degrees, or about −50 degrees relative to horizontal, including all ranges and values derivable therebetween. In particular embodiments, air enters the first, second, third, fourth, fifth, and/or sixth functional unit at a velocity of about 1.0 m/s to about 25.0 m/s, about 2.5 m/s to about 25.0 m/s, about 3.0 m/s to about 20.0 m/s, about 4.0 m/s to about 15.0 m/s, about 5.0 m/s to about 12.5 m/s, about 5.0 m/s to about 10.0 m/s, about 10.0 m/s to about 25.0 m/s, about 10.0 m/s to about 20.0 m/s, about 10.0 m/s to about 15.0 m/s, about 15.0 m/s to about 25.0 m/s, about 15.0 m/s to about 20.0 m/s, about 1.0 m/s to about 10.0 m/s, about 1.0 m/s to about 5.0 m/s, about 1.0 m/s, about 2.0 m/s, about 2.5 m/s, about 3.0 m/s, about 4.0 m/s, about 5.0 m/s, about 6.0 m/s, about 7.0 m/s, about 8.0 m/s, about 9.0 m/s, about 10.0 m/s, about 11.0 m/s, about 12.0 m/s, about 13.0 m/s, about 14.0 m/s, about 15.0 m/s, about 16.0 m/s, about 17.0 m/s, about 18.0 m/s, about 19.0 m/s, about 20.0 m/s, about 21.0 m/s, about 22.0 m/s, about 23.0 m/s, about 24.0 m/s, or about 25.0 m/s, including all ranges and values derivable therebetween.

In certain embodiments, the methods provided by the present disclosure may further comprise introducing the preparation into the first functional unit from a vibratory feeding unit. The vibratory feeding unit may, in some embodiments, be structurally connected to the vertical chamber, and the vibratory feeding unit may produce a vibratory motion that causes movement of the preparation into the first functional unit. The vibratory motion of the vibratory feeding unit may, in certain embodiments, comprise a substantially horizontal vibratory motion. In certain embodiments, the method comprises introducing the preparation to the first chamber at a rate of about 1 g/min to about 70 g/min, about 10 g/min to about 60 g/min, about 20 g/min to about 50 g/min, about 30 g/min to about 40 g/min, about 1 g/min, about 5 g/min, about 10 g/min, about 15 g/min, about 20 g/min, about 25 g/min, about 30 g/min, about 35 g/min, or about 40 g/min, including all ranges and values derivable therebetween.

In some embodiments, the first, second, third, fourth, fifth, and/or sixth functional unit has an average horizontal cross-sectional area of about 32.258 cm² to about 645.16 cm², about 32.258 cm² to about 516.128 cm², about 32.258 cm² to about 387.096 cm², about 32.258 cm² to about 322.58 cm², about 64.516 cm² to about 645.16 cm², about 64.516 cm² to about 516.128 cm², about 64.516 cm² to about 387.096 cm², about 64.516 cm² to about 322.58 cm², about 96.774 cm² to about 258.064 cm², about 96.774 cm² to about 225.806 cm², about 129.032 cm² to about 193.548 cm², or about 129.032 cm² to about 161.29 cm², including all ranges and values derivable therebetween.

### F. Width and Thickness Separation

Embodiments of the present disclosure may include an apparatus for purifying dry embryo explants, wherein the apparatus comprises a vibratory screen comprising a plurality of openings as described herein. As used herein the term “screen” refers to a generally planar member which comprises a plurality of openings through which some particles of a preparation comprising particles having various lengths, widths, and/or thicknesses may be passed in order to separate them from other particles present in the preparation. In some embodiments, the screen may be generally oriented along a single horizontal, vertical, or diagonal plane. In some embodiments, the screen may be vibrated to produce a screen motion and the screen motion may comprise a horizontal and/or a vertical vibratory component. A screen for use according to the present disclosure may be made from any material which allows for the separation of embryo explants from debris material without causing damage to the embryo explants. Non-limiting examples of which include stainless steel, steel, tin, aluminum, and brass. In some embodiments, each opening of the screen may comprise an opening size and an opening shape. The shape of each of the plurality of openings may, in certain embodiments, be any geometric shape, non-limiting examples of which include a rectangle, a square, a circle, or an oval. In specific embodiments, the opening shape may be circular or oblong. As used herein an opening shape is considered to be “oblong,” when the shape of the opening is elongated. Non-limiting examples of an oblong shape include an oval and a rectangle. The size of a circular screen opening may be, in certain embodiments, about 1.3 mm to about 1.6 mm, about 1.4 mm to about 1.5 mm, about 1.3 mm to about 1.5 mm, or about 1.4 mm to about 1.6 mm in diameter, or about 1.3 mm, about 1.4 mm, about 1.5 mm, or about 1.6 mm in diameter. The size of oblong screen opening may be, in particular embodiments, about 5 mm to about 15 mm, about 6 mm to about 14 mm, about 8 mm to about 12 mm, about 8 mm to about 10 mm, about 9 mm to about 11 mm, about 10 mm to about 12 mm in length, or about 8 mm, about 9 mm, about 10 mm, about 11 mm, or about 12 mm in length, and from about 0.6 mm to about 0.8 mm, about 0.6 mm to about 0.7 mm, or about 0.7 mm to about 0.8 mm in width, or about 0.6 mm, about 0.65 mm, about 0.7 mm, about 0.75 mm, or about 0.8 mm in width. The opening size and the opening shape of the plurality of openings of the vibratory screen may be modified according to the characteristics of the explants to be purified. A vibratory screen comprising a plurality of circular openings may, in specific embodiments, comprise from about 50 to about 200 openings per 6.4516 cm², about 100 to about 200 openings per 6.4516 cm², or about 125 to about 150 openings per 6.4516 cm². A vibratory screen comprising a plurality of oblong openings may, in particular embodiments, comprise about 5 to about 100 openings per 6.4516 cm², about 10 to about 50 openings per 6.4516 cm², or about 15 to about 35 openings per 6.4516 cm².

In particular embodiments, the present disclosure provides an apparatus for purifying dry embryo explants, the apparatus comprising a first and a second vibratory screen. The first vibratory screen and the second vibratory screen may be, in some embodiments, be structurally connected and move in unison. As used herein screens which are “structurally connected” are screens which are in direct or indirect contact with each other. Two or more screens may be considered structurally connected, for example, if they are each in contact with one or more shared structural components of an apparatus. As used herein screens which move in “unison” vibrate simultaneously. In some embodiments, screens which move in unison may also have screen motions comprising approximately the same horizontal and/or vertical vibratory components. The plane of the second vibratory screen may, in some embodiments, be parallel to the plane of the first vibratory screen. The position of the first vibratory screen, in certain embodiments, may be directly above the position of the second vibratory screen. In particular embodiments, the screen motion of the first vibratory screen and the second vibratory screen is the same. In particular embodiments, the screen motion of the first vibratory screen and the second vibratory screen is different.

In further embodiments, the present disclosure provides an apparatus comprising a first and/or a second vibratory screen as described herein; and a motion generator structurally connected to at least one weight and the first and/or second vibratory screen. As used herein components are structurally connected when they are in direct or indirect contact with each other. The motion generator may be considered structurally connected to the at least one weight and the vibratory screen, for example, if each of the motion generator, the at least one weight, and the vibratory screen are in contact with one or more shared structural components of the apparatus. In specific embodiments, the motion generator comprises a motor comprising an axis of rotation, wherein the axis of rotation is perpendicular to the plane of the first and/or second vibratory screen, wherein the motion generator is structurally connected with a first weight and a second weight, and wherein the first weight is positioned above the motion generator and the second weight is positioned below the motion generator.

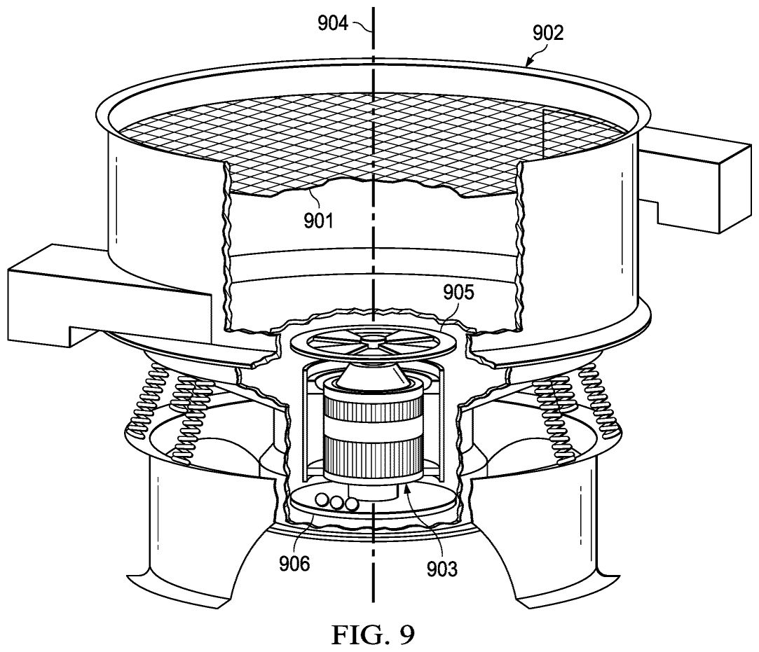

FIG. 9 is a diagram showing an apparatus having vibratory screen 901 to separate a fraction of embryo explants from a portion of the debris material, wherein the vibratory screen is attached to the interior surface of a housing unit 902 configured to provide structural support to the vibratory screen 901; a motion generator 903 attached to the housing unit 902 configured to rotate about an axis of rotation 904, wherein the motion generator is attached to a first wheel 905 and a second wheel 906. In certain embodiments, the first wheel 905 and/or the second wheel 906 may be attached to one or more weights. When one or more weights are attached to the first wheel 905 and the weights are rotated about the axis of rotation 904, the rotation produces a screen motion comprising a horizontal vibratory component. When one or more weights are attached to the second wheel 906, the weights tilt the vibratory screen 901 to create a vertical vibratory component of the screen motion.

In one aspect, the present disclosure provides a method of purifying genetically modifiable dry embryo explants comprising contacting a preparation comprising a population of dry embryo explants and debris material with a first and/or a second vibratory screen, having a screen motion, wherein the screen motion comprises a horizontal and/or a vertical vibratory component. In particular embodiments, the plane of the first and/or second vibratory screen is horizontal relative to the ground and the horizontal vibratory component of the screen motion is in the plane of the first and/or second vibratory screen and changes direction within the plane over time. In certain embodiments, the plane of the first and/or second vibratory screen is horizontal relative to the ground and the vertical vibratory component of the screen motion is perpendicular to the plane of the vibratory screen. In some embodiments, the horizontal vibratory component of the screen motion comprises a displacement amplitude from about 4.0 mm to about 6.0 mm, about 4.5 mm to about 5.75 mm, about 4.7 mm to about 5.6 mm, about 4.7 mm, about 4.75 mm, about 4.8 mm, about 4.85 mm, about 4.9 mm, about 4.95 mm, about 5.0 mm, about 5.05 mm, about 5.1 mm, about 5.15 mm, about 5.2 mm, about 5.25 mm, about 5.3 mm, about 5.35 mm, about 5.4 mm, about 5.45 mm, about 5.5 mm, about 5.55 mm, or about 5.6 mm, including all ranges and values derivable therebetween. In some embodiments, the vertical component of the screen motion comprises a displacement amplitude from about 4.0 mm to about 8.0 mm, about 4.0 mm to about 7.5 mm, about 4.5 mm to about 8.0 mm, about 4.5 mm to about 7.5 mm, about 4.7 mm to about 7.2 mm, or about 4.7 mm, about 4.75 mm, about 4.8 mm, about 4.85 mm, about 4.9 mm, about 4.95 mm, about 5.0 mm, about 5.05 mm, about 5.1 mm, about 5.15 mm, about 5.2 mm, about 5.25 mm, about 5.3 mm, about 5.35 mm, about 5.4 mm, about 5.45 mm, about 5.5 mm, about 5.55 mm, about 5.6 mm, about 5.65 mm, about 5.7 mm, about 5.75 mm, about 5.8 mm, about 5.85 mm, about 5.9 mm, about 5.95 mm, about 6.0 mm, about 6.05 mm, about 6.1 mm, about 6.15 mm, about 6.2 mm, about 6.25 mm, about 6.3 mm, about 6.35 mm, about 6.4 mm, about 6.45 mm, about 6.5 mm, about 6.55 mm, about 6.6 mm, about 6.65 mm, about 6.7 mm, about 6.75 mm, about 6.8 mm, about 6.85 mm, about 6.9 mm, about 6.95 mm, about 7.0 mm, about 7.05 mm, about 7.1 mm, about 7.15 mm, or about 7.2 mm, including all ranges and values derivable therebetween. In specific embodiments, the methods of purifying genetically modifiable dry embryo explants provided herein comprise vibrating the first and/or second vibratory screen at a frequency of about 1 Hz to about 200 Hz, about 2 Hz to about 200 Hz, about 2 Hz to about 175 Hz, about 4 Hz to about 170 Hz, about 5 Hz to about 150 Hz, about 5 Hz to about 125 Hz, about 5 Hz to about 100 Hz, about 5 Hz to about 90 Hz, about 5 Hz to about 80 Hz, about 5 Hz to about 70 Hz, about 5 Hz to about 60 Hz, about 5 Hz to about 50 Hz, about 5 Hz to about 40 Hz, about 5 Hz to about 30 Hz, about 5 Hz to about 25 Hz, about 10 Hz to about 25 Hz, about 5 Hz, about 10 Hz, about 15 Hz, about 20 Hz, about 25 Hz, about 30 Hz, about 35 Hz, about 40 Hz, about 45 Hz, about 50 Hz, about 55 Hz, or about 60 Hz. In some embodiments, the screen motion of the first and/or second vibratory screen may comprise a horizontal vibratory component and a vertical vibratory component and the horizontal and vertical vibratory components may have the same vibration frequency.

In one aspect, the present disclosure provides a method of purifying genetically modifiable dry embryo explants comprising contacting a preparation of dry plant embryo explants with an apparatus provided by the present disclosure comprising a first and/or a second vibratory screen and a motion generator structurally connected to at least one weight and the first and/or second vibratory screen; and vibrating the preparation comprising with the first and/or a second vibratory screen, wherein the vibrating comprises rotating at least one weight about the center of a motion generator. In specific embodiments, the motion generator comprises a motor comprising an axis of rotation, wherein the axis of rotation is perpendicular to the plane of the first and/or second vibratory screen, wherein the motion generator is structurally connected with a first weight and a second weight, and wherein the first weight is positioned above the motion generator and the second weight is positioned below the motion generator. In some embodiments, the lead angle between the first weight and the second weight is from about 0° to about 90°, from about 15° to about 75°, from about 30° to about 60°, from about 40° to about 50°, about 10°, about 15°, about 20°, about 25°, about 30°, about 35°, about 40°, about 45°, about 50°, about 55°, about 60°, about 65°, about 70°, about 75°, about 80°, about 85°, or about 90°. In particular embodiments, the motion generator may be structurally connected with a first, second, third, and fourth weight, wherein the first and third weights are positioned above the motion generator shaft and the second and fourth weights are positioned below the motion generator shaft. Rotation of the first and/or third weights about the axis of the rotation of the motor, in certain embodiments, produces a screen motion comprising a horizontal vibratory component. The horizontal vibratory component of the screen motion may, in some embodiments, cause debris material to move across the vibratory screen to the periphery. The presence of the second and/or fourth weights, positioned below the motion generator shaft, may, in certain embodiments, tilt the vibratory screen to create a vertical vibratory component of the screen motion. The present disclosure provides, in some embodiments, a method of purifying dry embryo explants comprising vibrating a preparation with a vibratory screen as described herein by rotating the at least one weight about the center of a motion generator at about 400 rpm to about 10,000 rpm, about 400 rpm to about 8,000 rpm, about 400 rpm to about 6,000 rpm, about 400 rpm to about 4,000 rpm, about 400 to about 3,600 rpm, about 400 to about 2,500 rpm, about 2,500 rpm to about 10,000 rpm, about 1,000 rpm to about 8,000 rpm, about 1,000 rpm to about 6,000 rpm, about 1,000 rpm to about 4,000 rpm, about 1,000 rpm to about 2,000 rpm, about 500 rpm to about 1,500 rpm, or about 1,160 rpm.

In particular embodiments of the present disclosure, methods are provided herein for separating a first and/or second fraction of embryo explants from debris material. In some embodiments, the first and/or second fraction of embryo explants may be passed through the plurality of openings of the first and/or second vibratory screen, while the first and/or second portion of the debris material is retained on the surface of the first and/or second vibratory screen. In some embodiments, the first and/or second portion of the debris material is passed through the plurality of openings of the first and/or second vibratory screen, while the first and/or second fraction of embryo explants is retained on the surface of the first and/or second vibratory screen. In specific embodiments, the first and/or second vibratory screen is contacted with a preparation comprising a population of dry embryo explants and debris material near the center of the screen.

### G. Separation Using a Friction Table

In another aspect, the present disclosure provides a method of purifying genetically modifiable dry embryo explants, comprising contacting a preparation of dry plant embryo explants comprising meristematic tissue or a fraction thereof with a textured surface of a vibratory platform, wherein the textured surface of the vibratory platform is substantially planar, and wherein the preparation or the fraction thereof comprises a population of dry plant embryo explants and debris material; vibrating the vibratory platform to produce a platform motion; and separating a fraction of the plant embryo explants from a portion of the debris material according to a displacement of the fraction relative to a displacement of the portion of the debris material on the textured surface of the vibratory platform.

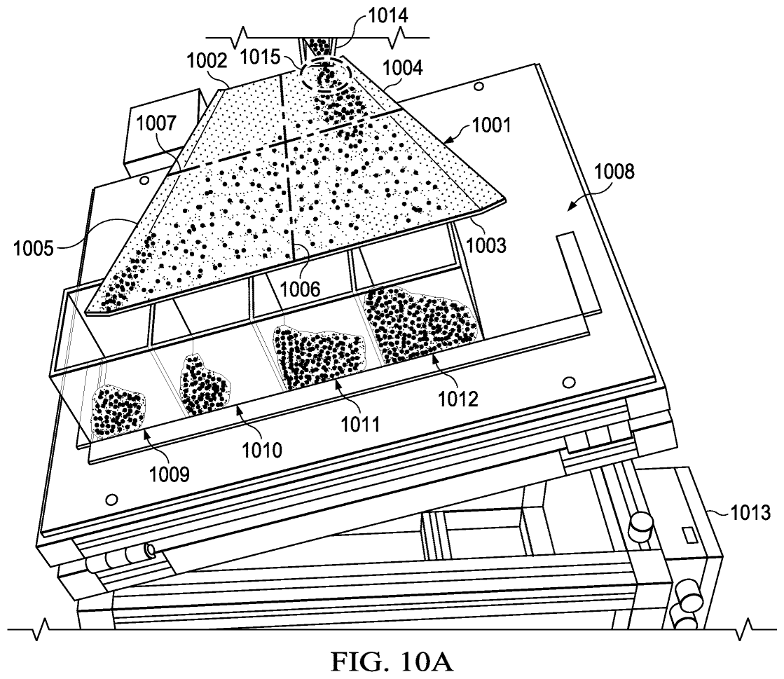

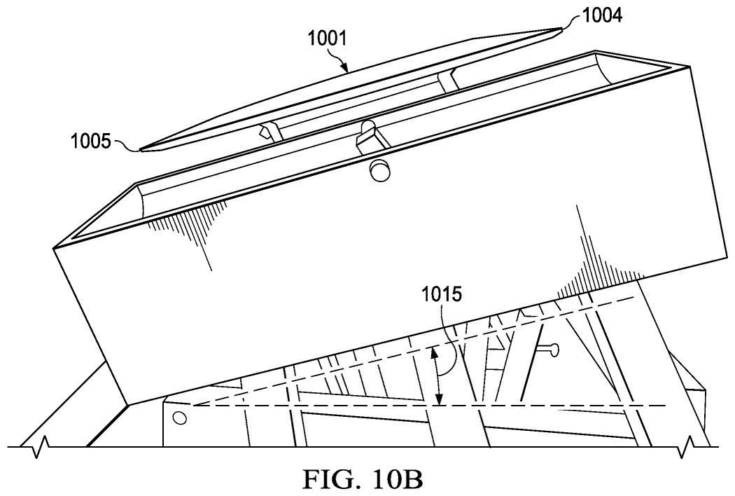

FIG. 10A is a diagram showing an apparatus having vibratory platform 1001, comprising a proximal edge 1002, a distal edge 1003, an upper edge 1004, a lower edge 1005, a pitch axis 1006, and a tilt axis 1007, wherein the top surface of the vibratory platform comprises a textured surface configured to produce an altered displacement of a fraction of embryo explants compared to a portion of the debris material; a base member 1008 attached to a first collection compartment 1009, a second collection compartment 1010, a third collection compartment 1011, and a fourth collection compartment 1012, wherein the first 1009, second 1010, third 1011, and fourth 1006 collection compartments are configured to collect a fraction of embryo explants or a portion of the debris material comprised in a preparation; and an electromagnet 1013 attached to the base member 1008 configured to vibrate the vibratory platform 1001; and a feeding unit 1014 configured to transfer the preparation to the textured surface of the vibratory platform. In some embodiments, the preparation may be first contacted with the vibratory platform at a contact location 1015 at or near the proximal edge 1002 of the vibratory platform 1001. FIG. 10B is a diagram showing an apparatus having a vibratory platform 1001 having an upper edge 1004 and a lower edge 1005, wherein the upper edge 1004 is elevated relative to the lower edge 1005 to produce a tilt angle 1015. FIG. 10C is a diagram having a vibratory platform 1001 having a feeding unit 1014, a proximal edge 1002 and a distal edge 1003, wherein the proximal edge 1002 is elevated relative to the distal edge 1003 to produce a pitch angle 1016.

As used herein the term “platform” refers to a substantially planar member comprising a top surface and a bottom surface, wherein the top surface of the platform comprises a textured surface. The substantially planar shape of the vibratory platform may be any polygonal or non-polygonal shape, non-limiting examples of which include a square, a rectangle, a rhombus, a triangle, a trapezoid, a circle, and an oval. As used herein the term “textured surface” refers to any surface that is not a smooth surface. Textured surfaces may, for example, may be rough or uneven. The textured surface of the vibratory platform may comprise any material which separates a fraction of embryo explants from a portion of the debris material by their relative displacement on the vibratory platform without damaging the embryo explants. Any textured surface known in the art may be used according to the embodiments of the present disclosure, non-limiting examples of which include a sandpaper surface, a vinyl surface, a plasma coated surface, a cork surface, a fabric surface, a rubber surface, and a plastic surface. In particular embodiments, the textured surface may comprise an 80-150 grit sandpaper, or an 80-grit sandpaper, a 90-grit sandpaper, a 100-grit sandpaper, a 110-grit sandpaper, a 120-grit sandpaper, a 130-grit sandpaper, a 140-grit sandpaper, or a 150-grit sandpaper. The textured surface, in specific embodiments, may be structurally adhered to the top surface of a platform as described herein. For example, in some embodiments, a textured surface which is structurally adhered to the top surface of the vibratory platform may become a fixed and integral part of the vibratory platform. In certain embodiments, the textured surface of the platform may be configured to produce an altered displacement of a fraction of embryo explants present in a preparation relative to a displacement of the debris material present in the preparation. The textured surface comprises, in some embodiments, a plurality of granules, each granule having a granule size and a granule shape. The granule shape may be any three-dimensional geometric shape known in the art, non-limiting examples of which include a rectangular prism, a cube, a sphere, or an ovoid. In certain embodiments, a platform provided by the present disclosure may be structurally connected to the plurality of granules. As used herein the term “granule” refers to an element having characteristics, such as size, shape, and texture, configured to produce a textured surface as described herein. A platform, as provided by the present disclosure, may comprise about 50 to about 400, about 50 to about 350, about 50 to about 300, about 50 to about 250, about 60 to about 200, about 80 to about 150, about 200, about 190, about 180, about 170, about 160, about 150, about 140, about 130, about 120, about 110, about 100, about 90, about 80, about 70, about 60, or about 50 granules per 6.4516 cm², including all ranges and values derivable therebetween. The textured surface of the platform may, in some embodiments, comprise granules having an average diameter, width, length, and/or depth of about 25 μm to about 400 μm, about 50 μm to about 300 μm, about 50 μm to about 250 μm, about 50 μm to about 200 μm, about 90 μm to about 190 μm, about 50 μm, about 60 μm, about 70 μm about 80 μm, about 90 μm, about 100 μm, about 110 μm, about 115 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm, including all ranges and values derivable therebetween. The textured surface of the vibratory platform may be modified according to the characteristics of the embryo explants to be purified. In particular embodiments, the preparation or a fraction thereof may comprise corn embryo explants, and the textured surface or may comprise granules having an average diameter, width, length, or depth of about 90 μm to about 190 μm, about 90 μm, about 100 μm, about 110 μm, about 120 μm, about 130 μm, about 140 μm, about 150 μm, about 160 μm, about 170 μm, about 180 μm, or about 190 μm, including all ranges and values derivable therebetween. In some embodiments, the preparation or a fraction thereof may comprise soybean, cotton, or wheat embryo explants and the textured surface may comprise granules having an average diameter, width, length, or depth of about 50 μm to about 250 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, or about 250 μm, including all ranges and values derivable therebetween.

In some embodiments, a vibratory platform of the present disclosure may comprise a proximal edge, a distal edge, an upper edge, and a lower edge. In particular embodiments, the upper edge of the vibratory platform or the lower edge of the vibratory platform is upwardly curled. The methods provided by the present disclosure may, in particular embodiments, comprise initially contacting the vibratory platform at a contact location. The contact location may be, in some embodiments, at or near the proximal edge of the vibratory platform. The contact location may, in certain embodiments, be at or near the lower edge of the platform, near the center point between the proximal edge and the distal edge. The proximal edge of the vibratory platform may, in certain embodiments, be elevated relative to the distal edge. The upper edge of the vibratory platform may, in particular embodiments, be elevated relative to the lower edge of the vibratory platform. In some embodiments, a vibratory platform provided by the present disclosure may comprise a pitch axis and a tilt axis. As used herein a “pitch axis” refers to an axis that intersects the proximal edge and the distal edge of the vibratory platform. In certain embodiments, the pitch axis intersects the proximal edge and the distal edge of the vibratory platform through a center point. As used herein the term “center point” refers to the geometric center of the area of a vibratory platform. As used herein a “tilt axis” refers to an axis which is perpendicular to the pitch axis. The tilt axis, for example, may refer to the axis that intersects the upper edge and the lower edge of the vibratory platform. In certain embodiments, the tilt axis intersects the upper edge and the lower edge of the vibratory platform through the center point. In some embodiments, the vibratory platform may be positioned at a compound angle relative to the ground, wherein the compound angle comprises a pitch angle and a tilt angle, wherein the pitch angle is along the pitch axis, and wherein the tilt angle is along the tilt axis. In certain embodiments, the vibratory platform may be positioned at a tilt angle of about 8.0 degrees to about 25.0 degrees, about 9.0 degrees to about 25.0 degrees, about 10.0 degrees to about 25.0 degrees, about 8.0 degrees to about 22.0 degrees, about 10.0 degrees to about 20.0 degrees, about 11.0 degrees to about 19.0 degrees, about 12.7 degrees to about 14.7 degrees, about 15.8 degrees to about 16.6 degrees, about 11.6 degrees to about 12.0 degrees, about 16.2 degrees to about 18.3 degrees, about 11.6 degrees to about 14.2 degrees, about 12.5 degrees to about 17.3 degrees, about 10.9 degrees to about 14.9 degrees, about 17.5 degrees to about 21.5 degrees, about 8.0 degrees, about 8.5 degrees, about 9.0 degrees, about 9.5 degrees, about 10.0 degrees, about 10.5 degrees, about 11.0 degrees, about 11.5 degrees, about 11.8 degrees, about 12.0 degrees, about 12.5 degrees, about 12.9 degrees, about 13.0 degrees, about 13.5 degrees, about 13.7 degrees, about 14.0 degrees, about 14.5 degrees, about 15.0 degrees, about 15.5 degrees, about 16.0 degrees, about 16.5 degrees, about 17.0 degrees, about 17.2 degrees, about 17.5 degrees, about 18.0 degrees, about 18.5 degrees, about 19.0 degrees, about 19.5 degrees, about 20.0 degrees, about 20.5 degrees, about 21.0 degrees, about 21.5 degrees, about 22.0 degrees, about 22.5 degrees, about 23.0 degrees, about 23.5 degrees, about 24.0 degrees, about 24.5 degrees, or about 25.0 degrees, including all ranges and values derivable therebetween. The vibratory platform may be positioned, in some embodiments, at a pitch angle of about 1.0 degrees to about 10.0 degrees, about 1.4 degrees to about 9.0 degrees, about 1.5 degrees to about 8.0 degrees, about 2.1 degrees to about 2.6 degrees, about 4.3 degrees to about 7.5 degrees, about 1.9 degrees to about 3.25 degrees, about 2.4 degrees to about 4.9 degrees, about 1.8 degrees to about 3.25 degrees, about 2.0 degrees to about 6.0 degrees, about 1.0 degrees to about 4.2 degrees, about 1.5 degrees to about 4.5 degrees, about 1.0 degrees, about 1.5 degrees, about 2.0 degrees, about 2.3 degrees, about 2.5 degrees, about 2.6 degrees, about 3.0 degrees, about 3.5 degrees, about 3.6 degrees, about 4.0 degrees, about 4.5 degrees, about 5.0 degrees, about 5.5 degrees, about 5.9 degrees, about 6.0 degrees, about 6.5 degrees, about 7.0 degrees, about 7.5 degrees about 8.0 degrees, about 8.5 degrees, about 9.0 degrees, about 9.5 degrees, or about 10.0 degrees, including all ranges and values derivable therebetween. The tilt angle and the pitch angle of the vibratory platform may be modified according to the characteristics of the embryo explants to be purified.

In further embodiments, the population may comprise corn embryo explants and the tilt angle may be about 10.0 degrees to about 20.0 degrees, about 10.0 degrees to about 17.0 degrees, about 12.5 degrees to about 15.0 degrees, about 12.7 degrees to about 14.7 degrees or about 13.7 degrees, including all ranges and values derivable therebetween, or the pitch angle may be about 1.5 degrees to about 3.5 degrees, about 2.0 degrees to about 3.0 degrees, about 2.1 degrees to about 2.6 degrees, about 2.3 degrees, or about 2.4 degrees, including all ranges and values derivable therebetween. In particular embodiments, the population may comprise soybean embryo explants and the tilt angle may be about 10.0 degrees to about 20.0 degrees, about 10.0 degrees to about 18.0 degrees, about 14.0 degrees to about 20.0 degrees, about 11.0 degrees to about 17.0 degrees, about 11.6 degrees to about 16.6 degrees, about 11.6 degrees to about 12.0 degrees, about 15.8 degrees to about 16.6 degrees, about 11.8 degrees, or about 16.2 degrees, including all ranges and values derivable therebetween, or the pitch angle may be about 1.5 degrees to about 8.0 degrees, about 1.9 degrees to about 7.5 degrees, about 1.9 degrees to about 3.3 degrees, about 4.3 degrees to about 7.5 degrees, about 2.5 degrees, about 2.6 degrees, or about 5.9 degrees, including all ranges and values derivable therebetween. In certain embodiments, the population may comprise cotton embryo explants and the tilt angle may be about 10.0 degrees to about 22.0 degrees, about 10.0 degrees to about 20.0 degrees, about 10.0 degrees to about 19.0 degrees, about 15.0 degrees to about 22.0 degrees, about 11.0 degrees to about 19.0 degrees, about 11.0 degrees to about 15.0 degrees, about 11.6 degrees to about 14.2 degrees, about 16.0 degrees to about 19.0 degrees, about 16.2 degrees to about 18.3 degrees, about 12.9 degrees, about 17.2 degrees, or about 17.3 degrees, including all ranges and values derivable therebetween, or the pitch angle may be about 1.5 degrees to about 6.0 degrees, about 1.5 degrees to about 5.0 degrees, about 1.8 degrees to about 4.9 degrees, about 1.8 degrees to about 3.3 degrees, about 2.4 degrees to about 4.9 degrees, about 2.5 degrees, about 2.6 degrees, about 3.6 degrees, or about 3.7 degrees, including all ranges and values derivable therebetween. In some embodiments, the population may comprise wheat embryo explants and the tilt angle may be about 10.0 degrees to about 20.0 degrees, about 10.0 degrees to about 19.0 degrees, about 12.0 degrees to about 17.0 degrees, about 13.0 degrees to about 16.0 degrees, about 14.0 degrees to about 15.0 degrees, or about 14.5 degrees, including all ranges and values derivable therebetween, or the pitch angle may be about 1.5 degrees to about 8.0 degrees, about 2.0 degrees to about 6.0 degrees, about 3.0 degrees to about 5.0 degrees, about 3.5 degrees to about 4.5 degrees, or about 4.0 degrees, including all ranges and values derivable therebetween. In certain embodiments, the population may comprise wheat embryo explants and the tilt angle may be about 10.0 degrees to about 16.0 degrees, about 10.0 degrees to about 15.0 degrees, about 11.0 degrees to about 15.0 degrees, about 12.0 degrees to about 14.0 degrees, about 12.5 degrees to about 13.5 degrees, about 12.7 degrees to about 13.1 degrees, or about 12.9 degrees, including all ranges and values derivable therebetween, or the pitch angle may be about 1.5 degrees to about 5.0 degrees, about 1.0 degrees to about 4.0 degrees, about 1.0 degrees to about 3.0 degrees, about 1.5 degrees to about 3.0 degrees, about 1.8 degrees to about 2.6 degrees, or about 2.2 degrees, including all ranges and values derivable therebetween.

In particular embodiments, the vibratory platform as described herein comprises a pitch dimension and a tilt dimension. As used herein a “pitch dimension” refers to a dimension measured from the proximal edge to the distal edge through a center point of the vibratory platform and along or parallel to the pitch axis. As used herein the term “tilt dimension” refers to a dimension measured from the upper edge to the lower edge through the center point of the vibratory platform and along or parallel to the tilt axis. In certain embodiments, the pitch dimension of a vibratory platform as described herein may be about 12.7 cm to about 127 cm, about 12.7 cm to about 76.2 cm, about 12.7 cm to about 50.8 cm, about 12.7 cm to about 38.1 cm, about 12.7 cm to about 25.4 cm, or about 25.4 cm to about 38.1 cm, including all ranges and values derivable therebetween. In some embodiments, the tilt dimension of a vibratory platform as described herein, may be about 12.7 cm to about 127 cm, about 12.7 cm to about 76.2 cm, about 12.7 cm to about 63.5 cm, about 12.7 cm to about 50.8 cm, about 25.4 cm to about 50.8 cm, or about 25.4 cm to about 38.1 cm, including all ranges and values derivable therebetween. In some embodiments, the pitch dimension:tilt dimension ratio of the vibratory platform may be about 1:1, less than about 1:1, or greater than about 1:1. A distance measured, in certain embodiments, from the upper edge to the lower edge of a vibratory platform at or near the proximal edge of the vibratory platform may be less than a distance measured from the upper edge to the lower edge of the vibratory platform at or near the distal edge of the vibratory platform. In some embodiments, a distance measured from the upper edge to the lower edge of a vibratory platform at or near the proximal edge of the vibratory platform is greater than a distance measured from the upper edge to the lower edge of the vibratory platform at or near the distal edge of the vibratory platform.

In certain embodiments, the displacement of a fraction of plant embryo explants comprises a fraction displacement range, wherein the fraction displacement range comprises a fraction pitch distance component and a fraction tilt distance component. As used herein the term “fraction displacement range” refers to the movement of the fraction of embryo explants on or over the surface a vibratory platform, as described herein, from the platform contact location during a displacement time. As used herein the term “fraction pitch distance component” refers to a movement along or parallel to a pitch axis, as described herein. For example, in some embodiments, the pitch fraction distance component may refer to the range of movement of a fraction of embryo explants in a pitch direction along or parallel to the pitch axis from a platform contact location and toward a distal edge of a vibratory platform during a displacement time. As used herein the term “fraction tilt distance component” refers to a movement along or parallel to a tilt axis, as described herein. For example, in certain embodiments, the fraction tilt distance component is the range of movement of a fraction of embryo explants in a tilt direction along or parallel to a tilt axis from a platform contact location and toward the lower edge of a vibratory platform during a displacement time.

In some embodiments, the displacement of the portion of the debris material comprises a portion displacement range, wherein the portion displacement range comprises a portion pitch distance component and a portion tilt distance component. As used herein the term “portion displacement range” refers to a movement of a portion of the debris material on or over a textured surface of a vibratory platform, as described herein, from the platform contact location during a displacement time. As used herein the term “portion pitch distance component” refers to a movement along or parallel to a pitch axis, as described herein. For example, in some embodiments, the portion pitch distance component may be the range of movement of a portion of the debris material in a pitch direction from the platform contact location and toward a distal edge of a vibratory platform during a displacement time. As used herein the term “portion tilt distance component” refers to a movement along or parallel to a tilt axis, as described herein. For example, in certain embodiments, the portion tilt distance component may be the range of movement of a portion of the debris material in a tilt direction from the platform contact location and toward a lower edge of a vibratory platform during a displacement time.

In particular embodiments, the portion pitch distance component may be less than the fraction pitch distance component, or the portion pitch distance component may be greater than the fraction pitch distance component. In certain embodiments, the portion tilt distance component may be less than the fraction tilt distance component, or the portion tilt distance component may be greater than the fraction tilt distance component.

In specific embodiments, the present disclosure provides a method comprising vibrating the vibratory platform at a frequency of about 1 Hz to about 500 Hz, about 10 Hz to about 400 Hz, about 20 Hz to about 300 Hz, about 30 Hz to about 250 Hz, about 40 Hz to about 200 Hz, about 50 Hz to about 150 Hz, about 55 Hz to about 125 Hz, about 60 Hz to about 120 Hz, about 50 Hz, about 60 Hz, about 70 Hz, about 80 Hz, about 90 Hz, about 100 Hz, about 110 Hz, about 120 Hz, or about 130 Hz. In certain embodiments, the present disclosure provides a method comprising contacting the preparation or a fraction thereof with the platform at a rate of about 0.5 g/min to about 10.0 g/min, about 1.0 g/min to about 9.0 g/min, about 1.0 g/min to about 8.0 g/min, about 1.0 g/min to about 7.0 g/min, about 1.0 g/min to about 6.0 g/min, about 1.0 g/min to about 5.0 g/min, about 1.0 g/min to about 4.0 g/min, about 2.0 g/min to about 4.0 g/min, about 0.5 g/min, about 1.0 g/min, about 2.0 g/min, about 3.0 g/min, about 4.0 g/min, about 5.0 g/min, about 6.0 g/min, about 7.0 g/min, about 8.0 g/min, about 9.0 g/min, or about 10.0 g/min, including all ranges and values derivable therebetween. In certain embodiments, a platform motion as described herein may comprise a substantially horizontal vibratory component. The platform motion, in certain embodiments, may be a linear motion, a circular motion, or an elliptical motion. In particular embodiments, the platform motion may be along the tilt axis, along the pitch axis, or may be at an angle of about 5 degrees to about 85 degrees, about 10 degrees to about 80 degrees, about 15 degrees to about 15 degrees to about 75 degrees, about 20 degrees to about 70 degrees, about 25 degrees to about 65 degrees, about 30 degrees to about 60 degrees, about 35 degrees to about 55 degrees, about 40 degrees to about 50 degrees, about 5 degrees to about 45 degrees, about 10 degrees to about 40 degrees, about 15 degrees to about 35 degrees, about 20 degrees to about 30 degrees, about 45 degrees to about 85 degrees, about 50 degrees to about 80 degrees, about 55 degrees to about 75 degrees, about 60 degrees to about 70 degrees, about 5 degrees to about 15 degrees, about 15 degrees to about 25 degrees, about 25 degrees to about 35 degrees, about 35 degrees to about 45 degrees, about 45 degrees to about 55 degrees, about 55 degrees to about 65 degrees, about 65 degrees to about 75 degrees, or about 75 degrees to about 85 degrees relative to the pitch axis or relative to the tilt axis, including all ranges and values derivable therebetween. In particular embodiments, a platform motion as described herein may have a vibrational amplitude of greater than 0 mm and less than 2.0 mm. The platform motion may have, for example, a vibrational amplitude of about 0.05 mm to about 2.0 mm, about 0.05 mm to about 1.9 mm, about 0.05 to about 1.8 mm, about 0.05 mm to about 1.7 mm, about 0.05 mm to about 1.6 mm, about 0.05 mm to about 1.5 mm, about 0.05 mm to about 1.4 mm, about 0.05 mm to about 1.3 mm, about 0.05 mm to about 1.2 mm, about 0.05 mm to about 1.1 mm, about 0.05 mm to about 1.0 mm, about 0.05 mm to about 0.9 mm, about 0.05 mm to about 0.8 mm, about 0.05 mm to about 0.7 mm, about 0.05 mm to about 0.6 mm, about 0.05 mm to about 0.5 mm, about 0.05 mm to about 0.4 mm, about 0.05 mm to about 0.3 mm, about 0.05 mm to about 0.2 mm, about 0.1 mm to about 0.5 mm, about 0.05 mm, about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm, including all ranges and values derivable therebetween.

In some embodiments, the present disclosure provides a method further comprising collecting a fraction of embryo explants. The fraction of embryo explants may, in some embodiments, be collected at or near the distal end of the platform, at or near the proximal end of the platform, or at or near a center portion of the platform. In certain embodiments, collecting a fraction of plant embryo explants may comprise collecting the fraction in a fraction collector. The fraction of plant embryo explants may, in particular embodiments, fall into the fraction collector from a fraction distal location on the distal edge of the vibratory platform. In certain embodiments, the methods provided by the present disclosure may further comprise collecting a portion of the debris material. The portion of the debris material may, in some embodiments, be collected in a portion collector. The portion of the debris material may, in particular embodiments, fall into the portion collector from a portion distal location on the distal edge of the vibratory platform. In some embodiments, the fraction distal location is positioned closer to the lower edge of the vibratory platform than is the portion distal location. In particular embodiments, the fraction distal location is positioned closer to the platform contact location of the vibratory platform than is the portion distal location. The fraction distal location may, in some embodiments, be positioned closer to the upper edge of the vibratory platform than is the portion distal location. The portion distal location, in particular embodiments, may be positioned closer to the lower edge of the vibratory platform than is the fraction distal location. In certain embodiments, the portion distal location is positioned closer to the platform contact location of the vibratory platform than is the fraction distal location. In certain embodiments, the portion distal location is positioned closer to the upper edge of the vibratory platform than is the fraction distal location.

The present disclosure further provides, in certain embodiments, methods of purifying genetically modified embryo explants which may comprise contacting a first fraction of plant embryo explants with a second textured surface of a second vibratory platform, wherein the second textured surface of the second vibratory platform is substantially planar; vibrating the second vibratory platform to produce a second platform motion; and separating a second fraction of the plant embryo explants of the first fraction from a second portion of the debris material according to a displacement of the second fraction relative to a displacement of the second portion of the debris material on the second textured surface of the second vibratory platform. In particular embodiments, the second vibratory platform may comprise a different textured surface compared to the first vibratory platform. In some embodiments, the second vibratory platform may comprise the same textured surface as the first vibratory platform. In specific embodiments, the second vibratory platform may be the same platform as the first vibratory platform, but the first fraction may be subjected to a second run on the platform. Any embodiments described herein relating to a vibratory platform or methods of purifying genetically modifiable embryo explants comprising contacting plant embryo explants with a textured surface of a vibratory platform relate to a first and/or a second vibratory platform and the use thereof.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description.

### Example 1

### Preparation of Corn Dried Excised Explants

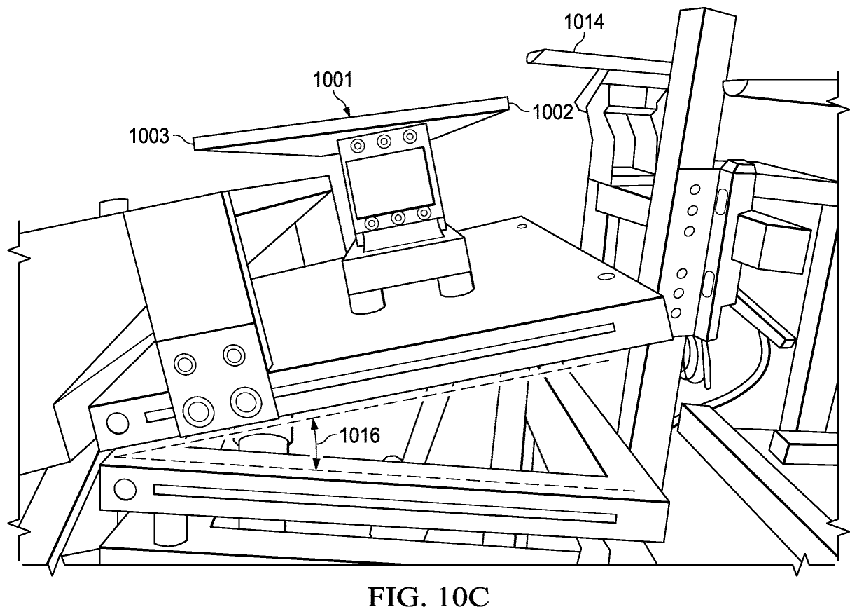

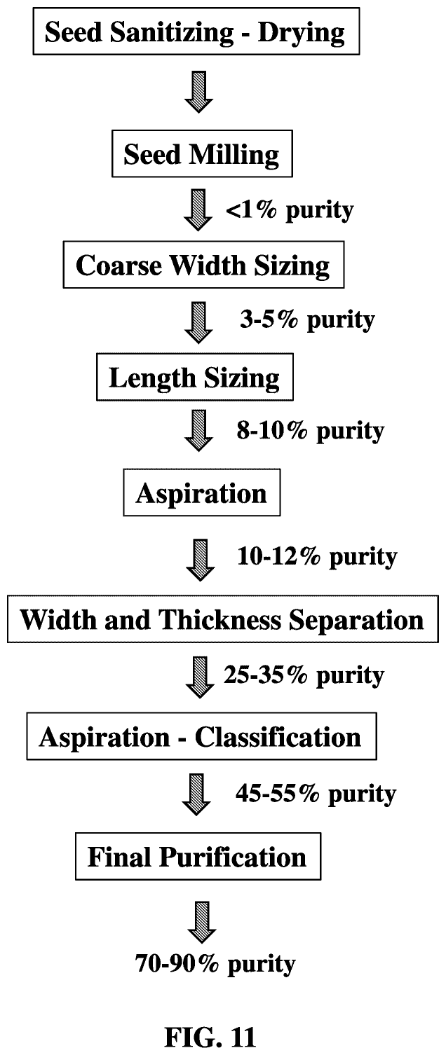

This example describes a process by which mature corn embryo explants are excised from dry seeds and purified using a series of steps involving the use of multiple devices and methods to achieve production of a high purity explant preparation for transformation. FIG. 11 illustrates the mature corn embryo explant preparation and purification workflow that will be described further in this example. All the steps described below may be performed under clean room and ionization conditions with air filtered through HEPA filters to reduce contamination and static charges.

Mature corn seeds were harvested, dried to a desired moisture content of about 8% to about 12% and stored in containers or bags prior to explant preparation. Corn seeds were transferred to a seed sanitizer and dryer to prepare the seeds for milling. The sanitization and drying system comprises: (1) an IPEC solution mixing skid; (2) a Munters® dryer; and (3) a Spray Dyanics® coating drum. The IPEC solution skid is custom equipment designed to mix and prepare a solution of Reverse Osmosis De-ionized (RODI) water, bleach, and TWEEN®, which is used to sanitize the seed. For corn seeds, the solution comprises RODI, 5,500 PPM bleach, and 0.02% TWEEN® for a total volume of about 340 gallons. Following sanitization, the seeds were dried (corresponding to Seed Sanitizing-Drying step in FIG. 11) using the Munters® desiccant dryer (model HCD-2250-DGA-SCBMPCS, Amesbury, MA 01913, USA), which is a stand-alone dehumidifier. For the preparation of mature corn embryo explants, the desiccant dryer was fitted with a cooling coil, reheat section, and HEPA filters. The seeds were dried with the aid of the Munters® desiccant dryer while rotating in a Spray Dyanics® coating drum. The Spray Dyanics® coating drum (Union, MO 63084, USA) is often used in food preparation to apply seasoning and other flavorings to food. The coating drum was configured to operate using a batch process instead of a continuous process. Dry air was supplied by the Munters® dryer and the seeds were processed for about 1 to about 2 days. When a relative humidity (RH) over the set RH threshold is detected, the desiccant wheel powers on and forces dry air over the seeds. The parameters for the Air Flow and Drying Steps are provided in Table 1 below.

TABLE 1
Air Flow and Drying Step parameters.
High Air Flow Drying

| Duration | 30 | min |
|---|---|---|
| RH % Desiccant Wheel On | 50% | |
| Air Flow | 4,000 SCFM (Standard Cubic Feet per | |
| | Minute) | |
| Process Air Temp | 85° | F. |

Drying Step 1

| RH % Desiccant Wheel On | 50% | |
|---|---|---|
| Air Flow | 2,200 | SCFM |
| Process Air Temp | 85° | F. |
| Change Relative Humidity Max | 75% | |
| Change Temp Minimum | 72° | F. |

Drying Step 2

| RH % Desiccant Wheel On | 1% | |
|---|---|---|
| Air Flow | 2,200 | SCFM |
| Process Air Temp | 100° | F. |
| Change Relative Humidity Max | 15% | |
| Change Temp Minimum | 86° | F. |

Drying Step 3

| RH % Desiccant Wheel On | 1% | |
|---|---|---|
| Air Flow | 2,200 | SCFM |
| Process Air Temp | 103° | F. |
| Change Relative Humidity Max | 15% | |
| Change Temp Minimum | 86° | F. |

| | |
|---|---|
| Target Moisture | 8%-12% |

The sanitized and dried corn seeds were transferred to a roller mill (Model IMD 79, Modern Process Equipment, Chicago, IL 60623, USA; corresponding to Seed Milling step in FIG. 11), where the seed embryos comprising meristematic tissue were released from the remainder of the seed during the milling process. The IMD 79 roller mill was designed and fitted with two sets of grinding rollers. See, for example, FIG. 1 (A-C). In this example, the grinding rollers measured about 15.24 cm in diameter and about 45.72 cm in length and were made of stainless steel. Grinding rollers made of other materials including, but not limited to, ceramic and steel may also be used. In some embodiments, the grinding rollers may be about 7.62 cm to about 50.8 cm in diameter and about 5.08 cm to about 121.92 cm in length. The rollers may have teeth, which can be described by their roll cut identification, such as 8AS or 20ST. In a roll cut identification, the number indicates the number of teeth per 2.54 cm of the two rollers at the point of near contact and the letters indicate the shape of the teeth. For example, 8AS would equal 8 teeth per 2.54 cm of the two rollers at the point of near contact, or 4 teeth per 2.54 cm on roller 1 and 4 teeth per 2.54 cm on roller 2. With regard to the shape of the teeth, AS indicates Alice Sharp teeth, which have a scalene shape, and ST indicates Saw Tooth teeth, which have an isosceles or equilateral triangle shape. In this example, the roll cut identification of the roller teeth was 8AS. The roll cut identification may be adjusted depending on the characteristics of the seed to be milled. In certain embodiments, the roll cut identification may be, for example, AS, multiple AS, ST, flat, or other shape with about 4 to about 20 teeth per 2.54 cm. In this example, the rotation rate ratio was set to 1.1:1, the rate of rotation was set between about 194 rpm and about 213 rpm, and the roll cut orientation was set to sharp to dull (FIG. 2B). The rotation rate ratio, rate of rotation, and roll cut orientation may also be adjusted depending on the characteristics of the seed to be milled. The rotation rate ratio may be, for example, about 1:1 to about 4:1, the rate of rotation may be, for example, about 50 rpm to about 1200 rpm, and the roll cut orientation may be set to, for example, sharp to dull, sharp to sharp, dull to sharp, or dull to dull. For mature corn embryo explant preparations, the gap for the top rollers was set to about 2.54 mm and the gap for the bottom rollers was set to about 1.27 mm. The roll gaps may be adjusted, depending on the size of the seed, to maximize seed milling and minimize explant damage. The roll gap may be adjusted to, for example, about 0.381 mm to about 7.62 mm for corn seeds. The seeds were fed into the roller mill at a continuous speed such that no material accumulated on top of the rollers and subsequently milled. Following milling, the embryo explant purity in this experiment was less than about 1% as a percentage of total particles.

The population of milled dry seeds and milled dry seed explants was then transferred to a Rotex® Siever (model A8G12L-3S, Cincinnati, OH 45223 USA; corresponding to Coarse Width Sizing step in FIG. 11) to separate the explants from larger and smaller debris particles. The Rotex® Siever uses a gyratory-reciprocating motion, which gradually changes along the length of the machine from a circular motion to an elliptical motion to an approximate straight-line motion at the discharge end. See, for example, FIG. 3. The circular motion at the feed end spreads the material across the full width of the sieve surface, allowing stratification of the material and conveying the material forward. The elliptical motion at the middle of the Rotex® Siever enhances the stratification of the material, and the straight-line motion at the discharge end provides a linear sifting motion, which removes near-size particles and improves screening efficiency. In this example, all stainless-steel wire mesh sieves were used. The top sieve had openings of about 1181 μm with three 3.175 cm rubber balls per pocket under the sieve. The bottom sieve had openings of about 812 μm with three 3.175 cm silicone balls per pocket under the sieve. The opening size and/or type of the top sieve and bottom sieve may be adjusted according to the characteristics of the embryo explant of the variety used. For example, the top sieve may have openings of about 800 μm to about 2000 μm with about one to about ten 3.175 cm rubber balls per pocket under the sieve. The bottom sieve may, in some embodiments, have openings of about 500 μm to about 1000 μm with about one to about ten 3.175 cm silicone balls per pocket under the sieve. The milled explants were fed into the Rotex® Siever at 54% VFD (˜65 V AC), which corresponds to a speed of about 2900 g/min. The exit end of the Rotex® Siever is connected through an aspiration connection to a dust collector. The aspiration connection has a total of three guillotine-style, damper blast gates. In this example, two of the blast gates, the top blast gate (nearest to the dust collector) and bottom gate (nearest to the explant material), were set in-line, parallel to the air flow, from the dry embryo explant material to the dust collector. The middle blast gate was set at 90 degrees to the air flow to supply relief air and to reduce the suction that the dry embryo explant material experiences. The aspiration may be increased or decreased by opening or closing the blast gates. For corn explant preparation, the top blast gate was fully open, and the bottom and middle blast gates were halfway open. The embryo explants in this example were retained on the bottom sieve. Following this step, the embryo explant purity was about 3% to about 5% as a percentage of total particles. This represents about a 3.0-fold to about a 5.0-fold increase in explant purity compared to the purity of the embryo explants following Seed Milling.

The corn embryo explant preparation was then transferred to an LA-T Laboratory Indent Cylinder Separator (Seedburro® Equipment Company, Des Plaines, IL 60018, USA) (corresponding to Length Sizing step in FIG. 1). See, for example, FIG. 5. The Indent Cylinder was configured to rotate at about 37 rpm to about 38 rpm. The indentation size of the cylinder may vary depending on the size of the embryo explant. For example, an indentation size of about 2.25 mm may be used for large flat corn explants and an indentation size of about 2.00 mm may be used for small flat corn explants. In certain embodiments, an indentation size of about 1.5 mm to about 3.0 mm may be used. The embryo explant preparation was initially fed into the Indent Cylinder at a rate of about 2000 g/min to load the cylinder. Once the cylinder was loaded the embryo explant preparation was fed into the Indent Cylinder at a rate of about 1000 to about 1300 g/min to balance the inflow and outflow of material. In this example, the radius of the Indent Cylinder was about 20.32 cm. The debris collector setting, and the position of the end gate determine how thick the material bed will become. In this example, the debris collector was raised about 5 cm above the cylinder floor and the highest depth of the retention arm was about 2.5 cm. Small debris material was removed in this step and following this step, the embryo explant purity was about 8% to about 10% as a percentage of total particles. This represents about a 1.6-fold to about a 3.3-fold increase to explant purity compared to the purity of the embryo explants following Coarse Width Sizing and about an 8-fold to about a 10-fold increase in explant purity compared to the purity of the embryo explants following Seed Milling.

Following processing in the Indent Cylinder, the embryo explant preparation in this example was transferred to an STS-MACS (Multiple Air Chamber System) Seed Separator (Model STS-MACS 104, SeedTech Systems, Elk Grove CA 95758-4151, USA, corresponding to Aspiration step in FIG. 11). The STS-MACS 104 is a multiple air chamber system which stratifies columns of air and uses this air to separate the dry embryo explants from debris. See, for example, FIG. 6. A vibratory feeder unit was used to feed the explant preparation into the first of four enclosed chambers at a rate of about 35 g/min. Air flow is calibrated by digitized variable frequency computers, which are controlled by the user. Each of the four successive chambers has increasing air flow and lift. The indicated VFD (Variable Frequency Drive) setting ranges for each chamber were as follows: Chamber #1=18.6-19.1 Hz (5.1 m/s-5.3 m/s); Chamber #2=19.3-20.0 Hz (5.9 m/s-6.1 m/s); Chamber #3=18.0-18.7 Hz (7.1 m/s-7.3 m/s); and Chamber #4=18.1-18.7 Hz (9.7 m/s-10.1 m/s). The enriched explant preparation exits the system through a collection chute while the separated debris exits the system through one of the discharge ports located in each chamber. Following this step, the embryo explant purity was about 10% to about 12% as a percentage of total particles. This represents about a 1.2-fold increase in explant purity compared to the purity of the embryo explants following Length Sizing and about a 10-fold to about a 12-fold increase in explant purity compared to the purity of the embryo explants following Seed Milling.

The enriched explant preparation was transferred from the STS-MACS to a SWECO® Vibro-Energy® Separator (Model numbers ZS24S686CBINP3P4SASDTLWS or ZS24S444CBP3SD, SWECO®, Florence, KY, USA, corresponding to Width and Thickness Separation step in FIG. 11) for further purification. See, for example, FIG. 9. The SWECO® separator is a screening device that vibrates about its center of mass. Vibration is accomplished by eccentric weights on the upper and lower ends of a motion-generator shaft. Rotation of the top weights create a vibration in the horizontal plane, which causes material to move across a screen to the periphery. The lower weights tilt the machine, causing vibration in the vertical and tangential planes. The frequency of vibrations can be set by the rpm of the motor and may, in some embodiments, be about 2 Hz to about 20 Hz, including all ranges and values derivable therebetween. To purify the corn embryo explant preparation, the motor was set to 1160 rpm. The upper and lower weights were adjusted away from 180 degrees to create imbalance. In this example, the upper weights were set in either the 4 and 4 positions or the 5 and 5 positions, and the lower weight lead angle was set to a range between 35 degrees to 45 degrees. This was achieved by placing the lower weights in the 0 and 70 positions or the 0 and 90 positions, respectively. The combination of these weight positions resulted in an indicated horizontal amplitude of about 4.7625 mm to about 5.5563 mm and an indicated vertical amplitude of about 4.7625 mm to about 7.1438 mm. The vibrated screen was made of stainless steel and comprised slotted holes measuring about 10 mm in length by about 0.6 to about 0.8 mm in width or round holes measuring about 1.3 to about 1.6 mm in diameter. The motion of the SWECO® separator causes the corn explants to land on their ends or side planes, which allows the explants to fall through the slots and be discharged via the undersize discharge spout. The waste fraction vibrates to the edge of the screen and is discharged via the oversize discharge spout. Discharge spouts on the upper and lower spacing frames were offset by 180 degrees. Explants were subject to two rounds of sieving in the SWECO® separator. In the first round, sieving was performed using a round hole screen with holes measuring about 1.3 mm to about 1.4 mm in diameter to achieve a width separation. In the second round, sieving was performed using a slotted hole screen with holes measuring about 0.6 mm to about 0.8 mm in width and about 10 mm in length to achieve a thickness separation. After two rounds of sieving in the SWECO® separator, the corn embryo explant purity in this experiment was about 25% to about 35% as a percentage of total particles. This represents about a 2-fold to about a 3.5-fold increase in explant purity compared to the purity of the embryo explants following Aspiration and about a 25-fold to about a 35-fold increase in explant purity compared to the purity of the embryo explants following Seed Milling.

The embryo explant preparation from the SWECO® separator was then passed through the STS-MACS seed processor a second time (corresponding to the Aspiration-Classification step in FIG. 11) using the same settings as provided above resulting in a purity of about 45% to about 55% as a percentage of total particles. See, for example, FIG. 6. This represents about a 1.3-fold to about a 2.2-fold increase in explant purity compared to the purity of the embryo explants following Width and Thickness Separation and about a 45-fold to about a 55-fold increase in explant purity compared to the purity of the embryo explants following Seed Milling. In some examples, the Air-Classification step may be performed using a Hoffman® HMC 67 blower (Model number 67-LKBLOWER, Hoffman®, Corvallis, OR, USA). The Hoffman® blower is a precision-built laboratory machine designed to separate light and heavy fractions for a large variety of seeds. See, for example, FIG. 7. The Hoffman® blower consists of a vertical aspiration chamber, the top end of which is in fluid communication with a turned segment and a waste collector. The turned segment is configured to transfer the debris material from the chamber to the waste collector and arcs at an angle of about 180 degrees before emptying into the mesh waste collector. The air flow is regulated using a calibrated vernier gate which may be precisely adjusted using a calibrated hand wheel. The embryo explant preparation is placed in the chamber and exposed to a vertical air velocity of about 2.0 m/s to about 10.0 m/s. At this air velocity, explants remain in the chamber while debris is blown upwards through the 180-degree arc into a mesh waste collector.

For the final purification step (corresponding to the Final Purification step in FIG. 11), the explants were further separated from remaining debris using a Friction Table. The Friction Table is a modified Oscillating Shape Sorter (Model OSCA-1000, Profile Industries, Rogers, MN 55374, USA) in which the surface of the vibratory platform is modified to have a textured surface and the base member of the Oscillating Shape Sorter is attached to a collection trough to collect the enriched explant fraction exiting the vibratory platform. See, for example, FIG. 10 (A-C). In this example, the textured surface was 80 to 150 grit sandpaper, which comprises particles having an average diameter of about 90 μm to about 190 μm. The Friction Table separates particles using linear oscillating vibrations. The vibratory platform can be set to a specific compound angle and as a result of the vibration, the debris tends to move up the slope of the vibratory platform and the mature corn embryo explants move down the slope of the vibratory platform into one or more collection compartments. In this example, material to be separated first contacted the textured surface at a point where the tilt elevation and the pitch elevation were near their highest. This may be, for example, about 2.54 cm from the proximal edge of the vibratory platform and/or about 5.08 cm approximately diagonal from the corner of the vibratory platform where the proximal edge and the upper edge intersect. The contact location, however, may be modified depending on the size and dimensions of the vibratory platform and the material to be sorted. In this example, the material was fed onto the vibratory platform at a rate of 3.0 g/min. The vibration amplitude may be adjusted using a voltage regulator and was set to 60-80 V AC maximum capacity in this example, resulting in a vibration frequency of about 60 Hz to about 120 Hz. The weight set bolt was set to close to minimum. The vibratory platform was positioned at a tilt angle of about 12.7 degrees to about 14.7 degrees and a pitch angle of about 2.1 degrees to about 2.6 degrees in this example. After this final step, the collected corn embryo explants were about 83% pure as a percentage of total particles and were ready to be used in transformation. This represents about a 1.5-fold to about a 1.8-fold increase in explant purity compared to the purity of the embryo explants following Aspiration Classification and about an 83-fold increase in explant purity compared to the purity of the embryo explants following Seed Milling. The target range for corn embryo explant purity using the methods described herein is above about 70% as a percentage of total particles.

Prior to transformation, the embryo explant preparation may go through an additional purification step wherein the explants are allowed to float in an aqueous solution or simply sterile RO/DI water, which allows debris to settle to the bottom of a petri dish. The explants can then be recovered from the top of the solution for transformation.

### Example 2

### Preparation of Soybean Dried Excised Explants

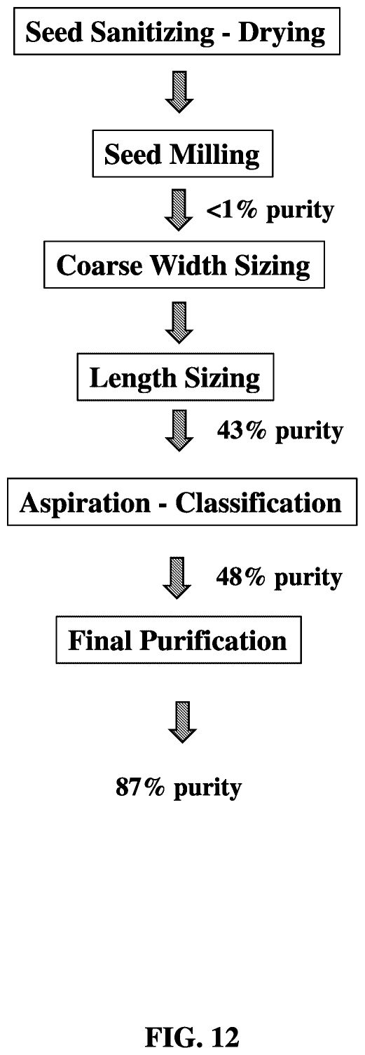

This example describes a process by which dry excised mature soybean embryo explants are isolated and purified using a series of steps involving the use of multiple devices and methods to achieve production of a high purity explant preparation for transformation. FIG. 12 illustrates the mature soybean embryo explant preparation and purification workflow that will be described further in this example. All the steps described below may be performed under clean room and ionization conditions with air filtered through HEPA filters to reduce contamination and static charges.

Mature soybean seeds were harvested, dried to a desired moisture content of about 4.5% to about 6.5% and stored in containers or bags prior to explant preparation. Soybean seeds were transferred to a seed sanitizer and dryer to prepare the seeds for milling as described in Example 1 (corresponding to Seed Sanitizing-Drying step in FIG. 12). For soybean seeds, the IPEC mix comprised RODI, 5,500 PPM bleach, and 0.02% TWEEN® for a total volume of about 340 gallons. The parameters for the Air Flow and Drying Steps in this example are provided in Table 2 below.

TABLE 2
Air Flow and Drying Step parameters.
High Air Flow Drying

| Duration | 60 | min |
|---|---|---|
| RH % Desiccant Wheel On | 50% | |
| Air Flow | 4,000 SCFM (Standard Cubic Feet per | |
| | Minute) | |
| Process Air Temp | 85° | F. |

Drying Step 1

| RH % Desiccant Wheel On | 50% | |
|---|---|---|
| Air Flow | 2,200 | SCFM |
| Process Air Temp | 85° | F. |
| Change Relative Humidity Max | 50% | |
| Change Temp Minimum | 67° | F. |

Drying Step 2

| RH % Desiccant Wheel On | 2% | |
|---|---|---|
| Air Flow | 2,200 | SCFM |
| Process Air Temp | 100° | F. |
| Change Relative Humidity Max | 40% | |
| Change Temp Minimum | 85° | F. |

Drying Step 3

| RH % Desiccant Wheel On | 1% | |
|---|---|---|
| Air Flow | 2,200 | SCFM |
| Process Air Temp | 103° | F. |
| Change Relative Humidity Max | 40% | |
| Change Temp Minimum | 85° | F. |

| | |
|---|---|
| Target Moisture | 4.5%-6.5% |

The sanitized and dried soybean seeds were transferred to a roller mill as described in Example 1 (corresponding to Seed Milling step in FIG. 12) to release the seed embryos comprising meristematic tissue from the remainder of the seed. See, for example, FIG. 1 (A-C). In this example, the grinding rollers measured about 15.24 cm in diameter and about 45.72 cm in length and were made of stainless steel. Grinding rollers made of other materials including, but not limited to, rubber and ceramic may also be used. In specific embodiments, a rice dehuller may also be used. In some embodiments, the grinding rollers may be about 7.62 cm to about 50.8 cm in diameter and about 5.08 cm to about 121.92 cm in length. For milling of soybean seeds, the top roll gap was set to about 4.2926 mm and the bottom roll gap was set to about 3.937 mm. All other milling settings were the same as those used for corn in Example 1. As described above, the roll cut identification may be adjusted depending on the characteristics of the seed to be milled. In certain embodiments, the roll cut identification may be, for example, AS, multiple AS, ST, flat, or other shape with about 4 to about 20 teeth per 2.54 cm. The rotation rate ratio, rate of rotation, and roll cut orientation may also be adjusted depending on the characteristics of the seed to be milled. The rotation rate ratio may be, for example, about 1:1 to about 4:1, the rate of rotation may be, for example, about 50 rpm to about 1200 rpm, and the roll cut orientation may be set to, for example, sharp to dull, dull to sharp, sharp to sharp, or dull to dull. The roll gaps may be adjusted, depending on the size of the seeds, to maximize seed milling and minimize explant damage. For milling of soybean seed, the roll gap may be, for example, about 0.762 mm to about 6.35 mm. After milling, the embryo explant purity in this experiment was about 5% as a percentage of total particles.

The milled soybean seed material was transferred to a Rotex® Siever (corresponding to Coarse Width Sizing step in FIG. 12) to separate the explants from the larger and smaller debris particles as described in Example 1. See, for example, FIG. 3. The stainless-steel wire mesh sieves used in this example, however, are different from those used in Example 1. In this example, the top sieve had openings of about 2032 μm with two 3.175 cm rubber balls per pocket under the sieve and the bottom sieve had openings of about 1181 μm with three 3.175 cm silicone balls per pocket under the sieve. The openings of the top sieve and bottom sieve may be adjusted according to the characteristics of the embryo explant of the variety used. For example, the top sieve may have openings of about 1600 μm to about 2600 μm with about one to about ten 3.175 cm rubber balls per pocket under the sieve. The bottom sieve, in some embodiments, may have openings of about 800 μm to about 1500 μm with about one to about ten 3.175 cm silicone balls per pocket under the sieve. The milled explants were fed into the Rotex® Siever at 54% VFD (˜65 V AC), which corresponds to a rate of about 2900 g/min. For soybean explant preparation, the top blast gate was fully open, and the bottom and middle blast gates were halfway open. The explants in this example were retained on the bottom sieve.

The sieved soybean explants were then transferred to an Indent Cylinder as previously described (corresponding to Length Sizing step in FIG. 12). See, for example, FIG. 5. The indentation size for the rotating cylinder used in this example ranged from about 2.0 mm to about 3.5 mm, depending upon the size of the explants from the specific soybean variety. In certain embodiments, an indentation size of about 2.0 mm to about 4.0 mm may be used. The Indent Cylinder was configured to rotate at about 37 rpm to about 38 rpm. The embryo explant preparation was initially fed into the Indent Cylinder at a rate of about 2000 g/min to load the cylinder. Once the cylinder was loaded the embryo explant preparation was fed into the Indent Cylinder at a rate of about 1000 to about 1300 g/min to balance the inflow and outflow of material. The debris collector setting, and the position of the end gate determine how thick the material bed will become. In this example, the waste container was raised about 5 cm above the cylinder floor and the highest depth of the retention arm was about 2.5 cm. Following this step, the embryo explant purity was up to about 43% as a percentage of total particles. This represents about an 8.6-fold increase in explant purity compared to the purity of the embryo explants following Seed Milling.

The dry embryo explant preparation was transferred to an STS-MACS Seed Separator (corresponding to Aspiration—Classification step in FIG. 12) as described in Example 1. See, for example, FIG. 6. The indicated VFD setting ranges for each chamber were as follows: Chamber #1=16.0-17.8 Hz (4.5 m/s-5.0 m/s); Chamber #2=18.0-20.8 Hz (5.5 m/s-6.4 m/s); Chamber #3=19.5-20.0 Hz (7.6 m/s-7.9 m/s); Chamber #4=20.0-22.0 Hz (10.8 m/s-11.9 m/s). In some examples, the Air-Classification step may be performed using a Hoffman® HMC 67 blower as described above. See, for example, FIG. 7. Following this step, the soybean explant preparation in this example was found to have a purity of about 48% as a percentage of total particles. This represents about a 1.1-fold increase in explant purity compared to the purity of the embryo explants following Length Sizing and about a 9.6-fold increase in explant purity compared to the purity of the embryo explants following Seed Milling.

Further purification of the soybean embryo explant preparation was performed using the Friction Table (corresponding to Final Purification step in FIG. 12) as described above in Example 1. See, for example, FIG. 10 (A-C). In this example, material to be separated first contacted the textured surface at a point where the tilt elevation and the pitch elevation were near their highest. This may be, for example, about 2.54 cm from the proximal edge of the vibratory platform and/or about 5.08 cm approximately diagonal from the corner of the vibratory platform where the proximal edge and the upper edge intersect. The contact location, however, may be modified depending on the size and dimensions of the vibratory platform and the material to be sorted. In this example, the material was fed onto the vibratory platform at a rate of 3.0 g/min. The vibration amplitude was adjusted using a voltage regulator and was set to 60-80 V AC maximum capacity, which corresponds to a vibration frequency of about 60 Hz to about 120 Hz. The weight set bolt was set to close to minimum. In this example, the textured surface of the vibratory platform was 120 grit sandpaper, which comprises particles having an average diameter of about 115 μm. The vibratory platform was positioned at a tilt angle of about 15.8 degrees to about 16.6 degrees and a pitch angle of about 4.3 degrees to about 7.45 degrees. Other textured surfaces may also be used, such as panoRama® Walk-&-Wall vinyl, a plasma coated surface, a cork surface, a fabric surface, a rubber surface, and a plastic surface. The vinyl surface panoRama® Walk-&-Wall vinyl is a 200 μm non-slip vinyl media with dimensionally stable fabric backing and repositionable adhesive. When the textured surface of the vibratory platform is panoRama® Walk-&-Wall vinyl, the vibratory platform may be positioned at a tilt angle of about 11.6 degrees to about 12.0 degrees and a pitch angle of about 1.9 degrees to about 3.25 degrees. Following this step, the purity of the soybean embryo explants was up to about 87% as a percentage of total particles. This represents about a 1.8-fold increase in explant purity compared to the purity of the embryo explants following Aspiration-Classification and about a 17.4-fold increase in explant purity compared to the purity of the embryo explants following Seed Milling. The target range for soybean embryo explant purity using the methods described herein is about 70% to about 90%, measured as a percentage of total particles.

Prior to transformation, the embryo explant preparation may go through an additional purification step wherein the explants are allowed to float in an aqueous solution or simply sterile RO/DI water, which allows debris to settle to the bottom of a petri dish. The embryo explants can then be recovered from the top of the solution for transformation.

### Example 3

### Preparation of Cotton Dried Excised Explants

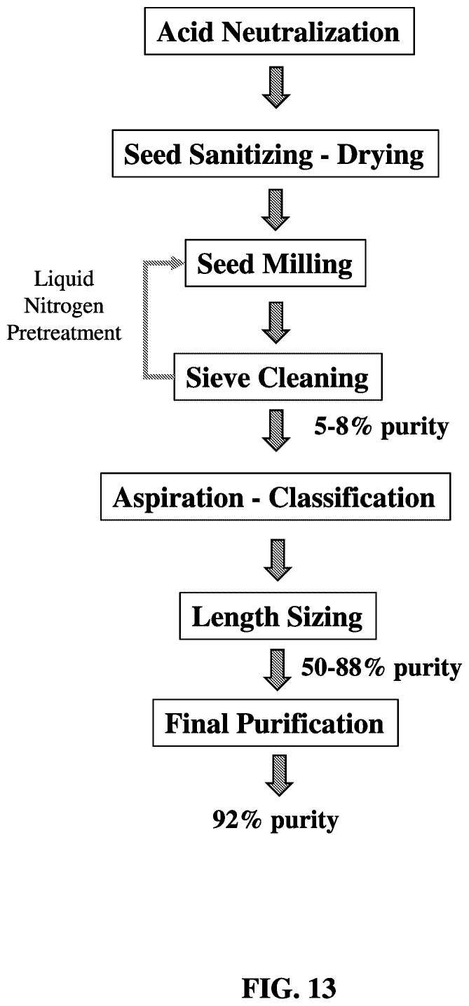

This example describes a process by which mature cotton embryo explants are excised from dry seeds and purified using a series of steps involving the use of multiple devices and methods to achieve production of a high purity explant preparation for transformation. FIG. 13 illustrates the mature cotton embryo explant preparation and purification workflow that will be described further in this example. All the steps described below may be performed under clean room and ionization conditions with air filtered through HEPA filters to reduce contamination and static charges.

Mature cotton seeds were harvested, delinted in the presence of sulfuric acid solution, dried to a desired moisture content of about 4.5% to about 6.5%, and stored in containers or bags prior to explant preparation. Prior to the sanitization and drying process, the cotton seeds were assayed for residual acid from the delinting process. If acid was found, the seeds were first neutralized (corresponding to Acid Neutralization step in FIG. 13) with potassium hydroxide or another acceptable base. Cotton seeds were transferred to the seed sanitizer and dryer to prepare the seed for milling as described above (corresponding to Seed Sanitizing-Drying step in FIG. 13). The IPEC solution, Air Flow, and Drying Steps were the same as described in Example 2.

The sanitized cotton seeds were then transferred to a Grinder (Model GP-140, Modern Process Equipment, Chicago, IL, USA, corresponding to Seed Milling step in FIG. 13). The GP-140 grinder uses two grinding plates, one which is stationary and one which rotates. See, for example, FIG. 1D. In this example, the Grinder was configured to run at about 400 rpm. The gap setting is established using two adjusters, an outer ring knob with a red arrow and a center screw. For cotton seed, the red arrow was set to 2.6, and the center screw was set to 3.5, which produces a gap size of about 3.0 mm to about 3.25 mm. The cotton seed was fed into the grinder at a rate of about 800 g/minute. Next the ground preparation was sieved (corresponding to Sieve Cleaning step in FIG. 13) to remove dust and some seed coat material using an Eclipse® 324 Seed and Grain Cleaner (Eclipse® Model 324, Clipper Separation Technologies, Bluffton, IN 46714, USA). See, for example, FIG. 4. The top moving plate of the Cleaner was a solid pan with holes near the bottom, and the bottom sieve had openings of about 1181 μm with three 35 mm rubber anti-blinding balls per section under the screen. The openings of the bottom sieve may be adjusted according to the characteristics of the embryo explant of the variety used. For example, the bottom sieve may have openings of about 700 μm to about 1300 μm with about one to about ten 35 mm rubber anti-blinding balls per pocket under the sieve. The blower was set to 90% maximum air flow, resulting in an air velocity of about 4.5 m/s to about 5.0 m/s where the explants are present, and the air baffles were set to the first position (10% open). A vacuum hose was attached to the upper aspiration location to remove light waste material. Next the sieved preparation was ground again. To prepare for this second grinding step, the ground seed material from the first round of milling and sieving was submerged in liquid nitrogen for about 20 to about 30 seconds and then immediately added to a second Grinder. This second Grinder was custom built to produce a precise gap setting of 1.5 mm. See, for example, FIG. 1D. The Grinder rotation was set to “forward” at a speed of 80% of the maximum, which produces a rotation speed of about 135 rpm. “Forward” refers to the rotational direction which moves the sharp side of the grinder teeth on the rotating plate toward the sharp side of the teeth on the stationary plate. The ground seed was placed overnight into a sealed 50-gallon barrel containing an inflow of about 7 standard cubic feet per minute (SCFM) of ultra-dry air. This step allows the ground seed to equilibrate to room temperature without the development of atmospheric condensation. Finally, the ground seed was subjected to a second sieving step using the Eclipse® 324 Seed and Grain Cleaner having a top sieve with openings of about 2032 μm with three 35 mm rubber anti-blinding balls per pocket under the sieve and a bottom sieve with openings of about 980 μm with three 35 mm rubber anti-blinding balls per pocket under the sieve. See, for example, FIG. 4. The openings of the top sieve and the bottom sieve may be adjusted according to the characteristics of the embryo explant of the variety used. In some embodiments, the top sieve may have openings of about 1600 μm to about 2500 μm with about one to about ten 35 mm rubber anti-blinding balls per pocket under the sieve and the bottom sieve may have openings of about 700 μm to about 1300 μm with about one to about ten 35 mm rubber anti-blinding balls per pocket under the sieve. The fan speed was set to 75% of the maximum, resulting in an approximate air flow of about 4.3 m/s to about 5 m/s where the explants are present. The air baffles were set to the second position (20% open). The cotton embryo explants were retained on the second sieve in both the first and second sieving steps. Following this step, the cotton embryo explant purity was about 5% to about 8%, measured as a percentage of the weight of embryo explants (g) per gram of preparation.

The cotton embryo explant preparation was then transferred to an STS-MACS Seed Separator (corresponding to Aspiration-Classification step in FIG. 13) as described above. The indicated VFD setting ranges for each chamber as follows: Chamber #1=21.0-25.5 Hz (5.9 m/s-7.2 m/s); Chamber #2=22.0-27.0 Hz (6.7 m/s-8.2 m/s); Chamber #3=22.5-30.0 Hz (8.8 m/s-11.9 m/s); Chamber #4=25.0-35.5 Hz (13.7 m/s-19.9 m/s). See, for example, FIG. 6. In some examples, the Air-Classification step may be performed using a Hoffman® HMC 67 blower as described above. See, for example, FIG. 7.

The sieved cotton explant preparation was then transferred to the Indent Cylinder for further purification (corresponding to Length Sizing step in FIG. 13) as described above. See, for example, FIG. 5. In this example, an indentation size of about 3.0 mm to about 4.0 mm was used for cotton embryo explants. In certain embodiments, an indentation size of about 2.0 mm to about 4.0 mm may be used. The Indent Cylinder was configured to rotate at about 37 rpm to about 38 rpm. The embryo explant preparation was initially fed into the Indent Cylinder at a rate of about 2000 g/min to load the cylinder. Once the cylinder was loaded the embryo explant preparation was fed into the Indent Cylinder at a rate of about 1000 g/min to about 1300 g/min to balance the inflow and outflow of material. The debris collector setting, and the position of the end gate determine how thick the material bed will become. In this example, the debris collector was raised about 5 cm above the cylinder floor, and the highest depth of the retention arm was about 2.5 cm. In some embodiments, the explant material may be processed multiple times through the Indent Cylinder using different indentation sizes to improve explant purity. Following this step, the cotton embryo explant purity was about 50% to about 88%, measured as a percentage of the weight of embryo explants (g) per gram of preparation. This represents about a 6.25-fold to about a 17.6-fold increase in explant purity compared to the purity of the embryo explants following Seed Milling.

Further purification of the cotton explants was performed on a Friction Table (corresponding to Final Purification step in FIG. 13) as described above. See, for example, FIG. 10 (A-C). In this example, material to be separated first contacted the textured surface at a point where the tilt elevation and the pitch elevation were near their highest. This may be, for example, about 2.54 cm from the proximal edge of the vibratory platform and/or about 5.08 cm approximately diagonal from the corner of the vibratory platform where the proximal edge and the upper edge intersect. The contact location, however, may be modified depending on the size and dimensions of the vibratory platform and the material to be sorted. Material was fed onto the vibratory platform at a rate of 3.0 g/min. The vibration amplitude was adjusted using a voltage regulator and was set to 60-80 V AC maximum capacity, corresponding to vibration frequency of about 60 Hz to about 120 Hz, including all ranges and values derivable therebetween. The weight set bolt was set to close to minimum. The textured surface of the vibratory platform in this example was either 120 grit sandpaper, which comprises particles have an average diameter of about 115 μm, or panoRama® Walk-&-Wall vinyl, which is a 200 μm non-slip vinyl media. Using the 120-grit sandpaper, the vibratory platform was positioned at a tilt angle of about 16.2 degrees to about 18.3 degrees and a pitch angle of about 2.4 degrees to about 4.9 degrees. Using the panoRama® Walk-&-Wall vinyl, the vibratory platform was positioned at a tilt angle of about 11.6 to about 14.2 degrees and a pitch angle of about 1.8 to about 3.25 degrees. Following this step, the purity of the cotton embryo explants was about 92%, measured as a percentage of the weight of embryo explants (g) per gram of preparation. This represents about a 1.04-fold to about a 1.84-fold increase in explant purity compared to the purity of the embryo explants following Length Sizing and about a 11.5-fold to about an 18.4-fold increase in explant purity compared to the purity of the embryo explants following Seed Milling.

Prior to transformation, the embryo explant preparation may go through an additional purification step wherein the explants are allowed to float in an aqueous solution or simply sterile RO/DI water, which allows debris to settle to the bottom of a petri dish. The explants can then be recovered from the top of the solution for transformation.

### Example 4

### Preparation of Wheat Dried Excised Explants

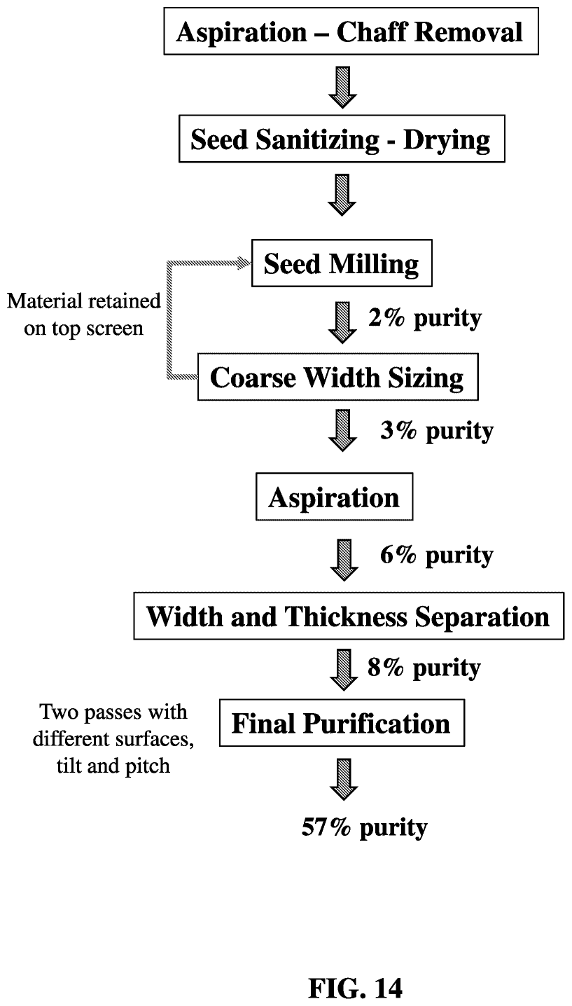

This example describes a process by which mature wheat embryo explants are excised from dry seeds and purified using a series of steps that involve the use of multiple devices and methods to achieve production of a high purity explant preparation for transformation. FIG. 14 illustrates the mature wheat embryo explant preparation and purification workflow that will be described further in this example. All the steps described below may be performed under clean room and ionization conditions with air filtered through HEPA filters to reduce contamination and static charges.

Prior to sanitization, bulk wheat seeds were aspirated to remove chaff (corresponding to Aspiration—Chaff Removal step in FIG. 14) using a Kice Multi-Aspirator® (Model 4F12 Multi Aspirator, Kice Industries, Wichita, KS, USA). See, for example, FIG. 8 (A-B). The Kice Multi-Aspirator® was elevated using a tall buckhorn stand to enable continuous flow. The Kice Multi-Aspirator® has a series of 6 functional units that allow the seeds to tumble back and forth under the force of gravity while flowing through the machine. At each functional unit ambient air was drawn through the aspirator, which lifts the lightweight particles into a discard port for removal. The wheat seeds and heavier particles tumbled down into the next selection chamber and were again exposed to the air flow selection process. This process was repeated six times. To provide a vacuum to collect the dust, a Camfil Farr® Dust Collector (Model GS6, Farr Air Pollution Control, Jonesboro, AR, USA) was connected to the Kice Multi-Aspirator®. The cleaned wheat seed was collected into a clean box using a temporary hose connected to the outflow of the aspirator.

The cleaned seeds were then sanitized (corresponding to Seed Sanitizing-Drying step in FIG. 14) as described in Example 1 using the same settings for the Munters® desiccant dryer and Spray Dyanics® coating drum. The parameters used for the Air Flow and Drying Steps are provided in Table 3 below.

TABLE 3
Air Flow and Drying Step parameters.
High Air Flow Drying

| Duration | 30 | min |
|---|---|---|
| RH % Desiccant Wheel On | 50% | |
| Air Flow | 4,000 SCFM (Standard Cubic Feet per | |
| | Minute) | |
| Process Air Temp | 85° | F. |

Drying Step 1

| RH % Desiccant Wheel On | 30% | |
|---|---|---|
| Air Flow | 2,100 | SCFM |
| Process Air Temp | 85° | F. |
| Change Relative Humidity Max | 50% | |
| Change Temp Minimum | 72° | F. |

Drying Step 2

| RH % Desiccant Wheel On | 1% | |
|---|---|---|
| Air Flow | 2,200 | SCFM |
| Process Air Temp | 100° | F. |
| Change Relative Humidity Max | 15% | |
| Change Temp Minimum | 86° | F. |

Drying Step 3

| RH % Desiccant Wheel On | 0.1% | |
|---|---|---|
| Air Flow | 2,200 | SCFM |
| Process Air Temp | 103° | F. |
| Change Relative Humidity Max | 30% | |
| Change Temp Minimum | 92° | F. |

| | |
|---|---|
| Target Moisture | 7%-8% |

The sanitized wheat seeds were then transferred to the Model IMD 79 roller mill as previously described (corresponding to Seed Milling step in FIG. 14). See, for example, FIG. 1A-C. The roll cut identification used for wheat embryo explant preparation was 8AS, and the roll speed ratio was set to 1.1:1 with a roll cut orientation of sharp to dull. The top roll gap was set to 1.2827 mm and the bottom roll gap was set to 0.3683 mm. The seeds were fed into the roller mill feeder and subsequently milled. After milling, the embryo explant purity in this experiment was about 2% as percentage of particles.

The embryo explant preparation was then transferred to a model A8G12L-3S Rotex® Siever (corresponding to Coarse Width Sizing step in FIG. 14) to separate the explants from the larger and smaller debris particles as described above. See, for example, FIG. 3. For wheat explant preparation, all stainless-steel wire mesh sieves were used. The top sieve had openings of 864 μm with three 3.175 cm rubber balls per pocket under the sieve. The bottom sieve had openings of 610 μm with three 3.175 cm silicone balls per pocket under the sieve. The opening size of the top sieve and bottom sieve may be adjusted according to the characteristics of the embryo explant of the variety used. For example, the top sieve may have openings of about 600 μm to about 1200 μm with about one to about ten 3.175 cm rubber balls per pocket under the sieve. The bottom sieve may, in some embodiments, have openings of about 300 μm to about 900 μm with about one to about ten 3.175 cm silicone balls per pocket under the sieve. The milled explants were fed into the Rotex® Siever at 54% VFD (˜65 V AC), which results in a feed rate of about 1500 g/min. For wheat explant preparation, all three blast gates were kept fully open, which reduces the aspiration experienced by the explants to just above zero. In this example, explants were retained on the bottom sieve. Following this step, the embryo explant purity in this experiment was about 3% as a percentage of total particles. This represents about a 1.5-fold increase in explant purity compared to the purity of the embryo explants following Seed Milling. All explant materials retained on the top Rotex® sieve were subject to a second round of milling in the roller mill using the same settings.

The explant preparation retained on the Rotex® bottom sieve was transferred to a Model STS-MACS 104 (corresponding to Aspiration step in FIG. 14) as described above. See, for example, FIG. 6. The indicated VFD setting ranges for each chamber are as follows: Chamber #1=11.50 Hz (3 m/s-3.3 m/s); Chamber #2=13.00 Hz (3.8 m/s-4.3 m/s); Chamber #3=13.50 Hz (5.1 m/s-5.5 m/s); Chamber #4=14.0 Hz (7.2 m/s-7.7 m/s). The enriched explant preparation exits the system through a collection chute while the separated debris exit the system through one of the discharge ports located in each chamber. Following this step, the embryo explant purity in this experiment was about 6% as a percentage of total particles. This represents about 2-fold increase in explant purity compared to the purity of the embryo explants following Coarse Width Sizing and about a 3-fold in explant purity compared to the purity of the embryo explants following Seed Milling.

The prepared explants were then transferred from the STS-MACS and further separated using a SWECO® Vibro-Energy® Separator (corresponding to Width and Thickness Separation step in FIG. 14). See, for example, FIG. 9. The frequency of vibrations can be set by the rpm of the motor and may, in some embodiments, be about 2 Hz to about 20 Hz, including all ranges and values derivable therebetween. To purify the wheat explants, the motor was set to 1160 rpm. The upper and lower weights were adjusted away from 180 degrees to create imbalance. The upper weights were set in either the 4 and 4 positions or the 5 and 5 positions, and the lower weight lead angle was set to an angle in a range from 35 degrees to 45 degrees. This was achieved by placing the lower weights in the 0 and 70 positions or 0 and 90 positions, respectively. The combination of these weight positions results in an indicated horizontal amplitude of about 4.7625 mm to about 5.5563 mm and an indicated vertical amplitude of about 4.7625 mm to about 7.1438 mm. The vibrated screen comprised slotted holes measuring about 10 mm in length by about 0.65 mm in width to achieve a thickness separation. The motion of the SWECO® separator causes the wheat explants to eventually land on their end or side planes, enabling the explants to fall through the slots and become discharged via the undersize discharge spout. The waste fraction vibrates to the edge of the screen and is discharged via the oversize discharge spout. Discharge spouts on the upper and lower spacing frames were offset by 180 degrees. Following this step, the embryo explant purity in this experiment was about 8% as a percentage of total particles. This represents about 1.3-fold increase in explant purity compared to the purity of the embryo explants following Aspiration and about a 4-fold in explant purity compared to the purity of the embryo explants following Seed Milling.

For the final purification step, the explants were further separated from the remaining debris using a Friction Table (corresponding to Final Purification step in FIG. 14). The Friction Table is a modified Model OSCA-1000 Oscillating Shape Sorter as described above in which the surface of the vibratory platform is modified to have a textured surface. See, for example, FIG. 10 (A-C). For wheat embryo explant purification, the wheat explants move in the opposite direction relative to the explants for corn and soybean. Therefore, the wheat explants were collected at the elevated side of the vibratory platform. In this example, material to be separated first contacted the textured surface of the vibratory platform about 2.54 cm from the lower edge of the platform and near the center point between the proximal edge and the distal edge. The vibration amplitude was adjusted using a voltage regulator and was set to 60-80 V AC maximum capacity, resulting in a vibration frequency of about 60 Hz to about 120 Hz. The weight set bolt was set to close to minimum. Material was fed onto the vibratory platform at a rate of about 3 g/min and two rounds of purification were performed. In the first round, 120-grit sandpaper, which comprises particles having an average diameter of about 115 μm, was used as the textured surface. The platform was positioned at a tilt angle of about 14.5 degrees and a pitch angle of about 4.0 degrees. The collection trough of the Friction Table is 60.96 cm wide and may be divided, in some embodiments, into collection compartments. By dividing the collection trough, one can select one or more collection compartments to retrieve the explants. In the first round, a divider was set in the middle of the collection trough and the wheat explants were collected from 30.48 cm of the upslope. The lower material containing debris was discarded. In the second round, panoRama® Walk & Wall material vinyl was used as the textured surface, and the platform was positioned at a tilt angle of about 12.9 degrees and a pitch angle of about 2.2 degrees. In the second round, a divider was set in the collection trough 45.72 cm from the downslope of the vibratory platform and the wheat explants were collected from 15.24 cm of the upslope. Following this step, the embryo explant purity in this experiment was about 57% as a percentage of total particles. This represents about 7.1-fold increase in explant purity compared to the purity of the embryo explants following Width and Thickness Separation and about a 28.5-fold in explant purity compared to the purity of the embryo explants following Seed Milling.

Prior to transformation, the wheat explant preparation may go through an additional purification step wherein the explants are allowed to float in an aqueous solution or simply sterile RO/DI water, which allows debris to settle to the bottom of a petri dish. The explants were then recovered from the top of the solution for transformation.

### Example 5

### Preparation of Canola Dried Excised Explants

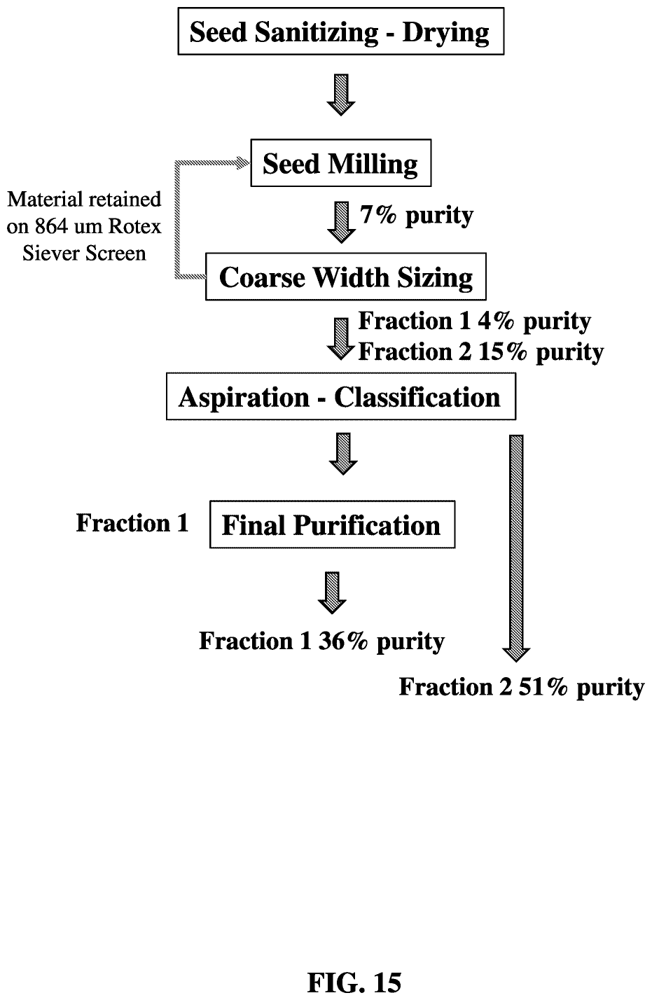

This example describes a process by which mature canola embryo explants are excised from dry seeds and purified using a series of steps that involve the use of multiple devices and methods to achieve production of a high purity explant preparation for transformation. FIG. 15 illustrates the mature canola embryo explant preparation and purification workflow that will be described further in this example. All the steps described below may be performed under clean room and ionization conditions with air filtered through HEPA filters to reduce contamination and static charges.

Mature canola seeds were first sanitized and then dried (corresponding to Seed Sanitizing—Drying step in FIG. 15). For sanitization, 16 Bry-Air® seed trays, measuring 96.774 cm², were lined with mesh inserts made from stainless steel screens having openings of about 635 μm. About 500 g of canola seed was placed on each Bry-Air® seed tray, which was submerged in a container containing 50% bleach by volume. The bleach solution was created by combining 1 gallon of 8.25% sodium hypochlorite and 1 gallon of reverse osmosis de-ionized (RODI) water. The solution was stirred for several minutes, and the seeds remained in the solution for about 10 minutes. The trays were then moved to a container containing RODI water, where the seeds were mixed for about 2 minutes to allow them to settle to the bottom. Seeds that continued to float were removed using a mesh scoop and discarded. The remaining seeds were then dried in a Bry-Air® Seed Dryer (Model VFB-3-E-DXA, Bry-Air Inc., Sunsbury, OH, USA). The Bry-Air® Seed Dryer consists of a chamber that holds the trays and a desiccant dehumidifier that continuously feeds dry air to the chamber. The dryer was set to 38° C. and 1% relative humidity and the trays of canola seeds remained in the chamber for about 24 hours.

Following sanitization and drying, the canola seed was milled (corresponding to Seed Milling step in FIG. 15) using a LPP 6.5 GRAN-U-LIZER® roller mill (Modern Process Equipment, Chicago, IL, USA). See, for example, FIG. 1 (A-C). The LPP 6.5 mill was configured with 20ST teeth, oriented sharp to sharp. The left roll was configured to rotate at about 345 rpm and the right roll was configured to rotate at about 138 rpm. The gap spacing was set to 0.8509 mm and the seed was fed at a rate of about 26% VFD, which corresponds to a rate of about 84 g/min. Following this step, the embryo explant purity in this experiment was found to be about 7% as a percentage of total particles.

The population of milled dry seeds or milled dry seed explants was then transferred to a Rotex® Siever (corresponding to Coarse Width Sizing step in FIG. 15). See, for example, FIG. 3. For the milled canola seed in this example, three sieves were employed. The top sieve had openings of 864 μm with three 3.175 cm rubber balls per pocket under the sieve. The middle sieve had openings of 812 μm with three 3.175 cm rubber balls per pocket under the sieve. The bottom sieve had openings of 503 μm with three 3.175 cm silicone balls per pocket under the sieve. The openings of the top sieve, middle sieve, and bottom sieve may be adjusted according to the characteristics of the embryo explant of the variety used. For example, the top sieve may have openings of about 600 μm to about 1100 μm with about one to about ten 3.175 cm rubber balls per pocket under the sieve. The middle sieve may have openings of about 600 μm to about 1000 μm with about one to about ten 3.175 cm rubber balls under the sieve, and the bottom sieve may, in some embodiments, have openings of about 300 μm to about 1000 μm with about one to about ten 3.175 cm silicone balls per pocket under the sieve. The milled explants were fed into the Rotex® Siever at 47% VFD (˜56 V AC), which corresponds to a rate of about 190 g/min. A container was configured to collect the explants, which bypassed the aspiration from the dust collector. The sieved canola seed material containing the embryo explants was then divided into two fractions. Material that was retained on the middle 812 μm sieve was designated as Fraction 1. Material that fell through the top and middle sieves and was retained on the bottom 503 μm sieve was designated as Fraction 2. Material that remained on the top 864 μm sieve was milled a second time, sieved a second time, and then fractioned as described above. During the second round of milling, the roller mill settings and roller were the same as above with the exception that the gap spacing was reduced to 0.6985 mm and the feed rate was increased to 28% VFD, which corresponds to a rate of about 175 g/min, to facilitate steady feeding. Material from the second round that was retained on the middle 812 μm sieve was added to Fraction 1. Material that fell through the top and middle sieves and was retained on the bottom 503 μm sieves was added to Fraction 2. Following this step, the embryo explant purity of Fraction 1 was about 4% and the embryo explant purity of Fraction 2 was about 15%, as a percentage of total particles. This represents about 2.1-fold increase in explant purity for Fraction 2 compared to the purity of the embryo explants following Seed Milling.

Each of the two combined fractions was then purified further using the Model STS-MACS 104 (corresponding to Aspiration step in FIG. 15) as described above. See, for example, FIG. 6. The indicated VFD setting ranges for each chamber were as follows: Chamber #1=13.00 Hz (3.6 m/s); Chamber #2=15.00 Hz (4.7 m/s); Chamber #3=15.00 Hz (6 m/s); Chamber #4=16.5 Hz (8.8 m/s). The enriched explant preparation exits the system through a collection chute, while the separated debris exits the system through one of the discharge ports located in each chamber. Following this step, the embryo explant purity of Fraction 2 was about 51% as a percentage of total particles. This represents about 3.4-fold increase in explant purity for Fraction 2 compared to the purity of the embryo explants following Coarse Width Sizing and about a 7.3-fold increase in explant purity for Fraction 2 compared to the purity of the embryo explants following Seed Milling.

As an additional purification step for Fraction 1 (corresponding to Final Purification step in FIG. 15), the canola explant preparation from Fraction 1 was hand sieved using a small 20.32 cm by 20.32 cm sieve comprising about 0.5 mm slotted holes. The sieve was tapped with a scoopula for about 30 seconds. The embryo explant material was retained on top of the sieve while the debris fell through. Following this step, the embryo explant purity of Fraction 1 was about 36% as a percentage of total particles. This represents about a 5.1-fold increase in explant purity for Fraction 1 compared to the purity of the embryo explants following Seed Milling.

Fraction 2 contained the majority of the regenerable canola explants produced by this method (i.e., about 1.97 million regenerable explants from the initial calculated 4,874,624 seeds) and had a purity of about 51% as a percentage of total particles with an 88% calculated regeneration from sampling. Fraction 1 yielded 389,452 explants with a purity of about 36% as a percentage of total particles.

Prior to transformation, the canola explant preparation may go through an additional purification step wherein the explants are allowed to float in an aqueous solution or simply sterile RO/DI water, which allows debris to settle to the bottom of a petri dish. The explants were then recovered from the top of the solution for transformation.

1. A method of purifying genetically modifiable dry plant embryo explants, the method comprising:
sanitizing a population of plant seeds;
milling the population of plant seeds to produce a preparation of dry plant embryo explants comprising meristematic tissue, wherein the preparation comprises a population of dry plant embryo explants and debris material;
aspirating the preparation of embryo explants to separate an aspirated fraction of the embryo explants from an aspirated portion of the debris material; and
purifying the genetically modifiable dry embryo explants.

2. The method of claim 1, wherein the dry plant embryo explants are selected from the group consisting of corn embryo explants, soybean embryo explants, cotton embryo explants, wheat embryo explants, and canola embryo explants.

3. The method of claim 1, wherein the population of plant seeds is a population of corn seeds, and wherein said milling comprises:
positioning a first grinding roller and a second grinding roller to define a first gap having a first gap distance between the first roller and the second roller;
rotating the first roller about a first axis of rotation and the second roller about a second axis of rotation;
passing the population of seeds through said first gap to produce a first preparation of plant embryo explants comprising meristematic tissue;
positioning a third grinding roller and a fourth grinding roller to define a second gap having a second gap distance between the third roller and the fourth roller;
rotating the third roller about a third axis of rotation and the fourth roller about a fourth axis of rotation; and
passing the first preparation of embryo explants through said second gap to produce a second preparation of plant embryo explants comprising meristematic tissue,
wherein the first gap distance is about 0.381 mm to about 7.62 mm, about 2.032 mm to about 2.794 mm, or is about 2.54 mm,
or wherein the second gap distance is about 0.381 mm to about 7.62 mm, about 0.762 mm to about 1.778 mm, or is about 1.27 cm.

4. The method of any one of claims 1-3, wherein the population of plant seeds is a population of corn seeds and said aspirating comprises:
aspirating within a first vertical chamber, a second vertical chamber, a third vertical chamber, and a fourth vertical chamber with a first upward air flow, a second upward air flow, a third upward air flow, and a fourth upward air flow,
wherein the first upward airflow has a first air flow velocity of about 4.5 m/s to about 6.0 m/s, about 5.0 m/s to about 5.5 m/s, or about 5.1 m/s to about 5.3 m/s,
wherein the second upward air flow has a second air flow velocity of about 5.5 m/s to about 6.5 m/s or about 5.9 m/s to about 6.3 m/s,
wherein the third upward air flow has a third air flow velocity of about 6.5 m/s to about 7.5 m/s, about 7.0 m/s to about 7.5 m/s, or about 7.0 m/s to about 7.3 m/s,
and wherein the fourth upward air flow has a fourth air flow velocity of about 9.5 m/s to about 10.5 m/s or about 9.8 m/s to about 10.2 m/s.

5. The method of any one of claims 1-4, wherein the population of plant seeds is a population of corn seeds and the method further comprises:
contacting the preparation of dry plant embryo explants comprising meristematic tissue with a first moving sieve, wherein said first moving sieve comprises a plurality of openings, each having a first physical opening size;
separating a first fraction of embryo explants from a first portion of the debris material by length, width, or thickness relative to the first physical opening size, or relative to a first effective opening size;
contacting the first fraction with a second moving sieve, wherein the second moving sieve comprises a plurality of openings, each having a second physical opening size; and
separating a second fraction of embryo explants from a second portion of the debris material by length, width, or thickness relative to the second physical opening size, or relative to a second effective opening size,
wherein the first moving sieve and the second moving sieve move in a circular, elliptical, or linear motion.

6. The method of claim 5, wherein:
the first physical opening size is about 500 μm to about 2000 μm, about 800 μm to about 2000 μm, or about 1181 μm; or
the second physical opening size is about 500 μm to about 1000 μm or about 812 μm.

7. The method of claim 5 or 6, the method further comprising:
contacting the second fraction of dry plant embryo explants comprising meristematic tissue with an interior surface of a rotating cylinder, wherein the interior surface comprises a plurality of indentations, the indentations having an indentation size and an indentation shape, and wherein the second fraction comprises a population of dry plant embryo explants and debris material;
rotating the rotating cylinder about an axis of rotation, wherein said axis of rotation is substantially parallel to the ground; and
separating a cylinder fraction of the plant embryo explants from a cylinder portion of the debris material.

8. The method of claim 7, wherein each indentation size comprises an indentation diameter, an indentation width, an indentation length, or an indentation depth, wherein the indentation diameter, the indentation width, or the indentation length is about 1.50 mm to about 2.75 mm, about 1.75 mm to about 2.50 mm, about 2.00 mm to about 2.25 mm, about 2.00 mm, or about 2.25 mm, or wherein the indentation depth is about 0.25 mm to about 2.00 mm, about 0.50 mm to about 1.75 mm, about 0.75 mm to about 1.25 mm, or about 1.00 mm.

9. The method of claims 1 and 4-8, wherein the population of plant seeds is a population of corn seeds and the method further comprises:
contacting the aspirated fraction with a first vibratory screen, wherein said first vibratory screen comprises a plurality of openings, each having a first opening size and a first opening shape, and
wherein the aspirated fraction comprises a population of dry plant embryo explants and debris material;
vibrating the first vibratory screen to produce a first screen motion, wherein the first screen motion comprises a first horizontal vibratory component;
separating a first screen fraction of embryo explants from a first screen portion of the debris material by length, width, or thickness relative to the first opening size or the first opening shape, or by a displacement of the first screen fraction relative to a displacement of the first screen portion of the debris material produced by the first screen motion;
contacting the first screen fraction with a second vibratory screen, wherein the second vibratory screen comprises a plurality of openings, each having a second opening size and a second opening shape;
vibrating the second vibratory screen to produce a second screen motion, wherein the second screen motion comprises a second horizontal vibratory component; and
separating a second screen fraction of embryo explants from a second screen portion of the debris material comprised in the first screen fraction by length, width, or thickness relative to the second opening size or the second opening shape, or by a displacement of the second screen fraction relative to a displacement of the second screen portion of the debris material produced by the second screen motion.

10. The method of claim 9, wherein:
the first opening shape or the second opening shape is circular, and wherein the first opening size or the second opening size is about 1.3 mm to about 1.6 mm, about 1.4 mm to about 1.5 mm, about 1.3 mm to about 1.5 mm, or about 1.4 mm to about 1.6 mm in diameter, or about 1.3 mm, about 1.4 mm, about 1.5 mm, or about 1.6 mm in diameter; or
the first opening shape or the second opening shape is oblong, and wherein the first opening size or the second opening size is about 5 mm to about 15 mm, about 6 mm to about 14 mm, about 8 mm to about 12 mm, about 8 mm to about 10 mm, about 9 mm to about 11 mm, about 10 mm to about 12 mm in length, or about 8 mm, about 9 mm, about 10 mm, about 11 mm, or about 12 mm in length, and from about 0.6 mm to about 0.8 mm, about 0.6 mm to about 0.7 mm, or about 0.7 mm to about 0.8 mm in width, or about 0.6 mm, about 0.65 mm, about 0.7 mm, about 0.75 mm, or about 0.8 mm in width.

11. The method of claim 9 or 10, the method further comprising aspirating the second screen fraction of embryo explants to separate a second aspirated fraction of the embryo explants from a second aspirated portion of the debris material, wherein said aspirating comprises:
aspirating within a first vertical chamber, a second vertical chamber, a third vertical chamber, and a fourth vertical chamber with a first upward air flow, a second upward air flow, a third upward air flow, and a fourth upward air flow,
wherein the first upward airflow has a first air flow velocity of about 4.5 m/s to about 6.0 m/s, about 5.0 m/s to about 5.5 m/s, or about 5.1 m/s to about 5.3 m/s,
wherein the second upward air flow has a second air flow velocity of about 5.5 m/s to about 6.5 m/s or about 5.9 m/s to about 6.3 m/s,
wherein the third upward air flow has a third air flow velocity of about 6.5 m/s to about 7.5 m/s, about 7.0 m/s to about 7.5 m/s, or about 7.0 m/s to about 7.3 m/s,
and wherein the fourth upward air flow has a fourth air flow velocity of about 9.5 m/s to about 10.5 m/s or about 9.8 m/s to about 10.2 m/s.

12. The method of any one of claims 1-11, wherein the population of plant seeds is a population of corn seeds and the purifying comprises:
contacting the aspirated fraction, the second fraction, the cylinder fraction, the second screen fraction, or the second aspirated fraction with a textured surface of a vibratory platform, wherein the textured surface of the vibratory platform is substantially planar, and wherein the aspirated fraction, the second fraction, the cylinder fraction, the second screen fraction, or the second aspirated fraction comprises a population of dry plant embryo explants and debris material;
vibrating the vibratory platform to produce a first platform motion; and
separating a platform fraction of the plant embryo explants from a platform portion of the debris material according to a displacement of the platform fraction relative to a displacement of the platform portion of debris material on the textured surface of the vibratory platform,
wherein the vibratory platform comprises a first tilt angle of about 10.0 degrees to about 20.0 degrees, about 10.0 degrees to about 17.0 degrees, about 12.5 degrees to about 15.0 degrees, about 12.7 degrees to about 14.7 degrees, or about 13.7 degrees, and a first pitch angle of about 1.5 degrees to about 3.5 degrees, about 2.0 degrees to about 3.0 degrees, about 2.1 degrees to about 2.6 degrees, about 2.3 degrees, or about 2.4 degrees.

13. The method of claim 1, wherein the population of plant seeds is a population of soybean seeds, and wherein said milling comprises:
positioning a first grinding roller and a second grinding roller to define a first gap having a first gap distance between the first roller and the second roller;
rotating the first roller about a first axis of rotation and the second roller about a second axis of rotation;
passing the population of seeds through said first gap to produce a first preparation of plant embryo explants comprising meristematic tissue;
positioning a third grinding roller and a fourth grinding roller to define a second gap having a second gap distance between the third roller and the fourth roller;
rotating the third roller about a third axis of rotation and the fourth roller about a fourth axis of rotation; and
passing the first preparation of embryo explants through said second gap to produce a second preparation of plant embryo explants comprising meristematic tissue,
wherein the first gap distance is about 0.762 mm to about 6.35 mm, about 3.81 mm to about 5.08 mm, or is about 4.2926 mm,
or wherein the second gap distance is about 0.762 mm to about 6.35 mm, about 3.556 mm to about 4.318 mm, or is about 3.937 mm.

14. The method of claim 1 or 13, wherein the population of plant seeds is a population of soybean seeds and said aspirating comprises:
aspirating within a first vertical chamber, a second vertical chamber, a third vertical chamber, and a fourth vertical chamber with a first upward air flow, a second upward air flow, a third upward air flow, and a fourth upward air flow,
wherein the first upward airflow has a first air flow velocity of about 4.0 m/s to about 5.5 m/s or about 4.2 m/s to about 4.9 m/s,
wherein the second upward air flow has a second air flow velocity of about 5.0 m/s to about 7.0 m/s or about 5.8 m/s to about 6.7 m/s,
wherein the third upward air flow has a third air flow velocity of is about 7.0 m/s to about 8.5 m/s, about 7.5 m/s to about 8.0 m/s, or about 7.7 m/s to about 7.9 m/s,
and wherein the fourth upward air flow has a fourth air flow velocity of about 10.5 m/s to about 12.5 m/s, about 10.5 m/s to about 12.0 m/s, or about 10.8 m/s to about 12.0 m/s.

15. The method of any one of claims 1, 13 and 14, wherein the population of plant seeds is a population of soybean seeds and the method further comprises:
contacting the preparation of dry plant embryo explants comprising meristematic tissue with a first moving sieve, wherein said first moving sieve comprises a plurality of openings, each having a first physical opening size;
separating a first fraction of embryo explants from a first portion of the debris material by length, width, or thickness relative to the first physical opening size, or relative to a first effective opening size;
contacting the first fraction with a second moving sieve, wherein the second moving sieve comprises a plurality of openings, each having a second physical opening size; and
separating a second fraction of embryo explants from a second portion of the debris material by length, width, or thickness relative to the second physical opening size, or relative to a second effective opening size,
wherein the first moving sieve and the second moving sieve move in a circular, elliptical, or linear motion.

16. The method of claim 15, wherein:
the first physical opening size is about 800 μm to about 2600 μm, about 1600 μm to about 2600 μm, or about 2032 μm; or
the second physical opening size is about 800 μm to about 1500 μm or about 1181 μm.

17. The method of claim 15 or 16, the method further comprising:
contacting the second fraction of dry plant embryo explants comprising meristematic tissue with an interior surface of a rotating cylinder, wherein the interior surface comprises a plurality of indentations, the indentations having an indentation size and an indentation shape, and wherein the second fraction comprises a population of dry plant embryo explants and debris material;
rotating the rotating cylinder about an axis of rotation, wherein said axis of rotation is substantially parallel to the ground; and
separating a cylinder fraction of the plant embryo explants from a cylinder portion of the debris material.

18. The method of claim 17, wherein each indentation size comprises an indentation diameter, an indentation width, an indentation length, or an indentation depth, wherein the indentation diameter, the indentation width, or the indentation length is about 2.25 mm to about 3.50 mm, about 2.50 mm to about 3.25 mm, about 2.75 mm to about 3.00 mm, about 2.75 mm, or about 3.00 mm, or wherein the indentation depth is about 0.25 mm to about 2.00 mm, about 0.50 mm to about 1.75 mm, about 0.75 mm to about 1.25 mm, or about 1.00 mm.

19. The method of any one of claims 1 and 13-18, wherein the population of plant seeds is a population of soybean seeds and the purifying comprises:
contacting the aspirated fraction, the second fraction, or the cylinder fraction with a textured surface of a vibratory platform, wherein the first textured surface of the vibratory platform is substantially planar, and wherein the aspirated fraction, the second fraction, or the cylinder fraction comprises a population of dry plant embryo explants and debris material;
vibrating the vibratory platform to produce a first platform motion; and
separating a platform fraction of the plant embryo explants from a platform portion of the debris material according to a displacement of the platform fraction relative to a displacement of the platform portion of debris material on the textured surface of the vibratory platform,
wherein the vibratory platform comprises a first tilt angle of about 10.0 degrees to about 20.0 degrees, about 10.0 degrees to about 18.0 degrees, about 14.0 degrees to about 20.0 degrees, about 11.0 degrees to about 17.0 degrees, about 11.6 degrees to about 16.6 degrees, about 11.6 degrees to about 12.0 degrees, about 15.8 degrees to about 16.6 degrees, about 11.8 degrees, or about 16.2 degrees, and a first pitch angle of about 1.5 degrees to about 8.0 degrees, about 1.9 degrees to about 7.5 degrees, about 1.9 degrees to about 3.3 degrees, about 4.3 degrees to about 7.5 degrees, about 2.5 degrees, about 2.6 degrees, or about 5.9 degrees.

20. The method of claim 1, wherein the population of plant seeds is a population of cotton seeds, and wherein said milling comprises:
positioning a first grinding plate and a second grinding plate to define a first gap having a first gap distance between the first plate and the second plate;
rotating the first plate or the second plate about an axis of rotation; and
contacting the population of plant seeds with an interior surface of the first plate and an interior surface of the second plate to produce a first preparation of embryo explants comprising meristematic tissue, wherein the first gap distance is about 2.5 mm to about 4.0 mm or about 3.0 mm to about 3.25 mm.

21. The method of claim 1 or 20, wherein the population of plant seeds is a population of cotton seeds and said aspirating comprises:
aspirating within a first vertical chamber, a second vertical chamber, a third vertical chamber, and a fourth vertical chamber with a first upward air flow, a second upward air flow, a third upward air flow, and a fourth upward air flow,
wherein the first upward airflow has a first air flow velocity of about 5.5 m/s to about 8.0 m/s, about 5.5 m/s to about 7.5 m/s, or about 5.6 m/s to about 7.3 m/s,
wherein the second upward air flow has a second air flow velocity of about 6.5 m/s to about 8.5 m/s or about 6.8 m/s to about 8.4 m/s,
wherein the third upward air flow has a third air flow velocity of about 8.0 m/s to about 12.5 m/s, about 8.5 m/s to about 12.0 m/s, or about 8.7 m/s to about 11.7 m/s,
and wherein the fourth upward air flow has a fourth air flow velocity of about 13.0 m/s to about 20.5 m/s, about 13.5 m/s to about 20.3 m/s, or about 13.7 m/s to about 20.1 m/s.

22. The method of any one of claims 1 and 20-21, wherein the population of plant seeds is a population of cotton seeds and the method further comprises:
contacting the first preparation of embryo explants with a moving plate, wherein the moving plate comprises a proximal end, a distal end, and a plurality of openings located near the distal end, each comprising a first physical opening size, and wherein the first preparation comprises a population of embryo explants and debris material;
passing the first preparation through the plurality of openings of the moving plate and contacting a first moving sieve with said first preparation, wherein the first moving sieve comprises a plurality of openings, each having a second physical opening size; and
separating a first fraction of embryo explants from a first portion of the debris material by length, width, or thickness relative to the second physical opening size,
wherein the moving plate and the first moving sieve move in a linear motion,
and wherein the first physical opening size is about 300 μm to about 5000 μm, and the second physical opening size is about 700 μm to about 1300 μm or about 1181 μm.

23. The method of claim 22, the method further comprising:
positioning a third grinding plate and a fourth grinding plate to define a second gap having a second gap distance between the third plate and the fourth plate;
rotating the third plate or the fourth plate about an axis of rotation; and
contacting the first fraction with an interior surface of the third plate and an interior surface of the fourth plate to produce a second preparation of embryo explants comprising meristematic tissue, wherein the second gap distance is about 0.5 mm to about 2.5 mm or about 1.5 mm.

24. The method of claim 23, the method further comprising:
contacting the second preparation with a second moving sieve comprising a plurality of openings, each having a third physical opening size;
separating a second fraction of embryo explants from a second portion of the debris material by length, width, or thickness relative to the third physical opening size;
contacting the second fraction with a third moving sieve comprising a plurality of openings, each comprising a fourth physical opening size; and
separating a third fraction of embryo explants from a third portion of the debris material by length, width, or thickness relative to the fourth physical opening size,
wherein the second moving sieve and the third moving sieve move in a linear motion,
and wherein the third physical opening size is about 1600 μm to about 2500 μm or about 2032 μm; and the fourth physical opening size is about 700 μm to about 1300 μm, or about 980 μm.

25. The method of claim 23, the method further comprising: applying a cryogenic treatment to the first fraction of embryo explants prior to contacting the first fraction with the third plate and the fourth plate.

26. The method of any one of claims 20-25, the method further comprising:
contacting the aspirated fraction of dry plant embryo explants comprising meristematic tissue with an interior surface of a rotating cylinder, wherein the interior surface comprises a plurality of indentations, the indentations having an indentation size and an indentation shape, and wherein the second fraction comprises a population of dry plant embryo explants and debris material;
rotating the rotating cylinder about an axis of rotation, wherein said axis of rotation is substantially parallel to the ground; and
separating a cylinder fraction of the plant embryo explants from a cylinder portion of the debris material.

27. The method of claim 26, wherein each indentation size comprises an indentation diameter, an indentation width, an indentation length, or an indentation depth, wherein the indentation diameter, the indentation width, or the indentation length is about 2.25 mm to about 3.50 mm, about 2.50 mm to about 3.25 mm, about 2.75 mm to about 3.00 mm, about 2.75 mm, or about 3.00 mm, or wherein the indentation depth is about 0.25 mm to about 2.00 mm, about 0.50 mm to about 1.75 mm, about 0.75 mm to about 1.25 mm, or about 1.00 mm.

28. The method of any one of claims 1 and 20-27, wherein the population of plant seeds is a population of cotton seeds and the purifying comprises:
contacting the aspirated fraction, the first fraction, the third fraction, or the cylinder fraction with a textured surface of a vibratory platform, wherein the first textured surface of the vibratory platform is substantially planar, and wherein the aspirated fraction, the first fraction, the third fraction, or the cylinder fraction comprises a population of dry plant embryo explants and debris material;
vibrating the vibratory platform to produce a first platform motion; and
separating a platform fraction of the plant embryo explants from a platform portion of the debris material according to a displacement of the platform fraction relative to a displacement of the platform portion of debris material on the textured surface of the vibratory platform,
wherein the vibratory platform comprises a first tilt angle of about 10.0 degrees to about 22.0 degrees, about 10.0 degrees to about 20.0 degrees, about 10.0 degrees to about 19.0 degrees, about 15.0 degrees to about 22.0 degrees, about 11.0 degrees to about 19.0 degrees, about 11.0 degrees to about 15.0 degrees, about 11.6 degrees to about 14.2 degrees, about 16.0 degrees to about 19.0 degrees, about 16.2 degrees to about 18.3 degrees, about 12.9 degrees, about 17.2 degrees, or about 17.3 degrees, and a first pitch angle of about 1.5 degrees to about 6.0 degrees, about 1.5 degrees to about 5.0 degrees, about 1.8 degrees to about 4.9 degrees, about 1.8 degrees to about 3.3 degrees, about 2.4 degrees to about 4.9 degrees, about 2.5 degrees, about 2.6 degrees, about 3.6 degrees, or about 3.7 degrees.

29. The method of claim 1, wherein the population of plant seeds is a population of wheat seeds, and wherein said milling comprises:
positioning a first grinding roller and a second grinding roller to define a first gap having a first gap distance between the first roller and the second roller;
rotating the first roller about a first axis of rotation and the second roller about a second axis of rotation;
passing the population of seeds through said first gap to produce a first preparation of plant embryo explants comprising meristematic tissue;
positioning a third grinding roller and a fourth grinding roller to define a second gap having a second gap distance between the third roller and the fourth roller;
rotating the third roller about a third axis of rotation and the fourth roller about a fourth axis of rotation; and
passing the first preparation of embryo explants through said second gap to produce a second preparation of plant embryo explants comprising meristematic tissue,
wherein the first gap distance is about 0.2032 mm to about 2.54 mm, about 0.762 mm to about 1.788 mm, or is about 1.2827 mm,
or wherein the second gap distance is about 0.2032 mm to about 2.54 mm, about 0.2286 mm to about 0.4572 mm, or is about 0.3683 mm.

30. The method of claim 1 or 29, wherein the population of plant seeds is a population of wheat seeds and said aspirating comprises:
aspirating within a first vertical chamber, a second vertical chamber, a third vertical chamber, and a fourth vertical chamber with a first upward air flow, a second upward air flow, a third upward air flow, and a fourth upward air flow,
wherein the first upward airflow has a first air flow velocity of about 2.5 m/s to about 4.0 m/s, about 3.0 m/s to about 3.5 m/s, or about 3.0 m/s to about 3.3 m/s,
wherein the second upward air flow has a second air flow velocity of about 3.0 m/s to about 5.0 m/s, about 3.5 m/s to about 4.5 m/s, or about 3.8 m/s to about 4.3 m/s,
wherein the third upward air flow has a third air flow velocity of about 4.5 m/s to about 6.0 m/s, about 5.0 m/s to about 6.0 m/s, or about 5.1 m/s to about 5.5 m/s,
and wherein the fourth upward air flow has a fourth air flow velocity of about 6.5 m/s to about 8.0 m/s, about 7.0 m/s to about 8.0 m/s, or about 7.2 m/s to about 7.7 m/s.

31. The method of any one of claims 1, 29 and 30, wherein the population of plant seeds is a population of wheat seeds and the method further comprises:
contacting the preparation of dry plant embryo explants comprising meristematic tissue with a first moving sieve, wherein said first moving sieve comprises a plurality of openings, each having a first physical opening size;
separating a first fraction of embryo explants from a first portion of the debris material by length, width, or thickness relative to the first physical opening size, or relative to a first effective opening size;
contacting the first fraction with a second moving sieve, wherein the second moving sieve comprises a plurality of openings, each having a second physical opening size; and
separating a second fraction of embryo explants from a second portion of the debris material by length, width, or thickness relative to the second physical opening size, or relative to a second effective opening size,
wherein the first moving sieve and the second moving sieve move in a circular, elliptical, or linear motion.

32. The method of claim 31, wherein:
the first physical opening size is about 300 μm to about 1200 μm, about 600 μm to about 1200 μm, or about 864 μm; or
the second physical opening size is about 300 μm to about 900 μm or about 610 μm.

33. The method of any one of claims 1 and 29-32, wherein the method further comprises:
contacting the aspirated fraction with a first vibratory screen, wherein said first vibratory screen comprises a plurality of openings, each having a first opening size and a first opening shape, and wherein the aspirated fraction comprises a population of dry plant embryo explants and debris material;
vibrating the first vibratory screen to produce a first screen motion, wherein the first screen motion comprises a first horizontal vibratory component; and
separating a first screen fraction of embryo explants from a first screen portion of the debris material by length, width, or thickness relative to the first opening size or the first opening shape, or by a displacement of the first screen fraction relative to a displacement of the first screen portion of the debris material produced by the first screen motion.

34. The method of claim 33, wherein
the first opening shape is oblong, and wherein the first opening size is about 5 mm to about 15 mm, about 6 mm to about 14 mm, about 8 mm to about 12 mm, about 8 mm to about 10 mm, about 9 mm to about 11 mm, about 10 mm to about 12 mm in length, or about 8 mm, about 9 mm, about 10 mm, about 11 mm, or about 12 mm in length, and from about 0.6 mm to about 0.8 mm, about 0.6 mm to about 0.7 mm, or about 0.7 mm to about 0.8 mm in width, or about 0.6 mm, about 0.65 mm, about 0.7 mm, about 0.75 mm, or about 0.8 mm in width.

35. The method of any one of claims 1 and 29-34, wherein the population of plant seeds is a population of wheat seeds and the purifying comprises:
contacting the aspirated fraction, the second fraction, or the first screen fraction with a textured surface of a first vibratory platform, wherein the first textured surface of the first vibratory platform is substantially planar, and wherein the aspirated fraction, the second fraction, or the first screen fraction comprises a population of dry plant embryo explants and debris material;
vibrating the first vibratory platform to produce a first platform motion; and
separating a first platform fraction of the plant embryo explants from a first platform portion of the debris material according to a displacement of the platform fraction relative to a displacement of the platform portion of debris material on the textured surface of the first vibratory platform,
wherein the first vibratory platform comprises a first tilt angle of about 10.0 degrees to about 20.0 degrees, about 10.0 degrees to about 19.0 degrees, about 12.0 degrees to about 17.0 degrees, about 13.0 degrees to about 16.0 degrees, about 14.0 degrees to about 15.0 degrees, or about 14.5 degrees, and a first pitch angle of about 1.5 degrees to about 8.0 degrees, about 2.0 degrees to about 6.0 degrees, about 3.0 degrees to about 5.0 degrees, about 3.5 degrees to about 4.5 degrees, or about 4.0 degrees.

36. The method of claim 35, the method further comprising:
contacting the first platform fraction with a second textured surface of a second vibratory platform, wherein the second textured surface of the second vibratory platform is substantially planar;
vibrating the second vibratory platform to produce a second platform motion; and
separating a second platform fraction of the plant embryo explants of the first platform fraction from a second platform portion of the debris material according to a displacement of the second platform fraction relative to a displacement of the second platform portion of debris material on the second textured surface of the second vibratory platform,
wherein the second vibratory platform comprises a second tilt angle of about 10.0 degrees to about 16.0 degrees, about 10.0 degrees to about 15.0 degrees, about 11.0 degrees to about 15.0 degrees, about 12.0 degrees to about 14.0 degrees, about 12.5 degrees to about 13.5 degrees, about 12.7 degrees to about 13.1 degrees, or about 12.9 degrees, and a second pitch angle of about 1.5 degrees to about 5.0 degrees, about 1.0 degrees to about 4.0 degrees, about 1.0 degrees to about 3.0 degrees, about 1.5 degrees to about 3.0 degrees, about 1.8 degrees to about 2.6 degrees, or about 2.2 degrees.

37. The method of any one of claims 1 and 29-36, wherein the population of plant seeds is a population of wheat seeds, and the method further comprises aspirating the population of plant seeds prior to said sanitizing, wherein said aspirating comprises:
(a) aspirating within a first functional unit of a vertical chamber the population of plant seeds with a first air flow having a first air flow velocity, wherein the population of plant seeds comprises dry plant embryo explants comprising meristematic tissue and debris material;
(b) separating a first aspirated fraction of the plant embryo explants from a first aspirated portion of the debris material within the first functional unit of the vertical chamber according to a displacement of the first aspirated fraction relative to a displacement of the first aspirated portion of the debris material produced by the first air flow within the first functional unit, wherein the first air flow comprises a variable vertical component and a variable horizontal component,
wherein the first functional unit of the vertical chamber comprises a first lower partition, a first air input port, and a first air output port, wherein the first lower partition extends inward from a side wall of the vertical chamber to define a first lower advancement port between the first lower partition and an opposite side wall of the vertical chamber,
wherein the first air input port comprises an opening in the side wall of the vertical chamber below the first lower partition, and
wherein the first air flow at least partially enters the vertical chamber through the first air input port, travels through the first lower advancement port, and exits the vertical chamber through the first air output port;
(c) transferring the first aspirated fraction of the plant embryo explants through the first lower advancement port into a second functional unit, wherein the first lower advancement port is between the first functional unit and the second functional unit, and wherein the first functional unit is positioned above the second functional unit,
wherein the first aspirated portion of the debris material has been removed from said first aspirated fraction;
(d) aspirating within the second functional unit of the vertical chamber the first aspirated fraction of plant embryo explants with a second air flow having a second air flow velocity;
(e) separating a second aspirated fraction of the plant embryo explants comprised in the first aspirated fraction from a second aspirated portion of the debris material within the second functional unit of the vertical chamber according to a displacement of the second aspirated fraction relative to a displacement of the second aspirated portion of the debris material produced by the second air flow within the second functional unit, wherein the second air flow comprises a variable vertical component and a variable horizontal component,
wherein the second functional unit of the vertical chamber comprises a second lower partition, a second air input port, and a second air output port, wherein the second lower partition extends inward from the side wall of the vertical chamber to define a second lower advancement port between the second lower partition and the opposite side wall of the vertical chamber,
wherein the second air input port comprises an opening in the side wall of the vertical chamber below the second lower partition, and
wherein the second air flow at least partially enters the vertical chamber through the second air input port, travels through the second lower advancement port, and exits the vertical chamber through the second air output port;
(f) transferring the second aspirated fraction of the plant embryo explants through the second lower advancement port into a third functional unit, wherein the second lower advancement port is between the second functional unit and the third functional unit, and wherein the second functional unit is positioned above the third functional unit,
wherein the second aspirated portion of the debris material has been removed from said second aspirated fraction;
(g) aspirating within the third functional unit of the vertical chamber the second aspirated fraction of plant embryo explants with a third air flow having a third air flow velocity;
(h) separating a third aspirated fraction of the plant embryo explants comprised in the second aspirated fraction from a third aspirated portion of the debris material within the third functional unit of the vertical chamber according to a displacement of the third aspirated fraction relative to a displacement of the third aspirated portion of the debris material produced by the third air flow within the third functional unit, wherein the third air flow comprises a variable vertical component and a variable horizontal component,
wherein the third functional unit of the vertical chamber comprises a third lower partition, a third air input port, and a third air output port, wherein the third lower partition extends inward from the side wall of the vertical chamber to define a third lower advancement port between the third lower partition and the opposite side wall of the vertical chamber,
wherein the third air input port comprises an opening in the side wall of the vertical chamber below the third lower partition, and
wherein the third air flow at least partially enters the vertical chamber through the third air input port, travels through the third lower advancement port, and exits the vertical chamber through the third air output port;
(i) transferring the third aspirated fraction of the plant embryo explants through the third lower advancement port into a fourth functional unit, wherein the third lower advancement port is between the third functional unit and the fourth functional unit, and wherein the third functional unit is positioned above the fourth functional unit,
wherein the third aspirated portion of the debris material has been removed from said third aspirated fraction;
(j) aspirating within the fourth functional unit of the vertical chamber the third aspirated fraction of plant embryo explants with a fourth air flow having a fourth air flow velocity;
(k) separating a fourth aspirated fraction of the plant embryo explants comprised in the third aspirated fraction from a fourth aspirated portion of the debris material within the fourth functional unit of the vertical chamber according to a displacement of the fourth aspirated fraction relative to a displacement of the fourth aspirated portion of the debris material produced by the fourth air flow within the fourth functional unit, wherein the fourth air flow comprises a variable vertical component and a variable horizontal component,
wherein the fourth functional unit of the vertical chamber comprises a fourth lower partition, a fourth air input port, and a fourth air output port, wherein the fourth lower partition extends inward from the side wall of the vertical chamber to define a fourth lower advancement port between the fourth lower partition and the opposite side wall of the vertical chamber,
wherein the fourth air input port comprises an opening in the side wall of the vertical chamber below the fourth lower partition, and
wherein the fourth air flow at least partially enters the vertical chamber through the fourth air input port, travels through the fourth lower advancement port, and exits the vertical chamber through the fourth air output port;
(l) transferring the fourth aspirated fraction of the plant embryo explants through the fourth lower advancement port into a fifth functional unit, wherein the fourth lower advancement port is between the fourth functional unit and the fifth functional unit, and wherein the fourth functional unit is positioned above the fifth functional unit,
wherein the fourth aspirated portion of the debris material has been removed from said fourth aspirated fraction;
(m) aspirating within the fifth functional unit of the vertical chamber the fourth aspirated fraction of plant embryo explants with a fifth air flow having a fifth air flow velocity;
(n) separating a fifth aspirated fraction of the plant embryo explants comprised in the fourth aspirated fraction from a fifth aspirated portion of the debris material within the fifth functional unit of the vertical chamber according to a displacement of the fifth aspirated fraction relative to a displacement of the fifth aspirated portion of the debris material produced by the fifth air flow within the fifth functional unit, wherein the fifth air flow comprises a variable vertical component and a variable horizontal component,
wherein the fifth functional unit of the vertical chamber comprises a fifth lower partition, a fifth air input port, and a fifth air output port, wherein the fifth lower partition extends inward from the side wall of the vertical chamber to define a fifth lower advancement port between the fifth lower partition and the opposite side wall of the vertical chamber,
wherein the fifth air input port comprises an opening in the side wall of the vertical chamber below the fifth lower partition, and
wherein the fifth air flow at least partially enters the vertical chamber through the fifth air input port, travels through the fifth lower advancement port, and exits the vertical chamber through the fifth air output port;
(o) transferring the fifth aspirated fraction of the plant embryo explants through the fifth lower advancement port into a sixth functional unit, wherein the fifth lower advancement port is between the fifth functional unit and the sixth functional unit, and wherein the fifth functional unit is positioned above the sixth functional unit,
wherein the fifth aspirated portion of the debris material has been removed from said fifth aspirated fraction;
(p) aspirating within the sixth functional unit of the vertical chamber the fifth aspirated fraction of plant embryo explants with a sixth air flow having a sixth air flow velocity;
(q) separating a sixth aspirated fraction of the plant embryo explants comprised in the fifth aspirated fraction from a sixth aspirated portion of the debris material within the sixth functional unit of the vertical chamber according to a displacement of the sixth aspirated fraction relative to a displacement of the sixth aspirated portion of the debris material produced by the sixth air flow within the sixth functional unit, wherein the sixth air flow comprises a variable vertical component and a variable horizontal component,
wherein the sixth functional unit of the vertical chamber comprises a sixth lower partition, a sixth air input port, and a sixth air output port, wherein the sixth lower partition extends inward from the side wall of the vertical chamber to define a lower collection port between the sixth lower partition and the opposite side wall of the vertical chamber,
wherein the sixth air input port comprises an opening in the side wall of the vertical chamber below the sixth lower partition, and
wherein the sixth air flow at least partially enters the vertical chamber through the sixth air input port, travels through the lower collection port, and exits the vertical chamber through the sixth air output port; and
(r) collecting the sixth aspirated fraction of the plant embryo explants from the sixth functional unit, wherein the sixth aspirated portion of the debris material has been removed from said sixth aspirated fraction.

38. The method of claim 1, wherein the population of plant seeds is a population of canola seeds, and wherein said milling comprises:
positioning a first grinding roller and a second grinding roller to define a first gap having a first gap distance between the first roller and the second roller;
rotating the first roller about a first axis of rotation and the second roller about a second axis of rotation; and
passing the population of seeds through said first gap to produce a first preparation of plant embryo explants comprising meristematic tissue;
wherein the first gap distance is about 0.508 mm to about 1.016 mm, about 0.508 mm to about 0.762 mm, or is about 0.8509 mm.

39. The method of claim 1 or 38, wherein the population of plant seeds is a population of canola seeds and the method further comprises:
contacting the preparation of dry plant embryo explants comprising meristematic tissue with a first moving sieve, wherein said first moving sieve comprises a plurality of openings, each having a first physical opening size;
separating a first fraction of embryo explants from a first portion of the debris material by length, width, or thickness relative to the first physical opening size, or relative to a first effective opening size;
contacting the first fraction with a second moving sieve, wherein the second moving sieve comprises a plurality of openings, each having a second physical opening size;
separating a second fraction of embryo explants from a second portion of the debris material by length, width, or thickness relative to the second physical opening size, or relative to a second effective opening size;
contacting the second fraction with a third moving sieve, wherein the third moving sieve comprises a plurality of openings, each having a second physical opening size; and
separating a third fraction of embryo explants from a third portion of the debris material by length, width, or thickness relative to the second physical opening size, or relative to a second effective opening size,
wherein the first moving sieve, the second moving sieve, and the third moving sieve move in a circular, elliptical, or linear motion.

40. The method of claim 39, wherein:
the first physical opening size is about 300 μm to about 1100 μm, about 600 μm to about 1100 μm, about 300 μm to about 1000 μm, about 500 μm to about 1000 μm, or about 864 μm;
the second physical opening size is about 600 μm to about 1000 μm or about 812 μm; or
the third physical opening size is about 300 μm to about 900 μm or about 503 μm.

41. The method of claim 39 or 40, further comprising: separating the first preparation into a first top preparation fraction, a first middle preparation fraction, and a first bottom preparation fraction, wherein the first top preparation fraction is retained on the first moving sieve, the first middle preparation fraction is retained on the second moving sieve, and the first bottom preparation fraction is retained on the third moving sieve.

42. The method of claim 41, further comprising:
positioning the first grinding roller and the second grinding roller to define a first gap having a first gap distance between the first roller and the second roller;
rotating the first roller about a first axis of rotation and the second roller about a second axis of rotation; and
passing the first top preparation fraction through said first gap to produce a second preparation of plant embryo explants comprising meristematic tissue,
wherein the first gap distance is about 0.508 mm to about 1.016 mm, about 0.508 mm to about 0.762 mm, or is about 0.6985 mm.

43. The method of claim 42, further comprising: separating the second preparation into a second top preparation fraction, a second middle preparation fraction, and a second bottom preparation fraction, wherein the second top preparation fraction is retained on the first moving sieve, the second middle preparation fraction is retained on the second moving sieve, and the second bottom preparation fraction is retained on the third moving sieve.

44. The method of claim 43, further comprising:
combining the first middle preparation fraction with the second middle preparation fraction to produce a combined middle preparation fraction; or
combining the first bottom preparation fraction with the second bottom preparation fraction to produce a combined bottom preparation fraction.

45. The method of claim 44, wherein said purifying comprises aspirating the combined middle preparation fraction or the combined bottom preparation fraction.

46. The method of claim 45, said purifying further comprises:
contacting the aspirated combined middle preparation fraction or the aspirated combined bottom preparation fraction with a sieve, wherein said sieve comprises a plurality of openings, each having a physical opening size, and wherein the aspirated combined middle preparation fraction or the aspirated combined bottom preparation fraction comprises a population of dry plant embryo explants and debris material;
vibrating the sieve; and
separating a sieved fraction of embryo explants from a sieved portion of the debris material by length, width, or thickness relative to the physical opening size,
wherein the physical opening size is about 300 μm to about 900 μm, about 400 μm to about 800 μm, about 400 μm to about 700 μm, about 400 μm to about 600 μm, about 450 μm to about 550 μm or about 500 μm.

47. The method of any one of claims 1 and 38-46, wherein the population of plant seeds is a population of canola seeds and said aspirating comprises:
aspirating within a first vertical chamber, a second vertical chamber, a third vertical chamber, and a fourth vertical chamber with a first upward air flow, a second upward air flow, a third upward air flow, and a fourth upward air flow,
wherein the first upward airflow has a first air flow velocity of about 2.5 to about 4.0 m/s, about 3.0 m/s to about 4.0 m/s, about 3.4 m/s to about 3.8 m/s, or about 3.4 m/s to about 3.6 m/s,
wherein the second upward air flow has a second air flow velocity of about 4.0 m/s to about 5.5 m/s, about 4.0 m/s to about 5.0 m/s, about 4.3 m/s to about 4.9 m/s, or about 4.6 m/s to about 4.8 m/s,
wherein the third upward air flow has a third air flow velocity of about 5.0 m/s to about 7.0 m/s, about 5.5 m/s to about 6.5 m/s, or about 5.9 m/s to about 6.0 m/s,
and wherein the fourth upward air flow has a fourth air flow velocity of about 8.0 m/s to about 9.5 m/s, about 8.5 m/s to about 9.0 m/s, or about 8.8 m/s to about 8.9 m/s.

48. An apparatus for producing or purifying plant embryo explants from plant seeds, the apparatus comprising at least one component selected from the group consisting of: a seed roller mill, a seed grinder, a siever, a rotating cylinder, an aspirator, a vibratory screen, and a vibratory platform.

49. The apparatus of claim 48, wherein said apparatus comprising at least two components, at least three components, at least four components, at least five components, or at least six components selected from the group consisting of: a seed roller mill, a seed grinder, a siever, a rotating cylinder, an aspirator, a vibratory screen, and a vibratory platform.
